(19) United States
(12) Patent Application Publication
(10) Pub. No.: US 2024/0182503 A1
(43) Pub. Date: Jun. 6, 2024

(54) MACROCYCLE COMPLEMENT FACTOR B INHIBITORS

(71) Applicant: Alexion Pharmaceuticals, Inc., Boston, MA (US)

(72) Inventors: Jason Allan Wiles, Madison, CT (US); Venkat Rao Gadhachanda, Hamden, CT (US)

(73) Assignee: Alexion Pharmaceuticals, Inc., Boston, MA (US)

(21) Appl. No.: 18/271,736

(22) PCT Filed: Jan. 13, 2022
(86) PCT No.: PCT/US2022/012260

§ 371 Date: Jan. 26, 2024

Related U.S. Application Data
(60) Provisional application No. 63/137,710, filed on Jan. 14, 2021.

(51) Int. Cl. C07F 9/576; A61K 31/437; A61K 31/675; C07D 401/06; C07D 491/044; C07D 491/056; C07D 491/147; C07D 498/04; C07D 498/14

(52) U.S. Cl. CPC C07F 9/5765; A61K 31/437; A61K 31/675; C07D 401/06; C07D 491/044; C07D 491/056; C07D 491/147; C07D 498/04; C07D 498/14

(57) ABSTRACT

This disclosure relates to compounds, pharmaceutical compositions comprising them, and methods of using the compounds and compositions for treating diseases or disorders related to misregulation of the Complement cascade pathway. More particularly, this disclosure relates to macrocyclic compounds and pharmaceutical compositions thereof, methods of inhibiting Complement Factor B (CFB) expression with these compounds, and methods of treating diseases or disorders mediated by CFB.

### CROSS-REFERENCE TO RELATED APPLICATIONS

This application claims the benefit of priority of U.S. Provisional Patent Application No. 63/137,710, filed Jan. 14, 2021, which is incorporated herein by reference in its entirety.

### BACKGROUND OF DISCLOSURE

The Complement cascade is part of the host innate immune system involved in lysing foreign cells, enhancing phagocytosis of antigens, clumping antigen-bearing agents, and attracting macrophages and neutrophils. The dysfunction or excessive activation of Complement has been linked to some autoimmune, inflammatory, and neurodegenerative diseases, as well as ischemia-reperfusion injury and cancer. The Complement system is divided into three pathways—the classical, lectin, and alternative pathway—that converge at component C3 to generate an enzyme complex known as C3 convertase. For example, CFB plays an early and central role in activation of the alternative pathway. In turn, activation of the alternative pathway of the Complement cascade contributes to the production of C3a and C5a, both potent anaphylatoxins that also have roles in a number of inflammatory disorders. Therefore, decreasing the response of the Complement cascade pathway, and particularly the alternative pathway, would be beneficial in treatment of numerous diseases, such as age-related macular degeneration (AMD), paroxysmal nocturnal hemoglobinuria (PNH), multiple sclerosis (MS), rheumatoid arthritis, and others.

Currently, only one product associated with Complement cascade pathway, the anti-C5 monoclonal antibody eculizumab, has been approved in the U.S. Treatment with eculizumab; however, requires life-long intravenous injections and blood transfusions due to severe anemia that develops in some patients. Therefore, there is a need for novel and effective inhibitors of the Complement cascade pathway.

### SUMMARY OF THE DISCLOSURE

This disclosure relates to compounds, pharmaceutical compositions comprising them, and methods of using the compounds and compositions for treating diseases or disorders related to misregulation of the Complement cascade pathway. More particularly, this disclosure relates to novel Complement Factor B (CFB) inhibitor compounds and pharmaceutical compositions thereof, methods of inhibiting CFB with these compounds, and methods of treating diseases or disorders mediated by CFB.

Thus, one aspect of the disclosure provides a compound of formula (I):

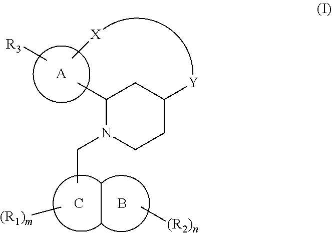

or a pharmaceutically acceptable salt thereof, wherein
ring A represents a phenyl or a naphthyl ring;
ring B and ring C form a bicyclic heteroaryl, bicyclic heterocyclyl, or bicyclic cycloalkyl moiety, where ring B is a monocyclic heteroaryl, monocyclic heterocyclyl, or monocyclic cycloalkyl ring, and ring C is a phenyl or monocyclic 6-membered heteroaryl ring;
m is an integer 0, 1, 2, or 3;
n is an integer 0, 1, or 2;
R₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₂ is independently selected from halogen, —CN, C₁-C₆ alkyl, C₁-C₆ haloalkyl, C₁-C₆ alkoxy, and C₁-C₆ haloalkoxy;
R₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₅, —SO₂OH, —SO₂(C₁-C₆ alkyl), —SO₂N(R₅)₂, —SO₂NH—COCH₃, —S(O)(NR₅)R₅, —NH—SO₂R₅, —NHCO—NHSO₂R₅, —PO(OH)₂, —PO(OH)R₅, pyrazolyl, and tetrazolyl, wherein
   each R₅ is independently selected from the group consisting of hydrogen, C₁-C₆ alkyl, C₁-C₆ haloalkyl, cycloalkyl optionally substituted with C₁-C₄ alkyl, phenyl, and monocyclic heteroaryl; and
X is CH₂, CH, O, S, or NH, and Y is O, S, or NH, where X and Y, together with the atoms to which they are attached, form a 10- to 16-member macrocycle, the macrocycle optionally substituted with one or more R₄, wherein
   each R₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
   or two R₄ groups, together with the carbon to which they are attached, form a ═O.

Another aspect of the disclosure provides compounds of formula (II) and (III):

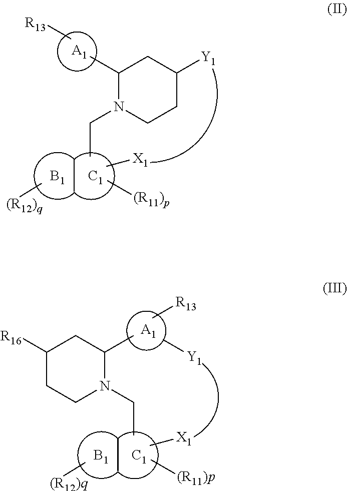

or a pharmaceutically acceptable salt thereof, wherein
ring A₁ represents a phenyl or a naphthyl ring;
ring B₁ and ring C₁ form a bicyclic heteroaryl, bicyclic heterocyclyl, or bicyclic cycloalkyl moiety, where ring B₁ is a monocyclic heteroaryl, monocyclic heterocyclyl, or monocyclic cycloalkyl ring, and ring C₁ is a phenyl or monocyclic 6-membered heteroaryl ring;
p is an integer 0, 1, or 2;
q is an integer 0, 1, or 2;
R₁₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₁₂ is independently selected from halogen, —CN, C₁-C₆ alkyl, C₁-C₆ haloalkyl, C₁-C₆ alkoxy, and C₁-C₆ haloalkoxy;
R₁₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₁₅, —SO₂OH, —SO₂(C₁-C₆ alkyl), —SO₂N(R₁₅)₂, —SO₂NH—COCH₃, —S(O)(NR₁₅)R₁₅, —NH—SO₂R₁₅, —NHCO—NHSO₂R₁₅, —PO(OH)₂, —PO(OH)R₁₅, pyrazolyl, and tetrazolyl, wherein
   each R₁₅ is independently selected from the group consisting of hydrogen, C₁-C₆ alkyl, C₁-C₆ haloalkyl, cycloalkyl optionally substituted with C₁-C₄ alkyl, phenyl, and monocyclic heteroaryl;
R₁₆ is —OH, C₁-C₆ alkoxy, —NH₂, —NH(C₁-C₆ alkyl), or —N(C₁-C₆ alkyl)₂; and
X is CH₂, CH, O, S, or NH, and Y is O, S, or NH, where X and Y, together with the atoms to which they are attached, form a 10- to 16-member macrocycle, the macrocycle optionally substituted with one or more R₁₄, wherein
   each R₁₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
   or two R₁₄ groups, together with the carbon to which they are attached, form a ═O.

Another aspect of the disclosure provides compounds of formula (VI), (V), and (VI):

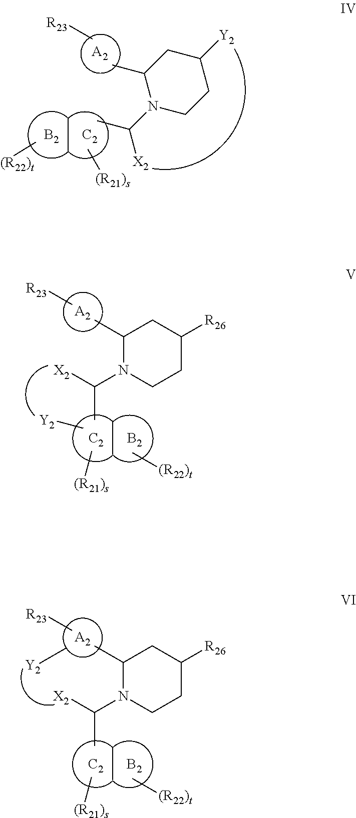

or a pharmaceutically acceptable salt thereof, wherein
ring A₂ represents a phenyl or a naphthyl ring;
ring B₂ and ring C₂ form a bicyclic heteroaryl, bicyclic heterocyclyl, or bicyclic cycloalkyl moiety, where ring B₂ is a monocyclic heteroaryl, monocyclic heterocyclyl, or monocyclic cycloalkyl ring, and ring C₂ is a phenyl or monocyclic 6-membered heteroaryl ring;
s is an integer 0, 1, or 2;
t is an integer 0, 1, or 2;
R₂₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₂₂ is independently selected from halogen, —CN, C₁-C₆ alkyl, C₁-C₆ haloalkyl, C₁-C₆ alkoxy, and C₁-C₆ haloalkoxy;
R₂₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₂₅, —SO₂OH, —SO₂(C₁-C₆ alkyl), —SO₂N(R₂₅)₂, —SO₂NH—COCH₃, —S(O)(NR₂₅)R₂₅, —NH—SO₂R₂₅, —NHCO—NHSO₂R₂₅, —PO(OH)₂, —PO(OH)R₂₅, pyrazolyl, and tetrazolyl, wherein
   each R₂₅ is independently selected from the group consisting of hydrogen, C₁-C₆ alkyl, C₁-C₆ haloalkyl;
R₂₆ is —OH, C₁-C₆ alkoxy, —NH₂, —NH(C₁-C₆ alkyl), or —N(C₁-C₆ alkyl)₂; and
X is CH₂, CH, O, S, or NH, and Y is O, S, or NH, where X and Y, together with the atoms to which they are attached, form a 10- to 16-member macrocycle, the macrocycle optionally substituted with one or more R₂₄, wherein
   each R₂₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
   or two R₂₄ groups, together with the carbon to which they are attached, form a ═O.

Another aspect of the disclosure provides a pharmaceutical composition including one or more compounds of the disclosure as described herein (e.g., compounds of any one of formulae (I)-(VI)) and a pharmaceutically acceptable carrier, solvent, adjuvant or diluent.

Another aspect of the disclosure provides a method of treating a disease or disorder mediated by Complement Factor B. Such methods include administering to a subject in need of such treatment one or more compounds of the disclosure as described herein or a pharmaceutical composition of the disclosure as described herein.

In certain embodiments of this aspect, the disease or disorder is age-related macular degeneration (AMD), geographic atrophy (GA), retinal degeneration, ophthalmic disease, multiple sclerosis, arthritis, chronic obstructive pulmonary disease (COPD), an ophthalmic disease, rheumatoid arthritis, paroxysymal nocturnal hemoglobinuria (PNH), a respiratory disease, a cardiovascular disease, atypical or typical hemolytic uremic syndrome (HUS), C3 glomerulopathy (3G), IgA nephropathy (IgAN), and other nephropathies with evidence of glomerular C3 deposition such as membranous nephropathy (MN) or E. coli induced hemolytic uremic syndrome (HUS).

Another aspect of the disclosure provides methods of inhibiting Complement Factor B, the method including administering one or more compounds of the disclosure as described herein or a pharmaceutical composition of the disclosure as described herein.

Other features and advantages of the present disclosure will become apparent from the following detailed description.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed materials and methods provide improvements in treatment of diseases or disorders associated with Complement cascade pathway, and with Complement Factor B in particular.

Accordingly, one aspect of the disclosure provides compounds of formula (I) as provided above.

In certain embodiments, the disclosure provides compounds of formula (I) wherein ring A represents a phenyl ring. For example, in certain embodiments, the compounds of formula (I) are of formula (1-1):

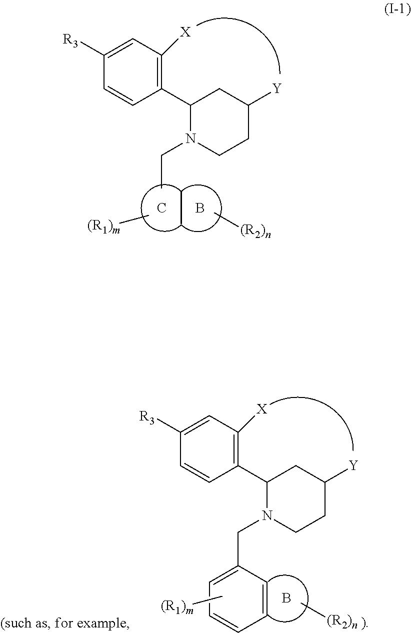

In certain embodiments, the disclosure provides compounds of formula (I) wherein ring A represents a naphthyl ring. For example, in certain embodiments, the compounds of formula (I) are of formula (1-2):

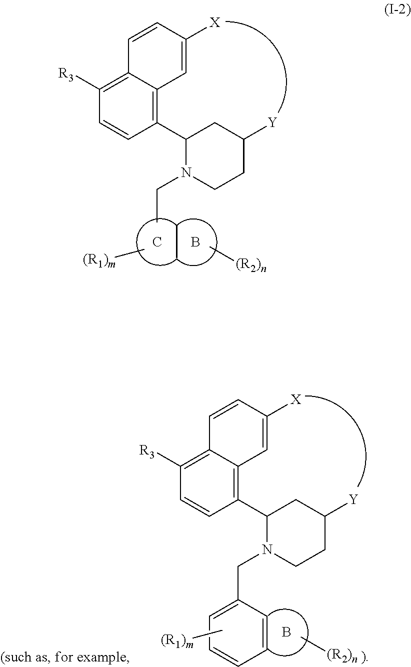

Another embodiment of the disclosure provides compounds of formula (I)-(I-2) as otherwise described herein wherein ring C is phenyl. Yet another embodiment of the disclosure provides compounds of formula (I)-(I-2) as otherwise described herein wherein ring C is a monocyclic 6-membered heteroaryl ring. In certain embodiments, the heteroaryl ring is a pyridinyl ring.

Another embodiment of the disclosure provides compounds of formula (I)-(I-2) as otherwise described herein wherein ring B is a monocyclic heteroaryl (e.g., a 5-membered heteroaryl). Examples of 5-membered heteroaryl B-rings include, but are not limited to, pyrrole, pyrazole, imidazole, isoxazole, or oxazole. In certain embodiments, ring B is pyrrole, for example, of formula (I-3), or formula (I-4) or (I-5):

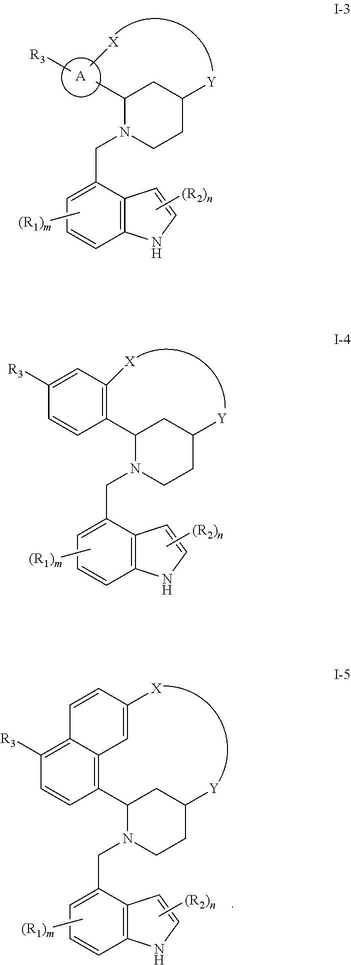

In certain embodiments, the disclosure provides compounds of formula (I)-(I-5) as otherwise described herein wherein m is 1 or 2. In certain embodiments, m is 2.

In certain embodiments, the disclosure provides compounds of formula (I)-(I-5) as otherwise described herein wherein R₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₁ is independently selected from halogen, C₁-C₆ alkyl, C₁-C₆ haloalkyl, —OH, and C₁-C₆ alkoxy.

In certain embodiments, R₁ is independently selected from bromo, methyl, ethyl, propyl, methoxy, and ethoxy.

In certain embodiments, the disclosure provides compounds of formula (I)-(I-5) as otherwise described herein wherein m is 2 and two R₁ are independently selected from methyl and methoxy.

In certain embodiments, the disclosure provides compounds of formula (I)-(I-5) as otherwise described herein wherein R₂ is independently selected from halogen, C₁-C₆ alkyl, C₁-C₆ haloalkyl, —OH, and C₁-C₆ alkoxy. Preferably, R₂ is at 3-position (e.g., β-position to the heteroatom on the B ring).

In certain embodiments, the disclosure provides compounds of formula (I)-(I-5) as otherwise described herein wherein n is 0 or 1. In certain embodiments, n is 0. For example, in certain embodiments, the compounds are of formula:

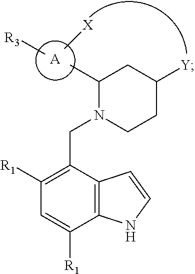

for example, of formulae:

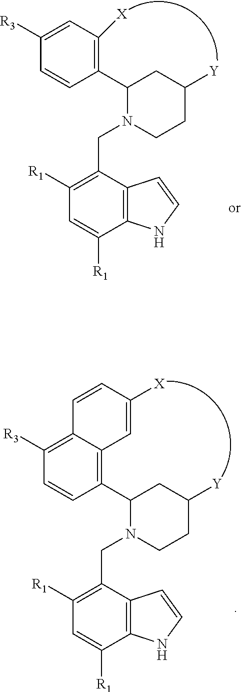

In certain embodiments, the compounds are of formula:

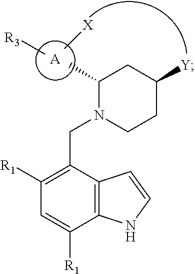

for example, of formulae:

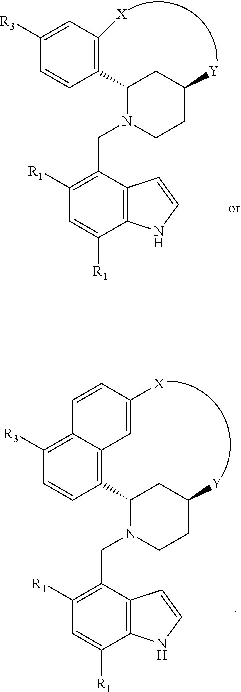

In certain embodiments, the compounds are of formula:

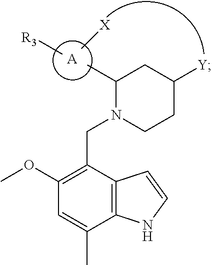

for example, of formulae

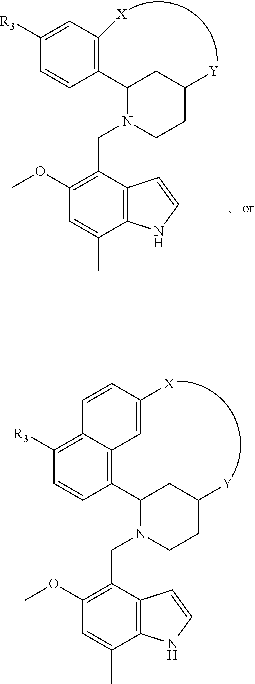

In certain example embodiments, the compounds are of formula

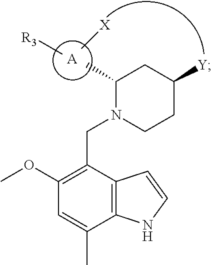

for example, of formulae:

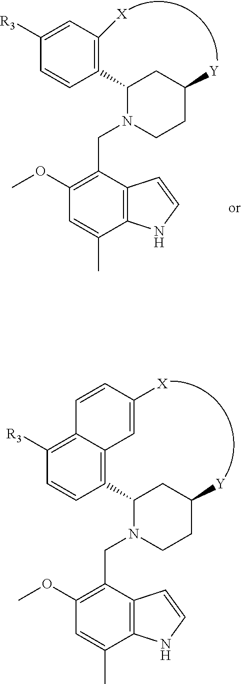

Another embodiment of the disclosure provides compounds with respect to any above-described formula wherein R₃ is selected from —CO₂H, —CO₂(C₁-C₆ alkyl), —CONH₂, —CONH(C₁-C₆ alkyl), —CON(C₁-C₆ alkyl)₂, —CONH—S(O)₂R₅, —SO₂OH, —SO₂(C₁-C₆ alkyl), —SO₂N(R₅)₂, —S(O)(NR₅)R₅, —NH—SO₂R₅, —NHCO—NHSO₂R₅, —PO(OH)₂, and —PO(OH)R₅. In certain embodiments, R₃ is selected from —CO₂H, —CO₂(C₁-C₆ alkyl), —CONH—S(O)₂R₅, —SO₂(C₁-C₆ alkyl), —SO₂N(R₅)₂, —S(O)(NR₅)R₅, —NHCO—NHSO₂R₅, —PO(OH)₂, and —PO(OH)R₅.

In certain embodiments, R₃ is —CO₂H. In certain embodiments, R₃ is —CONH—S(O)₂R₅, —SO₂H, —SO₂(C₁-C₆ alkyl), —SO₂N(R₅)₂, or —S(O)(NR₅)R₅. In certain embodiments, R₃ is —PO(OH)₂ or —PO(OH)R₅. In certain embodiments, R₃ is —CONH—S(O)₂R₅, wherein R₅ is hydrogen, C₁-C₃ alkyl, C₃-C₆ cycloalkyl optionally substituted with methyl, and phenyl.

As provided above, X and Y, together with the atoms to which they are attached, form a 10- to 16-member macrocycle. In certain embodiments, the macrocycle is a 12- to 14-member macrocycle. The macrocycle may include heteroatoms, such as N, O, and S, at any available position on the macrocycle (i.e., instead of a carbon atom). The macrocycle may be optionally fused with a triazole and/or optionally substituted with one or more R₄ at any available atom, provided that the chemical valance is satisfied. Thus, in one embodiment, X and Y, together with the atoms to which they are attached, form a 12- to 14-member macrocycle optionally fused with a triazole, optionally substituted with one or more R₄.

Thus, in certain embodiments, if present, each R₄ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₄ groups, together with the carbon to which they are attached, form ═O. In certain embodiments, if present, each R₄ is independently selected from the group consisting of halogen, C₁-C₆ alkyl, C₁-C₆ haloalkyl, —OH, C₁-C₆ alkoxy, and C₁-C₆ haloalkoxy, or two R₄ groups, together with the carbon to which they are attached, form ═O.

In certain embodiments, the macrocycle is substituted with at least two R₄ groups. For example, in certain embodiments, two R₄ groups, together with the carbon to which they are attached, form ═O. In other embodiments, two R₄ groups are each halogen (e.g., fluoro).

In certain embodiments, the [symbol] portion of the macrocycle has the following structure:

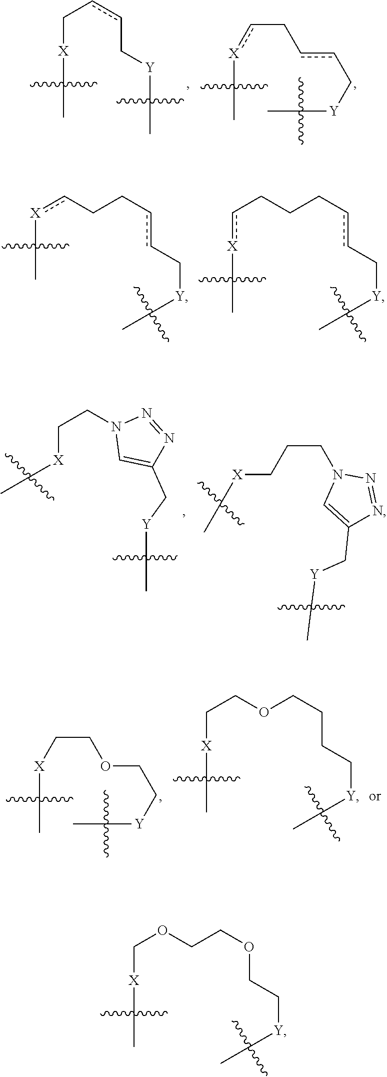

each optionally substituted with one or more R₄ (e.g., each optionally substituted with one or two halogens, such as fluoro).

In certain embodiments, X is O, S, or NH, and Y is O. In other embodiments, X and Y are both O. For example, the [symbol] portion is:

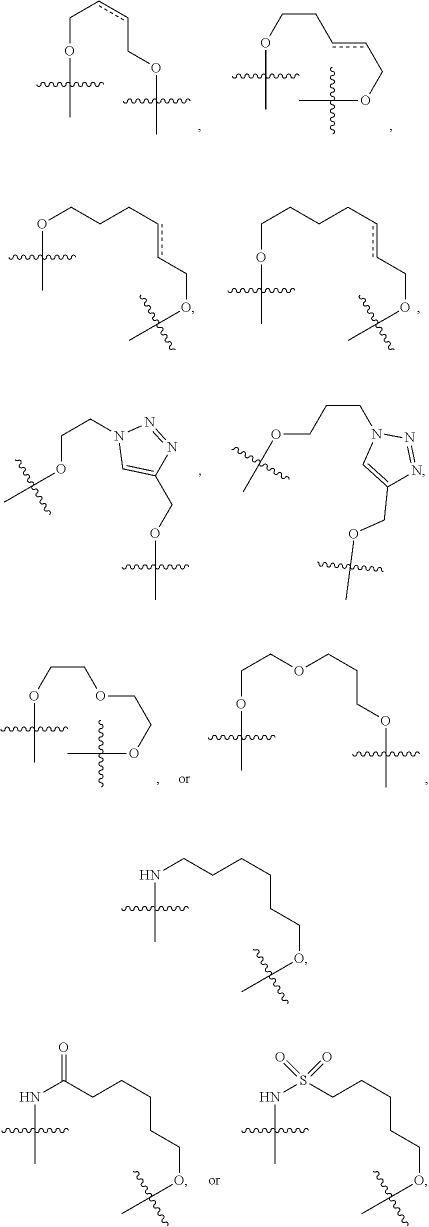

each optionally substituted with one or two halogens (e.g., fluoro).

In certain embodiments, X is OH or OH₂, and Y is O. For example, the [symbol] portion is:

each optionally substituted with one or two halogens (e.g., fluoro).

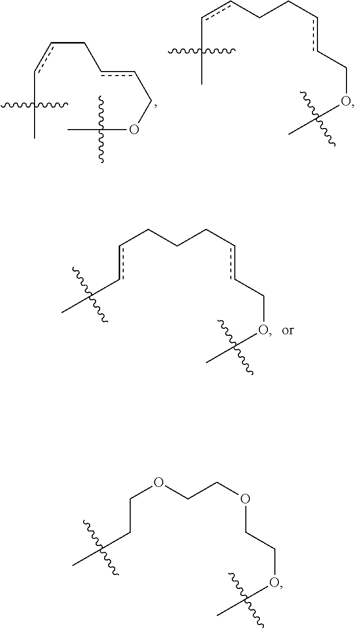

In certain embodiments, X is NH and Y is O. For example, the [symbol] portion is:

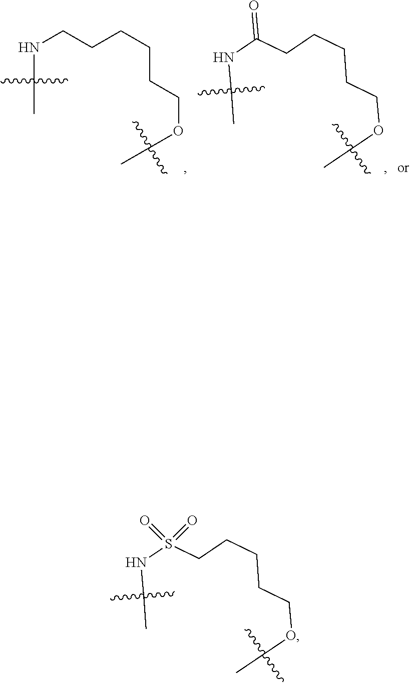 [SMILES: *NCCCCCCO* ; *NC(=O)CCCCCO* ; *NS(=O)(=O)CCCCCO*]

each optionally substituted with one or two halogens (e.g., fluoro).

In certain embodiments, X and Y, together with the atoms to which they are attached, form an optionally substituted 13-member macrocycle. X may be NH or O and Y may be O. In some embodiments, X and Y are both O. Such macrocycle may be substituted with at least two R₄ groups. For example, in certain embodiments, two R₄ groups, together with the carbon to which they are attached, form ═O. In other embodiments, two R₄ groups are each halogen (e.g., fluoro).

Another aspect of the disclosure provides compounds of formula (II) and formula (III) as provided above.

In certain embodiments, the disclosure provides compounds of formula (II) or (III) wherein ring A₁ represents a phenyl ring. For example, in certain embodiments, such compounds are of formula (II-1) or (III-1):

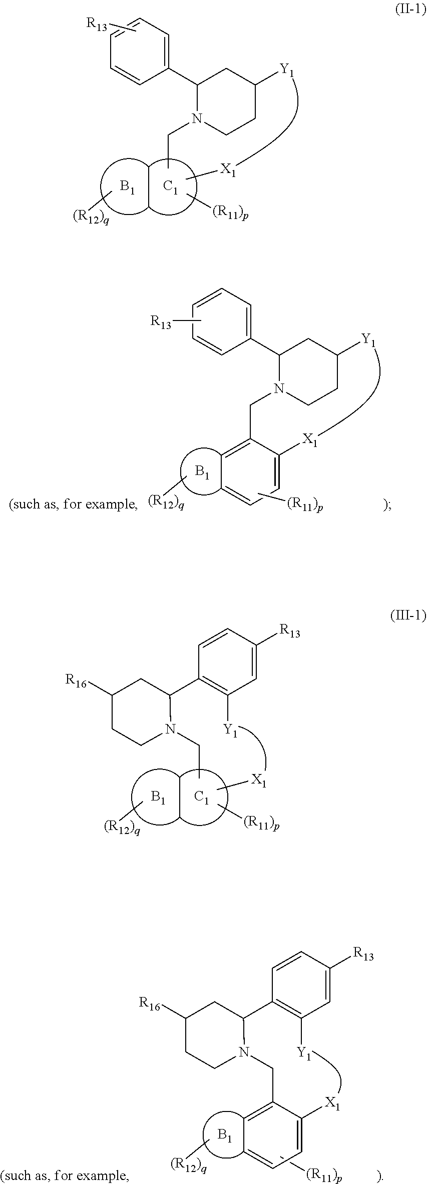

Another embodiment of the disclosure provides compounds of formula (II)-(II-1) or (III)-(III-1) as otherwise described herein wherein ring C₁ is phenyl. Yet another embodiment of the disclosure provides compounds of formula (II)-(II-1) or (III)-(III-1) as otherwise described herein wherein ring C₁ is a monocyclic 6-membered heteroaryl ring. In certain embodiments, the heteroaryl ring is a pyridinyl ring.

Another embodiment of the disclosure provides compounds of formula (II)-(II-1) or (III)-(III-1) as otherwise described herein wherein ring B₁ is a monocyclic heteroaryl (e.g., a 5-membered heteroaryl). Examples of 5-membered heteroaryl B₁-rings include, but are not limited to, pyrrole, pyrazole, imidazole, isoxazole, or oxazole. In certain embodiments, ring B₁ is pyrrole, for example, of formula (II-2) (e.g., formula (II-3)) or (III-2) (e.g., formula III-3):

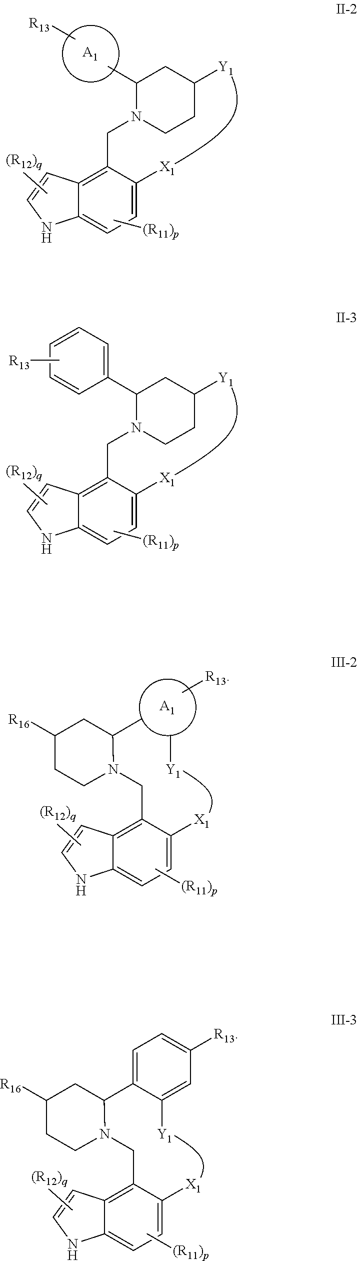

In certain embodiments, the disclosure provides compounds of formula (II)-(II-3) or (III)-(III-3) as otherwise described herein wherein p is 0 or 1. In certain embodiments, p is 1.

In certain embodiments, the disclosure provides compounds of formula (II)-(II-3) or (III)-(III-3) as otherwise described herein wherein R₁₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₁₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₁₁ is independently selected from halogen, C₁-C₆ alkyl, C₁-C₆ haloalkyl, —OH, and C₁-C₆ alkoxy. In certain embodiments, R₁₁ is independently selected from bromo, methyl, ethyl, propyl, methoxy, and ethoxy.

In certain embodiments, the disclosure provides compounds of formula (II)-(II-3) or (III)-(III-3) as otherwise described herein wherein p is 1 and R₁₁ is methyl.

In certain embodiments, the disclosure provides compounds of formula (II)-(II-3) or (III)-(III-3) as otherwise described herein wherein R₁₆ is —OH or C₁-C₆ alkoxy. In certain embodiments, R₁₆ is C₁-C₆ alkoxy. In certain embodiments, R₁₆ is methoxy or ethoxy.

In certain embodiments, the disclosure provides compounds of formula (II)-(II-3) or (III)-(III-3) as otherwise described herein wherein R₁₂ is independently selected from halogen, C₁-C₆ alkyl, C₁-C₆ haloalkyl, —OH, and C₁-C₆ alkoxy. Preferably, R₁₂ is at 3-position (e.g., β-position to the heteroatom on the B₁ ring).

In certain embodiments, the disclosure provides compounds of formula (II)-(II-3) or (III)-(III-3) as otherwise described herein wherein q is 0 or 1. In certain embodiments, q is 0.

For example, in certain embodiments, the compounds are of formula:

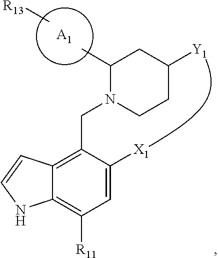

such as

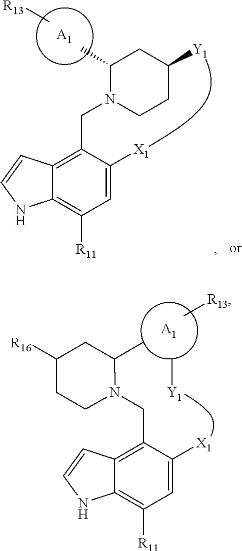

such as

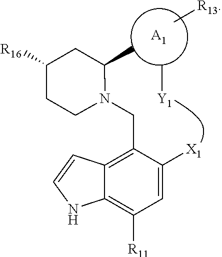

In certain embodiments, the compounds are of formula:

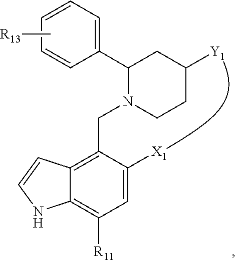

for example,

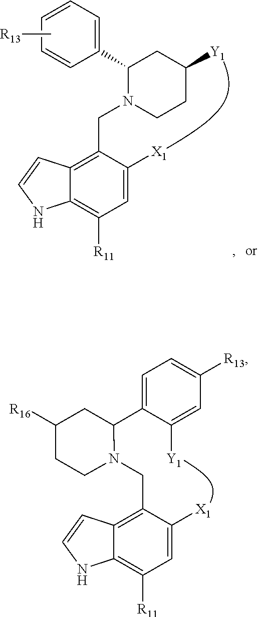

for example

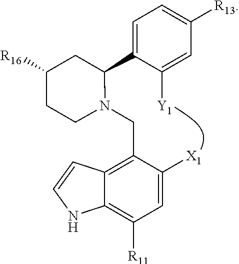

In certain example embodiments, R₁₁ may be methyl in these formulae.

Another embodiment of the disclosure provides compounds with respect to any above-described formula wherein R₁₃ is selected from —CO₂H, —CO₂(C₁-C₆ alkyl), —CONH₂, —CONH(C₁-C₆ alkyl), —CON(C₁-C₆ alkyl)₂, —CONH—S(O)₂R₅, —SO₂OH, —SO₂(C₁-C₆ alkyl), —SO₂N(R₁₅)₂, —S(O)(NR₁₅)R₁₅, —NH—SO₂R₁₅, —NHCO—NHSO₂R₁₅, —PO(OH)₂, and —PO(OH)R₁₅. In certain embodiments, R₁₃ is selected from —CO₂H, —CO₂(C₁-C₆ alkyl), —CONH—S(O)₂R₅, —SO₂(C₁-C₆ alkyl), —SO₂N(R₁₅)₂, —S(O)(NR₁₅)R₁₅, —NHCO—NHSO₂R₁₅, —PO(OH)₂, and —PO(OH)R₁₅. In certain embodiments, R₁₃ is —CO₂H. In certain embodiments, R₁₃ is —CONH—S(O)₂R₅, —SO₂H, —SO₂(C₁-C₆ alkyl), —SO₂N(R₁₅)₂, or —S(O)(NR₁₅)R₁₅. In certain embodiments, R₁₃ is —PO(OH)₂ or —PO(OH)R₁₅. In certain embodiments, R₁₃ is —CONH—S(O)₂R₅, wherein R₅ is hydrogen, C₁-C₃ alkyl, C₃-C₆ cycloalkyl optionally substituted with methyl, and phenyl.

As provided above, X₁ and Y₁, together with the atoms to which they are attached, form a 10- to 16-member macrocycle. In certain embodiments, the macrocycle is a 12- to 14-member macrocycle. The macrocycle may include heteroatoms, such as N, O, and S, at any available position on the macrocycle (i.e., instead of a carbon atom). The macrocycle may be optionally fused with a triazole and/or optionally substituted with one or more R₁₄ at any available atom, provided that the chemical valance is satisfied. Thus, in one embodiment, X₁ and Y₁, together with the atoms to which they are attached, form a 12- to 14-member macrocycle optionally fused with a triazole, optionally substituted with one or more R₁₄.

Thus, in certain embodiments, if present, each R₁₄ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₁₄ groups, together with the carbon to which they are attached, form ═O. In certain embodiments, if present, each R₁₄ is independently selected from the group consisting of halogen, C₁-C₆ alkyl, C₁-C₆ haloalkyl, —OH, C₁-C₆ alkoxy, and C₁-C₆ haloalkoxy, or two R₁₄ groups, together with the carbon to which they are attached, form ═O.

In certain embodiments, the macrocycle is substituted with at least two R₁₄ groups. For example, in certain embodiments, two R₁₄ groups, together with the carbon to which they are attached, form ═O. In other embodiments, two R₁₄ groups are each halogen (e.g., fluoro).

In certain embodiments, the [symbol] portion of the macrocycle has the following structure,

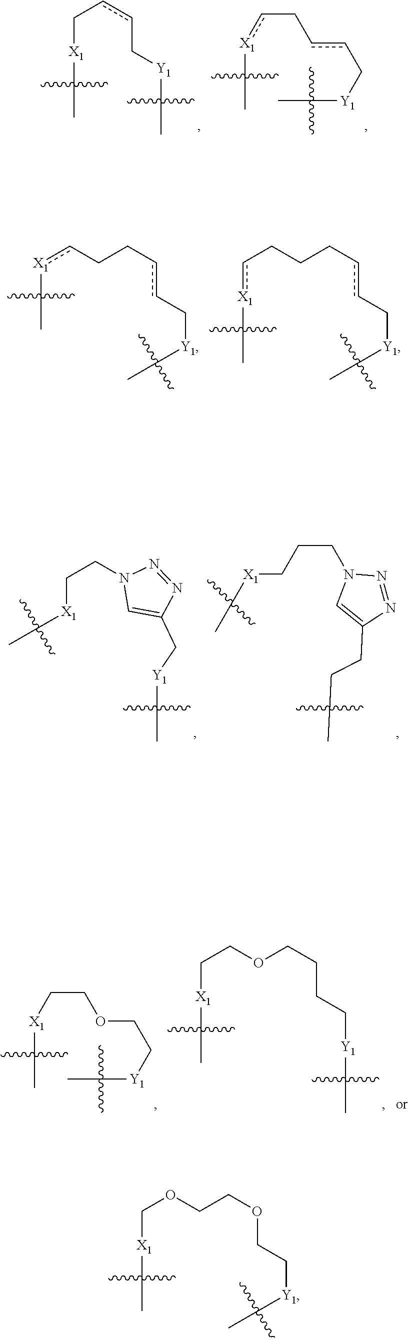

each optionally substituted with one or more R₁₄ (e.g., each optionally substituted with one or two halogens, such as fluoro).

In certain embodiments, X₁ is O, S, or NH, and Y₁ is O. In other embodiments, X₁ and Y₁ are both O. For example, the [symbol] portion is:

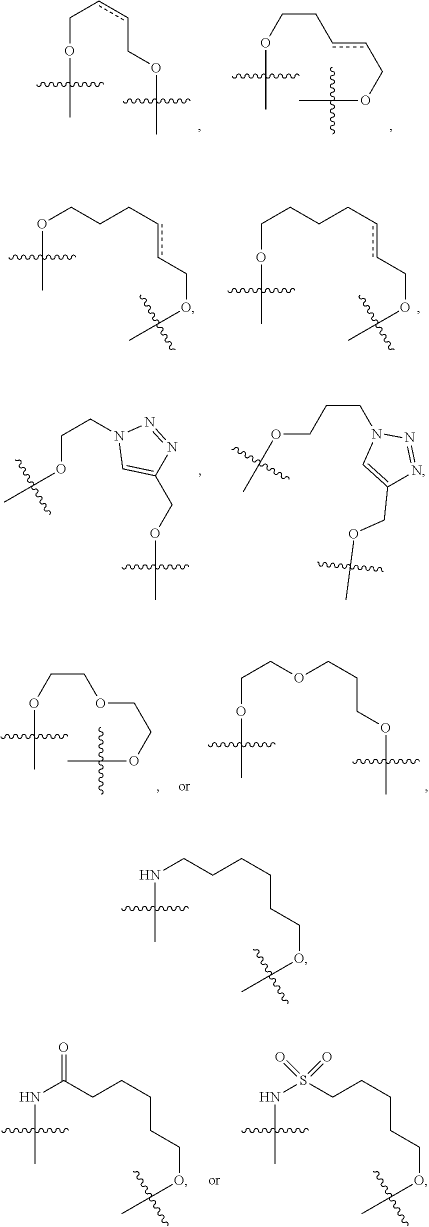

each optionally substituted with one or two halogens (e.g., fluoro).

In certain embodiments, X₁ is CH or CH₂, and Y₁ is O. For example, the [symbol] portion is:

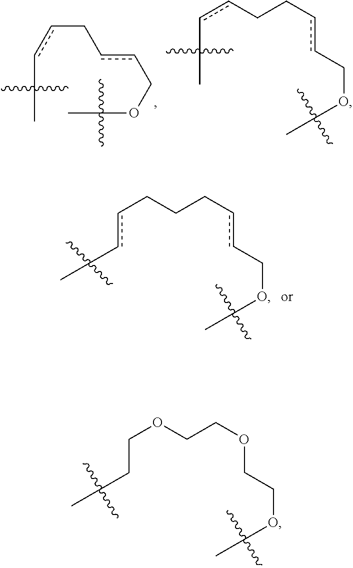

each optionally substituted with one or two halogens (e.g., fluoro).

In certain embodiments, X₁ is NH, and Y₁ is O. For example, the [symbol] portion is:

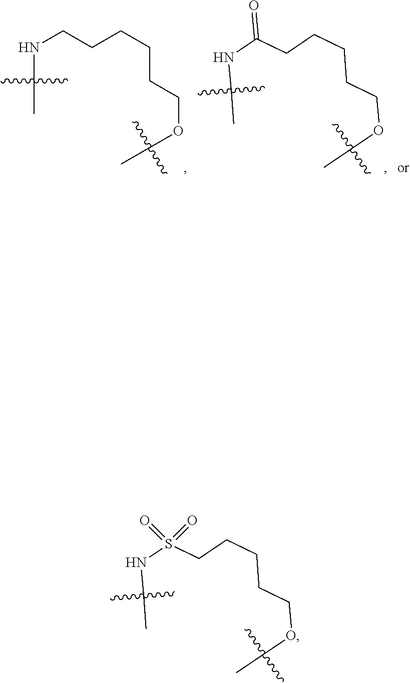 [SMILES: *NCCCCCCO* ; *NC(=O)CCCCCO* ; *NS(=O)(=O)CCCCCO*]

each optionally substituted with one or two halogens (e.g., fluoro).

In certain embodiments, X₁ and Y₁, together with the atoms to which they are attached, form an optionally substituted 13-member macrocycle. X₁ may be NH or O and Y₁ may be O. In some embodiments, X₁ and Y₁ are both O. Such macrocycle may be substituted with at least two R₁₄ groups. For example, in certain embodiments, two R₁₄ groups, together with the carbon to which they are attached, form ═O. In other embodiments, two R₁₄ groups are each halogen (e.g., fluoro).

Another aspect of the disclosure provides compounds of formula (IV), formula (V), and formula (VI) as provided above.

In certain embodiments, the disclosure provides compounds of formula (IV), (V) or (VI) wherein ring A₂ represents a phenyl ring. For example, in certain embodiments, such compounds are of formula (IV-1), (V-1), or (VI-1):

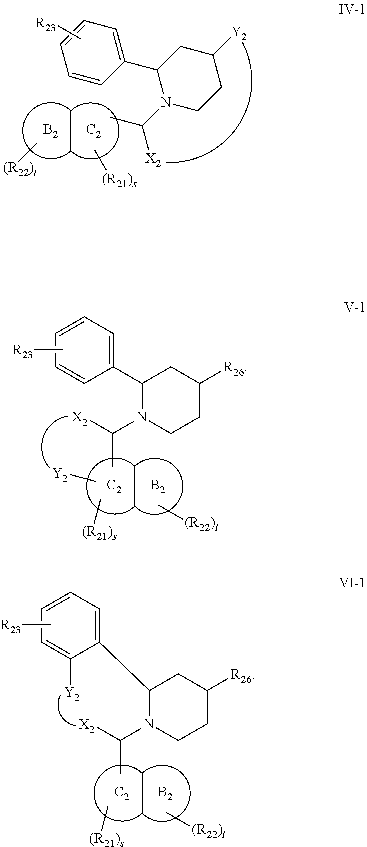

Another embodiment of the disclosure provides compounds of formula (IV)-(IV-1), (V)-(V-1) or (VI)-(VI-1) as otherwise described herein wherein ring C₂ is phenyl. Yet another embodiment of the disclosure provides compounds of formula (IV)-(IV-1), (V)-(V-1) or (VI)-(VI-1) as otherwise described herein wherein ring C₂ is a monocyclic 6-membered heteroaryl ring. In certain embodiments, the heteroaryl ring is a pyridinyl ring.

Another embodiment of the disclosure provides compounds of formula (IV)-(IV-1), (V)-(V-1) or (VI)-(VI-1) as otherwise described herein wherein ring B₂ is a monocyclic heteroaryl (e.g., a 5-membered heteroaryl). Examples of 5-membered heteroaryl B₂-rings include, but are not limited to, pyrrole, pyrazole, imidazole, isoxazole, or oxazole. In certain embodiments, ring B₂ is pyrrole, for example, of formula (VI-2) (e.g., formula (VI-3)), (V-2) (e.g., formula V-3), or (VI-2) (e.g., formula VI-3):

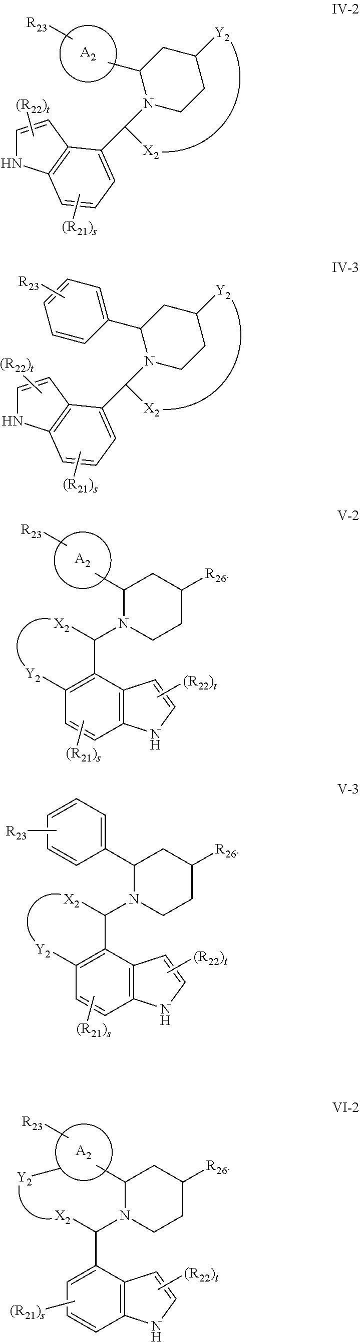

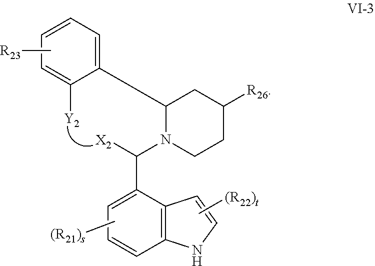

In certain embodiments, the disclosure provides compounds of formula (IV)-(IV-3), (V)-(V-3) or (VI)-(VI-3) as otherwise described herein wherein s is 0 or 1. In certain embodiments, s is 1.

In certain embodiments, the disclosure provides compounds of formula (IV)-(IV-3), (V)-(V-3) or (VI)-(VI-3) as otherwise described herein wherein R₂₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₂₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In certain embodiments, R₂₁ is independently selected from halogen, C₁-C₆ alkyl, C₁-C₆ haloalkyl, —OH, and C₁-C₆ alkoxy. In certain embodiments, R₂₁ is independently selected from bromo, methyl, ethyl, propyl, methoxy, and ethoxy.

In certain embodiments, the disclosure provides compounds of formula (IV)-(IV-3) or (VI)-(VI-3) as otherwise described herein wherein s is 1 and R₂₁ is methyl.

In certain embodiments, the disclosure provides compounds of formula (V)-(V-3) as otherwise described herein wherein s is 2 and each R₂₁ is independently methyl or methoxy.

In certain embodiments, the disclosure provides compounds of formula (V)-(V-3) or (VI)-(VI-3) as otherwise described herein wherein R₂₆ is —OH or C₁-C₆ alkoxy. In certain embodiments, R₂₆ is C₁-C₆ alkoxy. In certain embodiments, R₂₆ is methoxy or ethoxy.

In certain embodiments, the disclosure provides compounds of formula (IV)-(IV-3), (V)-(V-3) or (VI)-(VI-3) as otherwise described herein wherein R₂₂ is independently selected from halogen, C₁-C₆ alkyl, C₁-C₆ haloalkyl, —OH, and C₁-C₆ alkoxy. Preferably, R₂₂ is at 3-position (e.g., β-position to the heteroatom on the B₂ ring).

In certain embodiments, the disclosure provides compounds of formula (IV)-(IV-3), (V)-(V-3) or (VI)-(VI-3) as otherwise described herein wherein t is 0 or 1. In certain embodiments, t is 0. For example, in certain embodiments, the compounds are of formula:

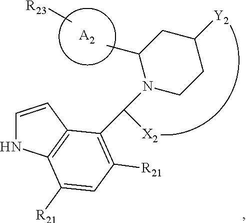

such as

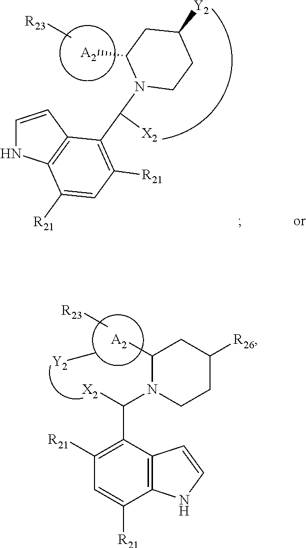

such as

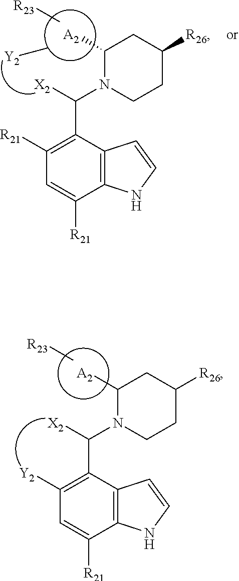

such as

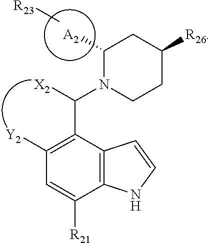

In certain embodiments, the compounds are of formula:

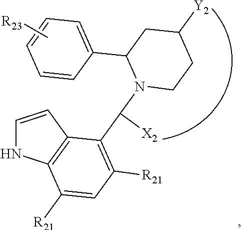

such as

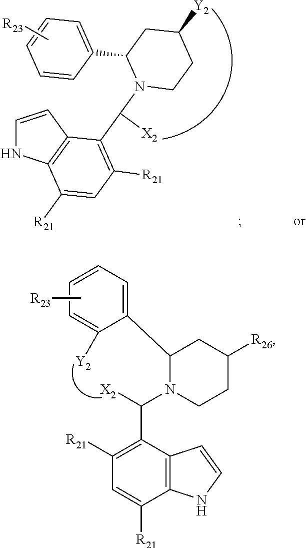

such as

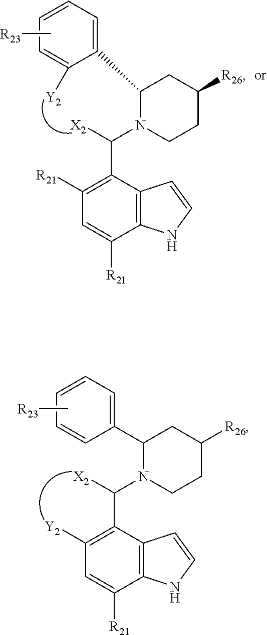

such as

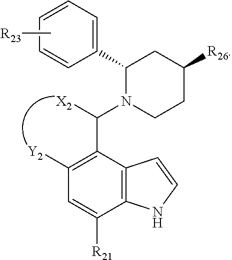

In certain example embodiments, R₂₁ may be methyl in these formulae. In certain example embodiments, one R₂₁ may be methyl and the other R₂₁ may be methoxy in these formulae.

Another embodiment of the disclosure provides compounds with respect to any above-described formula wherein R₂₃ is selected from —CO₂H, —CO₂(C₁-C₆ alkyl), —CONH₂, —CONH(C₁-C₆ alkyl), —CON(C₁-C₆ alkyl)₂, —SO₂OH, —SO₂(C₁-C₆ alkyl), —SO₂N(R₂₅)₂, —S(O)(NR₂₅)R₂₅, —NH—SO₂R₂₅, —NHCO—NHSO₂R₂₅, —PO(OH)₂, and —PO(OH)R₂₅. In certain embodiments, R₂₃ is selected from —CO₂H, —CO₂(C₁-C₆ alkyl), —SO₂(C₁-C₆ alkyl), —SO₂N(R₂₅)₂, —S(O)(NR₂₅)R₂₅, —NHCO—NHSO₂R₂₅, —PO(OH)₂, and —PO(OH)R₂₅. In certain embodiments, R₂₃ is —CO₂H. In certain embodiments, R₂₃ is —SO₂H, —SO₂(C₁-C₆ alkyl), —SO₂N(R₂₅)₂, or —S(O)(NR₁₅)R₂₅. In certain embodiments, R₂₃ is —PO(OH)₂ or —PO(OH)R₂₅.

As provided above, X₂ and Y₂, together with the atoms to which they are attached, form a 10- to 16-member macrocycle. In certain embodiments, the macrocycle is a 12- to 14-member macrocycle. The macrocycle may include heteroatoms, such as N, O, and S, at any available position on the macrocycle (i.e., instead of a carbon atom). The macrocycle may be optionally fused with a triazole and/or optionally substituted with one or more R₂₄ at any available atom, provided that the chemical valance is satisfied. Thus, in one embodiment, X₂ and Y₂, together with the atoms to which they are attached, form a 12- to 14-member macrocycle optionally fused with a triazole, optionally substituted with one or more R₂₄.

Thus, in certain embodiments, if present, each R₂₄ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₂₄ groups, together with the carbon to which they are attached, form ═O. In certain embodiments, if present, each R₂₄ is independently selected from the group consisting of halogen, C₁-C₆ alkyl, C₁-C₆ haloalkyl, —OH, C₁-C₆ alkoxy, and C₁-C₆ haloalkoxy, or two R₂₄ groups, together with the carbon to which they are attached, form ═O.

In certain embodiments, the [symbol] portion of the macrocycle has the following structure,

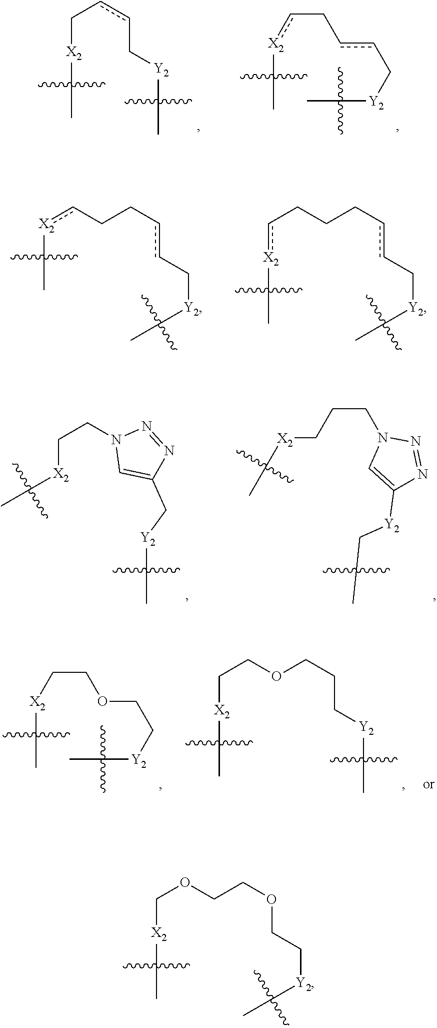

each optionally substituted with one or more R₂₄ (e.g., each optionally substituted with one or two halogens, such as fluoro).

In certain embodiments, X₂ is O, S, or NH, and Y₂ is O. In other embodiments, X₂ and Y₂ are both O. For example, the [symbol] portion is:

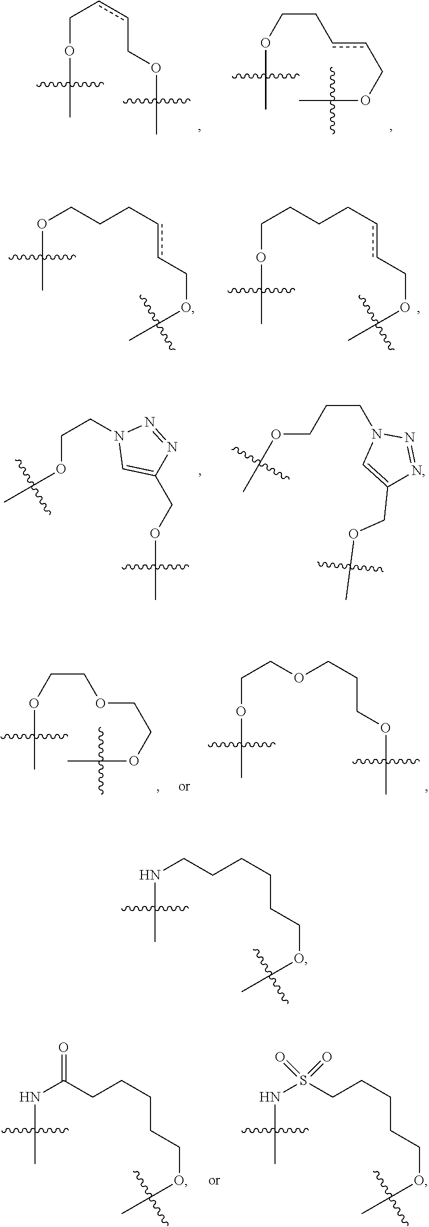

each optionally substituted with one or two halogens (e.g., fluoro).

In certain embodiments, X₂ is CH or CH₂, and Y₂ is O. For example, the [symbol] portion is:

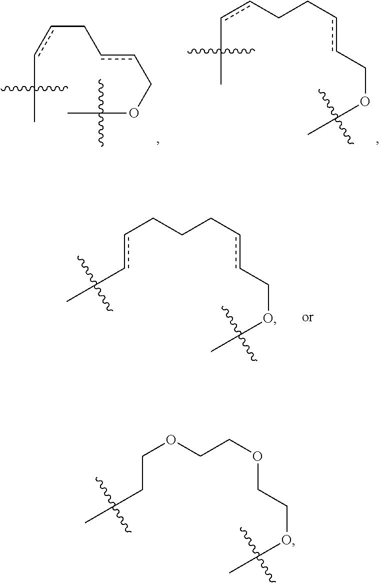

each optionally substituted with one or two halogens (e.g., fluoro).

In certain embodiments, X₂ is NH, and Y₂ is O. For example, the [symbol] portion is:

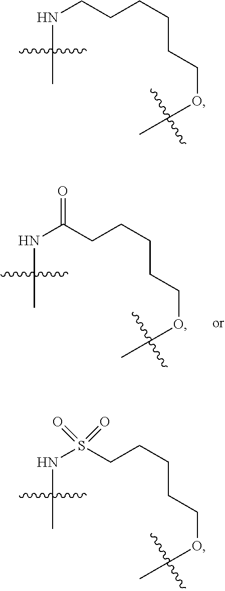 [SMILES: *NCCCCCCO* ; *NC(=O)CCCCCO* ; "or" ; *NS(=O)(=O)CCCCCO*]

each optionally substituted with one or two halogens (e.g., fluoro).

In certain embodiments, X₂ and Y₂, together with the atoms to which they are attached, form an optionally substituted 13-member macrocycle. X₂ may be NH or 0 and Y₂ may be O. In some embodiments, X₂ and Y₂ are both O. Such macrocycle may be substituted with at least two R₂₄ groups. For example, in certain embodiments, two R₂₄ groups, together with the carbon to which they are attached, form ═O. In other embodiments, two R₂₄ groups are each halogen (e.g., fluoro).

### Therapeutics Applications

There is a great need in the art to develop novel therapeutics for the treatment of disease or disorders associated with Complement cascade pathway. The present disclosure satisfies this and other needs by providing a novel CFB inhibitors.

In broad aspect, the disclosure provides methods of treating a disease or disorder mediated by the Complement cascade (including a dysfunctional cascade), a disorder or abnormality of a cell that adversely affects the ability of the cell to engage in or respond to normal Complement activity, or an inflammatory or immune condition in a subject.

Thus, in one aspect, the disclosure provides a method of inhibiting CFB. Another aspect of the disclosure provides a method of treating a disease or disorder mediated by Complement Factor B in a subject. Such methods include administering to a subject in need of such treatment an effective amount of one or more compounds of the disclosure as described herein (i.e., compounds of any one of formulas (I)-(VI)) or a pharmaceutical composition of the disclosure as described herein.

In certain embodiments, the disease or disorder is age-related macular degeneration (AMD) or geographic atrophy (GA). Some examples include, but are not limited to, wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration.

In certain embodiments, the disease or disorder is retinal degeneration, ophthalmic disease, multiple sclerosis, arthritis, or chronic obstructive pulmonary disease (COPD). In certain embodiments, the disease or disorder is an ophthalmic disease. In certain embodiments, the disease or disorder is rheumatoid arthritis. In certain embodiments, the disease or disorder is paroxysymal nocturnal hemoglobinuria (PNH). In certain embodiments, the disease or disorder is a respiratory disease. In certain embodiments, the disease or disorder is a cardiovascular disease. In certain embodiments, the disease or disorder is atypical or typical hemolytic uremic syndrome (HUS). In certain embodiments, the disease or disorder is C3 glomerulopathy (3G), IgA nephropathy (IgAN), and other nephropathies with evidence of glomerular C₃ deposition, such as membranous nephropathy (MN) or E. coli induced hemolytic uremic syndrome (HUS).

In certain embodiments, the disease or disorder is selected from fatty liver and conditions stemming from fatty liver, such as nonalcoholic steatohepatitis (NASH), liver inflammation, cirrhosis and liver failure. In certain embodiments, the disease or disorder is dermatomyositis. In certain embodiments, the disease or disorder is amyotrophic lateral sclerosis. In certain embodiments, the disease or disorder is abdominal aortic aneurysm, hemodialysis complications, hemolytic anemia, or hemodialysis.

In certain embodiments, the disease or disorder is episcleritis, idiopathic episcleritis, anterior episcleritis, or posterior episcleritis. In certain embodiments, the disease or disorder is idiopathic anterior uveitis, HLA-B27 related uveitis, herpetic keratouveitis, Posner Schlossman syndrome, Fuch's heterochromic iridocyclitis, or cytomegalovirus anterior uveitis. In certain embodiments, the disease or disorder is a C3 glomurenopathy, dense deposit disease (DDD) and C3 glomerulonephritis (C3GN).

Another aspect of the disclosure provides methods of inhibiting Complement Factor B, the method including administering one or more compounds of the disclosure as described herein or a pharmaceutical composition of the disclosure as described herein.

When administered as a combination, the compounds and compositions of the disclosure as described herein and the secondary therapeutic agents can be formulated as separate compositions that are given simultaneously or sequentially, or the therapeutic agents can be given as a single composition. In certain embodiments, the secondary therapeutic agent may be administered in an amount below its established half maximal inhibitory concentration (IC₅₀). For example, the secondary therapeutic agent may be administered in an amount less than 1% of, e.g., less than 10%, or less than 25%, or less than 50%, or less than 75%, or even less than 90% of the inhibitory concentration (IC₅₀).

### Pharmaceutical Compositions

In another aspect, the present disclosure provides compositions comprising one or more of compounds as described above with respect to formula (I)-(VI) and an appropriate carrier, solvent, adjuvant, or diluent. The exact nature of the carrier, solvent, adjuvant, or diluent will depend upon the desired use for the composition, and may range from being suitable or acceptable for veterinary uses to being suitable or acceptable for human use. The composition may optionally include one or more secondary therapeutic agents. In certain embodiments, the composition may include one or more secondary anticancer therapeutic agents.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect.

For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Definitions

The following terms and expressions used herein have the indicated meanings.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkylene” refers to a bivalent alkyl group. An “alkylene chain” is a polymethylene group, i.e., —(CH₂)ₙ-, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thioxo groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thioxo.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}-, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thioxo. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thioxo.

The term “halo” or “halogen” as used herein, means —C₁, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thioxo. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thioxo.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thioxo. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thioxo.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thioxo” as used herein means a ═S group.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Subject” refers to a warm blooded animal such as a mammal, preferably a human, or a human child, which is afflicted with, or has the potential to be afflicted with one or more diseases and disorders described herein.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Ed., Wiley-Interscience, 2001; or Vogel, A Textbook of Practical Organic Chemistry, Including Qualitative Organic Analysis, Fourth Ed., New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formulae (I)-(VI) can be prepared according to general procedures (below), and/or analogous synthetic procedures. One of skill in the art can adapt the reaction sequences of Examples 1-13 and general procedures to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### EXAMPLES

The preparation of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

### Comparative Example 1: 4-((2S,4S)-4-(2-acetamidoethoxy)-1-((5-methoxy-7-methyl-1H-indol-4-yl)methyl)piperidin-2-yl)benzoic acid

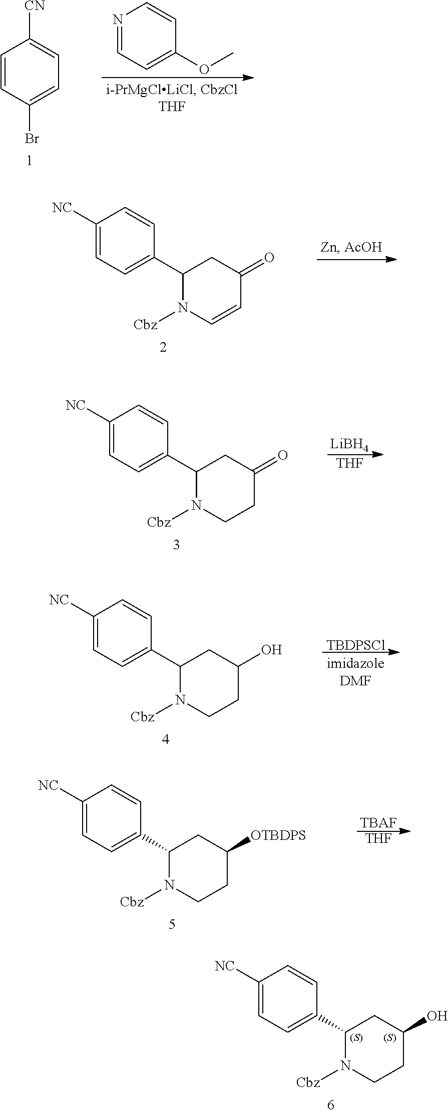

Step 1: benzyl 2-(4-cyanophenyl)-4-oxo-3,4-dihydropyridine-1(2H)-carboxylate (2): To a solution of compound 1 (18 g, 99.4 mmol) in THF (53 mL) was added iPrMgCl·LiCl (74 mL, 96.3 mmol) dropwise at room temperature under N₂ atmosphere. The mixture was stirred at room temperature for 2 hours. The mixture was diluted with THF (320 mL) and then cooled down to −5° C. 4-methoxypyridine (8.9 mL, 86.8 mmol) and Cbz-Cl (12.7 mL, 88.9 mmol) were added to the mixture while maintaining the internal temperature to no more than 0° C. The reaction was stirred at room temperature overnight. The mixture was quenched with 5M aq. HCl solution at 0° C. and stirred at room temperature for 30 minutes. The mixture was extracted with EtOAc twice and washed with water, 5% aq. NaHCO₃, and brine, dried over anhydrous Na₂SO₄ and concentrated to dryness under reduced pressure. The residue was purified by column chromatography on silica gel (PE:EtOAc=20:1 to 1:1) to give compound 2 (20.7 g, yield 60.6%) as yellow solid. LC/MS: m/z 333 (M+H)⁺.

Step 2: benzyl 2-(4-cyanophenyl)-4-oxopiperidine-1-carboxylate (3): To a solution of compound 2 (20.7 g, 62.3 mmol) in AcOH (80 mL) was added activated zinc (7.96 g, 121 mmol) at room temperature under N₂ atmosphere and the reaction was stirred at 100° C. overnight. The mixture was cooled down to room temperature and filtered through a plug of celite, washed with MTBE twice. The filtrate was washed with water, 5% aq. NaHCO₃, and brine respectively. The organic layer was dried over anhydrous Na₂SO₄, filtered and concentrated to dryness under reduced pressure. The residue was purified by column chromatography on silica gel (PE:EtOAc=10:1 to 1:1) to give compound 3 (16.6 g, yield 60.6%) as white solid. LC/MS: m/z 353 (M+H)⁺.

Step 3: benzyl 2-(4-cyanophenyl)-4-hydroxypiperidine-1-carboxylate(4): To a solution of compound 3 (6.6 g, 19.7 mmol) in THF (82 mL) was added LiBH₄ (16.5 mL, 33 mmol, 2 M in THF) dropwise at 0° C. under N₂ atmosphere. The reaction was stirred at room temperature for 2 hours and quenched with 50% aq. KHSO₄. The mixture was extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=10:1 to 1:1) to give compound 4 (6.2 g, yield 92.7%) as oil. LC/MS: m/z 337 (M+H)⁺.

Step 4: benzyl (2S,4S)-4-((tert-butyldiphenylsilyl)oxy)-2-(4-cyanophenyl) piperidine-1-carboxylate(5): To a solution of compound 4 (6.2 g, 18.4 mmol) in DMF (30 mL) was added imidazole (3.85 g, 56.6 mmol) and TBDPSCl (6.62 mL, 25.46 mmol) at 0° C. under N₂ atmosphere and the mixture was stirred at 40° C. for 6 hours. The mixture was diluted with ice-water and extracted with Ethyl acetate twice. The combined organic layers were washed with 5% aq. NaHCO₃ solution and brine, dried over anhydrous Na₂SO₄, filtered and concentrated to dryness under reduced pressure. The residue was purified by column chromatography on silica gel (PE:EtOAc=30:1 to 5:1) to give compound 5 (4.66 g, yield 43.9%) as oil. LC/MS(ESI) m/z: 575 (M+H)⁺.

Step 5: benzyl (2S,4S)-2-(4-cyanophenyl)-4-hydroxypiperidine-1-carboxylate (6): To a solution of compound 5 (4.66 g, 8.12 mmol) in THF was added TBAF (13.4 mL, 26.8 mmol, 2 M in THF) and the reaction was stirred at 60° C. for 2 hours. The reaction mixture was washed with 5% aq. NaHCO₃ solution and extracted with ethyl acetate. The organic layer was washed with brine, dried over anhydrous Na₂SO₄, filtered and concentrated to dryness. The residue was purified by column chromatography on silica gel (PE:EtOAc=10:1 to 2:1) and further purified by SFC to give compound 6 (1.0 g, yield 36.7%) as oil. LC/MS(ESI) m/z: 337 (M+H)⁺.

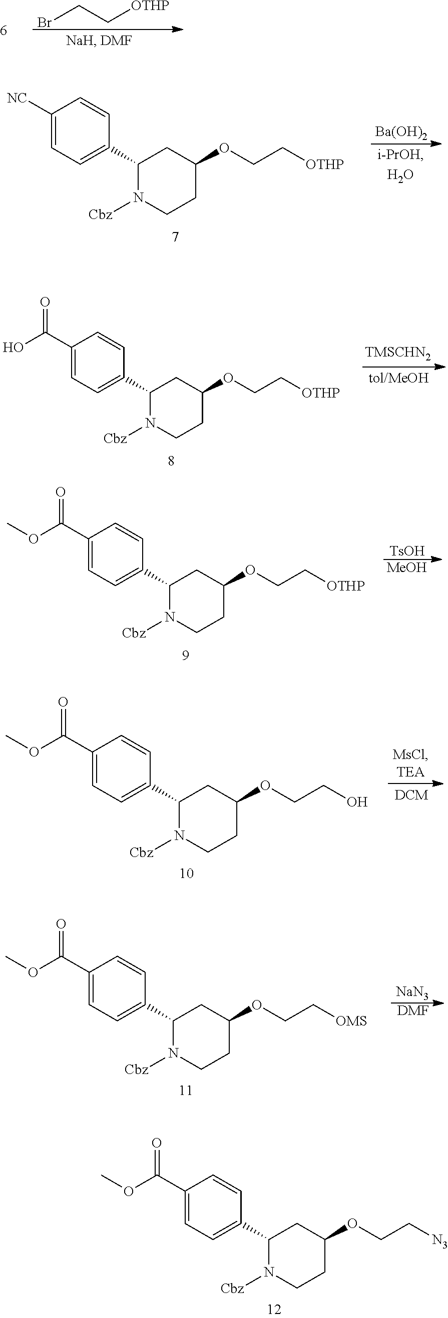

Step 6: benzyl (2S,4S)-2-(4-cyanophenyl)-4-(2-((tetrahydro-2H-pyran-2-yl)oxy) ethoxy)piperidine-1-carboxylate (7): To a solution of compound 6 (1.0 g, 2.98 mmol) in DMF (10 mL) was added NaH (357 mg, 8.93 mmol, 60% in mineral oil) at 0° C. under N₂ atmosphere and the mixture was stirred at room temperature for 30 minutes. 2-(2-bromoethoxy)tetrahydro-2H-pyran (2.49 g, 11.9 mmol) was added to the stirring reaction dropwise and the mixture was stirred overnight at 40° C. The mixture was quenched with ice-water and extracted with ethyl acetate twice. The combined organic layers were washed with brine, dried over anhydrous Na₂SO₄, filtered and concentrated to dryness under reduced pressure. The residue was purified by column chromatography on silica gel (PE EtOAc=30:1 to 1:1) to give compound 7 (980 mg, yield 70.9%) as oil. LC/MS(ESI) m/z: 381 (M−THP+1)⁺.

Step 7: 4-((2S,4S)-1-((benzyloxy)carbonyl)-4-(2-((tetrahydro-2H-pyran-2-yl)oxy) ethoxy)piperidin-2-yl)benzoic acid (8): To a solution of compound 7 (980 mg, 2.11 mmol) in i-PrOH (1.5 mL) and H₂O (5 mL) was added Ba(OH)₂ (1.8 g, 10.5 mmol) at room temperature and the reaction was stirred at 100° C. overnight. The mixture was cooled down to room temperature, filtered and washed with water. The water layer was acidified with 0.5 M aq. HCl solution and extracted with ethyl acetate twice. The organic layer was washed with brine, dried over anhydrous Na₂SO₄, filtered and concentrated to dryness under reduced pressure to give compound 8 (800 mg, yield 78.5%) as oil, which was used directly in the next step without further purification. LC/MS(ESI) m/z: 400 (M−THP+1)⁺.

Step 8: benzyl (2S,4S)-2-(4-(methoxycarbonyl)phenyl)-4-(2-((tetrahydro-2H-pyran-2-yl)oxy)ethoxy)piperidine-1-carboxylate (9): To a solution of compound 8 (800 mg, 1.66 mmol) in MeOH (2 mL) and toluene (7 mL) was added TMSCHN₂ (1.8 mL, 3.69 mmol, 2 M in hexane) dropwise and the reaction was stirred at room temperature for 1 hour.

The mixture was quenched with AcOH and concentrated to dryness under reduced pressure. The residue was purified by column chromatography on silica gel (PE:EtOAc=30:1 to 3:1) to give compound 9 (700 mg, yield 85.6%) as oil. LC/MS(ESI) m/z: 414 (M−THP+1)⁺.

Step 9: benzyl (2S,4S)-4-(2-hydroxyethoxy)-2-(4-(methoxycarbonyl)phenyl) piperidine-1-carboxylate (10): To a solution of compound 9 (700 mg, 1.41 mmol) in MeOH (5 mL) was added TsOH (24 mg, 0.14 mmol) and the reaction was stirred at room temperature for 3 hours. The mixture was diluted with ice-water and extracted with DCM twice. The combined organic layers were washed with brine, dried over anhydrous Na₂SO₄, filtered and concentrated to dryness. The residue was purified by column chromatography on silica gel (PE:EtOAc=10:1 to 1:2) to give compound 10 (570 mg, yield 97.9%) as yellow oil. LC/MS(ESI) m/z: 414 (M+H)⁺.

Step 10: benzyl (2S,4S)-2-(4-(methoxycarbonyl)phenyl)-4-(2-((methylsulfonyl)oxy) ethoxy)piperidine-1-carboxylae (11): To a solution of compound 10 (570 g, 1.38 mmol) in DCM (8 mL) was added TEA (557 mg, 5.52 mmol) and MsCl (317 mg, 2.76 mmol) at 0° C. under N₂ atmosphere. The reaction mixture was stirred at 0° C. for 1 hour. The mixture was quenched with ice-water and extracted with EtOAc twice. The combined organic layers were washed with brine, dried over anhydrous Na₂SO₄, filtered and concentrated to dryness under reduced pressure to give compound 11 (670 mg, yield 98.8%) as yellow oil, which was used directly in the next step without further purification. LC/MS(ESI) m/z: 492 (M+H)⁺.

Step 11: benzyl (2S,4S)-4-(2-azidoethoxy)-2-(4-(methoxycarbonyl)phenyl) piperidine-1-carboxylate (12): To a solution of compound 11 (670 mg, 1.36 mmol) in DMF (6 mL) was added NaN₃ (106 mg, 1.64 mmol) and the reaction was stirred at 45° C. overnight. The mixture was washed with ice-water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=30:1 to 5:1) to give compound 12 (368 mg, yield 61.6%) as oil. LC/MS(ESI) m/z: 439 (M+H)⁺.

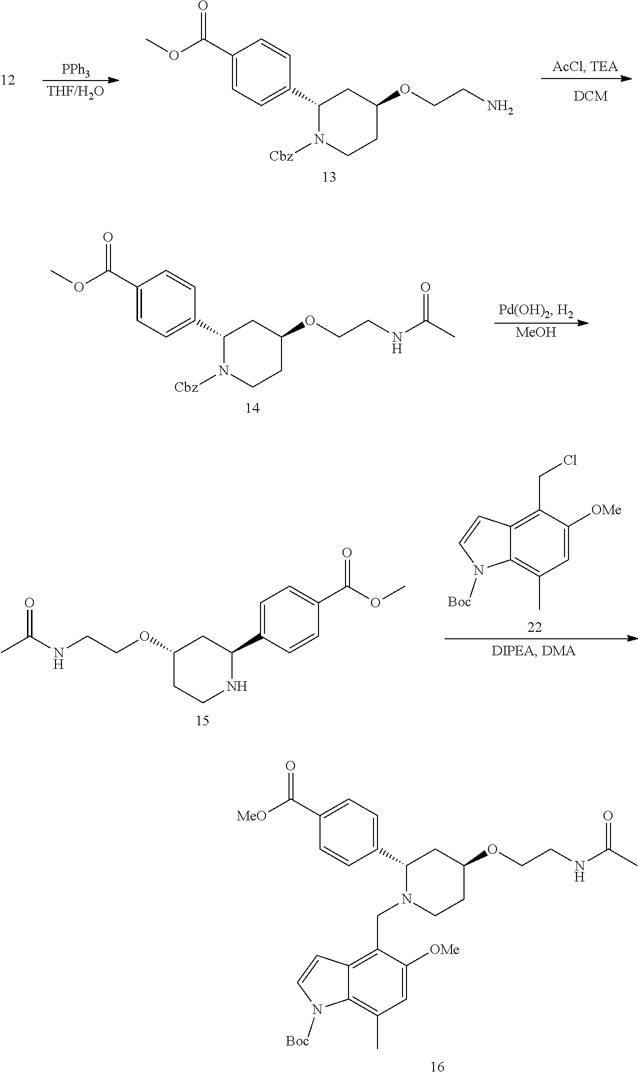

Step 12: benzyl (2S,4S)-4-(2-aminoethoxy)-2-(4-(methoxycarbonyl)phenyl) piperidine-1-carboxylate (13): To a solution of compound 12 (368 mg, 0.84 mmol) in THF (4 mL) and H₂O (0.4 mL) was added PPh₃ (663 mg, 2.52 mmol) and the reaction was stirred at 30° C. for 4 hours. The mixture was acidified with 0.5 M aq.HCl solution to pH˜5 and extracted with EtOAc twice. The aqueous layer was neutralized with sat. NaHCO₃ solution to pH˜8 and extracted with EtOAc twice. The organic layers were washed with brine, dried over anhydrous Na₂SO₄, filtered and concentrated to dryness under reduced pressure to give compound 13 (290 mg, yield 83.8%) as oil, which was used directly in the next step without further purification. LC/MS(ESI) m/z: 413 (M+H)⁺.

Step 13: benzyl (2S,4S)-4-(2-acetamidoethoxy)-2-(4-(methoxycarbonyl)phenyl) piperidine-1-carboxylate (14): To a solution of compound 13 (290 mg, 0.70 mmol) in DCM (4 mL) was added TEA (213 mg, 2.11 mmol) and acetyl chloride (82 mg, 1.05 mmol) at 0° C. under N₂ atmosphere. The reaction mixture was stirred at 0° C. for 1.5 hours. The mixture was quenched with water at 0° C. and extracted with DCM twice. The combined organic layers were washed with brine, dried over anhydrous Na₂SO₄, filtered and concentrated to dryness under reduced pressure. The residue was purified by column chromatography on silica gel (DCM:MeOH=80:1 to 30:1) to give compound 14 (290 mg, yield 90.9%) as yellow oil. LC/MS(ESI) m/z: 455 (M+H)⁺.

Step 14: methyl 4-((2S,4S)-4-(2-acetamidoethoxy)piperidin-2-yl)benzoate (15): To a solution of compound 14 (290 mg, 0.64 mmol) in MeOH (4 mL) was added Pd(OH)₂ (101 mg, 35% wt) at 0° C. under N₂ atmosphere and the mixture was stirred under H₂ atmosphere at 40° C. for 1.5 hours. The mixture was filtered, and the filtrate was concentrated to dryness under reduced pressure to give compound 15 (200 mg, yield 98.0%) as oil, which was used directly in the next step without further purification. LC/MS(ESI) m/z: 321 (M+H)⁺.

Step 15: tert-butyl 4-(((2S,4S)-4-(2-acetamidoethoxy)-2-(4-(methoxycarbonyl)phenyl)piperidin-1-yl)methyl)-5-methoxy-7-methyl-1H-indole-1-carboxylate(16): To a solution of compound 15 (120 mg, 0.38 mmol) in DMA (7 mL) was added DIPEA (490 mg, 3.8 mmol) and compound 22 (235 mg, 0.76 mmol) at 0° C. and the mixture was stirred at 100° C. for 3 hours. The mixture was cooled down to room temperature and concentrated to dryness under reduced pressure. The residue was purified by column chromatography on silica gel (DCM:MeOH=100:1 to 20:1) to give compound 16 (200 mg, yield 88.9%) as yellow solid. LC/MS (ESI) (m/z): 594 (M+H)⁺.

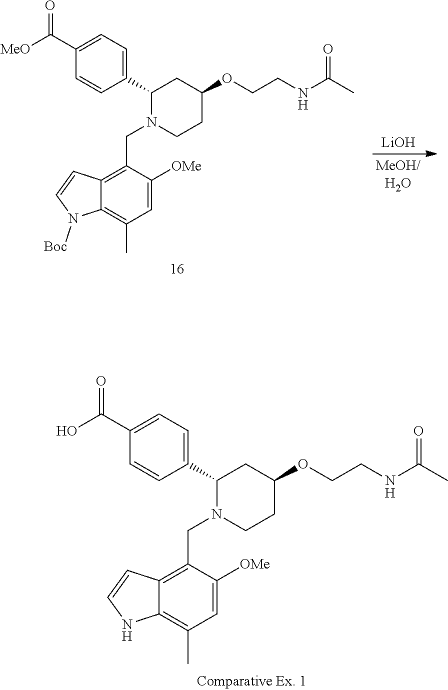 [SMILES: COC(=O)c1ccc([C@@H]2C[C@@H](OCCNC(C)=O)CCN2Cc2c(OC)cc(C)c3c2ccn3C(=O)OC(C)(C)C)cc1 ; "LiOH" ; "MeOH/H2O" ; COc1cc(C)c2[nH]ccc2c1CN1CC[C@H](OCCNC(C)=O)C[C@H]1c1ccc(C(=O)O)cc1]

Step 16: 4-((2S,4S)-4-(2-acetamidoethoxy)-1-((5-methoxy-7-methyl-1H-indol-4-yl)methyl)piperidin-2-yl)benzoic acid (Comparative Example 1): To a solution of compound 16 (100 mg, 0.17 mmol) in MeOH (4 mL) was added LiOH H₂O (1 mL, 1 mmol, 1 M in water) at 0° C. and the mixture was stirred at 40° C. overnight. The mixture was diluted with water and washed with MTBE twice. The aqueous layer was acidified with 0.5 M aq.

HCl solution and purified by prep-HPLC to give the title compound (11.4 mg, yield 14.1%) as white solid. ¹H NMR (400 MHz, CD₃OD) δ 8.43 (s, 1H), 8.18 (d, J=8.0 Hz, 2H), 7.67 (d, J=8.0 Hz, 2H), 7.33 (d, J=2.8 Hz, 1H), 6.78 (s, 1H), 6.34 (s, 1H), 4.80-4.71 (m, 1H), 4.36 (d, J=11.2 Hz, 1H), 4.18 (d, J=12.4 Hz, 1H), 3.89-3.83 (m, 1H), 3.77 (s, 3H), 3.64-3.45 (m, 6H), 2.52 (s, 3H), 2.31-2.23 (m, 2H), 2.12-2.03 (m, 2H), 2.02 (s, 3H). LC/MS (ESI) m/z: 480 (M+H)⁺.

### Synthesis of Intermediate: tert-butyl 4-(chloromethyl)-5-methoxy-7-methyl-1H-indole-1-carboxylate (22)

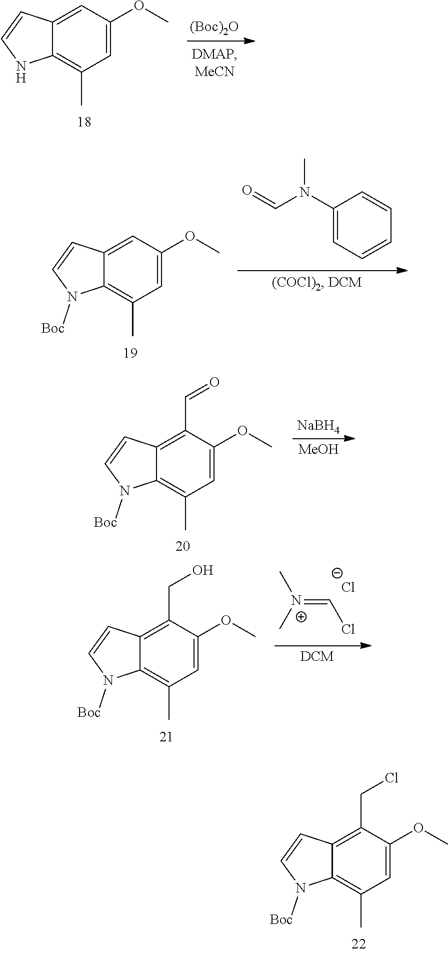

Step 1: tert-butyl 5-methoxy-7-methyl-1H-indole-1-carboxylate (19): To a solution of compound 18 (500 mg, 3.11 mmol) in MeCN (4 mL) was added Boc₂O (812 mg, 3.72 mmol) and DMAP (455 mg, 3.72 mmol) at 0° C. The reaction mixture was stirred at room temperature for 2 hours. The mixture was washed with water and extracted with DCM twice. The combined organic layers were washed with brine, dried over anhydrous Na₂SO₄, filtered and concentrated to dryness under reduced pressure. The residue was purified by column chromatography on silica gel (PE:EtOAc=100:1 to 5:1) to give compound 19 (790 mg, yield 97.5%) as white solid. LC/MS(ESI) m/z: 262 (M+H)⁺.

Step 2: tert-butyl 4-formyl-5-methoxy-7-methyl-1H-indole-1-carboxylate (20): To a solution of N-methylformanilide (471 mg, 3.48 mmol) in DCM (5 mL) was added oxalyl chloride (442 mg, 3.48 mmol) dropwise at room temperature under N₂ atmosphere and the reaction was stirred at room temperature overnight. The mixture was then added to a solution of compound 19 (700 mg, 2.68 mmol) dropwise in DCM (4 mL) at −14° C. under N₂ atmosphere. The reaction was stirred at −14° C. for 3.5 hours and quenched with ice-water. The mixture was extracted with DCM twice. The combined organic layers were washed with brine, dried over anhydrous Na₂SO₄, filtered and concentrated to dryness under reduced pressure. The residue was purified by column chromatography on silica gel (PE:EtOAc=50:1 to 5:1) to give compound 20 (530 mg, yield 68.38%) as white solid. LC/MS(ESI) m/z: 290 (M+H)⁺.

Step 3: tert-butyl 4-(hydroxymethyl)-5-methoxy-7-methyl-1H-indole-1-carboxylate (21): To a mixture of compound 20 (300 mg, 1.04 mmol) in MeOH (3 mL) was added NaBH₄ (79 mg, 2.08 mmol) at 0° C. The reaction was stirred at room temperature for 1.5 hours and quenched with ice-water. The mixture was extracted with DCM twice. The combined organic layers were washed with brine, dried over anhydrous Na₂SO₄, filtered and concentrated to dryness under reduced pressure. The residue was purified by column chromatography on silica gel (PE:EtOAc=20:1 to 1:1) to give compound 21 (300 mg, yield 99.3%) as white solid. LC/MS (ESI) m/z: 274 (M-OH)⁺.

Step 4: tert-butyl 4-(chloromethyl)-5-methoxy-7-methyl-1H-indole-1-carboxylate(22): To a solution of compound 21 (250 mg, 0.85 mmol) in DCM (4 mL) was added (chloromethylene)dimethyliminium chloride (198 mg, 1.55 mmol) at 0° C. under N₂ atmosphere and the reaction was stirred at 0° C. for 3 hours. The mixture was quenched with 5% aq.NaHCO₃ solution at 0° C. and extracted with EtOAc twice. The organic layer was washed with 0.2M aq. LiCI solution and brine, dried over anhydrous Na₂SO₄, filtered and concentrated to dryness under reduced pressure to give compound 22 (235 mg, yield 89.4%) as yellow oil, which was used directly in the next step without further purification.

### Comparative Example 2: N-((2-acetamidoethyl)sulfonyl)-4-((2S,4S)-4-ethoxy-1-((5-methoxy-7-methyl-1H-indol-4-yl)methyl)piperidin-2-yl)benzamide

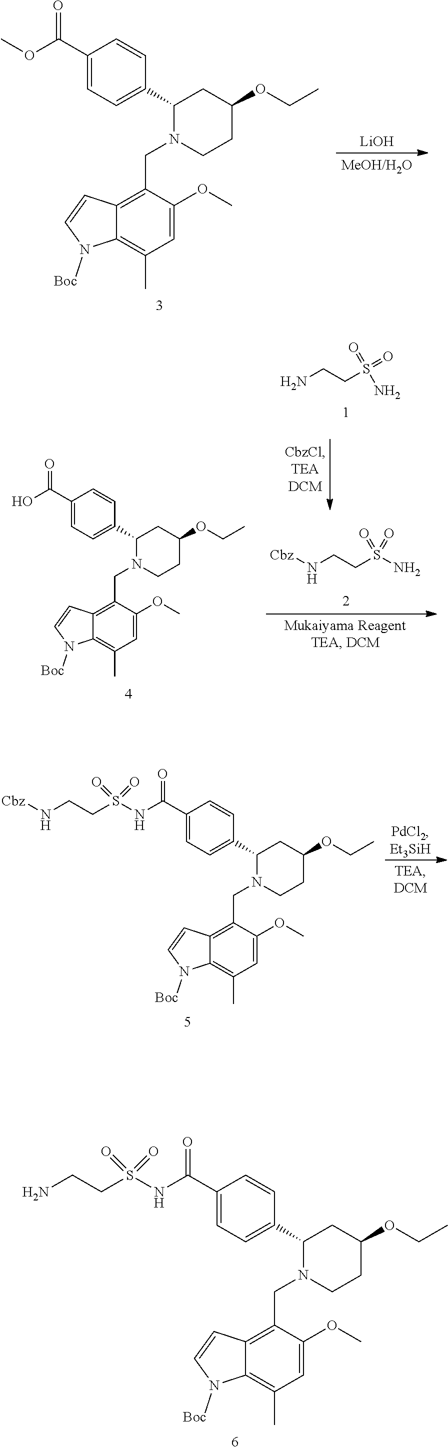

Step 1: benzyl (2-sulfamoylethyl)carbamate: To a solution of 2-aminoethane-1-sulfonamide (500 mg, 4.03 mmol) in DMF (5 mL) was added TEA (813 mg, 8.05 mmol) at 0° C. under N₂ atmosphere and the mixture was stirred at room temperature for 30 minutes. CbzCl (687 mg, 4.03 mmol) was added to the reaction mixture and the resulting mixture was stirred at 25° C. under N₂ atmosphere overnight. The mixture was quenched with ice-water and extracted with ethyl acetate twice. The combined organic layers were washed with brine, dried over anhydrous Na₂SO₄, filtered and concentrated under reduced pressure. The residue was purified by chromatography on silica gel (petroleum ether:ethyl acetate=30:1 to 3:1) to give compound 4 (290 mg, yield 28%) as white solid. LC/MS (ESI) m/z: 259 (M+H)⁺.

Step 2: 4-((2S,4S)-1-((1-(tert-butoxycarbonyl)-5-methoxy-7-methyl-1H-indol-4-yl)methyl)-4-ethoxypiperidin-2-yl)benzoic acid: To a solution of compound 3 (220 mg, 0.372 mmol) in methanol/water (4 mL, v/v=3/1) was added LiOH·H₂O (23 mg, 0.56 mmol) at 0° C. and the mixture was stirred at 25° C. for 3 hours. The mixture was diluted with water and washed with MTBE twice. The aqueous layer was acidified with 0.5 M aq.HCl solution and extracted with ethyl acetate twice. The combined organic layers were washed with brine, dried over anhydrous Na₂SO₄, filtered and concentrated to dryness under reduced pressure. The residue was purified by chromatography on silica gel (dichloromethane:methanol=30:1 to 10:1) to give compound 4 (120 mg, yield 62%) as brown solid. LC/MS (ESI) m/z: 523 (M+H)⁺.

Step 3: tert-butyl 4-(((2S,4S)-2-(4-(((2-(((benzyloxy)carbonyl)amino)ethyl)sulfonyl) carbamoyl)phenyl)-4-ethoxypiperidin-1-yl)methyl)-5-methoxy-7-methyl-1H-indole-1-carboxylate: To a suspension of compound 4 (82 mg, 0.156 mmol), compound 2 (81 mg, 0.312 mmol) and TEA (79 mg, 0.784 mmol) in dichloromethane (1 mL) was added 2-chloro-1-methylpyridinium iodide (120 mg, 0.470 mmol) at 0° C. under N₂ atmosphere and the mixture was stirred at room temperature for 16 hours. The mixture was concentrated to dryness under reduced pressure. The residue was purified by chromatography on silica gel (petroleum ether:ethyl acetate=30:1 to 1:1) to give compound 5 (100 mg, yield 84%) as white solid. LC/MS (ESI) m/z: 764 (M+H)⁺.

Step 4: tert-butyl 4-(((2S,4S)-2-(4-(((2-aminoethyl)sulfonyl)carbamoyl)phenyl)-4-ethoxypiperidin-1-yl)methyl)-5-methoxy-7-methyl-1H-indole-1-carboxylate: To a suspension of compound 5 (60 mg, 0.078 mmol), triethylsilane (36 mg, 0.312 mmol) and TEA (63 mg, 0.624 mmol) in dichloromethane (1 mL) was added PdCl₂ (18 mg, 0.156 mmol) at 0° C. under N₂ atmosphere and the mixture was stirred at room temperature for 1 hours. The mixture was concentrated under reduced pressure to give 130 mg of crude compound 6 as white solid, which was used directly in the next step without further purification. LC/MS (ESI) m/z: 629 (M+H)⁺.

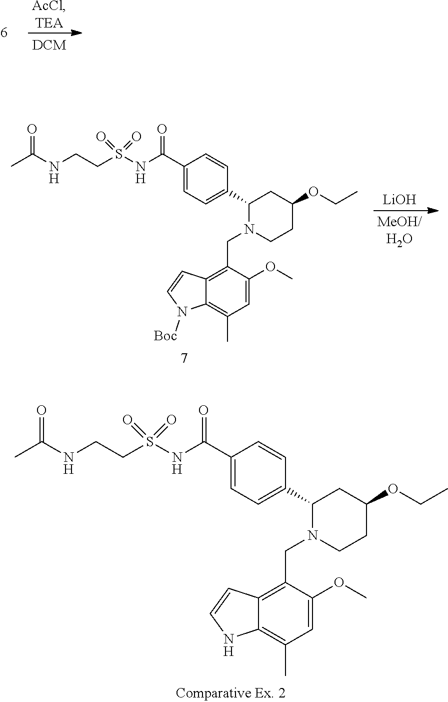

Step 5: tert-butyl 4-(((2S,4S)-2-(4-(((2-acetamidoethyl)sulfonyl)carbamoyl)phenyl)-4-ethoxypiperidin-1-yl)methyl)-5-methoxy-7-methyl-1H-indole-1-carboxylate: To a solution of compound 6 (130 mg, 0.078 mmol) in dichloromethane (1 mL) was added TEA (16 mg, 0.156 mmol) at 0° C. under N₂ atmosphere and the mixture was stirred at room temperature for 30 minutes. Acetyl chloride (9.1 mg, 0.117 mmol) was added to the reaction mixture and the resulting mixture was stirred at 25° C. under N₂ atmosphere for 30 minutes. The mixture was quenched with ice-water and extracted with dichloromethane twice. The combined organic layers were washed with brine, dried over anhydrous Na₂SO₄, filtered and concentrated under reduced pressure. The residue was purified by chromatography on silica gel (petroleum ether:ethyl acetate=30:1 to 1:1) to give compound 7 (30 mg, yield 57% in two steps) as brown solid. LC/MS (ESI) m/z: 671 (M+H)⁺.

Step 6: N-((2-acetamidoethyl)sulfonyl)-4-((2S,4S)-4-ethoxy-1-((5-methoxy-7-methyl-1H-indol-4-yl)methyl)piperidin-2-yl)benzamide (Comparative Example 2): To a solution of compound 7 (30 mg, 0.045 mmol) in methanol/water (1 mL, v/v=3/1) was added LiOH·H₂O (4 mg, 0.09 mmol) at 0° C. and the mixture was stirred at 40° C. for 6 hours. The mixture was diluted with water and washed with MTBE twice. The aqueous layer was acidified with 0.5 M aq. HCl solution and extracted with DCM twice. The combined organic layers were washed with brine, dried over anhydrous Na₂SO₄, filtered and concentrated to dryness under reduced pressure. The residue was purified by prep-HPLC to give the title compound (4 mg, yield 19%) as white solid. ¹H NMR (400 MHz, CD₃OD) δ 8.20 (d, J=8.1 Hz, 2H), 7.67 (d, J=8.2 Hz, 2H), 7.33 (d, J=3.1 Hz, 1H), 6.77 (s, 1H), 6.34 (d, J=3.1 Hz, 1H), 4.80-4.73 (m, 1H), 4.33 (d, J=12.7 Hz, 1H), 4.23 (d, J=12.8 Hz, 1H), 3.82 (s, 1H), 3.77 (s, 3H), 3.65-3.61 (m, 3H), 3.57 (dd, J=11.7, 5.9 Hz, 4H), 3.37 (d, J=12.6 Hz, 1H), 2.51 (s, 3H), 2.25 (d, J=7.4 Hz, 2H), 2.03 (dd, J=29.8, 13.9 Hz, 2H), 1.88 (s, 3H), 1.31 (t, J=7.0 Hz, 3H); LC/MS (ESI) m/z: 671 (M+H)⁺.

### Example 3 and 4: 4-((2S,4S)-4-ethoxy-1-((5-methoxy-7-methyl-1H-indol-4-yl)methyl)piperidin-2-yl)-3-methoxybenzoic acid

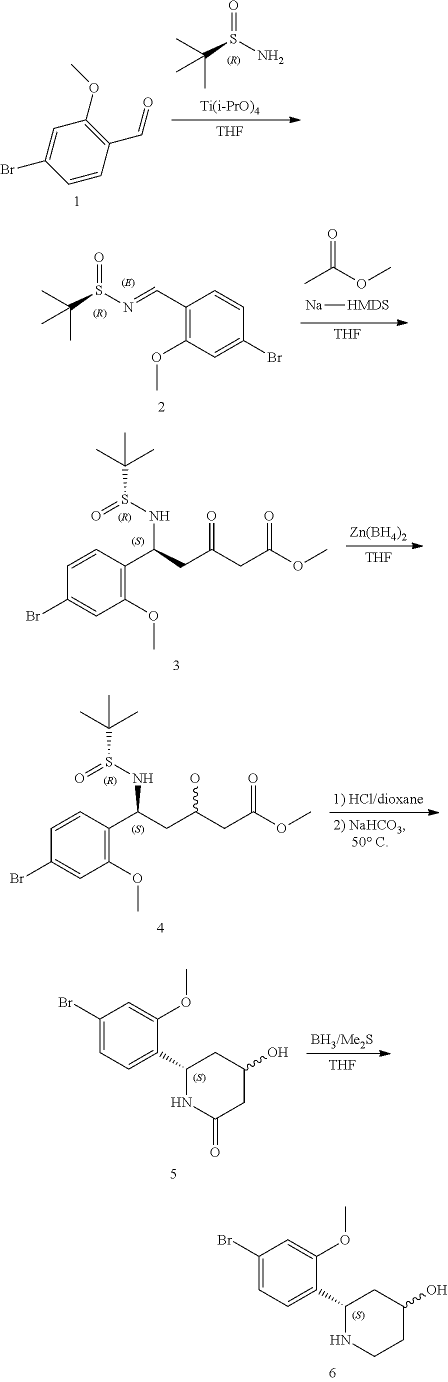

Step 1: (R,E)-N-(4-bromo-2-methoxybenzylidene)-2-methylpropane-2-sulfinamide (2): A solution of compound 1 (10.0 g, 46.4 mmol), titanium(IV) isopropoxide (139 mmol), (R)-2-methylpropane-2-sulfinamide (8.44 g, 69.7 mmol) in THF (100 mL) was stirred at 75° C. for 2 hours. The reaction was quenched with water and filtered. The resulting solution was partitioned between ethyl acetate and water. The organic layer was separated and washed with brine, dried over anhydrous Na₂SO₄, filtered and concentrated to dryness under reduced pressure. The residue was purified by flash column chromatography on silica gel (petroleum ether:ethyl acetate=1:0 to 1:1) to give compound 2 (12.4 g, Yield 79.6%) as yellow oil. LC/MS: m/z 317(M+H)⁺.

Step 2: methyl (S)-5-(4-bromo-2-methoxyphenyl)-5-(((R)-tert-butylsulfinyl)amino)-3-oxopentanoate (3): In a 500 mL one-necked round-bottomed flask was placed a solution of THF (120 mL) and NaHMDS (214 mmol) at −78° C. Methyl acetate (10.6 g, 143 mmol) was added slowly, and the solution was stirred for 1 h. Compound 2 (12.0 g, 35.8 mmol) in THF (20 mL) was added dropwise to the above solution. The reaction mixture was stirred at −78° C. for 3.5 hours and quenched with aq.NH₄Cl solution. The solution was washed with ethyl acetate, the organic layer was separated, dried over anhydrous Na₂SO₄ and concentrated to dryness under reduced pressure. The residue was further purified by flash chromatography (ethyl acetate:petroleum ether=1:3 to 1:1) give compound 3 as yellow oil. LC/MS: m/z 434 (M+H)⁺.

Step 3: methyl (3R,5S)-5-(4-bromo-2-methoxyphenyl)-5-(((R)-tert-butylsulfinyl)amino)-3-hydroxypentanoate (4): To a solution of compound 3 (9.0 g, 16.6 mmol) in tetrahydrofuran (100 mL) was added Zn(BH₄)₂(3.94 g, 41.4 mmol) in tetrahydrofuran dropwise at −78° C. under N₂ atmosphere. The reaction was stirred at −78° C. for 1 hours and quenched with methanol and aq.NH₄Cl solution. The mixture was extracted with ethyl acetate twice. The combined organic layers were washed with brine, dried over anhydrous Na₂SO₄, filtered and concentrated to dryness under reduced pressure. The residue was purified by column chromatography on silica gel (dichloromethane:ethyl acetate=7:1 to 7:3) to give compound 4 (7.0 g, Yield 87.1%) as oil. LC/MS: m/z 436.0 (M+H)⁺.

Step 4: (6S)-6-(4-bromo-2-methoxyphenyl)-4-hydroxypiperidin-2-one (5): A solution of compound 4 (10.0 g, 22.9 mmol) in HCl/1,4-dioxane (20 mL) was stirred for 30 minutes under nitrogen atmosphere. The reaction mixture was neutralized to pH=8. The reaction mixture was extracted with dichloromethane (200 mL) twice, dried over anhydrous Na₂SO₄ and concentrated to dryness under reduced pressure. The residue was further purified by flash column chromatography on silica gel to give compound 5 (6.0 g, Yield 82.8%) as white solid. LC/MS: m/z 299.9 (M+H)⁺.

Step 5: (2S)-2-(4-bromo-2-methoxyphenyl)piperidin-4-ol (6): To a solution of compound 5 (2.0 g, 6.67 mmol) in THF (20 mL) was added BH₃/DMS (1.90 mL, 19.0 mmol) dropwise at 0° C. under N₂ atmosphere. The reaction was stirred at 80° C. for 2 hours and quenched with 1 N HCl. The mixture was added aq.NaHCO₃ solution until pH=8. The mixture was concentrated to dryness under reduced pressure. The residue was washed with 10% MeOH in DCM, and the organic layer was separated, dried over anhydrous sodium acetate and concentrated to dryness under reduced pressure to give compound 6 (1.6 g, 5.31 mmol, Yield 83.9%) as white solid. LC/MS: m/z 285.9 (M+H)⁺.

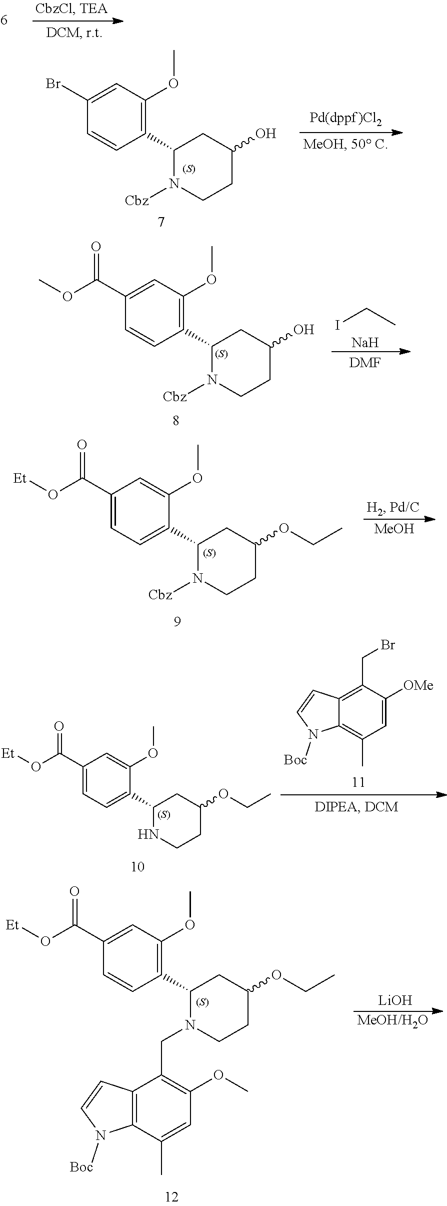

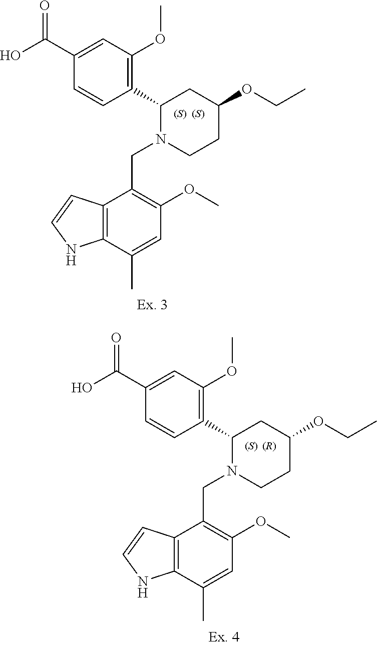

Step 6: benzyl (2S)-2-(4-bromo-2-methoxyphenyl)-4-hydroxypiperidine-1-carboxylate (7): To a solution of compound 7 and TEA (1.05 g, 10.4 mmol) in dichloromethane (15 mL) was added CbzCl (1.17 g, 6.92 mmol) dropwise at 0° C. under N₂ atmosphere. The reaction was stirred at room temperature for 0.5 hour and quenched with water. The mixture was extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=10:1 to 3:1) to give compound 7 (1.2 g, Yield 74.3%) as white solid. LC/MS: m/z 420.0 (M+H)⁺.

Step 7: benzyl (2S)-4-hydroxy-2-(2-methoxy-4-(methoxycarbonyl)phenyl) piperidine-1-carboxylate (8): To a mixture of compound 7 (1.0 g, 2.14 mmol) and TEA (1.08 g, 10.5 mmol) in methanol (4 mL) was added Pd(dppf)Cl₂ (0.76 g, 1.07 mmol) at room temperature under CO atmosphere and the mixture was stirred at 50° C. for 16 hours. The mixture was concentrated to dryness under reduced pressure. The residue was purified by flash column chromatography on silica gel (petroleum ether:ethyl acetate=5:1 to 1:1) to give compound 8 (600 mg, Yield 63.2%) as white solid. LC/MS: m/z 400 (M+H)⁺.

Step 8: benzyl (2S)-4-ethoxy-2-(4-(ethoxycarbonyl)-2-methoxyphenyl)piperidine-1-carboxylate (9): A solution of compound 8 (280 mg, 0.63 mmol) in DMF (4 mL) was added NaH (0.05 g, 1.26 mmol) at 0° C., and stirred for 0.5 hours under nitrogen. Ethyl iodide (300 mg, 1.89 mmol) was added and the reaction was stirred for additional 12 hours at 40° C. The reaction solution was concentrated under reduced pressure to give the crude product. The residue was purified by column chromatography on silica gel (petroleum ether:ethyl acetate=10:1 to 3:1) to give compound 9 (0.2 g, Yield 66.7%) as colorless oil. LC/MS: m/z 442.1 (M+H)⁺.

Step 9: ethyl 4-((2S)-4-ethoxypiperidin-2-yl)-3-methoxybenzoate (10): To a solution of compound 9 (200 mg, 0.45 mmol) in methanol (4 mL) was added Pd/C (30 mg) at room temperature under N₂ atmosphere and the mixture was stirred under H₂ atmosphere at 40° C. for 16 hours. The mixture was filtered, and the filtrate was concentrated to dryness under reduced pressure to give compound 10 (120 mg, yield 85.0%) as oil, which was used directly in the next step without further purification. LC/MS(ESI) m/z: 308.0 (M+H)⁺.

Step 10: tert-butyl 4-(((2S)-4-ethoxy-2-(4-(ethoxycarbonyl)-2-methoxyphenyl) piperidin-1-yl)methyl)-5-methoxy-7-methyl-1H-indole-1-carboxylate (12): A solution of compound 10 (80 mg, 0.26 mmol) in dichloromethane (8 mL) was added compound 11 (130 mg, 0.31 mmol) at 0° C. and stirred for 1.5 hours under nitrogen atmosphere. N,N-diisopropylethylamine (92 mg, 0.71 mmol) and methyl 4-[(2S,4S)-4-ethoxypiperidin-2-yl]benzoate (62 mg, 0.24 mmol) were added and the reaction was stirred for additional 2 hours under nitrogen at 0° C. The reaction solution was concentrated under reduced pressure to give the crude product. The residue was further purified by flash column chromatography on silica gel (petroleum ether:ethyl acetate=10:1 to 3:1) to give compound 12 (90 mg, Yield 67.2%) as white solid. LC/MS: m/z 581.0 (M+H)⁺.

Step 11: 4-((2S,4S)-4-ethoxy-1-((5-methoxy-7-methyl-1H-indol-4-yl)methyl)piperidin-2-yl)-3-methoxybenzoic acid (Example 3) and 4-((2S,4R)-4-ethoxy-1-((5-methoxy-7-methyl-1H-indol-4-yl)methyl)piperidin-2-yl)-3-methoxybenzoic acid (Example 4): A mixture of compound 12 (90 mg, 0.16 mmol) and LiOH H₂O (0.019 g, 0.47 mmol) in methanol (1.5 mL), water (0.5 mL) was stirred at 40° C. overnight. The mixture was diluted with water and extracted with ethyl acetate twice. The combined organic layers were washed with water and brine, dried over anhydrous Na₂SO₄, filtered and concentrated to dryness under reduced pressure. The residue was further purified by prep-HPLC to give Ex. 3 (25 mg, Yield: 35.6%) and Ex. 4 (10 mg, Yield 14.2%) as white solid. Ex. 3: ¹H NMR (400 MHz, CD₃OD) δ 7.79 (d, J=7.6 Hz, 1H), 7.72 (s, 1H), 7.63 (d, J=7.6 Hz, 1H), 7.31 (d, J=3.1 Hz, 1H), 6.77 (s, 1H), 6.35 (d, J=3.1 Hz, 1H), 4.39 (d, J=12.9 Hz, 1H), 4.29 (d, J=12.9 Hz, 1H), 4.02 (s, 3H), 3.80 (s, 4H), 3.60 (q, J=7.0 Hz, 3H), 2.50 (s, 3H), 2.23-1.93 (m, 4H), 1.31 (s, 3H); LC/MS: m/z 453 (M+H)⁺. Ex. 4: ¹H NMR (400 MHz, CD₃OD) δ 7.80 (s, 1H), 7.75 (s, 1H), 7.62 (s, 1H), 7.32 (d, J=3.1 Hz, 1H), 6.77 (s, 1H), 6.30 (d, J=3.1 Hz, 1H), 4.84-4.84 (m, 1H), 4.38 (d, J=12.6 Hz, 1H), 4.19 (d, J=12.8 Hz, 1H), 4.03 (s, 3H), 3.79 (s, 4H), 3.57 (d, J=7.0 Hz, 3H), 3.40 (d, J=13.7 Hz, 1H), 2.50 (s, 3H), 2.32 (d, J=13.5 Hz, 1H), 2.25 (s, 1H), 2.10-1.97 (m, 1H), 1.75 (d, J=14.9 Hz, 1H), 1.16 (t, J=7.0 Hz, 3H); LC/MS: m/z 453 (M+H)⁺.

### Example 5: (1²S,1⁴S)-11-((5-methoxy-7-methyl-1H-indol-4-yl)methyl)-3,10-dioxa-1(2,4)-piperidina-2(1,2)-benzenacyclodecaphane-2⁴-carboxylic acid

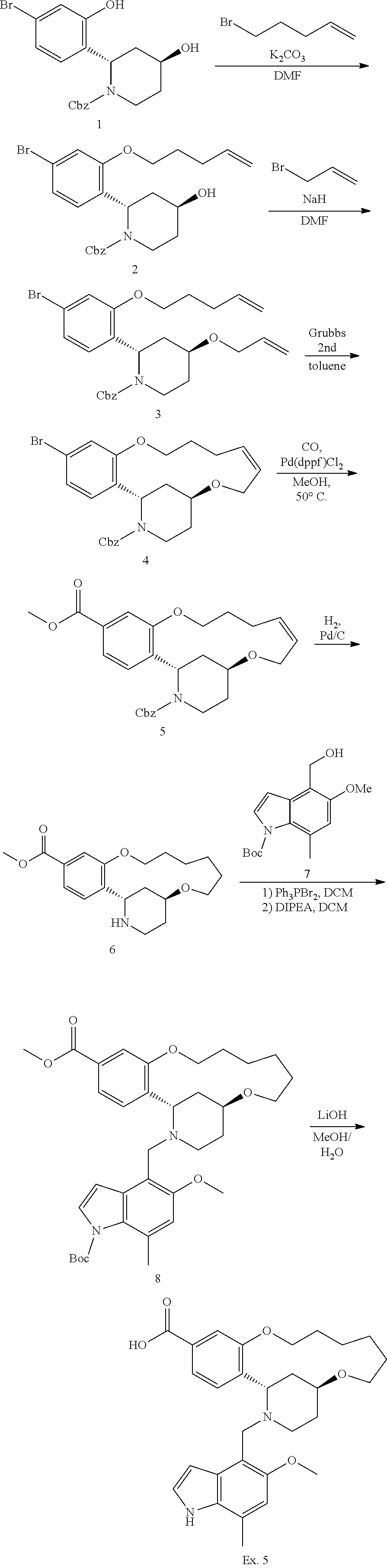

Step 1: benzyl (2S,4S)-2-[4-bromo-2-(pent-4-en-1-yloxy)phenyl]-4-hydroxy-piperidine (2): To a solution of benzyl (2S,4S)-2-(4-bromo-2-hydroxyphenyl)-4-hydroxy-piperidine-1-carboxylate (350 mg, 0.86 mmol) and K₂CO₃ (360 mg, 2.58 mmol) in DMF (5 mL) was added 5-bromopent-1-ene (257 mg, 1.72 mmol) and stirred at 25° C. for 1 hours under nitrogen. The reaction solution was concentrated under reduced pressure to give the crude product. The residue was further purified by column chromatography on silica gel (petroleum ether:ethyl acetate=10:1 to 3:1) to give benzyl (2S,4S)-2-[4-bromo-2-(pent-4-en-1-yloxy)phenyl]-4-hydroxypiperidine (360 mg, Yield 88.1%) as white solid. LC/MS: m/z 474.0 (M+H)⁺.

Step 2: benzyl (2S,4S)-2-(4-bromo-2-(pent-4-en-1-yloxy)phenyl)-4-hydroxy-piperidine-1-carboxylate (3): To a solution of benzyl (2S,4S)-2-[4-bromo-2-(pent-4-en-1-yloxy)phenyl]-4-hydroxypiperidine (360 mg, 0.76 mmol) in DMF (5 mL) was added NaH (91 mg, 2.28 mmol, 60% dispersion in mineral oil) at 0° C. and stirred for 0.5 hours under nitrogen. Then allyl bromide (275 mg, 2.28 mmol) was added and stirred for additional 0.5 hours at 25° C. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was further purified by flash column chromatography on silica gel (petroleum ether:ethyl acetate=10:1 to 3:1) to give benzyl (2S,4S)-2-(4-bromo-2-(pent-4-en-1-yloxy)phenyl)-4-hydroxypiperidine-1-carboxylate (360 mg, Yield 92.2%) as colorless oil. LC/MS: m/z 514.1 (M+H)⁺.

Step 3: benzyl (1²S,1⁴S, Z)-2⁴-bromo-3,10-dioxa-1(2,4)-piperidina-2(1,2)-benzenacyclodecaphan-7-ene-1′-carboxylate (4): To a solution of benzyl (2S,4S)-2-(4-bromo-2-(pent-4-en-1-yloxy)phenyl)-4-hydroxypiperidine-1-carboxylate (360 mg, 0.70 mmol) in DCM (400 mL) was added Grubbs 1^{st} (28.8 mg, 0.035 mmol). The mixture was stirred at room temperature under N₂ atmosphere overnight. The reaction was concentrated to dryness under reduced pressure. The residue was purified by flash column chromatography on silica gel (petroleum ether:ethyl acetate=10:1 to 1:1) to give benzyl (1²S,1⁴S,Z)-2⁴-bromo-3,10-dioxa-1(2,4)-piperidina-2(1,2)-benzenacyclodecaphan-7-ene-1′-carboxylate (260 mg, Yield 76.4%) as colorless oil. LC/MS: m/z 486.1 (M+H)⁺.

Step 4: 1¹-benzyl 2⁴-methyl (1²S,1⁴S, Z)-3,10-dioxa-1(2,4)-piperidina-2(1,2)-benzenacyclodecaphan-7-ene-1¹,2⁴-dicarboxylate (5): To a mixture of benzyl (1²S,1⁴S,Z)-2⁴-bromo-3,10-dioxa-1(2,4)-piperidina-2(1,2)-benzenacyclodecaphan-7-ene-1′-carboxylate (200 mg, 0.43 mmol) and TEA (216 mg, 2.14 mmol) in methanol (4 mL) was added Pd(dppf)Cl₂ (160 mg, 0.21 mmol) at room temperature under CO atmosphere and the mixture was stirred at 50° C. for 16 hours. The mixture was concentrated to dryness under reduced pressure. The residue was purified by flash column chromatography on silica gel (petroleum ether:ethyl acetate=5:1 to 1:1) to give benzyl 11-benzyl 2⁴-methyl (1²S,1⁴S, Z)-3,10-dioxa-1(2,4)-piperidina-2(1,2)-benzenacyclodecaphan-7-ene-1¹,2⁴-dicarboxylate (100 mg, Yield 52.6%) as white solid. LC/MS: m/z 466.1 (M+H)⁺.

Step 5: methyl (1²S,1⁴S)-3,10-dioxa-1(2,4)-piperidina-2(1,2)-benzenacyclodecaphane-2⁴-carboxylate (6):To a solution of 1¹-benzyl 2⁴-methyl (1²S,1⁴S, Z)-3,10-dioxa-1(2,4)-piperidina-2(1,2)-benzenacyclodecaphan-7-ene-1¹,2⁴-dicarboxylate (60 mg, 0.13 mmol) in ethyl acetate (2 mL) was added Pd/C (30 mg, 10% wt) at 25° C. under N₂ atmosphere and the mixture was stirred under H₂ atmosphere at 40° C. for 16 hours. The mixture was filtered and the filtrate was concentrated to dryness under reduced pressure to give methyl (1²S,1⁴S)-3,10-dioxa-1(2,4)-piperidina-2(1,2)-benzenacyclodecaphane-2⁴-carboxylate (16 mg, yield 37.2%) as oil, which was used directly in the next step without further purification. LC/MS(ESI) m/z: 334.1 (M+H)⁺.

Step 6: methyl (1²S,1⁴S)-1¹-((1-(tert-butoxycarbonyl)-5-methoxy-7-methyl-1H-indol-4-yl)methyl)-3,10-dioxa-1(2,4)-piperidina-2(1,2)-benzenacyclodecaphane-2⁴-carboxylate (8): To a solution of tert-butyl 4-(hydroxymethyl)-5-methoxy-7-methyl-1H-indole-1-carboxylate (16 mg, 0.053 mmol) in dichloromethane (2 mL) was added Ph₃PBr₂ (26 mg, 0.62 mmol) at 0° C. and stirred for 1.5 hours under nitrogen. N,N-diisopropylethylamine (19 mg, 0.14 mmol) and (1²S,1⁴S)-3,10-dioxa-1(2,4)-piperidina-2(1,2)-benzenacyclodecaphane-2⁴-carboxylate (16 mg, 0.048 mmol) were added and stirred for 2 hours under nitrogen at 0° C. The reaction was concentrated to dryness under reduced pressure to give the crude product. The residue was further purified by column chromatography on silica gel (petroleum ether:ethyl acetate=10:1 to 3:1) to give methyl (1²S,1⁴S)-11-((1-(tert-butoxycarbonyl)-5-methoxy-7-methyl-1H-indol-4-yl)methyl)-3,10-dioxa-1(2,4)-piperidina-2(1,2)-benzenacyclodecaphane-2⁴-carboxylate (16 mg, Yield 54.9%) as white solid. LC/MS: m/z 607.3 (M+H)⁺.

Step 7: (1²S,1⁴S)-11-((5-methoxy-7-methyl-1H-indol-4-yl)methyl)-3,10-dioxa-1(2,4)-piperidina-2(1,2)-benzenacyclodecaphane-2⁴-carboxylic acid (Example 5): To a mixture of methyl (1²S,1⁴S)-11-((1-(tert-butoxycarbonyl)-5-methoxy-7-methyl-1H-indol-4-yl)methyl)-3,10-dioxa-1(2,4)-piperidina-2(1,2)-benzenacyclodecaphane-2⁴-carboxylate (16 mg, 0.026 mmol) and LiOH·H₂O (3 mg, 0.079 mmol) in methanol (1 mL), water (0.3 mL) and the mixture was stirred at 40° C. overnight. The mixture was diluted with water and extracted with ethyl acetate twice. The combined organic layers were washed with water and brine, dried over anhydrous Na₂SO₄, filtered and concentrated to dryness. The residue was purified by prep-HPLC to give (1²S,1⁴S)-11-((5-methoxy-7-methyl-1H-indol-4-yl)methyl)-3,10-dioxa-1(2,4)-piperidina-2(1,2)-benzenacyclodecaphane-2⁴-carboxylic acid (3 mg, Yield 23.1%) as light-brown solid. ¹H NMR (400 MHz, CD₃OD) δ 7.81 (d, J=8.7 Hz, 1H), 7.81 (d, J=8.7 Hz, 1H), 7.71 (s, 1H), 7.71 (s, 1H), 7.66 (d, J=8.0 Hz, 1H), 7.66 (d, J=8.0 Hz, 1H), 7.33 (d, J=3.1 Hz, 1H), 7.33 (d, J=3.1 Hz, 1H), 6.81 (s, 1H), 6.81 (s, 1H), 6.45 (d, J=3.2 Hz, 1H), 6.45 (d, J=3.2 Hz, 1H), 5.41 (d, J=10.4 Hz, 1H), 4.54 (d, J=12.6 Hz, 1H), 4.51-4.45 (m, 1H), 4.15 (d, J=12.7 Hz, 1H), 3.94 (t, J=9.3 Hz, 1H), 3.87 (s, 3H), 3.73 (s, 1H), 3.63-3.52 (m, 3H), 3.44 (d, J=12.4 Hz, 1H), 2.52 (s, 3H), 2.33 (d, J=13.3 Hz, 1H), 2.09-1.90 (m, 6H), 1.81 (s, 1H), 1.64-1.45 (m, 4H); LC/MS: m/z 493.2 (M+H)⁺.

### Example 6: 3-(2-acetamidoethoxy)-4-((2S,4S)-4-ethoxy-1-((5-methoxy-7-methyl-1H-indol-4-yl) methyl)piperidin-2-yl)benzoic acid

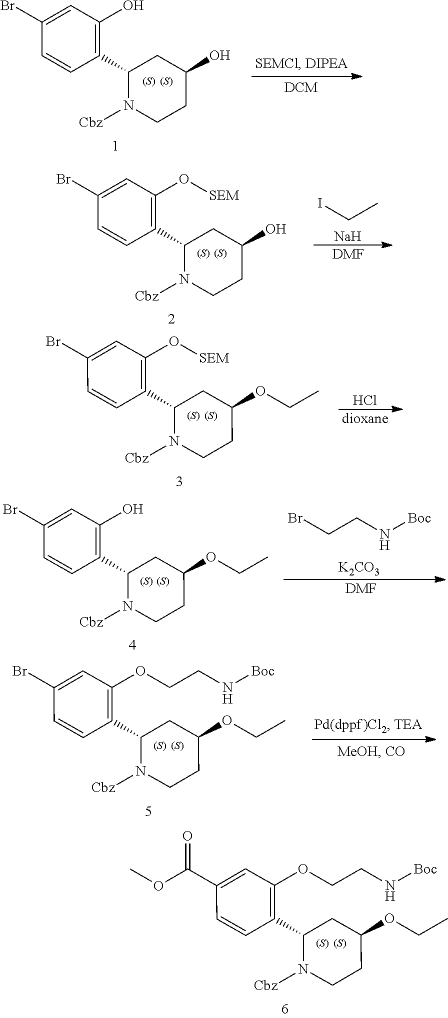

Step 1: benzyl (2S,4S)-2-(4-bromo-2-((2-(trimethylsilyl) ethoxy)methoxy)phenyl)-4-hydroxypiperidine-1-carboxylate (2): To a mixture of benzyl (2S,4S)-2-(4-bromo-2-hydroxyphenyl)-4-hydroxypiperidine-1-carboxylate (550 mg, 1.35 mmol) and DIPEA (349 mg, 2.70 mmol) in dichloromethane (6 mL) under nitrogen, (2-(chloromethoxy)ethyl)trimethylsilane (226 mg, 1.35 mmol) was added and the reaction mixture was stirred at 25° C. for 2 hours. The reaction solution was concentrated to dryness under reduced pressure to give the crude product. The residue was purified by column chromatography on silica gel (petroleum ether:ethyl acetate=20:1 to 3:1) to give benzyl (2S,4S)-2-(4-bromo-2-((2-(trimethylsilyl)ethoxy)methoxy)phenyl)-4-hydroxypiperidine-1-carboxylate (450 mg, Yield 62.3%) as colorless oil. LC/MS: m/z 536/538 (M+H/M+2+H)⁺.

Step 2: benzyl (2S,4S)-2-(4-bromo-2-((2-(trimethylsilyl)ethoxy)methoxy)phenyl)-4-ethoxypiperidine-1-carboxylate (3): To a mixture of benzyl (2S,4S)-2-(4-bromo-2-((2-(trimethylsilyl)ethoxy)methoxy)phenyl)-3-hydroxypiperidine-1-carboxylate (450 mg, 0.84 mmol) in DMF (5 mL) was added NaH (101 mg, 2.51 mmol, 60% dispersion in mineral oil) at 0° C. The mixture was stirred for 0.5 hours under nitrogen before iodoethane (196 mg, 1.26 mmol) was added and the reaction mixture was stirred for additional 1 hour at 25° C. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was purified by column chromatography on silica gel (petroleum ether:ethyl acetate=30:1 to 3:1) to give benzyl (2S,4S)-2-(4-bromo-2-((2-(trimethylsilyl)ethoxy)methoxy)phenyl)-4-ethoxypiperidine-1-carboxylate (260 mg, Yield 55.0%) as colorless oil. LC/MS: 564/566 (M+H/M+2+H)⁺.

Step 3: benzyl (2S,4S)-2-(4-bromo-2-hydroxyphenyl)-4-ethoxypiperidine-1-carboxylate (4): A solution of benzyl (2S,4S)-2-(4-bromo-2-((2-(trimethylsilyl)ethoxy)methoxy)phenyl)-4-ethoxypiperidine-1-carboxylate (260 mg, 0.46 mmol) in HCl/1,4-dioxane (1 mL) was stirred at 25° C. for 1 hour. The mixture was concentrated to dryness under reduced pressure to give benzyl (2S,4S)-2-(4-bromo-2-hydroxyphenyl)-4-ethoxypiperidine-1-carboxylate (180 mg, 90.4% yield) as colorless oil, which was used directly without purification. LC/MS (ESI) m/z: 434/436 (M+H/M+2+H)⁺.

Step 4: benzyl (2S,4S)-2-(4-bromo-2-(2-((tert-butoxycarbonyl)amino)ethoxy)phenyl)-4-ethoxypiperidine-1-carboxylate (5): To a mixture of benzyl (2S,4S)-2-(4-bromo-2-hydroxyphenyl)-4-ethoxypiperidine-1-carboxylate (180 mg, 0.41 mmol) and K₂CO₃ (171 mg, 1.24 mmol) in DMF (2 mL) under nitrogen, tert-butyl (2-bromoethyl)carbamate (138 mg, 0.61 mmol) was added and the reaction mixture was stirred at 25° C. for 2 hours. The mixture was quenched with saturated aq.NH₄Cl solution and extracted with ethyl acetate twice. The combined organic layers were washed with water and brine, dried over anhydrous Na₂SO₄, filtered and concentrated to dryness under reduced pressure. The residue was purified by column chromatography on silica gel (petroleum ether:ethyl acetate=20:1 to 3:1) to give benzyl (2S,4S)-2-(4-bromo-2-(2-((tert-butoxycarbonyl)amino)ethoxy)phenyl)-4-ethoxypiperidine-1-carboxylate (110 mg, Yield 46.7%) as colorless oil. LC/MS: m/z 577/579 (M+H/M+2+H)⁺.

Step 5: benzyl (2S,4S)-2-(2-(2-((tert-butoxycarbonyl)amino)ethoxy)-4-(methoxy-carbonyl)phenyl)-4-ethoxypiperidine-1-carboxylate (6): To a mixture of benzyl (2S,4S)-4-(allyloxy)-2-(4-bromo-2-(pent-4-en-1-yloxy)phenyl) piperidine-1-carboxylate (110 mg, 0.19 mmol) and TEA (57 mg, 0.57 mmol) in methanol (5 mL) was added Pd(dppf)Cl₂ (14 mg, 0.019 mmol) at room temperature under CO atmosphere and the mixture was stirred at 70° C. for 16 hours. The mixture was concentrated to dryness under reduced pressure. The residue was purified by column chromatography on silica gel (petroleum ether:ethyl acetate=5:1 to 1:1) to give benzyl (2S,4S)-2-(2-(2-((tert-butoxycarbonyl)amino)ethoxy)-4-(methoxycarbonyl)phenyl)-4-ethoxypiperidine-1-carboxylate (37 mg, Yield 34.9%) as colorless oil. LC/MS: m/z 557 (M+H)⁺.

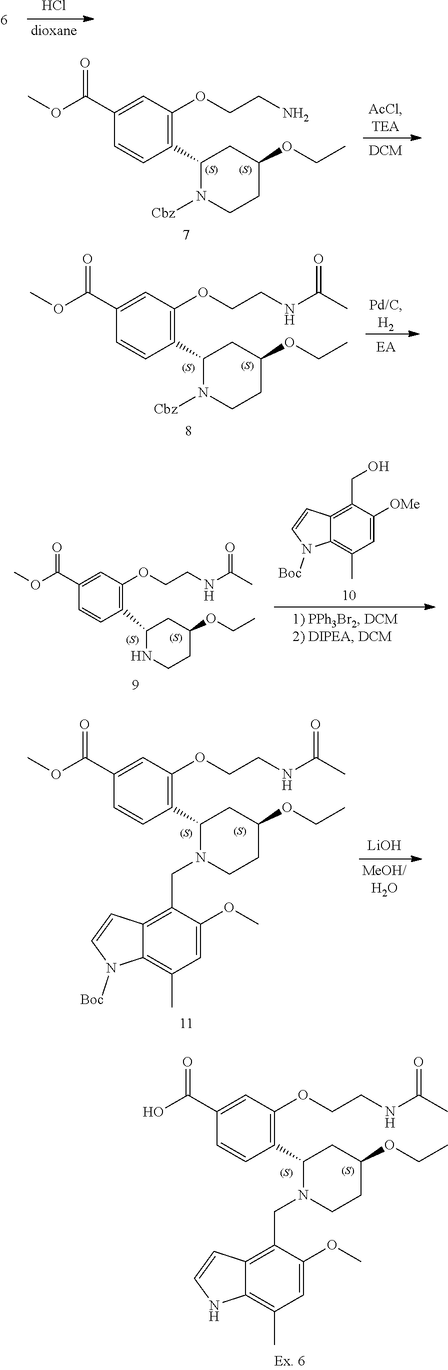

Step 6: benzyl (2S,4S)-2-(2-(2-aminoethoxy)-4-(methoxycarbonyl)phenyl)-4-ethoxypiperidine-1-carboxylate (7): A solution of benzyl (2S,4S)-2-(2-(2-((tert-butoxycarbonyl)amino)ethoxy)-4-(methoxycarbonyl)phenyl)-4-ethoxypiperidine-1-carboxylate (37 mg, 0.20 mmol) in HCl/1,4-dioxane (1 mL) was stirred at 25° C. for 1 hour. The mixture was concentrated to dryness under reduced pressure to give benzyl (2S,4S)-2-(2-(2-aminoethoxy)-4-(methoxycarbonyl)phenyl)-4-ethoxypiperidine-1-carboxylate (44 mg, 88.0% yield) as white solid, which was used directly without purification. LC/MS: m/z 457 (M+H)⁺.

Step 7: benzyl (2S,4S)-2-(2-(2-acetamidoethoxy)-4-(methoxycarbonyl)phenyl)-4-ethoxypiperidine-1-carboxylate (8): To a solution of benzyl (2S,4S)-2-(2-(2-aminoethoxy)-4-(methoxycarbonyl)phenyl)-4-ethoxypiperidine-1-carboxylate (37 mg, 0.08 mmol) and NaHCO₃ (20 mg, 0.24 mmol) in tetrahydrofuran (2 mL) and water (0.5 mL) was added acetyl chloride (12 mg, 0.16 mmol). The mixture was stirred at room temperature under N₂ atmosphere overnight. The reaction was quenched with 1N aq.HCl solution. The resulting mixture was extracted with ethyl acetate (2×50 mL). The combined organic layers were washed with water (5 mL) and brine (5 mL), dried over anhydrous Na₂SO₄, filtered and concentrated to dryness under reduced pressure and purified by column chromatography on silica gel (petroleum ether:ethyl acetate=20:1 to 1:1) to give benzyl (2S,4S)-2-(2-(2-acetamidoethoxy)-4-(methoxycarbonyl)phenyl)-4-ethoxypiperidine-1-carboxylate (25 mg, 62.6% yield) as white solid. LC/MS (ESI) m/z: 499 (M+H)⁺.

Step 8: methyl 3-(2-acetamidoethoxy)-4-((2S,4S)-4-ethoxypiperidin-2-yl)benzoate (9): To a solution of benzyl (2S,4S)-2-(2-(2-acetamidoethoxy)-4-(methoxycarbonyl)phenyl)-4-ethoxypiperidine-1-carboxylate (25 mg, 0.05 mmol) in ethyl acetate (1 mL) was added Pd/C (5 mg, 10% wt) at 25° C. under N₂ atmosphere and the mixture was stirred under H₂ atmosphere at 40° C. for 16 hours. The mixture was filtered and the filtrate was concentrated to dryness under reduced pressure to give methyl 3-(2-acetamidoethoxy)-4-((2S,4S)-4-ethoxypiperidin-2-yl)benzoate (14 mg, yield 76.9%) as light oil, which was used directly in the next step without further purification. LC/MS(ESI) m/z: 365 (M+H)⁺.

Step 9: tert-butyl 4-(((2S,4S)-2-(2-(2-acetamidoethoxy)-4-(methoxycarbonyl)phenyl)-4-ethoxypiperidin-1-yl)methyl)-5-methoxy-7-methyl-1H-indole-1-carboxylate (11): To a mixture of tert-butyl 4-(hydroxymethyl)-5-methoxy-7-methyl-1H-indole-1-carboxylate (17 mg, 0.057 mmol) in dichloromethane (2 mL) was added Ph₃PBr₂ (24 mg, 0.057 mmol) at 0° C. The mixture was stirred for 1.5 hours under nitrogen before N, N-diisopropylethylamine (19 mg, 0.14 mmol) and methyl 3-(2-acetamidoethoxy)-4-((2S,4S)-4-ethoxypiperidin-2-yl)benzoate (14 mg, 0.038 mmol) were added. The reaction was stirred for additional 2 hours under nitrogen at 0° C. before concentrated under reduced pressure to give the crude product. The residue was purified by column chromatography on silica gel (petroleum ether:ethyl acetate=10:1 to 3:1) to give tert-butyl 4-(((2S,4S)-2-(2-(2-acetamidoethoxy)-4-(methoxycarbonyl)phenyl)-4-ethoxypiperidin-1-yl)methyl)-5-methoxy-7-methyl-1H-indole-1-carboxylate (12 mg, Yield 49.5%) as white solid. LC/MS: m/z 638 (M+H)⁺.

Step 10: 3-(2-acetamidoethoxy)-4-((2S,4S)-4-ethoxy-1-((5-methoxy-7-methyl-1H-indol-4-yl)methyl)piperidin-2-yl)benzoic acid (Example 6): A mixture of tert-butyl 4-(((2S,4S)-2-(2-(2-acetamidoethoxy)-4-(methoxycarbonyl)phenyl)-4-ethoxypiperidin-1-yl)methyl)-5-methoxy-7-methyl-1H-indole-1-carboxylate (12 mg, 0.018 mmol) and LiOH (3 mg, 0.079 mmol) in methanol (1 mL)/water (0.3 mL) was stirred at room temperature overnight. The reaction was quenched with 1 N aq.HCl solution before diluted with water and extracted with ethyl acetate twice. The combined organic layers were washed with water and brine, dried over anhydrous Na₂SO₄, filtered and concentrated to dryness under reduced pressure. The residue was purified by prep-HPLC to give 3-(2-acetamidoethoxy)-4-((2S,4S)-4-ethoxy-1-((5-methoxy-7-methyl-1H-indol-4-yl)methyl)piperidin-2-yl)benzoic acid (2 mg, Yield 21.2%) as white solid. ¹H NMR (400 MHz, CD₃OD) δ 7.83-7.69 (m, 2H), 7.59 (d, J=17.2 Hz, 1H), 7.30 (t, J=4.4 Hz, 1H), 6.76 (d, J=10.9 Hz, 1H), 6.21 (s, 1H), 4.37-4.27 (m, 2H), 4.26-4.10 (m, 2H), 3.81 (d, J=27.2 Hz, 5H), 3.66-3.51 (m, 4H), 3.49-3.34 (m, 1H), 2.89-2.56 (m, 1H), 2.50 (s, 3H), 2.46-2.26 (m, 1H), 2.21-2.02 (m, 3H), 1.32 (t, J=7.0 Hz, 6H); LC/MS: m/z 524 (M+H)⁺.

### Example 7: (1²S,1⁴S)_1¹-((5-methoxy-7-methyl-1H-indol-4-yl)methyl)-3,11-dioxa-1(2,4)-piperidina-2(1,2)-benzenacycloundecaphane-2⁴-carboxylic acid

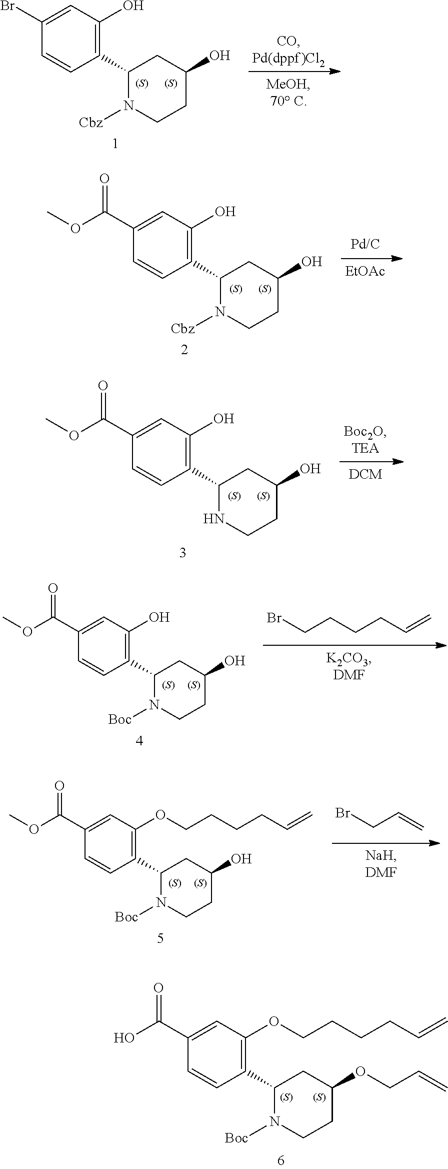

Step 1: benzyl (2S,4S)-4-hydroxy-2-(2-hydroxy-4-(methoxycarbonyl)phenyl) piperidine-1-carboxylate (2): To a mixture of benzyl (2S,4S)-2-(4-bromo-2-hydroxyphenyl)-4-hydroxypiperidine-1-carboxylate (1 g, 2.46 mmol) and TEA (746 mg, 7.48 mmol) in methanol (15 mL) was added Pd(dppf)Cl₂ (358 mg, 0.49 mmol) at room temperature under CO atmosphere and the mixture was stirred at 70° C. for 16 hours. The mixture was concentrated to dryness under reduced pressure. The residue was purified by column chromatography on silica gel (petroleum ether:ethyl acetate=5:1 to 1:1) to give benzyl (2S,4S)-4-hydroxy-2-(2-hydroxy-4-(methoxycarbonyl)phenyl)piperidine-1-carboxylate (400 mg, Yield 42.2%) as yellow oil. LC/MS (ESI) m/z: 386 (M+H)⁺.

Step 2: methyl 3-hydroxy-4-((2S,4S)-4-hydroxypiperidin-2-yl)benzoate (3): To a solution of benzyl (2S,4S)-4-hydroxy-2-(2-hydroxy-4-(methoxycarbonyl)phenyl)piperidine-1-carboxylate (350 mg, 0.91 mmol) in ethyl acetate (5 mL) was added Pd/C (30 mg) at 25° C. under N₂ atmosphere and the mixture was stirred under H₂ atmosphere at 30° C. for 3 hours.

The mixture was filtered and the filtrate was concentrated to dryness under reduced pressure to give methyl 3-hydroxy-4-((2S,4S)-4-hydroxypiperidin-2-yl)benzoate (220 mg, yield 96.4%) as oil, which was used directly in the next step without further purification. LC/MS(ESI) m/z: 252 (M+H)⁺.

Step 3: tert-butyl (2S,4S)-4-hydroxy-2-(2-hydroxy-4-(methoxycarbonyl)phenyl) piperidine-1-carboxylate (4): To a solution of methyl 3-hydroxy-4-((2S,4S)-4-hydroxypiperidin-2-yl)benzoate (220 mg, 0.87 mmol) in THF (3 mL) and H₂O(1 mL) was added NaHCO₃ (220 mg, 2.62 mmol) and Boc₂O (379 mg, 1.74 mmol). The mixture was stirred at 25° C. for 3 hours before concentrated to dryness under reduced pressure and the residue was purified by flash chromatography (DCM:MeOH=10:1) to give tert-butyl (2S,4S)-4-hydroxy-2-(2-hydroxy-4-(methoxycarbonyl)phenyl)piperidine-1-carboxylate (290 mg, 95.1% yield) as white solid. LC/MS (ESI) m/z: 352 (M+H)⁺.

Step 4: tert-butyl (2S,4S)-2-(2-(hex-5-en-1-yloxy)-4-(methoxycarbonyl)phenyl)-4-hydroxypiperidine-1-carboxylate (5): To a solution of tert-butyl (2S,4S)-4-hydroxy-2-(2-hydroxy-4-(methoxycarbonyl)phenyl) piperidine-1-carboxylate (140 mg, 0.40 mmol) in DMF (2 mL) was added K₂CO₃ (220 mg, 1.59 mmol) and 6-bromohex-1-ene (130 mg, 0.80 mmol) and the mixture was stirred at 25° C. for 18 hours. The mixture was concentrated to dryness under reduced pressure and the residue was purified by flash chromatography (petroleum ether:ethyl acetate=1:1) to give tert-butyl (2S,4S)-2-(2-(hex-5-en-1-yloxy)-4-(methoxycarbonyl)phenyl)-4-hydroxypiperidine-1-carboxylate (110 mg, 64.0% yield) as white solid. LC/MS (ESI) m/z: 434 (M+H)⁺.

Step 5: 4-((2S,4S)-4-(allyloxy)-1-(tert-butoxycarbonyl)piperidin-2-yl)-3-(hex-5-en-1-yloxy)benzoic acid (6); To a solution of tert-butyl (2S,4S)-2-(2-(hex-5-en-1-yloxy)-4-(methoxycarbonyl)phenyl)-4-hydroxypiperidine-1-carboxylate (110 mg, 0.25 mmol) in DMF (2 mL) was added NaH (40 mg, 1.01 mmol, 60% dispersion in mineral oil) at 0° C. The mixture was stirred for half an hour under nitrogen before 3-bromoprop-1-ene (46 mg, 0.38 mmol) was added and the mixture was stirred for 18 hours at 25° C. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was purified by column chromatography on silica gel (petroleum ether:ethyl acetate=1:1) to give 4-((2S,4S)-4-(allyloxy)-1-(tert-butoxycarbonyl)piperidin-2-yl)-3-(hex-5-en-1-yloxy)benzoic acid (110 mg, Yield 94.8%) as colorless oil. LC/MS: m/z 460 (M+H)⁺.

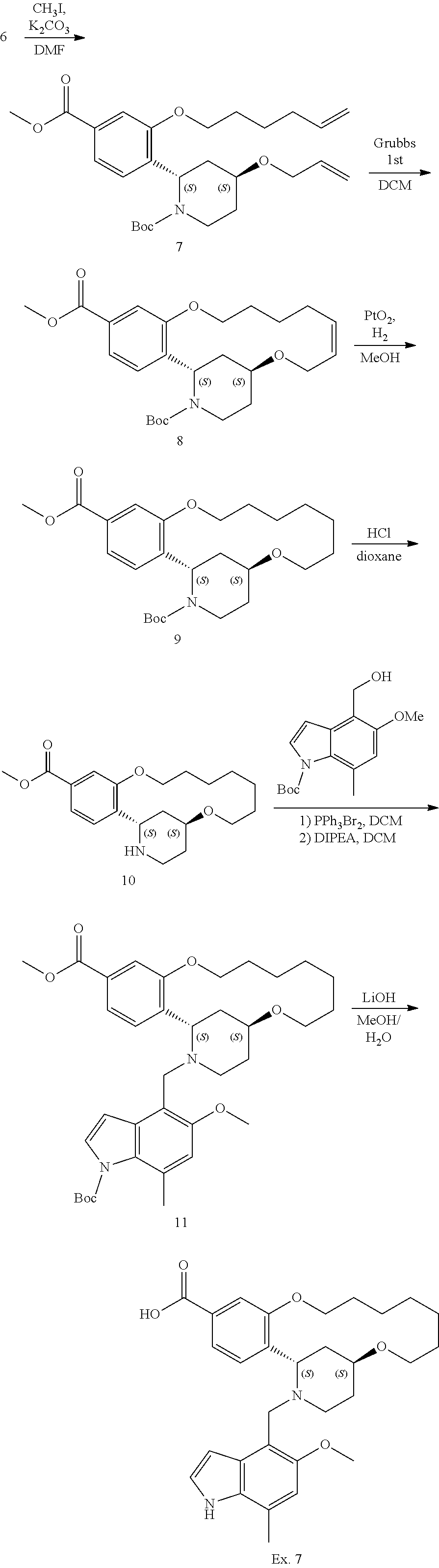

Step 6: tert-butyl (2S,4S)-4-(allyloxy)-2-(2-(hex-5-en-1-yloxy)-4-(methoxycarbonyl)phenyl)piperidine-1-carboxylate (7): To a mixture of 4-((2S,4S)-4-(allyloxy)-1-(tert-butoxycarbonyl)piperidin-2-yl)-3-(hex-5-en-1-yloxy)benzoic acid (110 mg, 0.24 mmol) in DMF (2 mL) was added K₂CO₃ (99 mg, 0.72 mmol) and iodomethane (204 mg, 1.44 mmol) and the mixture was stirred at 25° C. for 1 hours. The mixture was concentrated to dryness under reduced pressure and the residue was purified by flash chromatography (petroleum ether:ethyl acetate=3:1) to give tert-butyl (2S,4S)-4-(allyloxy)-2-(2-(hex-5-en-1-yloxy)-4-(methoxycarbonyl)phenyl)piperidine-1-carboxylate (93 mg, 81.5% yield) as colorless oil. LC/MS (ESI) m/z: 474 (M+H)⁺.

Step 7: 1¹-(tert-butyl) 2⁴-methyl (1²S,1⁴S,Z)-3,11-dioxa-1(2,4)-piperidina-2(1,2)-benzenacycloundecaphan-8-ene-1¹,2⁴-dicarboxylate (8): To a solution of tert-butyl (2S,4S)-4-(allyloxy)-2-(2-(hex-5-en-1-yloxy)-4-(methoxycarbonyl)phenyl)piperidine-1-carboxylate (93 mg, 0.20 mmol) in dry DCM (93 mL) was added Grubbs 1^{st} (32 mg, 0.039 mmol) and the mixture was stirred at 25° C. for 18 hours under nitrogen. The mixture was concentrated to dryness under reduced pressure and the residue was purified by flash chromatography (petroleum ether:ethyl acetate=3:1) to give compound 8 (63 mg, 72.4% yield) as brown oil. LC/MS (ESI) m/z: 446 (M+H)⁺.

Step 8: 1¹-(tert-butyl) 2⁴-methyl (1²S,1⁴S)-3,11-dioxa-1(2,4)-piperidina-2(1,2)-benzenacycloundecaphane-1¹,2⁴-dicarboxylate (9): To a solution of compound 8 (60 mg, 0.13 mmol) in methanol (2 mL) was added PtO₂ (6 mg) at 25° C. under nitrogen and the mixture was stirred under H₂ atmosphere at 30° C. for 30 minutes. The mixture was filtered and the filtrate was concentrated to dryness under reduced pressure to give 1¹-(tert-butyl) 2⁴-methyl (1²S,1⁴S)-3,11-dioxa-1(2,4)-piperidina-2(1,2)-benzenacycloundecaphane-1¹,2⁴-dicarboxylate (48 mg, yield 82.7%) as oil, which was used directly in the next step without further purification. LC/MS(ESI) m/z: 448 (M+H)⁺.

Step 9: methyl (1²S,1⁴S)-3,11-dioxa-1(2,4)-piperidina-2(1,2)-benzenacycloundecaphane-2⁴-carboxylate (10): A mixture of 1¹-(tert-butyl) 2⁴-methyl (1²S,1⁴S)-3,11-dioxa-1(2,4)-piperidina-2(1,2)-benzenacycloundecaphane-1¹,2⁴-dicarboxylate (48 mg, 0.11 mmol) in HCl/1,4-dioxane (1 mL) was stirred at 25° C. for 1 hour. The mixture was concentrated to dryness under reduced pressure to give methyl (1²S,1⁴S)-3,11-dioxa-1(2,4)-piperidina-2(1,2)-benzenacycloundecaphane-2⁴-carboxylate (36 mg, 94.9% yield) as white solid, which was used directly in the next step without further purification. LC/MS (ESI) m/z: 348 (M+H)⁺.

Step 10: methyl (1²S,1⁴S)-1¹-((1-(tert-butoxycarbonyl)-5-methoxy-7-methyl-1H-indol-4-yl)methyl)-3,11-dioxa-1(2,4)-piperidina-2(1,2)-benzenacycloundecaphane-2⁴-carboxylate (11): To a solution of tert-butyl 4-(hydroxymethyl)-5-methoxy-7-methyl-1H-indole-1-carboxylate (35 mg, 0.12 mmol) in dry DCM (1 mL) was added PPh₃Br₂ (63 mg, 0.15 mmol). The mixture was stirred for 2 hours at 0° C. under nitrogen before (1²S, 1⁴S) 3,11-dioxa-1(2,4)-piperidina-2(1,2)-benzenacycloundecaphane-2⁴-carboxylate (36 mg, 0.11 mmol) and DIPEA (45 mg, 0.35 mmol) in dry DCM (1 mL) was added. The mixture was stirred for 1.5 hours at 0° C. before concentrated to dryness under reduced pressure. The residue was purified by prep-HPLC to give methyl (1²S,1⁴S)-1′-((1-(tert-butoxycarbonyl)-5-methoxy-7-methyl-1H-indol-4-yl)methyl)-3,11-dioxa-1(2,4)-piperidina-2(1,2)-benzenacycloundecaphane-2⁴-carboxylate (34 mg, 50.0% yield) as colorless oil. LC/MS (ESI) m/z: 621 (M+H)⁺.

Step 11: (1²S,1⁴S)_1¹-((5-methoxy-7-methyl-1H-indol-4-yl)methyl)-3,11-dioxa-1(2,4)-piperidina-2(1,2)-benzenacycloundecaphane-2⁴-carboxylic acid (Example 7): A mixture of methyl (1²S,1⁴S)-11-((1-(tert-butoxycarbonyl)-5-methoxy-7-methyl-1H-indol-4-yl)methyl)-3,11-dioxa-1(2,4)-piperidina-2(1,2)-benzenacycloundecaphane-2⁴-carboxylate (17 mg, 0.027 mmol) and LiOH·H₂O (7 mg, 0.16 mmol) in methanol (1 mL)/water (0.3 mL) was stirred at room temperature overnight. The reaction was quenched with 1N aq.HCl solution.

The mixture was diluted with water and extracted with ethyl acetate twice. The combined organic layers were washed with water and brine, dried over anhydrous Na₂SO₄, filtered and concentrated to dryness under reduced pressure. The residue was purified by prep-HPLC to give (1², 1⁴S)-1¹-((5-methoxy-7-methyl-1H-indol-4-yl)methyl)-3,11-dioxa-1(2,4)-piperidina-2(1,2)-benzenacycloundecaphane-2⁴-carboxylic acid (3.7 mg, Yield 27.2%) as white solid. ¹H NMR (400 MHz,) δ 7.81 (dd, J=8.0, 1.2 Hz, 1H), 7.71 (s, 1H), 7.64 (d, J=8.0 Hz, 1H), 7.32 (d, J=3.1 Hz, 1H), 6.78 (s, 1H), 6.35 (d, J=3.2 Hz, 1H), 5.37 (dd, J=12.5, 2.5 Hz, 1H), 4.38 (d, J=12.7 Hz, 2H), 4.10 (d, J=12.6 Hz, 2H), 3.81 (s, 3H), 3.74 (s, 1H), 3.57 (t, J=8.3 Hz, 3H), 3.41 (d, J=12.4 Hz, 1H), 2.51 (s, 3H), 2.28 (d, J=12.8 Hz, 1H), 2.08 (d, J=13.0 Hz, 2H), 2.04-1.92 (m, 4H), 1.86-1.50 (m, 6H), 1.38 (s, 1H). LC/MS: m/z 507 (M+H)⁺.

### Example 8: (1²S,1⁴S)_1¹-((5-methoxy-7-methyl-1H-indol-4-yl)methyl)-3,9-dioxa-1(2,4)-piperidina-2(1,2)-benzenacyclononaphane-2⁴-carboxylic acid

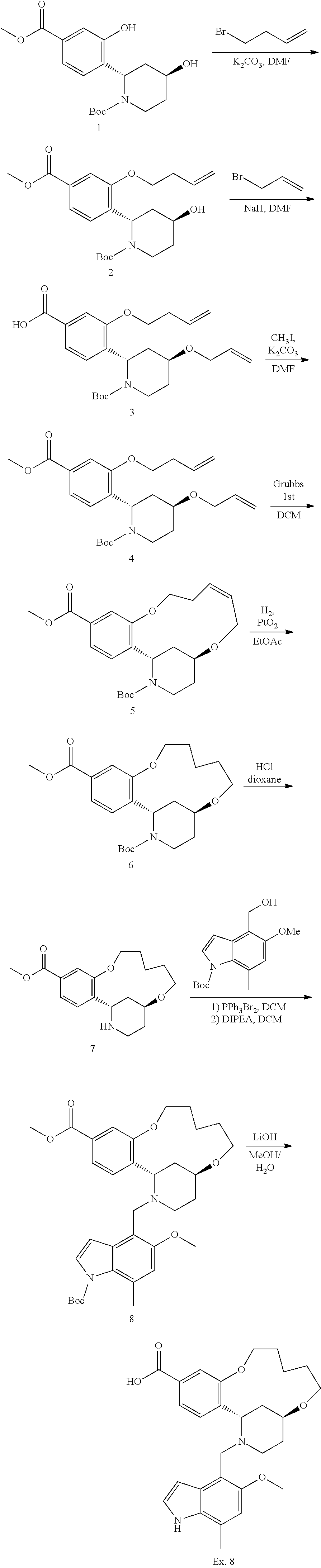

Step 1: tert-butyl (2S,4S)-2-(2-(but-3-en-1-yloxy)-4-(methoxycarbonyl)phenyl)-4-hydroxypiperidine-1-carboxylate: To a solution of tert-butyl (2S,4S)-4-hydroxy-2-(2-hydroxy-4-(methoxycarbonyl)phenyl) piperidine-1-carboxylate (160 mg, 0.45 mmol) in DMF (2 mL) was added K₂CO₃ (251 mg, 1.82 mmol) and 4-bromobut-1-ene (123 mg, 0.91 mmol) and the mixture was stirred at 25° C. for 18 hours. The mixture was concentrated to dryness under reduced pressure and the residue was purified by flash chromatography (petroleum ether:ethyl acetate=1:1) to give tert-butyl (2S,4S)-2-(2-(but-3-en-1-yloxy)-4-(methoxycarbonyl)phenyl)-4-hydroxypiperidine-1-carboxylate (110 mg, 60.3% yield) as white solid. LC/MS (ESI) m/z: 406 (M+H)⁺.

Step 2: 4-((2S,4S)-4-(allyloxy)-1-(tert-butoxycarbonyl)piperidin-2-yl)-3-(but-3-en-1-yloxy)benzoic acid: To a solution of tert-butyl (2S,4S)-2-(2-(but-3-en-1-yloxy)-4-(methoxycarbonyl)phenyl)-4-hydroxypiperidine-1-carboxylate (110 mg, 0.27 mmol) in DMF (2 mL) was added NaH (50 mg, 1.62 mmol, 60% dispersion in mineral oil) in-portions at 0° C., and the mixture was stirred under N₂ atmosphere for 0.5 hour. Then 3-bromoprop-1-ene (65 mg, 0.41 mmol) was added and the resulting mixture was stirred at 25° C. for 18 hours. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was purified by column chromatography on silica gel (petroleum ether:ethyl acetate=1:1) to give 4-((2S,4S)-4-(allyloxy)-1-(tert-butoxycarbonyl)piperidin-2-yl)-3-(but-3-en-1-yloxy)benzoic acid (110 mg, yield 94.8%) as colorless oil. LC/MS: m/z 431 (M+H)⁺.

Step 3: tert-butyl (2S,4S)-4-(allyloxy)-2-(2-(but-3-en-1-yloxy)-4-(methoxycarbonyl)phenyl) piperidine-1-carboxylate: To a solution of 4-((2S,4S)-4-(allyloxy)-1-(tert-butoxycarbonyl)piperidin-2-yl)-3-(but-3-en-1-yloxy)benzoic acid (110 mg, 0.26 mmol) in DMF (2 mL) was added K₂CO₃ (106 mg, 0.77 mmol) and iodomethane (219 mg, 1.54 mmol) and the mixture was stirred at 25° C. for 1 hour. The mixture was concentrated to dryness under reduced pressure and the residue was purified by flash chromatography (petroleum ether:ethyl acetate=3:1) to give tert-butyl (2S,4S)-4-(allyloxy)-2-(2-(but-3-en-1-yloxy)-4-(methoxycarbonyl)phenyl) piperidine-1-carboxylate (90 mg, 77.5% yield) as colorless oil. LC/MS (ESI) m/z: 446 (M+H)⁺.

Step 4: 1¹-(tert-butyl) 2⁴-methyl (1²S,1⁴S,Z)-3,9-dioxa-1(2,4)-piperidina-2(1,2)-benzenacyclononaphan-6-ene-1¹,2⁴-dicarboxylate: To a solution of tert-butyl (2S,4S)-4-(allyloxy)-2-(2-(but-3-en-1-yloxy)-4-(methoxycarbonyl)phenyl)piperidine-1-carboxylate (90 mg, 0.20 mmol) in dry DCM (90 mL) was added Grubbs 1^{st} catalyst (33 mg, 0.04 mmol) and the mixture was stirred under N₂ atmosphere at 25° C. for 18 hours. The mixture was concentrated to dryness under reduced pressure and the residue was purified by flash chromatography (petroleum ether:ethyl acetate=3:1) to give 1′-(tert-butyl) 2⁴-methyl (1²,1⁴S,Z)-3,9-dioxa-1(2,4)-piperidina-2(1,2)-benzenacyclononaphan-6-ene-1¹,2⁴-dicarboxylate (52 mg, 61.9% yield) as brown oil. LC/MS (ESI) m/z: 418 (M+H)⁺.

Step 5: 1¹-(tert-butyl) 2⁴-methyl (1²S,1⁴S)-3,9-dioxa-1(2,4)-piperidina-2(1,2)-benzenacyclononaphane-1¹,2⁴-dicarboxylate: To a solution of 1¹-(tert-butyl) 2⁴-methyl (1²,1⁴S,E)-3,9-dioxa-1(2,4)-piperidina-2(1,2)-benzenacyclononaphan-6-ene-1¹,2⁴-dicarboxylate (30 mg, 0.07 mmol) in methanol (2 mL) was added PtO₂ (6 mg) at 25° C. under N₂ atmosphere and the mixture was stirred under a H₂ balloon at 30° C. for 30 minutes. The mixture was filtered and the filtrate was concentrated to dryness under reduced pressure to give 1¹-(tert-butyl) 2⁴-methyl (1²S,1⁴S)-3,9-dioxa-1(2,4)-piperidina-2(1,2)-benzenacyclononaphane-1¹,2⁴-dicarboxylate (17 mg, 56.7% yield) as light-yellow oil, which was used directly in the next step without further purification. LC/MS (ESI) m/z: 421 (M+H)⁺.

Step 6: methyl (1²S,1⁴S)-3,9-dioxa-1(2,4)-piperidina-2(1,2)-benzenacyclononaphane-2⁴-carboxylate hydrochloride: A mixture of methyl 1¹-(tert-butyl) 2⁴-methyl (1²S,1⁴S)-3,9-dioxa-1(2,4)-piperidina-2(1,2)-benzenacyclononaphane-1¹,2⁴-dicarboxylate (17 mg, 0.04 mmol) in HCl/1,4-dioxane (1 mL) was stirred at 25° C. for 1 hour.

The mixture was concentrated to dryness under reduced pressure to give methyl (1²S,1⁴S) 3,9-dioxa-1(2,4)-piperidina-2(1,2)-benzenacyclononaphane-2⁴-carboxylate (11 mg, 90.6% yield) as white solid, which was used directly without further purification. LC/MS (ESI) m/z: 321 (M+H)⁺.

Step 7: methyl (1²S,1⁴S)-1¹-((1-(tert-butoxycarbonyl)-5-methoxy-7-methyl-1H-indol-4-yl)methyl)-3,9-dioxa-1(2,4)-piperidina-2(1,2)-benzenacyclononaphane-2⁴-carboxylate: To a solution of tert-butyl 4-(hydroxymethyl)-5-methoxy-7-methyl-1H-indole-1-carboxylate (12 mg, 0.041 mmol) in dry DCM (1 mL) was added PPh₃Br₂ (19 mg, 0.044 mmol) in portions at 0° C., and the mixture stirred for 2 hours. Then methyl (1²S,1⁴S)-3,9-dioxa-1 (2,4)-piperidina-2(1,2)-benzenacyclononaphane-2⁴-carboxylate (11 mg, 0.034 mmol) and DIPEA (13 mg, 0.10 mmol) in dry DCM (1 mL) was added and the mixture was stirred for 1.5 hours at 0° C. The mixture was concentrated to dryness under reduced pressure and the residue was purified by prep-HPLC to give methyl (1²S,1⁴S)-11-((1-(tert-butoxycarbonyl)-5-methoxy-7-methyl-1H-indol-4-yl)methyl)-3,9-dioxa-1(2,4)-piperidina-2(1,2)-benzenacyclononaphane-2⁴-carboxylate (15 mg, 75.0% yield) as colorless oil. LC/MS (ESI) m/z: 593 (M+H)⁺.

Step 8: (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (Example 8): To a solution of methyl (1²S,1⁴S)_11-((1-(tert-butoxycarbonyl)-5-methoxy-7-methyl-1H-indol-4-yl)methyl)-3,9-dioxa-1(2,4)-piperidina-2(1,2)-benzenacyclononaphane-2⁴-carboxylate (15 mg, 0.025 mmol) in methanol (1 mL)/water (0.3 mL) was added and LiOH H₂O (7 mg, 0.16 mmol) and the mixture was stirred at room temperature overnight. The mixture was acidified with 1 N aq.HCl to pH˜4 and extracted with ethyl acetate twice. The combined organic layers were washed with water and brine, dried over anhydrous Na₂SO₄, filtered and concentrated to dryness under reduced pressure. The residue was purified by prep-HPLC to give (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (2.2 mg, yield 18.3%) as white solid. ¹H NMR (400 MHz, CD₃OD) δ 8.38 (s, 1H), 7.88 (d, J=7.9 Hz, 1H), 7.83 (s, 1H), 7.63 (d, J=8.0 Hz, 1H), 7.33 (d, J=3.2 Hz, 1H), 6.80 (s, 1H), 6.42 (d, J=3.1 Hz, 1H), 5.55 (d, J=11.2 Hz, 1H), 4.58 (s, 1H), 4.43 (t, J=10.7 Hz, 2H), 4.25 (d, J=11.4 Hz, 1H), 4.13 (d, J=12.9 Hz, 1H), 3.84 (s, 3H), 3.80 (s, 1H), 3.69 (d, J=10.5 Hz, 1H), 3.60 (dd, J=16.1, 9.3 Hz, 2H), 3.44 (d, J=11.9 Hz, 1H), 2.51 (s, 3H), 2.50-2.41 (m, 1H), 2.36 (d, J=15.5 Hz, 1H), 2.15-2.07 (m, 1H), 2.01 (dd, J=13.5, 9.3 Hz, 2H), 1.93-1.85 (m, 1H), 1.69-1.62 (m, 3H). LC/MS (ESI) m/z: 479 (M+H)⁺.

### Example 9: (1²S,1⁴S,Z)-1¹-((5-methoxy-7-methyl-1H-indol-4-yl)methyl)-3,9-dioxa-1(2,4)-piperidina-2(1,2)-benzenacyclononaphan-6-ene-2⁴-carboxylic acid

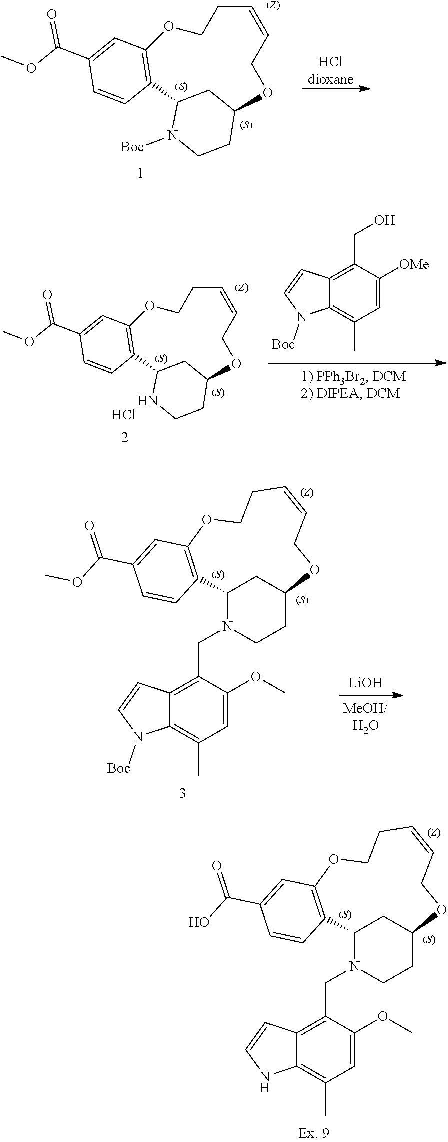

Step 1: methyl (1²S,1⁴S, Z)-3,9-dioxa-1(2,4)-piperidina-2(1,2)-benzenacyclononaphan-6-ene-2⁴-carboxylate hydrochloride: A solution of 1¹-(tert-butyl) 2⁴-methyl (1²S,1⁴S,E)-3,9-dioxa-1(2,4)-piperidina-2(1,2)-benzenacyclononaphan-6-ene-1¹,2⁴-dicarboxylate (20 mg, 0.05 mmol) in HCl/1,4-dioxane (1 mL) was stirred at 25° C. for 1 hour. The mixture was concentrated to dryness under reduced pressure to give methyl (1²S,1⁴S,E)-3,9-dioxa-1(2,4)-piperidina-2(1,2)-benzenacyclononaphan-6-ene-2⁴-carboxylate (18 mg, 100% yield) as yellow solid, which was used directly in the next step without further purification. LC/MS (ESI) m/z: 318 (M+H)⁺.

Step 2: methyl (1²S,1⁴S,Z)-11-((1-(tert-butoxycarbonyl)-5-methoxy-7-methyl-1H-indol-4-yl)methyl)-3,9-dioxa-1(2,4)-piperidina-2(1,2)-benzenacyclononaphan-6-ene-2⁴-carboxylate: To a solution of tert-butyl 4-(hydroxymethyl)-5-methoxy-7-methyl-1H-indole-1-carboxylate (16 mg, 0.056 mmol) in dry DCM (1 mL) was added PPh₃Br₂ (26 mg, 0.061 mmol) in portions at 0° C., and the mixture was stirred under N₂ atmosphere for 2 hours.

Then methyl (1²S,1⁴S,E)-3,9-dioxa-1(2,4)-piperidina-2(1,2)-benzenacyclononaphan-6-ene-2⁴-carboxylate hydrochloride (18 mg, 0.05 mmol) and DIPEA (18 mg, 0.14 mmol) in dry DCM (1 mL) was added and the mixture was stirred at 0° C. for 1.5 hours. The mixture was concentrated to dryness under reduced pressure and the residue was purified by prep-HPLC to give methyl (1²S,1⁴S,Z)-11-((1-(tert-butoxycarbonyl)-5-methoxy-7-methyl-1H-indol-4-yl)methyl)-3,9-dioxa-1(2,4)-piperidina-2(1,2)-benzenacyclononaphan-6-ene-2⁴-carboxylate (17 mg, 62.9% yield) as off-white solid. LC/MS (ESI) m/z: 592 (M+H)⁺.

Step 3: (1²S,1⁴S,Z)-11-((5-methoxy-7-methyl-1H-indol-4-yl)methyl)-3,9-dioxa-1(2,4)-piperidina-2(1,2)-benzenacyclononaphan-6-ene-2⁴-carboxylic acid (Example 9): To a mixture of methyl (1²S,1⁴S,Z)-11-((1-(tert-butoxycarbonyl)-5-methoxy-7-methyl-1H-indol-4-yl)methyl)-3,9-dioxa-1(2,4)-piperidina-2(1,2)-benzenacyclononaphan-6-ene-2⁴-carboxylate (17 mg, 0.029 mmol) and LiOH H₂O (4 mg, 0.086 mmol) in methanol (1 mL)/water (0.3 mL) and the mixture was stirred at room temperature overnight. The reaction was acidified with 1 N aq.HCl to pH˜4 and extracted with ethyl acetate twice. The combined organic layers were washed with water and brine, dried over anhydrous Na₂SO₄, filtered and concentrated to dryness under reduced pressure. The residue was purified by prep-HPLC to give (1²S,1⁴S,E)-11-((5-methoxy-7-methyl-1H-indol-4-yl)methyl)-3,9-dioxa-1(2,4)-piperidina-2(1,2)-benzenacyclononaphan-6-ene-24-carboxylic acid (2.4 mg, yield 18.5%) as white solid. ¹H NMR (400 MHz, CD₃OD) δ 8.44 (s, 1H), 7.81 (d, J=8.0 Hz, 1H), 7.74-7.63 (m, 2H), 7.35 (d, J=3.1 Hz, 1H), 6.81 (s, 1H), 6.51 (d, J=3.1 Hz, 1H), 6.02 (ddd, J=14.0, 10.5, 3.2 Hz, 1H), 5.72-5.57 (m, 1H), 5.32 (dd, J=12.4, 2.6 Hz, 1H), 4.65 (d, J=6.6 Hz, 1H), 4.57 (d, J=12.7 Hz, 1H), 4.42 (d, J=13.1 Hz, 1H), 4.21 (d, J=12.7 Hz, 1H), 3.91 (s, 1H), 3.88 (s, 3H), 3.87-3.81 (m, 1H), 3.77 (t, J=9.3 Hz, 1H), 3.64-3.55 (m, 1H), 3.35 (d, J=14.0 Hz, 1H), 2.74-2.61 (m, 1H), 2.53 (s, 3H), 2.47 (d, J=12.5 Hz, 2H), 2.04-1.96 (m, 1H), 1.89 (d, J=15.1 Hz, 1H), 1.72 (t, J=13.3 Hz, 1H).LC/MS (ESI) m/z: 477 (M+H)

### Example 10: (1²S,1⁴S,Z)-1¹-((5-methoxy-7-methyl-1H-indol-4-yl)methyl)-3,11-dioxa-1(2,4)-piperidina-2(1,2)-benzenacycloundecaphan-8-ene-2⁴-carboxylic acid

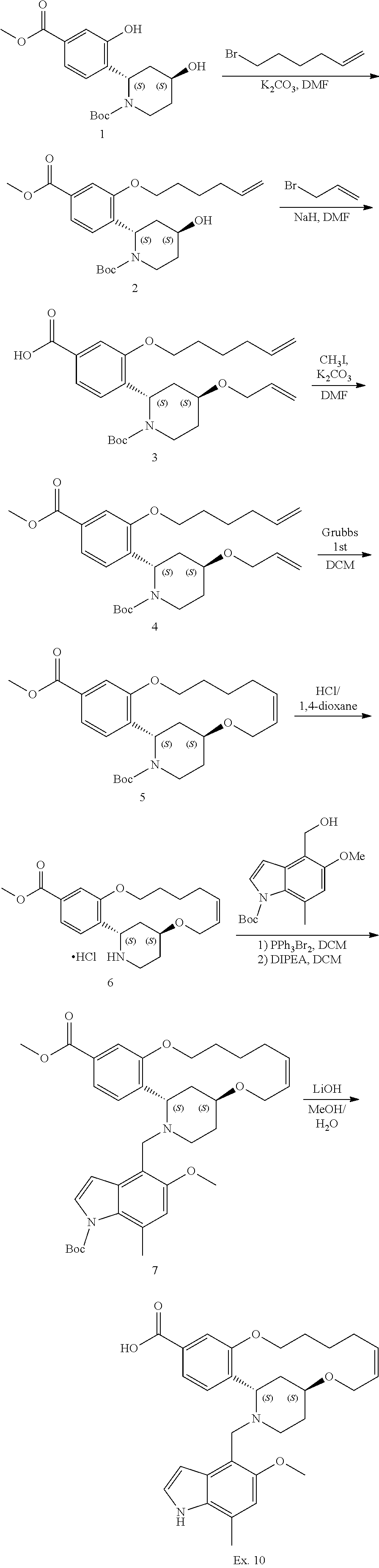

Step 1: tert-butyl (2S,4S)-2-(2-(hex-5-en-1-yloxy)-4-(methoxycarbonyl)phenyl)-4-hydroxypiperidine-1-carboxylate: To a solution of tert-butyl (2S,4S)-4-hydroxy-2-(2-hydroxy-4-(methoxycarbonyl)phenyl) piperidine-1-carboxylate (90 mg, 0.26 mmol) in DMF (2 mL) was added K₂CO₃ (141 mg, 1.02 mmol) and 6-bromohex-1-ene (83 mg, 0.51 mmol) and the mixture was stirred at 25° C. for 18 hours. The mixture was concentrated to dryness under reduced pressure and the residue was purified by flash chromatography (petroleum ether:ethyl acetate=1:1) to give tert-butyl (2S,4S)-2-(2-(hex-5-en-1-yloxy)-4-(methoxycarbonyl)phenyl)-4-hydroxypiperidine-1-carboxylate (75 mg, yield 67.7%) as white solid. LC/MS (ESI) m/z: 434 (M+H)⁺.

Step 2: 4-((2S,4S)-4-(allyloxy)-1-(tert-butoxycarbonyl)piperidin-2-yl)-3-(hex-5-en-1-yloxy)benzoic acid: To a solution of tert-butyl (2S,4S)-2-(2-(hex-5-en-1-yloxy)-4-(methoxycarbonyl)phenyl)-4-hydroxypiperidine-1-carboxylate (75 mg, 0.17 mmol) in DMF (2 mL) was added NaH (42 mg, 1.04 mmol, 60% dispersion in mineral oil) in portions at 0° C. The mixture was stirred under N₂ atmosphere for half an hour before 3-bromoprop-1-ene (31 mg, 0.26 mmol) was added and the resulting mixture was stirred at 25° C. for 18 hours. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give 4-((2S,4S)-4-(allyloxy)-1-(tert-butoxycarbonyl)piperidin-2-yl)-3-(hex-5-en-1-yloxy)benzoic acid (60 mg, yield 75.9%) as colorless oil. LC/MS: m/z 460 (M+H)⁺.

Step 3: tert-butyl (2S,4S)-4-(allyloxy)-2-(2-(hex-5-en-1-yloxy)-4-(methoxycarbonyl)phenyl) piperidine-1-carboxylate: To a solution of 4-((2S,4S)-4-(allyloxy)-1-(tert-butoxycarbonyl)piperidin-2-yl)-3-(hex-5-en-1-yloxy)benzoic acid (60 mg, 0.13 mmol) in DMF (2 mL) was added K₂CO₃ (60 mg, 0.41 mmol) and iodomethane (116 mg, 0.82 mmol) and the mixture was stirred at 25° C. for 1 hour. The mixture was concentrated to dryness under reduced pressure and the residue was purified by flash chromatography (petroleum ether:ethyl acetate=3:1) to give tert-butyl (2S,4S)-4-(allyloxy)-2-(2-(hex-5-en-1-yloxy)-4-(methoxycarbonyl)phenyl)piperidine-1-carboxylate (60 mg, yield 97.6%) as colorless oil. LC/MS (ESI) m/z: 474 (M+H)⁺.

Step 4: 1¹-(tert-butyl) 2⁴-methyl (1²S,1⁴S,Z)-3,11-dioxa-1(2,4)-piperidina-2(1,2)-benzenacycloundecaphan-8-ene-1¹,2⁴-dicarboxylate: To a solution of tert-butyl (2S,4S)-4-(allyloxy)-2-(2-(hex-5-en-1-yloxy)-4-(methoxycarbonyl)phenyl)piperidine-1-carboxylate (60 mg, 0.13 mmol) in dry DCM (60 mL) was added Grubbs 1^{st} catalyst (21 mg, 0.025 mmol) and the mixture was stirred under N₂ atmosphere at 25° C. for 18 hours. The mixture was concentrated to dryness under reduced pressure and the residue was purified by flash chromatography (petroleum ether:ethyl acetate=3:1) on silica gel to give 1¹-(tert-butyl) 2⁴-methyl (1²S,1⁴S,Z)-3,11-dioxa-1(2,4)-piperidina-2(1,2)-benzenacycloundecaphan-8-ene-1¹,2⁴-dicarboxylate (47 mg, yield 83.2%) as brown oil. LC/MS (ESI) m/z: 446 (M+H)⁺.

Step 5: methyl (1²S,1⁴S,Z)-3,11-dioxa-1(2,4)-piperidina-2(1,2)-benzenacycloundecaphan-8-ene-2⁴-carboxylate hydrochloride: A mixture of 1¹-(tert-butyl) 2⁴-methyl (1²,1⁴S,Z)-3,11-dioxa-1(2,4)-piperidina-2(1,2)-benzenacycloundecaphan-8-ene-1¹,2⁴-dicarboxylate (47 mg, 0.11 mmol) in HCl/1,4-dioxane (1 mL) was stirred at 25° C. for 1 hour. The mixture was concentrated to dryness under reduced pressure to give methyl (12S,14S,Z)-3,11-dioxa-1(2,4)-piperidina-2(1,2)-benzenacycloundecaphan-8-ene-24-carboxylate hydrochloride (40 mg, yield 95.2%) as white solid, which was used directly in the next step without further purification. LC/MS (ESI) m/z: 346 (M+H)⁺.

Step 6: methyl (1²S,1⁴S,Z)-11-((1-(tert-butoxycarbonyl)-5-methoxy-7-methyl-1H-indol-4-yl)methyl)-3,11-dioxa-1(2,4)-piperidina-2(1,2)-benzenacycloundecaphan-8-ene-2⁴-carboxylate: To a solution of tert-butyl 4-(hydroxymethyl)-5-methoxy-7-methyl-1H-indole-1-carboxylate (38 mg, 0.13 mmol) in dry DCM (1 mL) was added PPh₃Br₂ (60 mg, 0.14 mmol) in portions at 0° C. The mixture was stirred for 2 hours before methyl (1²S,1⁴S,Z)-3,10-dioxa-1(2,4)-piperidina-2(1,2)-benzenacyclodecaphan-7-ene-2⁴-carboxylate hydrochloride (40 mg, 0.11 mmol) and DIPEA (43 mg, 0.33 mmol) in dry DCM (1 mL) were added and the mixture was stirred for 1.5 hours at 0° C. The mixture was concentrated to dryness under reduced pressure, and the residue was purified by prep-HPLC to give methyl (1²S,1⁴S,Z)-1¹-((1-(tert-butoxycarbonyl)-5-methoxy-7-methyl-1H-indol-4-yl)methyl)-3,11-dioxa-1(2,4)-piperidina-2(1,2)-benzenacycloundecaphan-8-ene-2⁴-carboxylate (35 mg, yield 51.4%) as colorless oil. LC/MS (ESI) m/z: 619 (M+H)⁺.

Step 7: (1²S,1⁴S,Z)-11-((5-methoxy-7-methyl-1H-indol-4-yl)methyl)-3,11-dioxa-1(2,4)-piperidina-2(1,2)-benzenacycloundecaphan-8-ene-2⁴-carboxylic acid (Example 10): To a solution of methyl (1²S,1⁴S,Z)-11-((1-(tert-butoxycarbonyl)-5-methoxy-7-methyl-1H-indol-4-yl)methyl)-3,11-dioxa-1(2,4)-piperidina-2(1,2)-benzenacycloundecaphan-8-ene-2⁴-carboxylate (35 mg, 0.057 mmol) in methanol (1 mL)/water (0.3 mL) was added LiOH H₂O (15 mg, 0.34 mmol) and the mixture was stirred at room temperature overnight. The mixture was acidified with 1N aq.HCl to pH˜4 and extracted with ethyl acetate twice. The combined organic layers were washed with water and brine, dried over anhydrous Na₂SO₄, filtered and concentrated to dryness under reduced pressure. The residue was purified by prep-HPLC to give (1²S,1⁴S,Z)-1¹-((5-methoxy-7-methyl-1H-indol-4-yl)methyl)-3,11-dioxa-1(2,4)-piperidina-2(1,2)-benzenacycloundecaphan-8-ene-2⁴-carboxylic acid (11.6 mg, yield 40.4%) as white solid. ¹H NMR (400 MHz, CD₃OD) δ 8.34 (d, J=4.7 Hz, 1H), 7.87-7.66 (m, 3H), 7.31 (s, 1H), 6.78 (s, 1H), 6.38-6.29 (m, 1H), 5.87 (dd, J=23.9, 15.1 Hz, 1H), 5.76 (d, J=5.8 Hz, 1H), 5.37 (dd, J=28.4, 12.5 Hz, 1H), 4.46-4.18 (m, 3H), 4.15-4.04 (m, 2H), 3.90 (d, J=14.0 Hz, 1H), 3.80 (d, J=2.3 Hz, 3H), 3.62 (d, J=12.9 Hz, 1H), 3.40 (d, J=12.2 Hz, 1H), 2.50 (s, 4H), 2.26 (d, J=21.2 Hz, 1H), 2.04 (ddd, J=44.6, 27.8, 17.3 Hz, 6H), 1.79 (d, J=49.9 Hz, 3H).LC/MS (ESI) m/z: 479 (M+H)⁺.

### Example 11: (1²S,1⁴S,Z)-1′-((5-methoxy-7-methyl-1H-indol-4-yl)methyl)-3,10-dioxa-1(2,4)-piperidina-2(1,2)-benzenacyclodecaphan-7-ene-2⁴-carboxylic acid

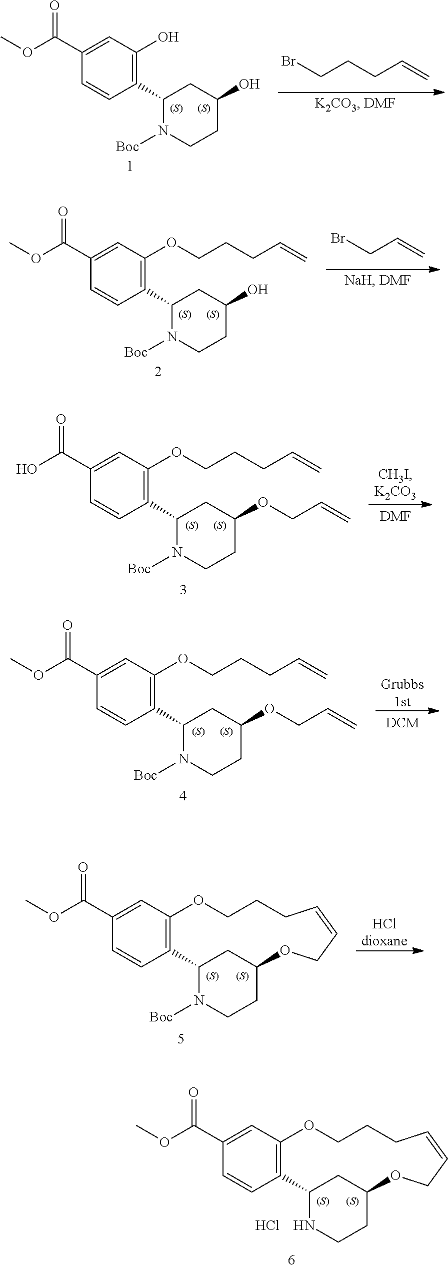

Step 1: tert-butyl (2S,4S)-4-hydroxy-2-(4-(methoxycarbonyl)-2-(pent-4-en-1-yloxy)phenyl)piperidine-1-carboxylate: To a solution of tert-butyl (2S,4S)-4-hydroxy-2-(2-hydroxy-4-(methoxycarbonyl)phenyl) piperidine-1-carboxylate (90 mg, 0.26 mmol) in DMF (2 mL) was added K₂CO₃ (141 mg, 1.02 mmol) and 5-bromopent-1-ene (76 mg, 0.51 mmol) and the mixture was stirred at 25° C. for 18 hours. The mixture was concentrated to dryness under reduced pressure and the residue was purified by flash chromatography (petroleum ether:ethyl acetate=1:1) to give tert-butyl (2S,4S)-4-hydroxy-2-(4-(methoxycarbonyl)-2-(pent-4-en-1-yloxy)phenyl)piperidine-1-carboxylate (65 mg, yield 60.2%) as white solid. LC/MS (ESI) m/z: 420 (M+H)⁺.

Step 2: 4-((2S,4S)-4-(allyloxy)-1-(tert-butoxycarbonyl)piperidin-2-yl)-3-(pent-4-en-1-yloxy)benzoic acid: To a solution of tert-butyl (2S,4S)-4-hydroxy-2-(4-(methoxycarbonyl)-2-(pent-4-en-1-yloxy)phenyl)piperidine-1-carboxylate (65 mg, 0.16 mmol) in DMF (2 mL) was added NaH (37 mg, 0.93 mmol, 60% dispersion in mineral oil) in portions at 0° C. The mixture was stirred under N₂ atmosphere for half an hour before 3-bromoprop-1-ene (28 mg, 0.23 mmol) was added and the resulting mixture was stirred at 25° C. for 18 hours. The mixture was quenched with saturated aq.NH₄Cl solution and extracted with ethyl acetate twice. The combined organic layers were washed with brine, dried over anhydrous Na₂SO₄, filtered and concentrated to dryness under reduced pressure. The residue was purified by flash column chromatography on silica gel (petroleum ether:ethyl acetate=1:1) to give 4-((2S,4S)-4-(allyloxy)-1-(tert-butoxycarbonyl)piperidin-2-yl)-3-(pent-4-en-1-yloxy)benzoic acid (60 mg, yield 86.9%) as colorless oil. LC/MS: m/z 446 (M+H)⁺.

Step 3: tert-butyl (2S,4S)-4-(allyloxy)-2-(4-(methoxycarbonyl)-2-(pent-4-en-1-yloxy)phenyl)piperidine-1-carboxylate: To a solution of 4-((2S,4S)-4-(allyloxy)-1-(tert-butoxycarbonyl)piperidin-2-yl)-3-(pent-4-en-1-yloxy)benzoic acid (60 mg, 0.13 mmol) in DMF (2 mL) was added K₂CO₃ (60 mg, 0.41 mmol) and iodomethane (116 mg, 0.82 mmol) and the mixture was stirred at 25° C. for 1 hour. The mixture was concentrated to dryness under reduced pressure and the residue was purified by flash chromatography (petroleum ether:ethyl acetate=3:1) to give tert-butyl (2S,4S)-4-(allyloxy)-2-(4-(methoxycarbonyl)-2-(pent-4-en-1-yloxy)phenyl)piperidine-1-carboxylate (56 mg, yield 96.5%) as colorless oil. LC/MS (ESI) m/z: 460 (M+H)⁺.

Step 4: 1¹-(tert-butyl) 2⁴-methyl (1²S,1⁴S,Z)-3,10-dioxa-1(2,4)-piperidina-2(1,2)-benzenacyclodecaphan-7-ene-1¹,2⁴-dicarboxylate: To a solution of tert-butyl (2S,4S)-4-(allyloxy)-2-(4-(methoxycarbonyl)-2-(pent-4-en-1-yloxy)phenyl)piperidine-1-carboxylate (54 mg, 0.12 mmol) in dry DCM (54 mL) was added Grubbs 1^{st} catalyst (20 mg, 0.024 mmol) and the mixture was stirred under N₂ atmosphere at 25° C. for 18 hours. The mixture was concentrated to dryness under reduced pressure and the residue was purified by flash chromatography (petroleum ether:ethyl acetate=3:1) to give 1′-(tert-butyl) 2⁴-methyl (1²,1⁴S,Z)-3,10-dioxa-1(2,4)-piperidina-2(1,2)-benzenacyclodecaphan-7-ene-1¹,2⁴-dicarboxylate (35 mg, yield 68.8%) as brown oil. LC/MS (ESI) m/z: 432 (M+H)⁺.

Step 5: methyl (1²S,1⁴S,Z)-3,10-dioxa-1(2,4)-piperidina-2(1,2)-benzenacyclodecaphan-7-ene-2⁴-carboxylate hydrochloride: A mixture of 1¹-(tert-butyl) 2⁴-methyl (1²,1⁴S,Z)-3,10-dioxa-1(2,4)-piperidina-2(1,2)-benzenacyclodecaphan-7-ene-1¹,2⁴-dicarboxylate (35 mg, 0.081 mmol) in HCl/1,4-dioxane (1 mL) was stirred at 25° C. for 1 hour. The mixture was concentrated to dryness under reduced pressure to give methyl (1²,1⁴S,Z)-3,10-dioxa-1(2,4)-piperidina-2(1,2)-benzenacyclodecaphan-7-ene-2⁴-carboxylate hydrochloride (30 mg, yield 99.8%) as white solid, which was used directly in the next step without further purification. LC/MS (ESI) m/z: 332 (M+H)⁺.

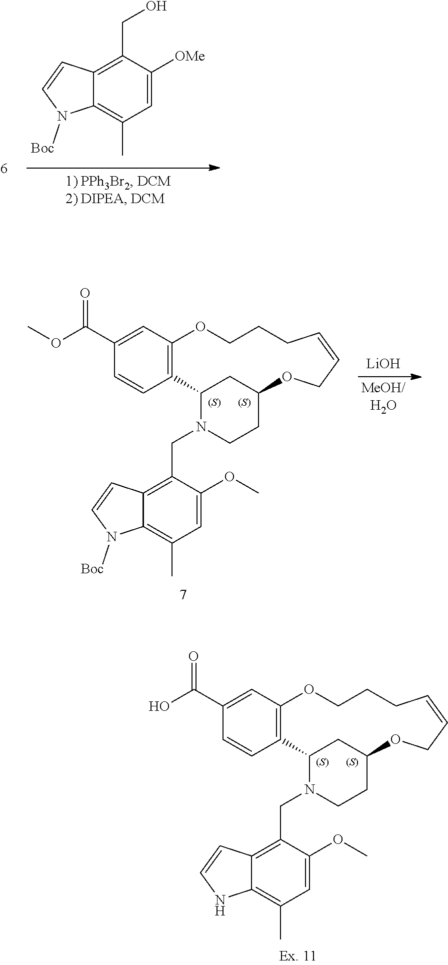

Step 6: methyl (1²S,1⁴S,Z)-1¹-((1-(tert-butoxycarbonyl)-5-methoxy-7-methyl-1H-indol-4-yl)methyl)-3,10-dioxa-1(2,4)-piperidina-2(1,2)-benzenacyclodecaphan-7-ene-2⁴-carboxylate: To a solution of tert-butyl 4-(hydroxymethyl)-5-methoxy-7-methyl-1H-indole-1-carboxylate (31 mg, 0.11 mmol) in dry DCM (1 mL) was added PPh₃Br₂ (49 mg, 0.12 mmol) in portions at 0° C. The mixture was stirred for 2 hours before methyl (1²S,1⁴S,Z)-3,10-dioxa-1(2,4)-piperidina-2(1,2)-benzenacyclodecaphan-7-ene-2⁴-carboxylate hydrochloride (30 mg, 0.09 mmol) and DIPEA (35 mg, 0.27 mmol) in dry DCM (1 mL) were added and the mixture was stirred for 1.5 hours at 0° C. The mixture was concentrated to dryness under reduced pressure, and the residue was purified by prep-HPLC to give methyl (1²S,1⁴S,Z)-1′-((1-(tert-butoxycarbonyl)-5-methoxy-7-methyl-1H-indol-4-yl)methyl)-3,10-dioxa-1(2,4)-piperidina-2(1,2)-benzenacyclodecaphan-7-ene-2⁴-carboxylate (30 mg, yield 55.6%) as colorless oil. LC/MS (ESI) m/z: 605 (M+H)⁺.

Step 7: (1²S,1⁴S,Z)-1¹-((5-methoxy-7-methyl-1H-indol-4-yl)methyl)-3,10-dioxa-1(2,4)-piperidina-2(1,2)-benzenacyclodecaphan-7-ene-2⁴-carboxylic acid (Example 11): To a solution of methyl (1²S,1⁴S,Z)-11-((1-(tert-butoxycarbonyl)-5-methoxy-7-methyl-1H-indol-4-yl)methyl)-3,10-dioxa-1(2,4)-piperidina-2(1,2)-benzenacyclodecaphan-7-ene-2⁴-carboxylate (30 mg, 0.05 mmol) in methanol (1 mL)/water (0.3 mL) was added LiOH H₂O (7 mg, 0.15 mmol) and the mixture was stirred at room temperature overnight. The mixture was acidified with 1 N aq.HCl to pH˜4 and extracted with ethyl acetate twice. The combined organic layers were washed with water and brine, dried over anhydrous Na₂SO₄, filtered and concentrated to dryness under reduced pressure. The residue was purified by prep-HPLC to give (1²S,1⁴S,Z)-1¹-((5-methoxy-7-methyl-1H-indol-4-yl)methyl)-3,10-dioxa-1(2,4)-piperidina-2(1,2)-benzenacyclodecaphan-7-ene-2⁴-carboxylic acid (10.4 mg, yield 42.4% yield) as white solid. ¹H NMR (400 MHz, CD₃OD) δ 8.33 (s, 1H), 7.79 (d, J=8.1 Hz, 1H), 7.70-7.59 (m, 2H), 7.33 (d, J=3.1 Hz, 1H), 6.80 (d, J=8.5 Hz, 1H), 6.45 (dd, J=12.0, 3.2 Hz, 1H), 6.09 (d, J=15.3 Hz, 1H), 5.77 (d, J=15.4 Hz, 1H), 5.45 (d, J=12.3 Hz, 1H), 4.39 (d, J=15.5 Hz, 3H), 4.16 (s, 2H), 3.90-3.80 (m, 4H), 3.76 (dd, J=14.3, 7.0 Hz, 1H), 3.68-3.57 (m, 1H), 3.41 (d, J=12.4 Hz, 1H), 2.51 (s, 4H), 2.22 (dd, J=23.2, 14.1 Hz, 3H), 1.98 (dd, J=28.5, 15.5 Hz, 4H).LC/MS (ESI) m/z: 491 (M+H)⁺.

### Example 12: (2²S,2⁴S,Z)-2¹-((5-methoxy-7-methyl-1H-indol-4-yl)methyl)-5′H-3,8-dioxa-2(2,4)-piperidina-5(4,1)-triazola-1(1,2)-benzenacyclooctaphane-1⁴-carboxylic acid

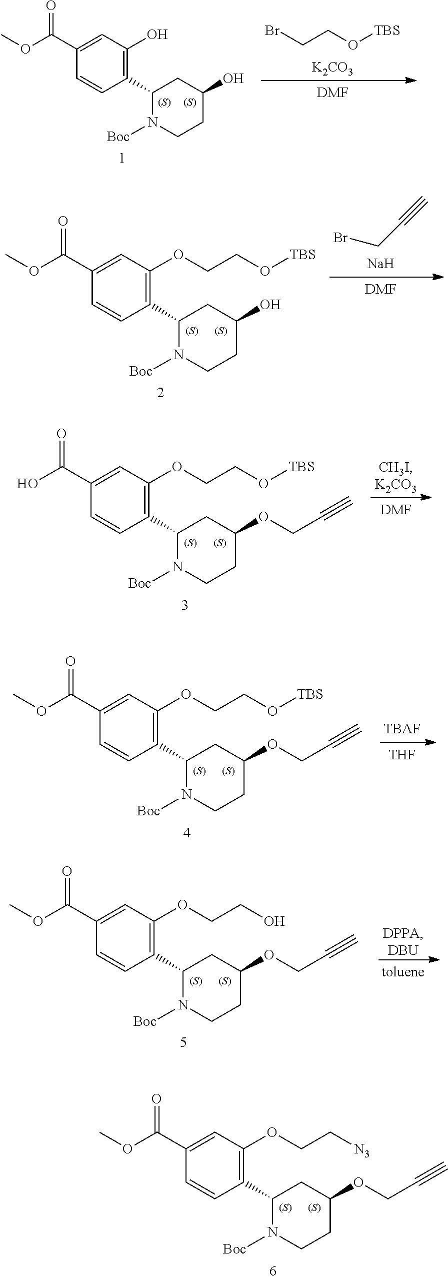

Step 1: tert-butyl (2S,4S)-2-(2-(2-((tert-butyldimethylsilyl)oxy)ethoxy)-4-(methoxycarbonyl)phenyl)-4-hydroxypiperidine-1-carboxylate: A mixture of tert-butyl (2S,4S)-4-hydroxy-2-[2-hydroxy-4-(methoxycarbonyl)phenyl] piperidine-1-carboxylate (120 mg, 0.34 mmol), (2-bromoethoxy)(tert-butyl)dimethylsilane (160 mg, 0.68 mmol) and K₂CO₃ (140 mg, 1.02 mmol) in DMF (2 mL) was stirred at room temperature under N₂ atmosphere for 2 hours. The mixture was concentrated to dryness under reduced pressure and purified by flash column chromatography on silica gel (petroleum ether:ethyl acetate=3:1) to give tert-butyl (2S,4S)-2-(2-{2-[(tert-butyldimethylsilyl)oxy]ethoxy}-4-(methoxycarbonyl)phenyl)-4-hydroxypiperidine-1-carboxylate (130 mg, Yield 74.7%) as white solid. LC/MS (ESI) m/z: 510.2 (M+H)⁺.

Step 2: 4-((2S,4S)-1-(tert-butoxycarbonyl)-4-(prop-2-yn-1-yloxy)piperidin-2-yl)-3-(2-((tert-butyldimethylsilyl)oxy)ethoxy)benzoic acid: To a solution of tert-butyl (2S,4S)-2-(2-{2-[(tert-butyldimethylsilyl)oxy]ethoxy}-4-(methoxycarbonyl)phenyl)-4-hydroxypiperidine-1-carboxylate (70 mg, 0.13 mmol) in DMF (2 mL) was added NaH (10 mg, 0.26 mmol, 60% dispersion in mineral oil) in portions at 0° C. The mixture was stirred under N₂ atmosphere for 0.5 hour before 3-bromo-1-propyne (47 mg, 0.39 mmol) was added and the resulting mixture was stirred at 25° C. for 18 hours. The mixture was quenched with saturated aq.NH₄Cl solution and extracted with ethyl acetate. The organic layer was washed with brine, dried over Na₂SO₄, filtered and concentrated to dryness under reduced pressure. The residue was purified by flash column chromatography on silica gel (petroleum ether:ethyl acetate=5:1) to give 4-[(2S,4S)-1-(tert-butoxycarbonyl)-4-(prop-2-yn-1-yloxy)piperidin-2-yl]-3-{2-[(tert-butyldimethylsilyl)oxy]ethoxy}benzoic acid (35 mg, Yield 50.2%) as colorless oil. LC/MS: m/z 534.2 (M+H)⁺.

Step 3: tert-butyl (2S,4S)-2-(2-(2-((tert-butyldimethylsilyl)oxy)ethoxy)-4-(methoxycarbonyl)phenyl)-4-(prop-2-yn-1-yloxy)piperidine-1-carboxylate: To a solution of 4-[(2S,4S)-1-(tert-butoxycarbonyl)-4-(prop-2-yn-1-yloxy)piperidin-2-yl]-3-{2-[(tert-butyldimethylsilyl)oxy]ethoxy}benzoic acid (30 mg, 0.056 mmol) in DMF (2 mL) was added K₂CO₃ (16 mg, 0.11 mmol) and iodomethane (32 mg, 0.22 mmol) and the mixture was stirred at 25° C. for 1 hour. The mixture was concentrated to dryness under reduced pressure and the residue was purified by flash chromatography (petroleum ether:ethyl acetate=3:1) to give tert-butyl (2S,4S)-2-(2-{2-[(tert-butyldimethylsilyl)oxy]ethoxy}-4-(methoxycarbonyl)phenyl)-4-(prop-2-yn-1-yloxy)piperidine-1-carboxylate (30 mg, Yield 97.4%) as colorless oil. LC/MS (ESI) m/z: 548.3 (M+H)⁺.

Step 4: tert-butyl (2S,4S)-2-(2-(2-hydroxyethoxy)-4-(methoxycarbonyl)phenyl)-4-(prop-2-yn-1-yloxy)piperidine-1-carboxylate: To a solution of tert-butyl (2S,4S)-2-(2-{2-[(tert-butyldimethylsilyl)oxy]ethoxy}-4-(methoxycarbonyl)phenyl)-4-(prop-2-yn-1-yloxy)piperidine-1-carboxylate (60 mg, 0.11 mmol) in THF (2 mL) was added TBAF (53 mg, 0.22 mmol) at 25° C. and the mixture was stirred under N₂ atmosphere at 25° C. for 1 hour. The mixture was concentrated to dryness under reduced pressure to give the crude product which was further purified by flash column chromatography on silica gel (petroleum ether:ethyl acetate=3:1) to give tert-butyl (2S,4S)-2-[2-(2-hydroxyethoxy)-4-(methoxycarbonyl)phenyl]-4-(prop-2-yn-1-yloxy)piperidine-1-carboxylate (40 mg, Yield 84.3%) as white solid. LC/MS (ESI) m/z: 434.2 (M+H)⁺.

Step 5: tert-butyl (2S,4S)-2-(2-(2-azidoethoxy)-4-(methoxycarbonyl)phenyl)-4-(prop-2-yn-1-yloxy)piperidine-1-carboxylate: To a solution of tert-butyl (2S,4S)-2-[2-(2-hydroxyethoxy)-4-(methoxycarbonyl)phenyl]-4-(prop-2-yn-1-yloxy)piperidine-1-carboxylate (30 mg, 0.069 mmol), DBU (16 mg, 0.10 mmol) in toluene (0.5 mL) was added DPPA (23 mg, 0.083 mmol) at 25° C. under N₂ atmosphere and the mixture was stirred under N₂ atmosphere at 25° C. for 1 hour. The mixture was concentrated to dryness under reduced pressure to give the crude product which was further purified by flash column chromatography on silica gel (petroleum ether:ethyl acetate=3:1) to give tert-butyl (2S,4S)-2-[2-(2-azidoethoxy)-4-(methoxycarbonyl)phenyl]-4-(prop-2-yn-1-yloxy)piperidine-1-carboxylate (30 mg, Yield 94.5%) as colorless oil. LC/MS (ESI) m/z: 459.2 (M+H)⁺.

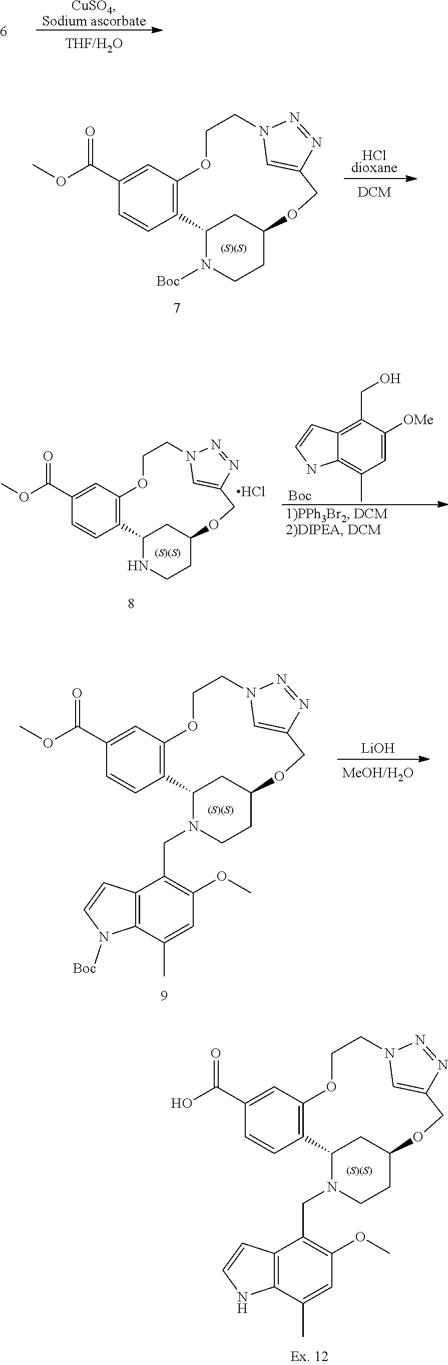

Step 6: 2¹-(tert-butyl) 1⁴-methyl (2²S,2⁴S,Z)-5′H-3,8-dioxa-2(2,4)-piperidina-5(4,1)-triazola-1(1,2)-benzenacyclooctaphane-1⁴,2¹-dicarboxylate: A solution of tert-butyl (2S,4S)-2-[2-(2-azidoethoxy)-4-(methoxycarbonyl)phenyl]-4-(prop-2-yn-1-yloxy)piperidine-1-carboxylate (32 mg, 0.07 mmol), L-ascorbic acid sodium salt (3 mg, 0.017 mmol), CuSO₄ (4 mg, 0.017 mmol) in THF (0.6 mL), water (0.3 mL) was stirred at 25° C. under N₂ atmosphere for 2 hours. The reaction solution was concentrated to dryness under reduced pressure to give the crude product which was further purified by flash column chromatography on silica gel (petroleum ether:ethyl acetate=1:3) to give 2¹-(tert-butyl) 1⁴-methyl (2²S,2⁴S,Z)-5¹H-3,8-dioxa-2(2,4)-piperidina-5(4,1)-triazola-1(1,2)-benzenacyclooctaphane-1⁴,2¹-dicarboxylate (20 mg, Yield 62.5%) as colorless oil. LC/MS (ESI) m/z: 459.2 (M+H)⁺.

Step 7: methyl (2²S,2⁴S,Z)-5′H-3,8-dioxa-2(2,4)-piperidina-5(4,1)-triazola-1(1,2)-benzenacyclooctaphane-1⁴-carboxylate: To a mixture of 2¹-(tert-butyl) 1⁴-methyl (2²S,2⁴S,Z)-5¹H-3,8-dioxa-2(2,4)-piperidina-5(4,1)-triazola-1(1,2)-benzenacyclooctaphane-1⁴,2¹-dicarboxylate (20 mg, 0.044 mmol) and HCl/1,4-dioxane (1 mL) in DCM (1 mL) was added at room temperature and the mixture was stirred at 25° C. for 1 hour. The mixture was concentrated to dryness under reduced pressure to give methyl (2²S,2⁴S,Z)-5¹H-3,8-dioxa-2(2,4)-piperidina-5(4,1)-triazola-1(1,2)-benzenacyclooctaphane-1⁴-carboxylate (16 mg, Yield 98.2%) as white solid. LC/MS (ESI) m/z: 359.1 (M+H)⁺.

Step 8: methyl (2²S,2⁴S,Z)-2¹-((1-(tert-butoxycarbonyl)-5-methoxy-7-methyl-1H-indol-4-yl)methyl)-5¹H-3,8-dioxa-2(2,4)-piperidina-5(4,1)-triazola-1(1,2)-benzenacyclooctaphane-1⁴-carboxylate: To a solution of tert-butyl 4-(hydroxymethyl)-5-methoxy-7-methylindole-1-carboxylate (13 mg, 0.045 mmol) in dichloromethane (4 mL) was added PPh₃Br₂ (17 mg, 0.058 mmol) and the mixture was stirred for 1.5 hours under nitrogen atmosphere before methyl (2²S,2⁴S,Z)-5¹H-3,8-dioxa-2(2,4)-piperidina-5(4,1)-triazola-1(1,2)-benzenacyclooctaphane-1⁴-carboxylate (16 mg, 0.045 mmol) and DIPEA (17 mg, 0.13 mmol) were added and the mixture was stirred for additional 2 hours The reaction mixture was concentrated to dryness under reduced pressure to give the crude product which was further purified by prep-TLC to give methyl (2²S,2⁴S,Z)-2¹-((1-(tert-butoxycarbonyl)-5-methoxy-7-methyl-1H-indol-4-yl)methyl)-5¹H-3,8-dioxa-2(2,4)-piperidina-5(4,1)-triazola-1(1,2)-benzenacyclooctaphane-1⁴-carboxylate as white solid. LC/MS (ESI) m/z: 632.3 (M+H)⁺.

Step 9: (2²S,2⁴S,Z)-21-((5-methoxy-7-methyl-1H-indol-4-yl)methyl)-5¹H-3,8-dioxa-2(2,4)-piperidina-5(4,1)-triazola-1(1,2)-benzenacyclooctaphane-1⁴-carboxylic acid (Example 12): To a solution of methyl (2²S,2⁴S,Z)-2¹-((1-(tert-butoxycarbonyl)-5-methoxy-7-methyl-1H-indol-4-yl)methyl)-5¹H-3,8-dioxa-2(2,4)-piperidina-5(4,1)-triazola-1(1,2)-benzenacyclooctaphane-1⁴-carboxylate (14 mg, 0.022 mmol) in MeOH (0.6 mL)/water (0.2 mL) was added LiOH (2.8 mg, 0.066 mmol) and the mixture was stirred at room temperature overnight. The mixture was acidified with 1N aq.HCl to pH˜4 and extracted with ethyl acetate twice. The combined organic layers were washed with water and brine, dried over anhydrous Na₂SO₄, filtered and concentrated to dryness under reduced pressure. The residue was purified by prep-HPLC to give (2²S,2⁴S,Z)-21-((5-methoxy-7-methyl-1H-indol-4-yl)methyl)-5¹H-3,8-dioxa-2(2,4)-piperidina-5(4,1)-triazola-1(1,2)-benzenacyclooctaphane-14-carboxylic acid (4.0 mg, Yield 34.9%) as white solid. LC/MS (ESI) m/z: 518.2 (M+H)⁺.

### Example 13: (2²S,2⁴S,Z)-2¹-((5-methoxy-7-methyl-1H-indol-4-yl) methyl)-5′H-3,9-dioxa-2(2,4)-piperidina-5(4,1)-triazola-1(1,2)-benzenacyclononaphane-1⁴-carboxylic acid

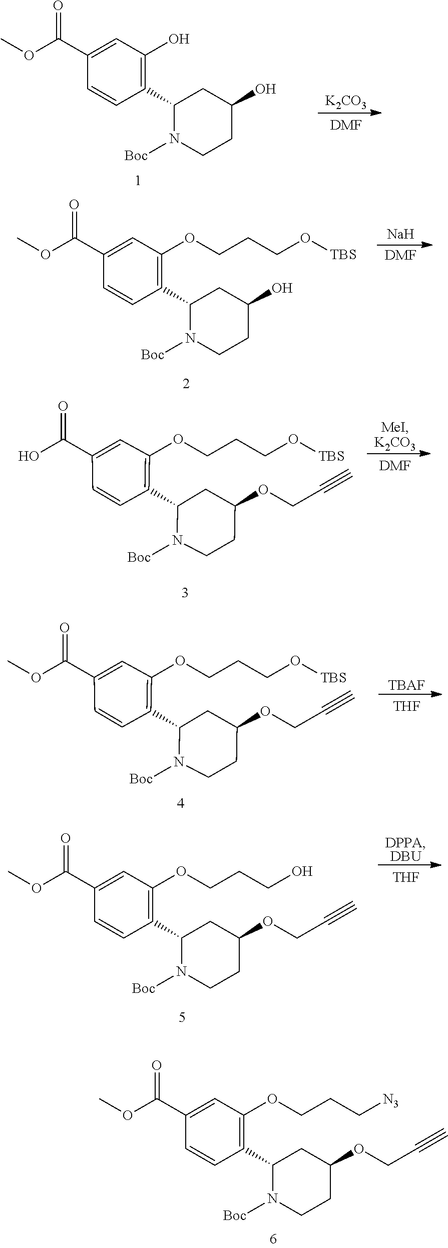

Step 1: tert-butyl (2S,4S)-2-(2-(3-((tert-butyldimethylsilyl)oxy)propoxy)-4-(methoxycarbonyl)phenyl)-4-hydroxypiperidine-1-carboxylate (2): A mixture of tert-butyl (2S,4S)-4-hydroxy-2-(2-hydroxy-4-(methoxycarbonyl)phenyl) piperidine-1-carboxylate (150 mg, 0.43 mmol) in DMF (4 mL) at room temperature was added (3-bromopropoxy)(tert-butyl)dimethylsilane (215 mg, 0.86 mmol) and K₂CO₃ (230 mg, 1.72 mmol) under N₂ atmosphere and the mixture was stirred at 25° C. for 2 hours. The mixture was concentrated to dryness under reduced pressure and purified by flash column chromatography on silica gel (petroleum ether:ethyl acetate=1:1) to give tert-butyl (2S,4S)-2-(2-(3-((tert-butyldimethylsilyl)oxy)propoxy)-4-(methoxycarbonyl)phenyl)-4-hydroxypiperidine-1-carboxylate (150 mg, Yield 67.1%) as a white solid. LC/MS (ESI) m/z: 524 (M+H)⁺.

Step 2: 4-((2S,4S)-1-(tert-butoxycarbonyl)-4-(prop-2-yn-1-yloxy)piperidin-2-yl)-3-(3-((tert-butyldimethylsilyl)oxy)propoxy)benzoic acid (3): To a solution of tert-butyl (2S,4S)-2-(2-(3-((tert-butyldimethylsilyl)oxy)propoxy)-4-(methoxycarbonyl)phenyl)-4-hydroxypiperidine-1-carboxylate (150 mg, 0.28 mmol) in DMF (2 mL) was added NaH (45 mg, 1.12 mmol, 60% dispersion in mineral oil) in portions at 0° C., and the mixture was stirred under an N₂ atmosphere for 0.5 hour before adding 3-bromo-1-propyne (66 mg, 0.56 mmol). The resulting mixture was stirred at 25° C. for 18 hours before being quenched with saturated aq. NH₄Cl solution and extracted twice with ethyl acetate. The combined organic layers were washed with brine, dried over anhydrous Na₂SO₄, filtered and concentrated to dryness under reduced pressure to give 4-((2S,4S)-1-(tert-butoxycarbonyl)-4-(prop-2-yn-1-yloxy)piperidin-2-yl)-3-(3-((tert-butyldimethylsilyl)oxy)propoxy)benzoic acid (100 mg, Yield 64.1%) as a colorless oil. LC/MS: m/z 548 (M+H)⁺.

Step 3: tert-butyl (2S,4S)-2-(2-(3-((tert-butyldimethylsilyl)oxy)propoxy)-4-(methoxycarbonyl)phenyl)-4-(prop-2-yn-1-yloxy)piperidine-1-carboxylate (4): To a solution of 4-((2S,4S)-1-(tert-butoxycarbonyl)-4-(prop-2-yn-1-yloxy)piperidin-2-yl)-3-(3-((tert-butyldimethylsilyl)oxy)propoxy)benzoic acid (100 mg, 0.18 mmol) in DMF (2 mL) was added K₂CO₃ (50 mg, 0.36 mmol) and methyl iodide (51 mg, 0.36 mmol) and the mixture was stirred at 25° C. for 1 hour. The mixture was concentrated to dryness under reduced pressure and the residue was purified by flash column chromatography (petroleum ether:ethyl acetate=3:1) to give tert-butyl (2S,4S)-2-(2-(3-((tert-butyldimethylsilyl)oxy)propoxy)-4-(methoxycarbonyl)phenyl)-4-(prop-2-yn-1-yloxy)piperidine-1-carboxylate (70 mg, Yield 68.6%) as a colorless oil. LC/MS (ESI) m/z: 562 (M+H)⁺.

Step 4: tert-butyl (2S,4S)-2-(2-(3-hydroxypropoxy)-4-(methoxycarbonyl)phenyl)-4-(prop-2-yn-1-yloxy)piperidine-1-carboxylate (5): To a solution of tert-butyl (2S,4S)-2-(2-(3-((tert-butyldimethylsilyl)oxy)propoxy)-4-(methoxycarbonyl)phenyl)-4-(prop-2-yn-1-yloxy)piperidine-1-carboxylate (70 mg, 0.12 mmol) in THF (2 mL) was added TBAF (53 mg, 0.22 mmol) at 25° C., and the mixture was stirred under an N₂ atmosphere at 25° C. for 1 hour. The mixture was concentrated to dryness under reduced pressure to give the crude product which was purified by flash column chromatography on silica gel (petroleum ether:ethyl acetate=3:1) to give tert-butyl (2S,4S)-2-(2-(3-hydroxypropoxy)-4-(methoxycarbonyl)phenyl)-4-(prop-2-yn-1-yloxy)piperidine-1-carboxylate (40 mg, Yield 72.7%) as a white solid. LC/MS (ESI) m/z: 448 (M+H)⁺.

Step 5: tert-butyl (2S,4S)-2-(2-(3-azidopropoxy)-4-(methoxycarbonyl)phenyl)-4-(prop-2-yn-1-yloxy)piperidine-1-carboxylate (6): To a solution of tert-butyl (2S,4S)-2-(2-(3-hydroxypropoxy)-4-(methoxycarbonyl)phenyl)-4-(prop-2-yn-1-yloxy)piperidine-1-carboxylate (30 mg, 0.07 mmol) and DBU (1,8-Diazabicyclo[5.4.0]undec-7-ene, 16 mg, 0.10 mmol) in toluene (0.5 mL) was added DPPA (diphenylphosphoryl azide, 23 mg, 0.083 mmol) at 25° C. under an N₂ atmosphere and the mixture was stirred under an N₂ atmosphere at 80° C. for 1 hour. The mixture was concentrated to dryness under reduced pressure to give the crude product. The residue was further purified by flash column chromatography on silica gel (petroleum ether:ethyl acetate=3:1) to give tert-butyl (2S,4S)-2-(2-(3-azidopropoxy)-4-(methoxycarbonyl)phenyl)-4-(prop-2-yn-1-yloxy)piperidine-1-carboxylate (30 mg, Yield 94.7%) as colorless oil. LC/MS (ESI) m/z: 473(M+H)⁺.

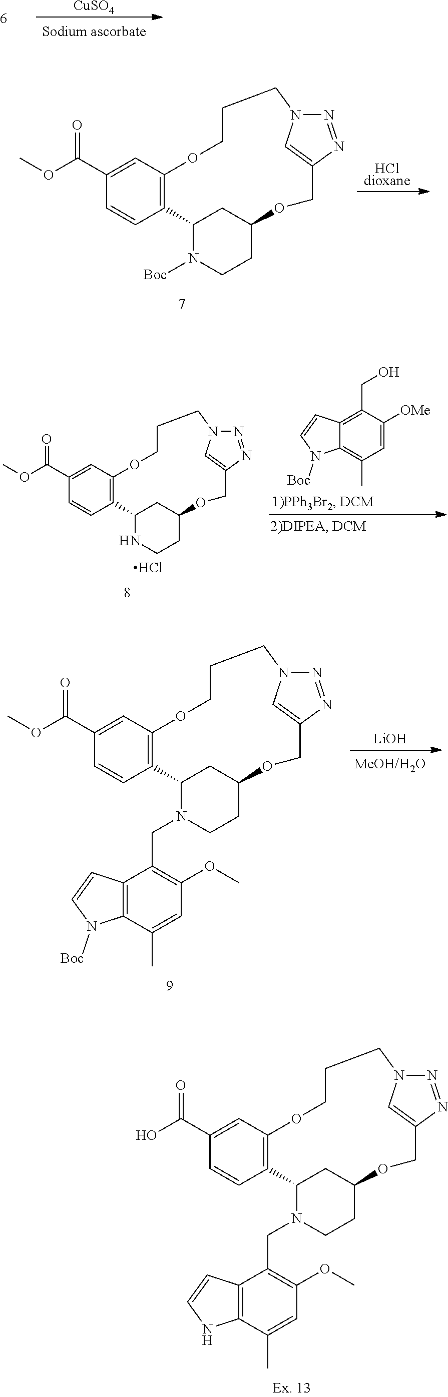

Step 6: 2¹-(tert-butyl) 1⁴-methyl (2²S,2⁴S,Z)-5¹H-3,9-dioxa-2(2,4)-piperidina-5(4,1)-triazola-1(1,2)-benzenacyclononaphane-1⁴,2¹-dicarboxylate (7): A solution of tert-butyl (2S,4S)-2-(2-(3-azidopropoxy)-4-(methoxycarbonyl)phenyl)-4-(prop-2-yn-1-yloxy)piperidine-1-carboxylate (30 mg, 0.07 mmol), sodium L-ascorbic acid (3 mg, 0.017 mmol) and CuSO₄ (4 mg, 0.017 mmol) in THF (0.6 mL), water (0.3 mL) was stirred at 25° C. under N₂ atmosphere for 2 hours. The mixture was concentrated to dryness under reduced pressure to give the crude product. The residue was further purified by flash column chromatography on silica gel (petroleum ether:ethyl acetate=1:3) to give 2¹-(tert-butyl) 1⁴-methyl (2²S,2⁴S,Z)-5¹H-3,9-dioxa-2(2,4)-piperidina-5(4,1)-triazola-1 (1,2)-benzenacyclononaphane-1⁴,2¹-dicarboxylate (20 mg, Yield 66.6%) as a colorless oil. LC/MS (ESI) m/z: 473 (M+H)⁺.

Step 7: methyl (2²S,2⁴S,Z)-5¹H-3,9-dioxa-2(2,4)-piperidina-5(4,1)-triazola-1(1,2)-benzenacyclononaphane-1⁴-carboxylate (8): A mixture of 2¹-(tert-butyl) 1⁴-methyl (2²S,2⁴S,Z)-5¹H-3,9-dioxa-2(2,4)-piperidina-5(4,1)-triazola-1 (1,2)-benzenacyclononaphane-1⁴,2¹-dicarboxylate (20 mg, 0.044 mmol) and HCl/1,4-dioxane (1 mL) in DCM (1 mL) was stirred at 25° C. for 1 hour. The mixture was concentrated to dryness under reduced pressure to give methyl (2²S,2⁴S,Z)-5¹H-3,9-dioxa-2(2,4)-piperidina-5(4,1)-triazola-1 (1,2)-benzenacyclononaphane-1⁴-carboxylate (16 mg, Yield 98.2%) as a white solid. LC/MS (ESI) m/z: 373 (M+H)⁺.

Step 8: methyl (2²S,2⁴S,Z)-2¹-((1-(tert-butoxycarbonyl)-5-methoxy-7-methyl-1H-indol-4-yl)methyl)-5′H-3,9-dioxa-2(2,4)-piperidina-5(4,1)-triazola-1(1,2)-benzenacyclononaphane-14-carboxylate(9): To a solution of tert-butyl 4-(hydroxymethyl)-5-methoxy-7-methylindole-1-carboxylate (13 mg, 0.045 mmol) in dichloromethane (4 mL) was added PPh₃Br₂ (17 mg, 0.058 mmol) at 0° C., and the mixture was stirred for 1.5 hours before methyl (2²S,2⁴S,Z)-5¹H-3,9-dioxa-2(2,4)-piperidina-5(4,1)-triazola-1(1,2)-benzenacyclononaphane-1⁴-carboxylate (16 mg, 0.045 mmol) and DIPEA (17 mg, 0.13 mmol) were added. The mixture was stirred for additional 2 hours before concentrating to dryness under reduced pressure to give the crude product which was further purified by prep-TLC to give methyl (2²S,2⁴S,Z)-2¹-((1-(tert-butoxycarbonyl)-5-methoxy-7-methyl-1H-indol-4-yl)methyl)-5¹H-3,9-dioxa-2(2,4)-piperidina-5(4,1)-triazola-1(1,2)-benzenacyclononaphane-14-carboxylate (20 mg, Yield 74.0%) as white solid. LC/MS (ESI) m/z: 646 (M+H)⁺.

Step 9: (2²S,2⁴S,Z)-2¹-((5-methoxy-7-methyl-1H-indol-4-yl)methyl)-5¹H-3,9-dioxa-2(2,4)-piperidina-5(4,1)-triazola-1(1,2)-benzenacyclononaphane-1⁴-carboxylic acid (Example 13): To a solution of methyl (2²S,2⁴S,Z)-2¹-((1-(tert-butoxycarbonyl)-5-methoxy-7-methyl-1H-indol-4-yl)methyl)-5¹H-3,9-dioxa-2(2,4)-piperidina-5(4,1)-triazola-1(1,2)-benzenacyclononaphane-14-carboxylate (14 mg, 0.022 mmol) in methanol (0.6 mL)/water (0.2 mL) was added LiOH (2.8 mg, 0.066 mmol) and the mixture was stirred at room temperature overnight. The mixture was acidified with 1 N aq. HCl to pH˜4 and extracted with ethyl acetate twice. The combined organic layers were washed with water and brine, dried over anhydrous Na₂SO₄, filtered and concentrated to dryness under reduced pressure. The residue was further purified by preparatory HPLC to give (2²S,2⁴S,Z)-2¹-((5-methoxy-7-methyl-1H-indol-4-yl)methyl)-5¹H-3,9-dioxa-2(2,4)-piperidina-5(4,1)-triazola-1(1,2)-benzenacyclononaphane-1⁴-carboxylic acid (1.8 mg, Yield 11.2%) as white solid. ¹H NMR (400 MHz, CD₃OH) δ 8.42 (s, 2H), 7.76 (d, J=8.0 Hz, 1H), 7.61 (s, 1H), 7.51 (d, J=8.0 Hz, 1H), 7.30 (d, J=3.1 Hz, 1H), 6.71 (s, 1H), 6.23 (d, J=3.1 Hz, 1H), 5.07 (d, J=14.3 Hz, 1H), 4.76-4.69 (m, 2H), 4.62 (s, 1H), 4.52 (d, J=14.3 Hz, 1H), 4.44-4.36 (m, 2H), 4.33 (d, J=4.8 Hz, 1H), 4.10 (d, J=12.6 Hz, 2H), 4.00 (d, J=12.7 Hz, 1H), 3.66 (s, 3H), 3.56 (dd, J=13.3, 8.8 Hz, 1H), 2.62 (d, J=8.5 Hz, 1H), 2.48 (s, 3H), 2.35 (dd, J=42.4, 12.8 Hz, 2H), 2.01 (d, J=14.6 Hz, 3H). LC/MS (ESI) m/z: 532 (M+H)⁺.

### Examples 14-34

The following compounds are prepared substantially according to the procedures described above:

| Ex. | | |
|---|---|---|
| No. | Chemical structure | Chemical name |
| 14 | 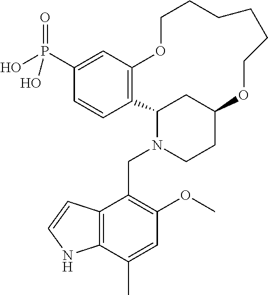 [SMILES: COc1cc(C)c2[nH]ccc2c1CN1CC[C@H]2C[C@H]1c1ccc(P(=O)(O)O)cc1OCCCCCCO2] | ((1²S,1⁴S)-1¹-((5-methoxy-7-methyl-1H-indol- 4-yl)methyl)-3,10-dioxa-1(2,4)-piperidina- 2(1,2)-benzenacyclodecaphane-2⁴- yl)phosphonic acid |
| 15 | 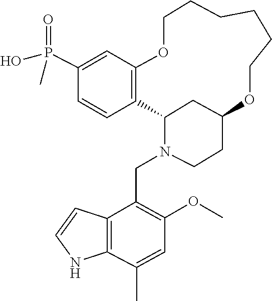 [SMILES: COc1cc(C)c2[nH]ccc2c1CN1CC[C@H]2C[C@H]1c1ccc(P(C)(=O)O)cc1OCCCCCCO2] | ((1²S,1⁴S)-1¹-((5-methoxy-7-methyl-1H-indol- 4-yl)methyl)-3,10-dioxa-1(2,4)-piperidina- 2(1,2)-benzenacyclodecaphane-2⁴- yl)(methyl)phosphinic acid |
| 16 | 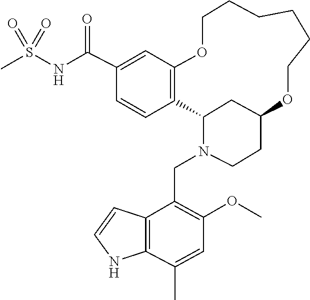 [SMILES: COc1cc(C)c2[nH]ccc2c1CN1CC[C@H]2C[C@H]1c1ccc(C(=O)NS(C)(=O)=O)cc1OCCCCCCO2] | (1²S,1⁴S)-1¹-((5-methoxy-7-methyl-1H-indol- 4-yl)methyl)-N-(methylsulfonyl)-3,10-dioxa- 1(2,4)-piperidina-2(1,2)- benzenacyclodecaphane-2⁴-carboxamide |
| 17 | 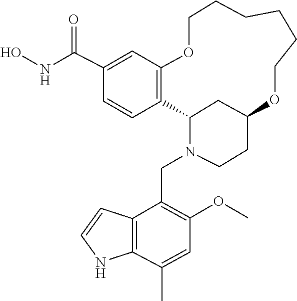 [SMILES: COc1cc(C)c2[nH]ccc2c1CN1CC[C@H]2C[C@H]1c1ccc(C(=O)NO)cc1OCCCCCCO2] | (1²S,1⁴S)-N-hydroxy-1¹-((5-methoxy-7- methyl-1H-indol-4-yl)methyl)-3,10-dioxa- 1(2,4)-piperidina-2(1,2)- benzenacyclodecaphane-2⁴-carboxamide |
| 18 | 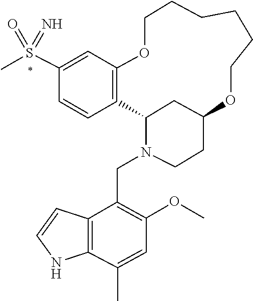 [SMILES: COc1cc(C)c2[nH]ccc2c1CN1CC[C@H]2C[C@H]1c1ccc(S(C)(=N)=O)cc1OCCCCCCO2] | imino((1²S,1⁴S)-1¹-((5-methoxy-7-methyl-1H- indol-4-yl)methyl)-3,10-dioxa-1(2,4)- piperidina-2(1,2)-benzenacyclodecaphane-2⁴- yl)(methyl)-λ⁶-sulfanone |
| 19 | 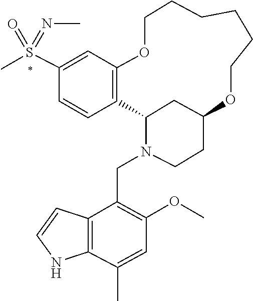 [SMILES: CN=S(C)(=O)c1ccc2c(c1)OCCCCCCO[C@H]1CCN(Cc3c(OC)cc(C)c4[nH]ccc34)[C@H]2C1] | ((1²S,1⁴S)-1¹-((5-methoxy-7-methyl-1H-indol- 4-yl)methyl)-3,10-dioxa-1(2,4)-piperidina- 2(1,2)-benzenacyclodecaphane-2⁴- yl)(methyl)(methylimino)-λ⁶-sulfanone |
| 20 | 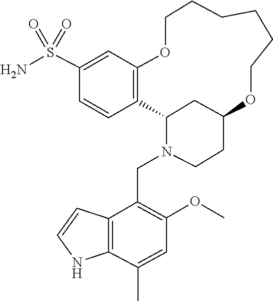 [SMILES: COc1cc(C)c2[nH]ccc2c1CN1CC[C@H]2C[C@H]1c1ccc(S(N)(=O)=O)cc1OCCCCCCO2] | (1²S,1⁴S)-1¹-((5-methoxy-7-methyl-1H-indol- 4-yl)methyl)-3,10-dioxa-1(2,4)-piperidina- 2(1,2)-benzenacyclodecaphane-2⁴- sulfonamide |
| 21 | 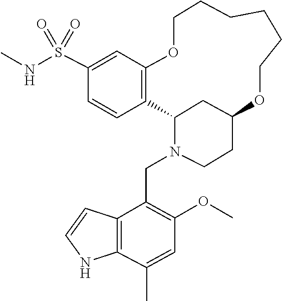 [SMILES: CNS(=O)(=O)c1ccc2c(c1)OCCCCCCO[C@H]1CCN(Cc3c(OC)cc(C)c4[nH]ccc34)[C@H]2C1] | (1²S,1⁴S)-1¹-((5-methoxy-7-methyl-1H-indol- 4-yl)methyl)-N-methyl-3,10-dioxa-1(2,4)- piperidina-2(1,2)-benzenacyclodecaphane-2⁴- sulfonamide |
| 22 | 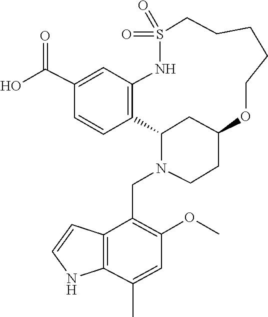 [SMILES: COc1cc(C)c2[nH]ccc2c1CN1CC[C@H]2C[C@H]1c1ccc(C(=O)O)cc1NS(=O)(=O)CCCCCO2] | (1²S,1⁴S)-1¹-((5-methoxy-7-methyl-1H-indol- 4-yl)methyl)-10-oxa-4-thia-3-aza-1(2,4)- piperidina-2(1,2)-benzenacyclodecaphane-2⁴- carboxylic acid 4,4-dioxide |
| 23 | 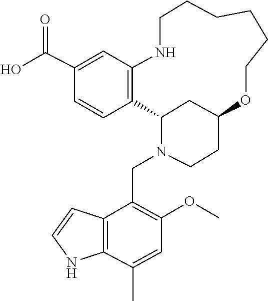 [SMILES: COc1cc(C)c2[nH]ccc2c1CN1CC[C@H]2C[C@H]1c1ccc(C(=O)O)cc1NCCCCCCO2] | (1²S,1⁴S)-1¹-((5-methoxy-7-methyl-1H-indol- 4-yl)methyl)-10-oxa-3-aza-1(2,4)-piperidina- 2(1,2)-benzenacyclodecaphane-2⁴-carboxylic acid |
| 24 | 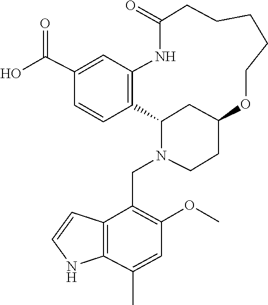 [SMILES: COc1cc(C)c2[nH]ccc2c1CN1CC[C@H]2C[C@H]1c1ccc(C(=O)O)cc1NC(=O)CCCCCO2] | (1²S,1⁴S)-1¹-((5-methoxy-7-methyl-1H-indol- 4-yl)methyl)-4-oxo-10-oxa-3-aza-1(2,4)- piperidina-2(1,2)-benzenacyclodecaphane-2⁴- carboxylic acid |
| 25 | 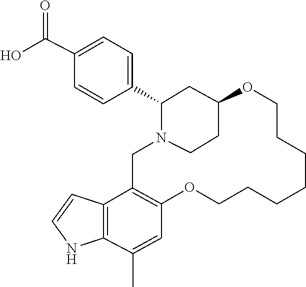 [SMILES: Cc1cc2c(c3cc[nH]c13)CN1CC[C@@H](C[C@H]1c1ccc(C(=O)O)cc1)OCCCCCCCO2] | 4-((3²R,3⁴R)-1⁷-methyl-1¹H-4,12-dioxa-1(4,5)- indola-3(1,4)-piperidinacyclododecaphane-3²- yl)benzoic acid |
| 26 | 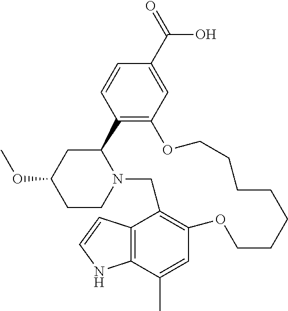 [SMILES: CO[C@H]1CCN2Cc3c(cc(C)c4[nH]ccc34)OCCCCCCCOc3cc(C(=O)O)ccc3[C@@H]2C1] | (18bS,20S)-20-methoxy-4-methyl- 3,8,9,10,11,12,13,18b,19,21,22,24- dodecahydro-7H,20H- benzo[7,8]pyrido[1′,2′:5,6][1,9]dioxa[5]azacyclo- hexadecino[3,2-e]indole-16-carboxylic acid |
| 27 | 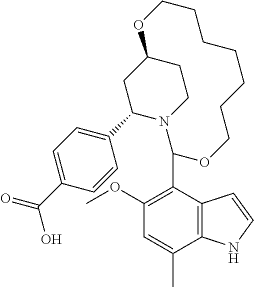 [SMILES: COc1cc(C)c2[nH]ccc2c1C1OCCCCCCCO[C@H]2CCN1[C@H](c1ccc(C(=O)O)cc1)C2] | 4-((1²S,1⁴S)-2-(5-methoxy-7-methyl-1H- indol-4-yl)-3,11-dioxa-1- azabicyclo[10.2.2]hexadecan-14-yl)benzoic acid |
| 28 | 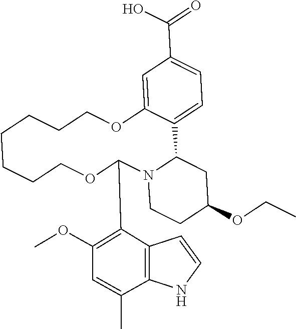 [SMILES: CCO[C@H]1CCN2C(c3c(OC)cc(C)c4[nH]ccc34)OCCCCCCCOc3cc(C(=O)O)ccc3[C@@H]2C1] | (2S,19bS)-2-ethoxy-6-(5-methoxy-7-methyl- 1H-indol-4-yl)-1,3,4,9,10,11,12,13,14,19b- decahydro-2H,6H,8H-benzo[e]pyrido[1,2- c][1,7]dioxa[3]azacyclotetradecine-17- carboxylic acid |
| 29 | 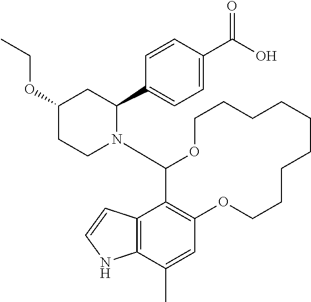 [SMILES: CCO[C@H]1CCN(C2OCCCCCCCCCOc3cc(C)c4[nH]ccc4c32)[C@H](c2ccc(C(=O)O)cc2)C1] | 4-((2S,4S)-4-ethoxy-1-(14-methyl- 1,4,5,6,7,8,9,10,11,15-decahydro-3H- [1,5]dioxacyclotetradecino[3,2-e]indol-1- yl)piperidin-2-yl)benzoic acid |
| 30 | 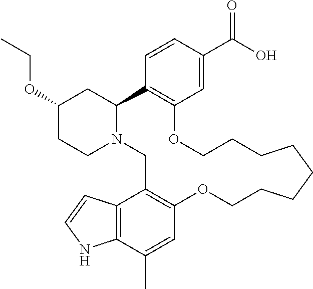 [SMILES: CCO[C@H]1CCN2Cc3c(cc(C)c4[nH]ccc34)OCCCCCCCCOc3cc(C(=O)O)ccc3[C@@H]2C1] | (19bS,21S)-21-ethoxy-4-methyl- 7,8,9,10,11,12,13,14,19b,20,21,22,23,25- tetradecahydro-3H- benzo[7,8]pyrido[1′,2′:5,6][1,9,5]dioxaazacyclo- heptadecino[3,2-e]indole-1⁷-carboxylic acid |
| 31 | 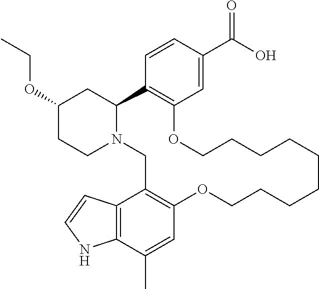 [SMILES: CCO[C@H]1CCN2Cc3c(cc(C)c4[nH]ccc34)OCCCCCCCCCOc3cc(C(=O)O)ccc3[C@@H]2C1] | (20bS,22S)-22-ethoxy-4-methyl- 3,7,8,9,10,11,12,13,14,15,20b,21,22,23,24,26- hexadecahydrobenzo[7,8]pyrido[1′,2′:5,6][1,9, 5]dioxaazacyclooctadecino[3,2-e]indole-18- carboxylic acid |
| 32 | 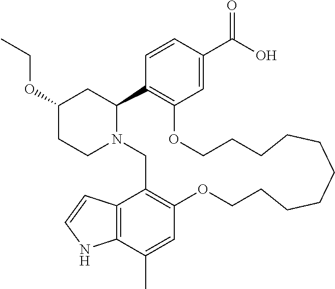 [SMILES: CCO[C@H]1CCN2Cc3c(cc(C)c4[nH]ccc34)OCCCCCCCCCCOc3cc(C(=O)O)ccc3[C@@H]2C1] | (21bS,23S)-23-ethoxy-4-methyl- 7,8,9,10,11,12,13,14,15,16,21b,22,23,24,25,2 7-hexadecahydro-3H- benzo[7,8]pyrido[1′,2′:5,6][1,9,5]dioxaazacyclo- nonadecino[3,2-e]indole-19-carboxylic acid |
| 33 | 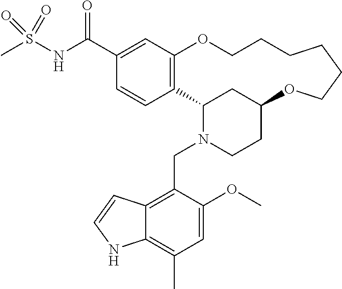 [SMILES: COc1cc(C)c2[nH]ccc2c1CN1CC[C@H]2C[C@H]1c1ccc(C(=O)NS(C)(=O)=O)cc1OCCCCCCO2] | (1²S,1⁴S)-1¹-((5-methoxy-7-methyl-1H-indol- 4-yl)methyl)-N-(methylsulfonyl)-3,10-dioxa- 1(2,4)-piperidina-2(1,2)- benzenacyclodecaphane-2⁴-carboxamide |
| 34 | 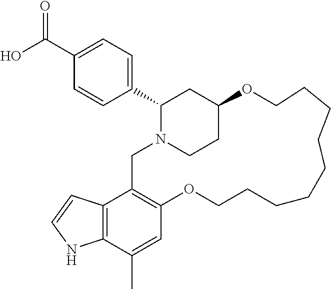 [SMILES: Cc1cc2c(c3cc[nH]c13)CN1CC[C@@H](C[C@H]1c1ccc(C(=O)O)cc1)OCCCCCCCCCO2] | rac-4-((3²R,3⁴R)-1⁷-methyl-11H-4,14-dioxa- 1(4,5)-indola-3(1,4)- piperidinacyclotetradecaphane-3²-yl)benzoic acid |
| 35 | 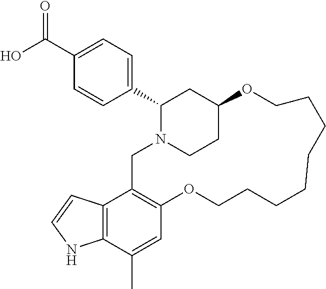 [SMILES: Cc1cc2c(c3cc[nH]c13)CN1CC[C@@H](C[C@H]1c1ccc(C(=O)O)cc1)OCCCCCCCCO2] | 4-((3²R,3⁴R)-1⁷-methyl-11H-4,13-dioxa- 1(4,5)-indola-3(1,4)- piperidinacyclotridecaphane-3²-yl)benzoic acid |
| 36 | 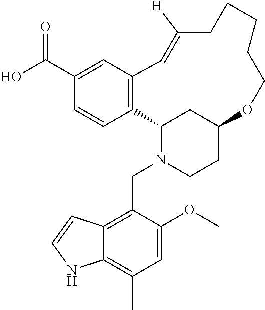 [SMILES: COc1cc(C)c2[nH]ccc2c1CN1CC[C@H]2C[C@H]1c1ccc(C(=O)O)cc1/C=C/CCCCCO2] | (1²R,1⁴R,E)-1¹-((5-methoxy-7-methyl-1H- indol-4-yl)methyl)-10-oxa-1(2,4)-piperidina- 2(1,2)-benzenacyclodecaphan-3-ene-2⁴- carboxylic acid |
| 37 | 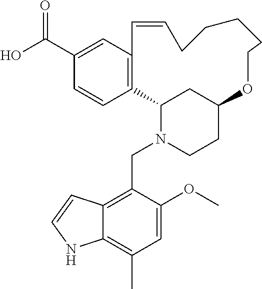 [SMILES: COc1cc(C)c2[nH]ccc2c1CN1CC[C@H]2C[C@H]1c1ccc(C(=O)O)cc1/C=C\CCCCCO2] | (1²R,1⁴R,Z)-1¹-((5-methoxy-7-methyl-1H- indol-4-yl)methyl)-10-oxa-1(2,4)-piperidina- 2(1,2)-benzenacyclodecaphan-3-ene-2⁴- carboxylic acid |
| 38 | 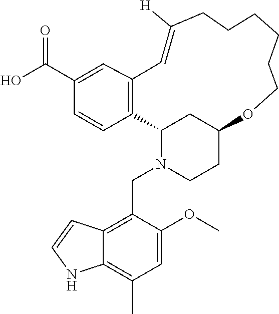 [SMILES: COc1cc(C)c2[nH]ccc2c1CN1CC[C@H]2C[C@H]1c1ccc(C(=O)O)cc1/C=C/CCCCCCO2] | (1²R,1⁴R,E)-11-((5-methoxy-7-methyl-1H- indol-4-yl) methyl)-1¹-oxa-1(2,4)-piperidina- 2(1,2)-benzenacycloundecaphan-3-ene-2⁴- carboxylic acid |
| 39 | 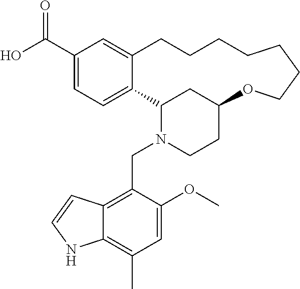 | (1²S,1⁴RS)-1¹-((5-methoxy-7-methyl-1H-indol- 4-yl)methyl)-10-oxa-1(2,4)-piperidina-2(1,2)- benzenacyclodecaphane-2⁴-carboxylic acid |
| 40 | 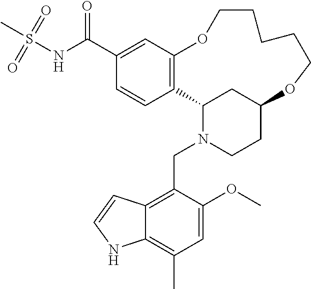 [SMILES: COc1cc(C)c2[nH]ccc2c1CN1CC[C@H]2C[C@H]1c1ccc(C(=O)NS(C)(=O)=O)cc1OCCCCCO2] | (1²R,1⁴R)-1¹-((5-methoxy-7-methyl-1H-indol- 4-yl)methyl)-N-(methylsulfonyl)-3,9-dioxa- 1(2,4)-piperidina-2(1,2)- benzenacyclononaphane-2⁴-carboxamide |
| 41 | 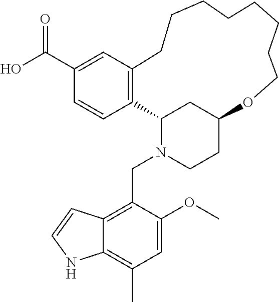 [SMILES: COc1cc(C)c2[nH]ccc2c1CN1CC[C@H]2C[C@H]1c1ccc(C(=O)O)cc1CCCCCCCCO2] | (1²R,1⁴R)-1¹-((5-methoxy-7-methyl-1H-indol- 4-yl) methyl)-11-oxa-1(2,4)-piperidina-2(1,2)- benzenacycloundecaphane-2⁴-carboxylic acid |
| 42 | 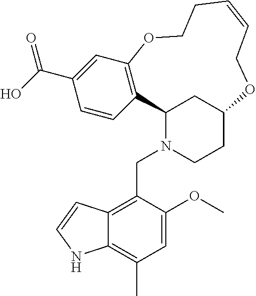 [SMILES: COc1cc(C)c2[nH]ccc2c1CN1CC[C@@H]2C[C@@H]1c1ccc(C(=O)O)cc1OCC/C=C\CO2] | (1²R,1⁴R,Z)-1¹-((5-methoxy-7-methyl-1H- indol-4-yl)methyl)-3,9-dioxa-1(2,4)-piperidina- 2(1,2)-benzenacyclononaphan-6-ene-2⁴- carboxylic acid |
| 43 | 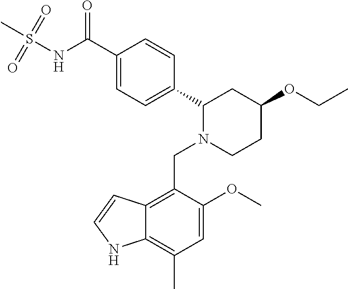 | 4-((2S,4S)-4-ethoxy-1-((5-methoxy-7-methyl- 1H-indol-4-yl)methyl)piperidin-2-yl)-N- (methylsulfonyl)benzamide |
| 44 | 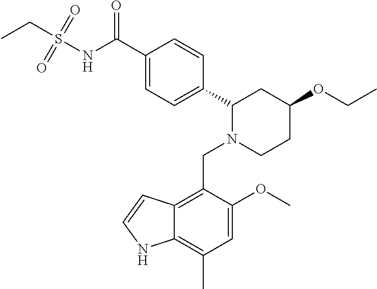 | 4-((2S,4S)-4-ethoxy-1-((5-methoxy-7-methyl- 1H-indol-4-yl)methyl)piperidin-2-yl)-N- (ethylsulfonyl)benzamide |
| 45 | 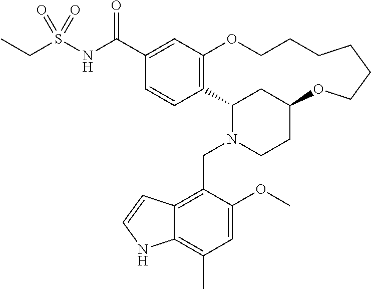 [SMILES: CCS(=O)(=O)NC(=O)c1ccc2c(c1)OCCCCCCO[C@H]1CCN(Cc3c(OC)cc(C)c4[nH]ccc34)[C@H]2C1] | (1²S,1⁴S)-N-(ethylsulfonyl)-1¹-((5-methoxy-7- methyl-1H-indol-4-yl)methyl)-3,10-dioxa- 1(2,4)-piperidina-2(1,2)- benzenacyclodecaphane-2⁴-carboxamide |
| 46 | 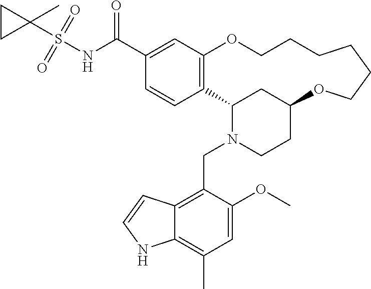 [SMILES: COc1cc(C)c2[nH]ccc2c1CN1CC[C@H]2C[C@H]1c1ccc(C(=O)NS(=O)(=O)C3(C)CC3)cc1OCCCCCCO2] | (1²S,1⁴S)-1¹-((5-methoxy-7-methyl-1H-indol- (4-yl)methyl)-N-((1- methylcyclopropyl)sulfonyl)-3,10-dioxa-1(2,4)- piperidina-2(1,2)-benzenacyclodecaphane-2⁴- carboxamide |
| 47 | 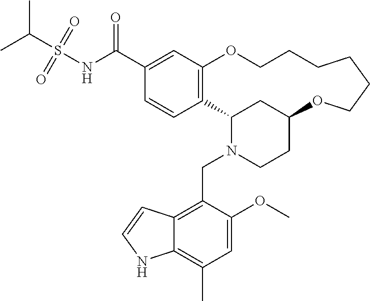 [SMILES: COc1cc(C)c2[nH]ccc2c1CN1CC[C@H]2C[C@H]1c1ccc(C(=O)NS(=O)(=O)C(C)C)cc1OCCCCCCO2] | (1²S,1⁴S)-N-(isopropylsulfonyl)-11-((5- methoxy-7-methyl-1H-indol-4-yl)methyl)-3,10- dioxa-1(2,4)-piperidina-2(1,2)- benzenacyclodecaphane-2⁴-carboxamide |

### Examples 48 and 49: (1²S,1⁴S,E)-1¹-((5-methoxy-7-methyl-1H-indol-4-yl)methyl)-3,10-dioxa-1(2,4)-piperidina-2(1,7)-naphthalenacyclodecaphan-7-ene-24-carboxylic acid and (1²S,1⁴S)-1¹-((5-methoxy-7-methyl-1H-indol-4-yl)methyl)-3,10-dioxa-1(2,4)-piperidina-2(1,7)-naphthalenacyclodecaphane-24-carboxylic acid

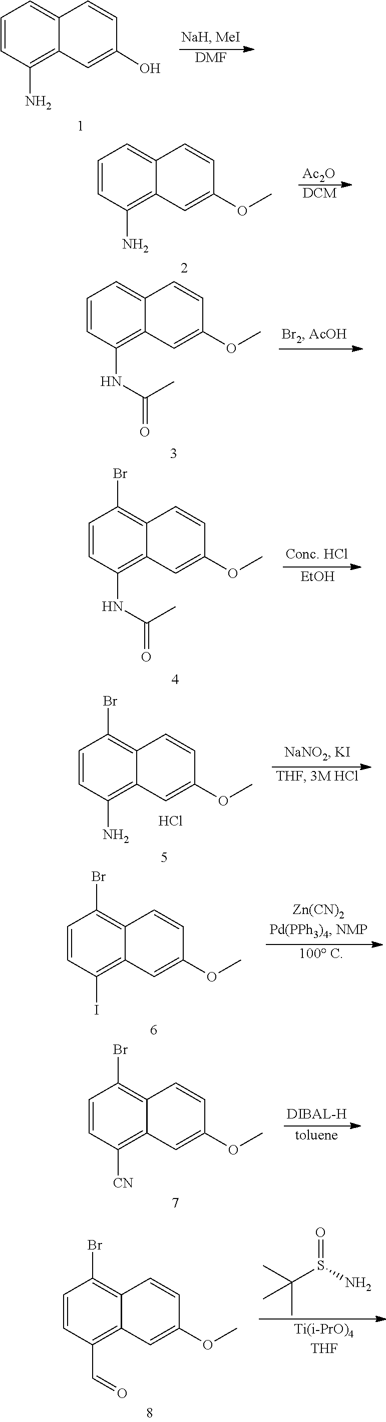

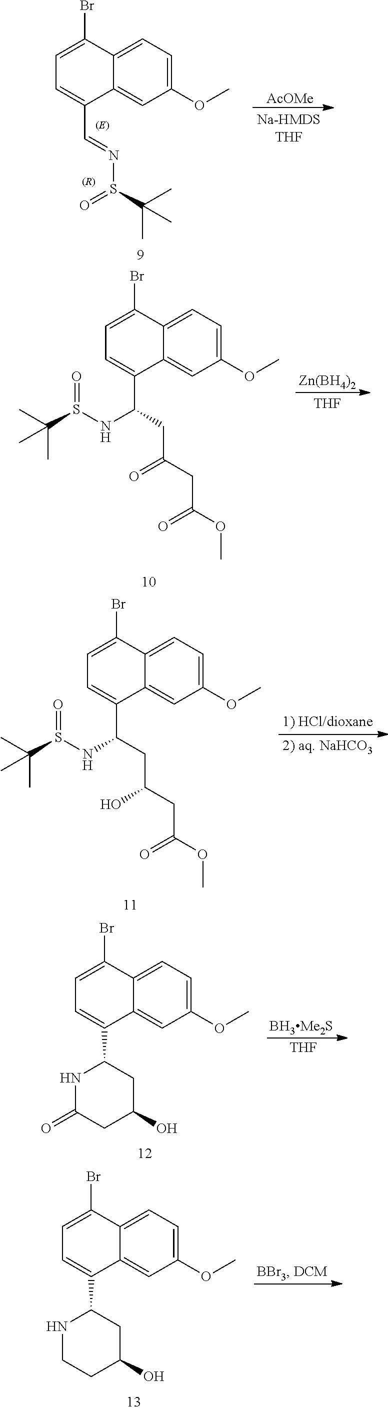

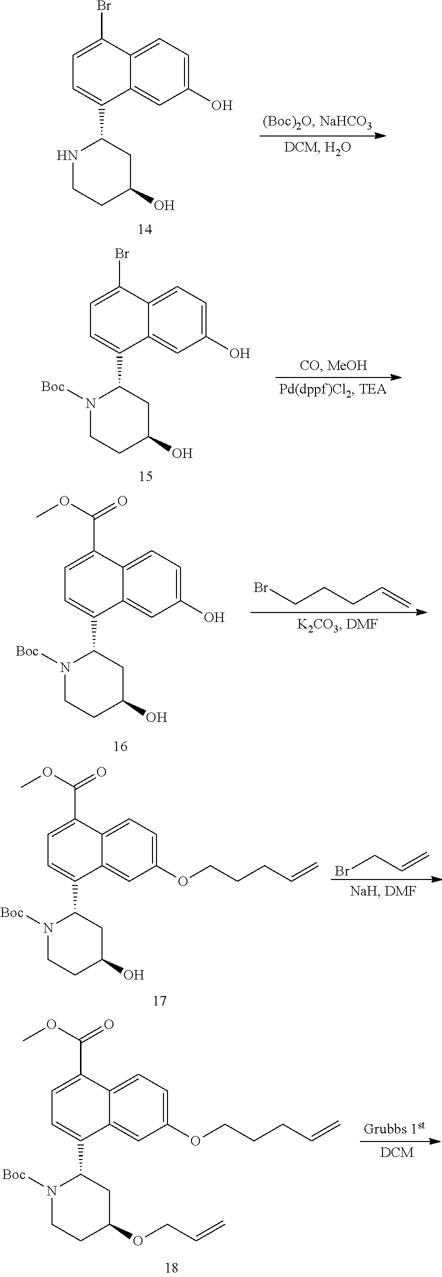

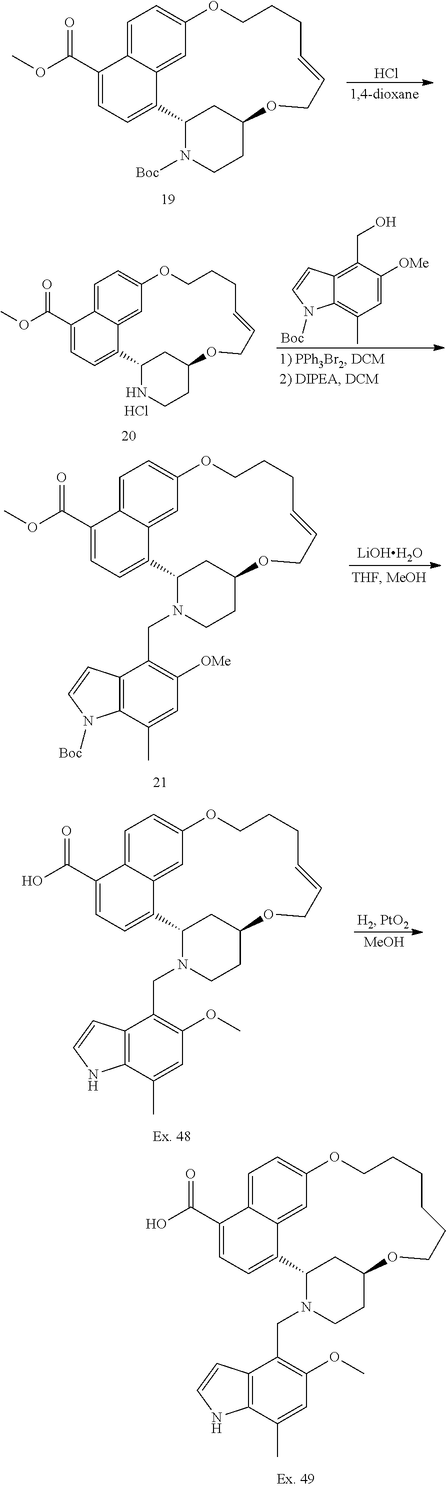

Step 1: 7-methoxynaphthalen-1-amine (2): At 0° C., to a solution of 8-aminonaphthalen-2-ol (50 g, 314 mmol) in DMF (500 mL) was added NaH (18.85 g, 472 mmol, 60% dispersion in mineral oil) portion-wise and the mixture was stirred under N₂ atmosphere at 0° C. for 1 hour. Then MeI (44.6 g, 314 mmol) was added and the resulting mixture was stirred at 0° C. for another 1 hour. The mixture was quenched with saturated aq.NH₄Cl solution and extracted with EtOAc twice. The combined organic layers were washed with brine, dried over Na₂SO₄, filtered and concentrated to dryness. The residue was purified by column chromatography on silica gel (PE:EtOAc=10:1) to give 7-methoxynaphthalen-1-amine (55 g, 100% yield) as brown solid. LC/MS (ESI) (m/z): 174 (M+H)⁺.

Step 2: N-(7-methoxynaphthalen-1-yl)acetamide (3): To a mixture of 7-methoxynaphthalen-1-amine (55 g, 314 mmol) in DCM (550 mL) was added acetic anhydride (34 g, 333 mmol) and the mixture was stirred at r.t. overnight. The mixture was quenched with saturated aq.NH₄Cl solution and extracted with DCM twice. The combined organic layers were washed with brine, dried over Na₂SO₄, filtered and concentrated to dryness. The residue was purified by column chromatography on silica gel (PE:EtOAc=10:1) to give N-(7-methoxy-naphthalen-1-yl)acetamide (58 g, 85.8% yield) as brown solid. LC/MS (ESI) (m/z): 216 (M+H)⁺.

Step 3: N-(4-bromo-7-methoxynaphthalen-1-yl)acetamide (4): At 0° C., to a solution of N-(7-methoxynaphthalen-1-yl)acetamide (55 g, 256 mmol) in acetic acid (300 mL) was added Br₂ (36.8 g, 230 mmol in acetic acid) drop-wisely and the mixture was stirred under N₂ atmosphere for 30 minutes. The mixture was quenched with saturated aq.NaHCO₃ solution and extracted with EtOAc twice. The combined organic layers were washed with brine, dried over anhydrous Na₂SO₄, filtered and concentrated to dryness. The residue was purified by column chromatography on silica gel (PE: DCM=4:1) to give N-(4-bromo-7-methoxynaphthalen-1-yl)acetamide (66 g, yield 87.6%) as brown solid. LC/MS (ESI) (m/z): 294 (M+H)⁺.

Step 4: 4-bromo-7-methoxynaphthalen-1-amine hydrochloride (5): To a solution of N-(4-bromo-7-methoxynaphthalen-1-yl)acetamide (65 g, 221 mmol) in EtOH (650 mL) was added conc.HCl (184 mL, 2.21 mol) and the mixture was stirred at 80° C. for 6 hours. The mixture was concentrated to dryness to give 4-bromo-7-methoxynaphthalen-1-amine hydrochloride (51.5 g, yield 80.8%) as brown solid. LC/MS (ESI) (m/z): 252 (M+H)⁺.

Step 5: 1-bromo-4-iodo-6-methoxynaphthalene (6): To a solution of 4-bromo-7-methoxynaphthalen-1-amine hydrochloride (45 g, 178.5 mmol) in 3N aq.HCl (250 mL) and THF (250 mL) was added a solution of NaNO₂ (13.25 g, 192 mmol) in water (100 mL) drop-wisely at 0° C. and the mixture was stirred at 0° C. for 2 hours. A solution of KI (88.9 g, 535.5 mmol) in water (200 mL) was added to the above mixture at 0° C. and the resulting mixture was stirred at room temperature for 2 hours. The mixture was extracted with EtOAc twice and the combined organic layers were washed brine, dried over anhydrous Na₂SO₄, filtered and concentrated to dryness. The residue was purified by column chromatography on silica gel (PE: DCM=4:1) to give 1-bromo-4-iodo-6-methoxynaphthalene (31.5 g, yield 47.7%) as brown solid. ¹H NMR (400 MHz, CDCl₃) δ 8.12 (d, J=9.2 Hz, 1H), 7.86 (d, J=7.9 Hz, 1H), 7.41 (d, J=2.4 Hz, 1H), 7.35 (d, J=7.9 Hz, 1H), 7.26-7.23 (m, 1H), 3.99 (s, 3H).

Step 6: 4-bromo-7-methoxy-1-naphthonitrile (7): To a mixture of 1-bromo-4-iodo-6-methoxynaphthalene (23.5 g, 64.4 mmol) in NMP (200 mL) was added Zn(CN)₂ (7.91 g, 67.6 mmol) and Pd(PPh₃)₄(7.4 g, 6.4 mmol), the mixture was degassed under N₂ atmosphere for three times and stirred at 100° C. for 1 hour. The mixture was diluted with EtOAc, washed with water and brine, dried over anhydrous Na₂SO₄, filtered and concentrated to dryness. The residue was purified by column chromatography on silica gel (PE:EtOAc=3:1) to give 4-bromo-7-methoxy-1-naphthonitrile (16.5 g, yield 97.8%) as yellow solid. ¹HNMR (400 MHz, CDCl₃) δ 8.20 (d, J=9.3 Hz, 1H), 7.69-7.64 (m, 2H), 7.44 (d, J=2.5 Hz, 1H), 7.33 (dd, J=9.3, 2.5 Hz, 1H), 4.00 (s, 3H).

Step 7: 4-bromo-7-methoxy-1-naphthaldehyde (8): To a mixture of 4-bromo-7-methoxy-1-naphthonitrile (16.5 g, 62.9 mmol) in toluene (170 mL) was added DIBAL-H (126 mL, 126 mmol, 1M in hexane) drop-wisely at −78° C. and the mixture was stirred under N₂ atmosphere at −78° C. for 2 hours. The mixture was quenched with water and extracted with EtOAc twice. The combined organic layers were washed with brine, dried over anhydrous Na₂SO₄, filtered and concentrated to dryness. The residue was purified by column chromatography on silica gel (PE:EtOAc=3:1) to give 4-bromo-7-methoxy-1-naphthonitrile (13.4 g, yield 80.3%) as yellow solid.

Step 8: (R,E)-N-((4-bromo-7-methoxynaphthalen-1-yl)methylene)-2-methylpropane-2-sulfinamide (9): To a solution of 4-bromo-7-methoxy-1-naphthaldehyde (13.3 g, 50.2 mmol) in THF (150 mL) was added (R)-2-methylpropane-2-sulfinamide (15.2 g, 125.4 mmol) and Ti(i-PrO)₄ (64.1 g, 280.9 mmol) and the mixture was stirred at 70° C. overnight. The mixture was quenched with water and extracted with EtOAc twice. The combined organic layers were washed with brine, dried over anhydrous Na₂SO₄, filtered and concentrated to dryness. The residue was purified by column chromatography on silica gel (PE:EtOAc=1:1) to give (R,E)-N-((4-bromo-7-methoxynaphthalen-1-yl)methylene)-2-methylpropane-2-sulfinamide (17.85 g, yield 96.6%) as yellow solid. LC/MS (ESI) m/z: 368 (M+H)⁺.

Step 9: methyl (S)-5-(4-bromo-7-methoxynaphthalen-1-yl)-5-(((R)-tert-butylsulfinyl)amino)-3-oxopentanoate (10): To a solution of NaHMDS (97 mL, 291 mmol, 3.0 M in THF) in THF (180 mL) was added methyl acetate (14.32 g, 193.3 mmol) drop-wisely at −65° C. and the mixture was stirred at −65° C. for 1 hour. A solution of (R,E)-N-((4-bromo-7-methoxynaphthalen-1-yl)methylene)-2-methylpropane-2-sulfinamide (17.8 g, 48.3 mmol) in THF (260 mL) was added to the above mixture drop-wisely at −65° C. and the resulting mixture was stirred at −20° C. for 2 hours. The mixture was quenched with saturated aq.NH₄Cl solution and extracted with EtOAc twice. The combined organic layers were washed with brine, dried over anhydrous Na₂SO₄, filtered and concentrated to dryness. The residue was purified by column chromatography on silica gel (PE:EtOAc=1:1) to give (3R,5S)-5-(4-bromo-7-methoxynaphthalen-1-yl)-5-(((R)-tert-butylsulfinyl)amino)-3-hydroxypentanoate (18.1 g, yield 77.3%) as yellow solid. LC/MS (ESI) m/z: 484 (M+H)⁺.

Step 10: methyl (3R,5S)-5-(4-bromo-7-methoxynaphthalen-1-yl)-5-(((R)-tert-butylsulfinyl)amino)-3-hydroxypentanoate (11): To a solution of (3R,5S)-5-(4-bromo-7-methoxynaphthalen-1-yl)-5-(((R)-tert-butylsulfinyl)amino)-3-hydroxypentanoate (18.0 g, 37.2 mmol) in THF (200 mL) was added Zn(BH₄)₂(75 mL, 75 mmol, 1.0 M in THF) drop-wisely at −65° C. and the mixture was stirred at −65° C. for 3 hours. The mixture was quenched with saturated aq.NH₄Cl solution and extracted with EtOAc twice. The combined organic layers were washed with brine, dried over anhydrous Na₂SO₄, filtered and concentrated to dryness.

The residue was purified by column chromatography on silica gel (DCM:MeOH=10:1) to give methyl (3R,5S)-5-(4-bromo-7-methoxynaphthalen-1-yl)-5-(((R)-tert-butylsulfinyl)amino)-3-hydroxypentanoate (13.8 g, yield 76.3%) as yellow solid. LC/MS (ESI) m/z: 486 (M+H)⁺.

Step 11: (4R,6S)-6-(4-bromo-7-methoxynaphthalen-1-yl)-4-hydroxypiperidin-2-one (12): A mixture of methyl (3R,5S)-5-(4-bromo-7-methoxynaphthalen-1-yl)-5-(((R)-tert-butylsulfinyl)amino)-3-hydroxypentanoate (13.6 g, 28.0 mmol) in HCl/1,4-dioxane (28 mL, 4M) was stirred at 25° C. for 1 hour. The mixture was concentrated to dryness and the residue was basified with 5% aq.NaHCO₃ solution to pH˜8 and diluted with THF (70 mL). The resulting mixture was stirred at room temperature overnight. The mixture was diluted with EtOAc, washed with water and brine, dried over anhydrous Na₂SO₄, filtered and concentrated under reduced pressure to dryness. The residue was purified by flash chromatography (DCM:MeOH=10:1) to give (4R,6S)-6-(4-bromo-7-methoxynaphthalen-1-yl)-4-hydroxypiperidin-2-one (8.7 g, yield 88.9%) as yellow solid. LC/MS (ESI) m/z: 350 (M+H)⁺.

Step 12: (2S,4S)-2-(4-bromo-7-methoxynaphthalen-1-yl)piperidin-4-ol (13): To a solution of (4R,6S)-6-(4-bromo-7-methoxynaphthalen-1-yl)-4-hydroxypiperidin-2-one (8.57 g, 24.5 mmol) in THF (100 mL) was added BH₃·Me₂S (12.3 mL, 123 mmol, 10.0 M in Me₂S) drop-wisely at 0° C. and the mixture was stirred at 50° C. for 18 hours. The reaction was quenched with 1N aq.HCl and the mixture was concentrated under reduced pressure to dryness. The residue was purified by flash chromatography (DCM:MeOH=10:1) to give (2S,4S)-2-(4-bromo-7-methoxynaphthalen-1-yl)piperidin-4-ol (6.4 g, yield 77.7%) as yellow solid. LC/MS (ESI) m/z: 336 (M+H)⁺.

Step 13: (2S,4S)-2-(4-bromo-7-hydroxynaphthalen-1-yl)piperidin-4-ol (14): To a solution of (2S,4S)-2-(4-bromo-7-methoxynaphthalen-1-yl)piperidin-4-ol (6.0 g, 17.8 mmol) in DCM (60 mL) was added BBr₃ (36.0 mL, 36.0 mmol, 1.0 M in DCM) drop-wisely at −78° C. After addition, the mixture was stirred at −78° C. for 2 hours. The reaction was quenched with saturated NaHCO₃ solution and extracted with DCM twice. The combined organic layers were concentrated to dryness and the residue was purified by flash chromatography (DCM:MeOH=5:1) to give (2S,4S)-2-(4-bromo-7-hydroxynaphthalen-1-yl)piperidin-4-ol (4.5 g, yield 78.3%) as yellow solid. LC/MS (ESI) m/z: 322 (M+H)⁺.

Step 14: tert-butyl (2S,4S)-2-(4-bromo-7-hydroxynaphthalen-1-yl)-4-hydroxypiperidine-1-carboxylate (15): To a solution of (2S,4S)-2-(4-bromo-7-hydroxynaphthalen-1-yl)piperidin-4-ol (4.5 g, 16.9 mmol) in THF (80 mL) and water (20 mL) was added Boc₂O (7.3 g, 33.7 mmol) and NaHCO₃ (4.3 g, 50.7 mmol) and the mixture was stirred at 25° C. for 3 hours. The mixture was diluted with EtOAc, washed with water and brine, dried over anhydrous Na₂SO₄, filtered and concentrated to dryness. The residue was purified by column chromatography on silica gel (PE:EtOAc=1:1) and further purified by SFC to give tert-butyl (2S,4S)-2-(4-bromo-7-hydroxynaphthalen-1-yl)-4-hydroxypiperidine-1-carboxylate (1.96 g, 33.2% yield) as white solid. LC/MS (ESI) m/z: 422 (M+H)⁺.

Step 15: tert-butyl (2S,4S)-4-hydroxy-2-(7-hydroxy-4-(methoxycarbonyl)naphthalen-1-yl)piperidine-1-carboxylate (16): To a solution of tert-butyl (2S,4S)-2-(4-bromo-7-hydroxynaphthalen-1-yl)-4-hydroxypiperidine-1-carboxylate (1.7 g, 4.03 mmol) and in MeOH (40 mL) was added Pd(dppf)Cl₂ (146 mg, 0.2 mmol) and TEA (1.22 g, 12.08 mmol) and the mixture was stirred under 30 psi of CO at 50° C. for 16 hours. The mixture was diluted with EtOAc, washed with water and brine, dried over anhydrous Na₂SO₄, filtered and concentrated under reduced pressure to dryness. The residue was purified by flash chromatography on silica gel (PE:EtOAc=50:1 to 4:1) to give tert-butyl (2S,4S)-4-hydroxy-2-(7-hydroxy-4-(methoxycarbonyl)naphthalen-1-yl)piperidine-1-carboxylate (1.56 g, yield 96.4%) as white solid. LC/MS (ESI) m/z: 402 (M+H)⁺.

Step 16: tert-butyl (2S,4S)-4-hydroxy-2-(4-(methoxycarbonyl)-7-(pent-4-en-1-yloxy)naphthalen-1-yl)piperidine-1-carboxylate (17): To a mixture of tert-butyl (2S,4S)-4-hydroxy-2-(7-hydroxy-4-(methoxycarbonyl)naphthalen-1-yl)piperidine-1-carboxylate (280 mg, 0.7 mmol) in DMF (3 mL) was added K₂CO₃ (145 mg, 1.05 mmol) and 5-bromopent-1-ene (126 mg, 0.84 mmol) and the mixture was stirred at 25° C. overnight. The mixture was diluted with EtOAc, washed with water and brine, dried over Na₂SO₄, filtered and concentrated under reduced pressure to dryness. The residue was purified by flash chromatography (PE:EtOAc=3:1) to give tert-butyl (2S,4S)-4-hydroxy-2-(4-(methoxycarbonyl)-7-(pent-4-en-1-yloxy)naphthalen-1-yl)piperidine-1-carboxylate (218 mg, yield 66.3%) as white solid. LC/MS (ESI) m/z: 470 (M+H)⁺.

Step 17: tert-butyl (2S,4S)-4-(allyloxy)-2-(4-(methoxycarbonyl)-7-(pent-4-en-1-yloxy)naphthalen-1-yl)piperidine-1-carboxylate (18): At 0° C., to a solution of tert-butyl (2S,4S)-4-hydroxy-2-(4-(methoxycarbonyl)-7-(pent-4-en-1-yloxy)naphthalen-1-yl)piperidine-1-carboxylate (209 mg, 0.45 mmol) in DMF (2 mL) was added NaH (36 mg, 0.90 mmol, 60% dispersion in mineral oil) in portions and the mixture was stirred under N₂ atmosphere at 0° C. for 0.5 hour. Then 3-bromoprop-1-ene (81 mg, 0.67 mmol) was added and the resulting mixture was stirred at 25° C. for 1 hour. The mixture was quenched with saturated aq.NH₄Cl solution and extracted with EtOAc twice. The combined organic layers were washed with water and brine, dried over Na₂SO₄, filtered and concentrated to dryness. The residue was purified by column chromatography on silica gel (PE:EtOAc=3:1) to give tert-butyl (2S,4S)-4-(allyloxy)-2-(4-(methoxycarbonyl)-7-(pent-4-en-1-yloxy)naphthalen-1-yl)piperidine-1-carboxylate (209 mg, yield 92.1%) as white solid. LC/MS: m/z 510 (M+H)⁺.

Step 18: 1¹-(tert-butyl) 2⁴-methyl (1²S,1⁴S,E)-3,10-dioxa-1(2,4)-piperidina-2(1,7)-naphthalenacyclodecaphan-7-ene-1¹,2⁴-dicarboxylate (19): To a solution of tert-butyl (2S,4S)-4-(allyloxy)-2-(4-(methoxycarbonyl)-7-(pent-4-en-1-yloxy)naphthalen-1-yl)piperidine-1-carboxylate (187 mg, 0.37 mmol) in dry DCM (190 mL) was added Grubbs 1^{st} catalyst (38 mg) and the mixture was stirred under N₂ atmosphere at 25° C. for 18 hours. The mixture was concentrated under reduced pressure to dryness and the residue was purified by flash chromatography (PE:EtOAc=10:1) to give 1′-(tert-butyl) 2⁴-methyl (1²S,1⁴S,E)-3,10-dioxa-1(2,4)-piperidina-2(1,7)-naphthalenacyclodecaphan-7-ene-1¹,2⁴-dicarboxylate (133 mg, yield 75.3%) as brown solid. LC/MS (ESI) m/z: 482 (M+H)⁺.

Step 19: methyl (1²S,1⁴S,E)-3,10-dioxa-1(2,4)-piperidina-2(1,7)-naphthalenacyclodecaphan-7-ene-2⁴-carboxylate hydrochloride (20): A mixture of 11-(tert-butyl) 2⁴-methyl (1²,1⁴S,E)-3,10-dioxa-1(2,4)-piperidina-2(1,7)-naphthalenacyclodecaphan-7-ene-1¹,2⁴-dicarboxylate (120 mg, 0.25 mmol) in HCl/1,4-dioxane (1 mL, 4.0 mmol) was stirred at 25° C. for 1 hour. The mixture was concentrated to dryness to give methyl (1²,1⁴S,E)-3,10-dioxa-1(2,4)-piperidina-2(1,7)-naphthalenacyclodecaphan-7-ene-2⁴-carboxylate hydrochloride (110 mg, yield 100%) as white solid, which was used directly without purification. LC/MS (ESI) m/z: 382 (M+H)⁺.

Step 20: Methyl (1²S,1⁴S,E)-11-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate (21): At 0° C., to a solution of tert-butyl 4-(hydroxymethyl)-5-methoxy-7-methyl-1H-indole-1-carboxylate (88 mg, 0.3 mmol) in dry DCM (1 mL) was added PPh₃Br₂ (138 mg, 0.33 mmol) in portions and the mixture stirred under N₂ atmosphere at 0° C. for 2 hours. Then methyl (1²S,1⁴S,Z)-10-oxa-1(2,4)-piperidina-2(1,2)-benzenacyclodecaphan-3-ene-2⁴-carboxylate hydrochloride (100 mg, 0.24 mmol) and DIPEA (97 mg, 0.75 mmol) in dry DCM (1 mL) were added and the mixture was stirred at 0° C. for 1.5 hours. The mixture was concentrated to dryness. The residue was purified by flash chromatography on silica gel (DCM:MeOH=97:3) to give methyl (1²S,1⁴S,E)-1¹-((1-(tert-butoxycarbonyl)-5-methoxy-7-methyl-1H-indol-4-yl)methyl)-3,10-dioxa-1 (2,4)-piperidina-2(1,7)-naphthalenacyclodecaphan-7-ene-2⁴-carboxylate (84 mg, yield 53.5%) as white solid. LC/MS (ESI) m/z: 655 (M+H)*.

Step 21: (1²S,1⁴S,E)-1¹-((5-methoxy-7-methyl-1H-indol-4-yl)methyl)-3,10-dioxa-1(2,4)-piperidina-2(1,7)-naphthalenacyclodecaphan-7-ene-2⁴-carboxylic acid (Example 48). To a mixture of methyl (1²S,1⁴S,E)-11-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate (84 mg, 0.13 mmol) and LiOH·H₂O (17 mg, 0.39 mmol) in MeOH (4 mL)/water (1 mL) and the mixture was stirred at 60° C. overnight. The reaction mixture was acidified with 1 N aq.HCl solution to pH˜4 and extracted with EtOAc twice. The combined organic layers were washed with brine, dried over anhydrous Na₂SO₄, filtered and concentrated to dryness.

The residue was purified by prep-HPLC to give (1²S,1⁴S,E)-1¹-((5-methoxy-7-methyl-1H-indol-4-yl)methyl)-3,10-dioxa-1 (2,4)-piperidina-2(1,7)-naphthalenacyclodecaphan-7-ene-2⁴-carboxylic acid (45 mg, yield 64.0%) as white solid. ¹H NMR (400 MHz, CD₃OD) b 8.58 (d, J=9.3 Hz, 1H), 8.33 (s, 1H), 7.84 (s, 2H), 7.45 (d, J=2.2 Hz, 1H), 7.28 (d, J=3.1 Hz, 1H), 7.20 (dd, J=9.3, 2.3 Hz, 1H), 6.74 (s, 1H), 6.40 (d, J=3.1 Hz, 1H), 6.14-5.99 (m, 1H), 5.78 (dt, J=15.5, 4.8 Hz, 1H), 5.58 (d, J=10.0 Hz, 1H), 4.70-4.62 (m, 1H), 4.37 (dd, J=14.2, 5.0 Hz, 1H), 4.23 (d, J=7.8 Hz, 3H), 3.93 (dd, J=14.3, 4.3 Hz, 1H), 3.86 (s, 1H), 3.80 (s, 3H), 3.76 (s, 1H), 3.45 (d, J=10.8 Hz, 1H), 2.49 (s, 3H), 2.27 (d, J=15.3 Hz, 2H), 2.27 (d, J=15.3 Hz, 2H), 2.17-2.10 (m, 2H), 2.06 (d, J=15.5 Hz, 2H), 1.97 (dd, J=19.3, 11.0 Hz, 2H). LC/MS (ESI) m/z: 541 (M+H)⁺.

Step 22: (1²S,14S)-1¹-((5-methoxy-7-methyl-1H-indol-4-yl)methyl)-3,10-dioxa-1(2,4)-piperidina-2(1,7)-naphthalenacyclodecaphane-2⁴-carboxylic acid (Example 49). To a solution of (1²S,1⁴S,E)-1¹-((5-methoxy-7-methyl-1H-indol-4-yl)methyl)-3,10-dioxa-1(2,4)-piperidina-2(1,7)-naphthalenacyclodecaphan-7-ene-2⁴-carboxylic acid (16 mg, 0.03 mmol) in MeOH (4 mL) was added PtO₂ (4 mg) at 25° C. under N₂ atmosphere and the mixture was stirred under a H₂ balloon at 25° C. for 2 hours. The mixture was filtered and the filtrate was concentrated to dryness. The residue was purified by prep-HPLC to give to give (1²S,14S)-1¹-((5-methoxy-7-methyl-1H-indol-4-yl)methyl)-3,10-dioxa-1 (2,4)-piperidina-2(1,7)-naphthalenacyclodecaphane-2⁴-carboxylic acid (6.0 mg, yield 36.9%) as white solid. ¹H NMR (400 MHz, CD₃OD) δ 10.71 (s, 1H), 8.61 (d, J=9.3 Hz, 1H), 8.38 (s, 1H), 7.88 (s, 2H), 7.65 (d, J=2.1 Hz, 1H), 7.34 (d, J=3.1 Hz, 1H), 7.24 (dd, J=9.3, 2.2 Hz, 1H), 6.79 (s, 1H), 6.47 (d, J=3.1 Hz, 1H), 5.66 (d, J=9.8 Hz, 1H), 4.37 (d, J=4.2 Hz, 2H), 4.34-4.30 (m, 1H), 3.85 (s, 3H), 3.78 (s, 2H), 3.50 (t, J=9.0 Hz, 2H), 2.53 (s, 3H), 2.24 (d, J=14.5 Hz, 1H), 2.21-2.10 (m, 2H), 2.07 (s, 2H), 1.96 (d, J=7.5 Hz, 1H), 1.89-1.74 (m, 4H), 1.74-1.47 (m, 4H), 1.33 (m, 1H). LC/MS (ESI) m/z: 543 (M+H)⁺.

### Example 50: (12S,14S)—N-(cyclopropylsulfonyl)-11-((5-methoxy-7-methyl-1H-indol-4-yl)methyl)-3,10-dioxa-1(2,4)-piperidina-2(1,2)-benzenacyclodecaphane-24-carboxamide

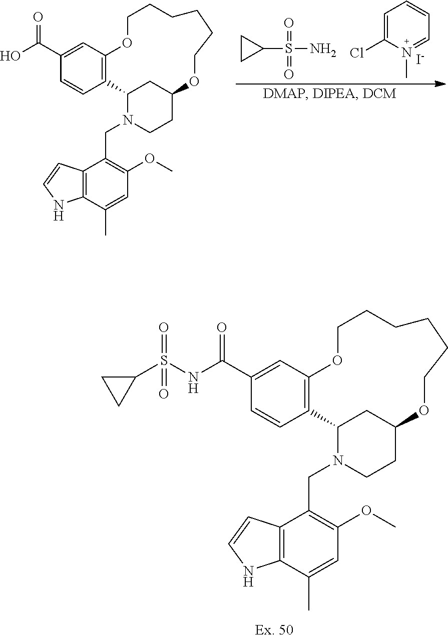

At 0° C., to a solution of (1²S,1⁴S)-11-((5-methoxy-7-methyl-1H-indol-4-yl)methyl)-3,10-dioxa-1(2,4)-piperidina-2(1,2)-benzenacyclodecaphane-24-carboxylic acid (4 mg, 8 μmol) in anhydrous DCM (1 mL) was added cyclopropanesulfonamide (2 mg, 10.5 μmol), 2-chloro-1-methylpyridin-1-ium iodide (3 mg, 10.8 umol), DIPEA (3 mg, 24 umol) and DMAP (1 mg, 8 umol) and the mixture was stirred room temperature overnight. The mixture was concentrated to dryness. The residue was purified by prep-HPLC to give (12S,14S)—N-(cyclopropylsulfonyl)-11-((5-methoxy-7-methyl-1H-indol-4-yl)methyl)-3,10-dioxa-1(2,4)-piperidina-2(1,2)-benzenacyclodecaphane-24-carboxamide (1.0 mg, yield 21.0%) as white solid. ¹HNMR (400 MHz, CD₃OD) δ 8.32 (s, 1H), 7.85 (d, J=8.1 Hz, 1H), 7.77 (s, 1H), 7.65 (d, J=8.0 Hz, 1H), 7.33 (d, J=3.1 Hz, 1H), 6.81 (s, 1H), 6.44 (d, J=3.1 Hz, 1H), 5.41 (d, J=10.9 Hz, 1H), 4.51 (dd, J=22.9, 10.9 Hz, 3H), 4.14 (d, J=12.7 Hz, 1H), 3.95 (t, J=9.0 Hz, 1H), 3.87 (s, 3H), 3.73 (s, 1H), 3.65-3.51 (m, 4H), 3.43 (d, J=10.4 Hz, 1H), 3.10 (dd, J=8.8, 4.1 Hz, 1H), 2.52 (s, 3H), 2.33 (d, J=13.4 Hz, 1H), 2.03 (s, 3H), 1.93 (t, J=6.8 Hz, 4H), 1.57 (s, 2H), 1.17 (dd, J=4.8, 2.1 Hz, 2H), 0.94 (dd, J=8.0, 2.4 Hz, 2H). LC/MS (ESI) m/z: 596 (M+H)⁺.

### Example 51 and 52: (1²R,1⁴R,E)-1¹-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylic acid and (1²R,1⁴R)-1′-((5-methoxy-7-methyl-1H-indol-4-yl)methyl)-3,10-dioxa-1(2,4)-piperidina-2(1,7)-naphthalenacyclodecaphane-2⁴-carboxylic acid

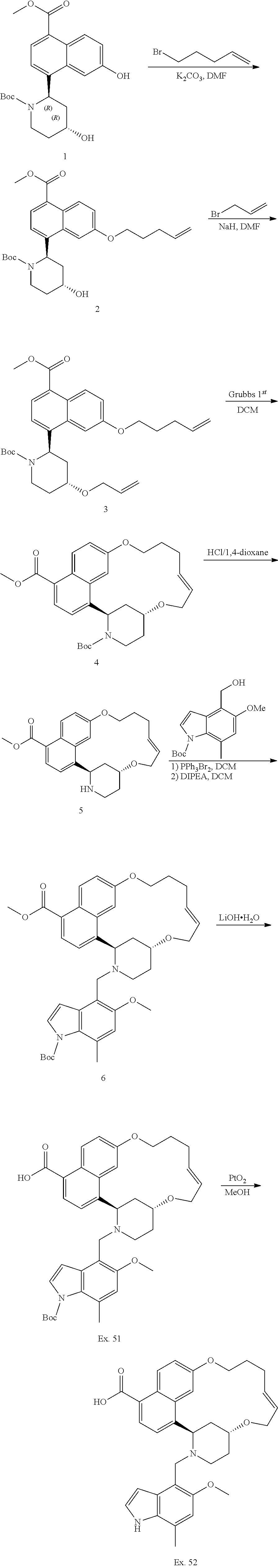

Step 1: tert-butyl (2R,4R)-4-hydroxy-2-(4-(methoxycarbonyl)-7-(pent-4-en-1-yloxy)naphthalen-1-yl)piperidine-1-carboxylate (2): To a mixture of tert-butyl (2R,4R)-4-hydroxy-2-(7-hydroxy-4-(methoxycarbonyl)naphthalen-1-yl)piperidine-1-carboxylate (208 mg, 0.52 mmol) in DMF (3 mL) was added K₂CO₃ (286 mg, 2.07 mmol) and 5-bromopent-1-ene (169 mg, 1.04 mmol) and the mixture was stirred at 25° C. overnight. The mixture was diluted with EtOAc, washed with water and brine, dried over anhydrous Na₂SO₄, filtered and concentrated under reduced pressure to dryness. The residue was purified by flash chromatography (PE:EtOAc=3:1) to give tert-butyl (2R,4R)-4-hydroxy-2-(4-(methoxycarbonyl)-7-(pent-4-en-1-yloxy)naphthalen-1-yl)piperidine-1-carboxylate (188 mg, yield 77.4%) as colorless oil. LC/MS (ESI) m/z: 470 (M+H)⁺.

Step 2: tert-butyl (2R,4R)-4-(allyloxy)-2-(4-(methoxycarbonyl)-7-(pent-4-en-1-yloxy)naphthalen-1-yl)piperidine-1-carboxylate (3). At 0° C., to a solution of tert-butyl (2R,4R)-4-hydroxy-2-(4-(methoxycarbonyl)-7-(pent-4-en-1-yloxy)naphthalen-1-yl)piperidine-1-carboxylate (166 mg, 0.35 mmol) in DMF (2 mL) was added NaH (42 mg, 1.06 mmol, 60% dispersion in mineral oil) in portions and the mixture was stirred under N₂ atmosphere at 0° C. for 0.5 hour. Then 3-bromoprop-1-ene (85 mg, 0.71 mmol) was added and the resulting mixture was stirred at 25° C. for 1 hour. The mixture was quenched with saturated NH₄Cl solution and extracted with EtOAc twice. The combined organic layers were washed with water and brine, dried over Na₂SO₄, filtered and concentrated to dryness. The residue was purified by column chromatography on silica gel (PE:EtOAc=3:1) to give tert-butyl (2R,4R)-4-(allyloxy)-2-(4-(methoxycarbonyl)-7-(pent-4-en-1-yloxy)naphthalen-1-yl)piperidine-1-carboxylate (110 mg, yield 61.0%) as colorless oil. LC/MS: m/z 510 (M+H)⁺.

Step 3: 1¹-(tert-butyl) 2⁴-methyl (12R,14R,E)-3,10-dioxa-1(2,4)-piperidina-2(1,7)-naphthalenacyclodecaphan-7-ene-1¹,2⁴-dicarboxylate (4): To a solution of (2R,4R)-tert-butyl 4-(allyloxy)-2-(4-(methoxycarbonyl)-7-(pent-4-en-1-yloxy)naphthalen-1-yl)piperidine-1-carboxylate (100 mg, 0.196 mmol) in dry DCM (100 mL) was added Grubbs 1^{st} catalyst (32 mg, 0.04 mmol) and the mixture was stirred under N₂ atmosphere at 25° C. for 18 hours. The mixture was concentrated under reduced pressure to dryness and the residue was purified by flash chromatography (PE:EtOAc=10:1) to give 1′-(tert-butyl) 2⁴-methyl (12R,14R,E)-3,10-dioxa-1(2,4)-piperidina-2(1,7)-naphthalenacyclodecaphan-7-ene-1¹,2⁴-dicarboxylate (70 mg, yield 74.0%) as brown solid. LC/MS (ESI) m/z: 482 (M+H)⁺.

Step 4: methyl (1²R,1⁴R,E)-3,10-dioxa-1(2,4)-piperidina-2(1,7)-naphthalenacyclodecaphan-7-ene-2⁴-carboxylate hydrochloride (5): A mixture of 11-(tert-butyl) 2⁴-methyl (12R,14R,E)-3,10-dioxa-1(2,4)-piperidina-2(1,7)-naphthalenacyclodecaphan-7-ene-1¹,2⁴-dicarboxylate (60 mg, 0.12 mmol) in HCl/1,4-dioxane (1 mL, 4.0 mmol) was stirred at 25° C. for 1 hour. The mixture was concentrated to dryness to give methyl (1²R,1⁴R,E)-3,10-dioxa-1(2,4)-piperidina-2(1,7)-naphthalenacyclodecaphan-7-ene-2⁴-carboxylate hydrochloride (55 mg, yield 100%) as white solid, which was used directly without purification. LC/MS (ESI) m/z: 382 (M+H)⁺.

Step 5: methyl (1²R,1⁴R,E)-11-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate (6). At 0° C., to a solution of tert-butyl 4-(hydroxymethyl)-5-methoxy-7-methyl-1H-indole-1-carboxylate (38 mg, 0.13 mmol) in dry DCM (1 mL) was added PPh₃Br₂ (63 mg, 0.15 mmol) in portions and the mixture stirred under N₂ atmosphere at 0° C. for 2 hours. Then methyl (1²R,1⁴R,E)-3,10-dioxa-1(2,4)-piperidina-2(1,7)-naphthalenacyclodecaphan-7-ene-2⁴-carboxylate hydrochloride (55 mg, 0.13 mmol) and DIPEA (26 mg, 0.2 mmol) in dry DCM (1 mL) was added and the mixture was stirred at 0° C. for 1.5 hours. The mixture was concentrated to dryness. The residue was purified by flash chromatography on silica gel (DCM:MeOH=97:3) to give methyl (1²R,1⁴R,E)-1′-((1-(tert-butoxycarbonyl)-5-methoxy-7-methyl-1H-indol-4-yl)methyl)-3,10-dioxa-1 (2,4)-piperidina-2(1,7)-naphthalenacyclodecaphan-7-ene-2⁴-carboxylate (50 mg, yield 53.5%) as white solid. LC/MS (ESI) m/z: 655 (M+H)⁺.

Step 6: (1²R,1⁴R,E)-11-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylic acid (Example 51). A mixture of methyl (1²R,1⁴R,E)-1¹-((1-(tert-butoxycarbonyl)-5-methoxy-7-methyl-1H-indol-4-yl)methyl)-3,10-dioxa-1 (2,4)-piperidina-2(1,7)-naphthalenacyclodecaphan-7-ene-2⁴-carboxylate (50 mg, 0.08 mmol) and LiOH H₂O (10 mg, 0.39 mmol) in MeOH (2 mL)/water (0.5 mL) was stirred at 60° C. overnight. The reaction mixture was acidified with 1 N aq.HCl solution to pH˜4 and extracted with EtOAc twice. The combined organic layers were washed with brine, dried over anhydrous Na₂SO₄, filtered and concentrated to dryness. The residue was purified by prep-HPLC to give (1²R,1⁴R,E)-1′-((1-(tert-butoxycarbonyl)-5-methoxy-7-methyl-1H-indol-4-yl)methyl)-3,10-dioxa-1 (2,4)-piperidina-2(1,7)-naphthalenacyclodecaphan-7-ene-2⁴-carboxylic acid (40 mg, yield 64.0%) as white solid. ¹HNMR (400 MHz, CD₃OD) δ 8.56 (d, J=9.3 Hz, 1H), 8.34 (s, 1H), 7.83 (s, 2H), 7.46 (d, J=2.3 Hz, 1H), 7.28 (d, J=3.1 Hz, 1H), 7.20 (dd, J=9.3, 2.3 Hz, 1H), 6.75 (s, 1H), 6.41 (d, J=3.1 Hz, 1H), 6.07 (dd, J=13.8, 6.9 Hz, 1H), 5.82-5.74 (m, 1H), 5.59 (d, J=10.0 Hz, 1H), 4.67 (dt, J=12.0, 8.1 Hz, 1H), 4.37 (dd, J=14.3, 5.3 Hz, 1H), 4.27 (t, J=8.0 Hz, 2H), 4.23-4.17 (m, 1H), 3.97-3.91 (m, 1H), 3.87 (s, 1H), 3.81 (s, 3H), 3.79-3.72 (m, 1H), 3.48-3.42 (m, 1H), 2.49 (s, 3H), 2.34-2.22 (m, 2H), 2.15 (d, J=9.1 Hz, 2H), 2.11 (s, 1H), 2.03 (d, J=14.1 Hz, 2H), 1.94 (d, J=8.4 Hz, 1H). LC/MS (ESI) m/z: 541 (M+H)⁺.

Step 7: (1²R,1⁴R)-1¹-((5-methoxy-7-methyl-1H-indol-4-yl)methyl)-3,10-dioxa-1(2,4)-piperidina-2(1,7)-naphthalenacyclodecaphane-2⁴-carboxylic acid (Example 52). To a solution of (1²R,1⁴R,E)-11-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylic acid (21 mg, 0.04 mmol) in MeOH (4 mL) was added PtO₂ (4 mg) at 25° C. under N₂ atmosphere and the mixture was stirred under a H₂ balloon at 25° C. for 2 hours. The mixture was filtered, and the filtrate was concentrated to dryness. The residue was purified by prep-HPLC to give to give (1²R,1⁴R)-1¹-((5-methoxy-7-methyl-1H-indol-4-yl)methyl)-3,10-dioxa-1 (2,4)-piperidina-2(1,7)-naphthalenacyclodecaphane-2⁴-carboxylic acid (6.0 mg, yield 36.9%) as white solid. ¹H NMR (400 MHz, CD₃OD) δ 8.59 (d, J=9.3 Hz, 1H), 8.35 (s, 1H), 7.87-7.80 (m, 2H), 7.62 (d, J=2.1 Hz, 1H), 7.31 (d, J=3.1 Hz, 1H), 7.20 (dd, J=9.3, 2.3 Hz, 1H), 6.76 (s, 1H), 6.45 (d, J=3.2 Hz, 1H), 5.64 (d, J=9.7 Hz, 1H), 4.85-4.77 (m, 2H), 4.72 (dd, J=14.6, 6.5 Hz, 1H), 4.36 (t, J=8.1 Hz, 2H), 4.32-4.25 (m, 1H), 3.83 (s, 3H), 3.78-3.71 (m, 2H), 3.48 (dd, J=14.6, 6.1 Hz, 2H), 2.50 (s, 3H), 2.23 (d, J=15.2 Hz, 1H), 2.13 (d, J=13.5 Hz, 1H), 2.06 (d, J=15.3 Hz, 2H), 1.98-1.90 (m, 1H), 1.87-1.71 (m, 4H), 1.64 (s, 1H), 1.60-1.50 (m, 2H). LC/MS (ESI) m/z: 543 (M+H)⁺.

### Example 53: (1²S,1⁴S)_11-((5-methoxy-7-methyl-1H-indol-4-yl)methyl)-3,10-dioxa-1(2,4)-piperidina-2(1,7)-naphthalenacyclodecaphane-2⁴-carboxylic acid

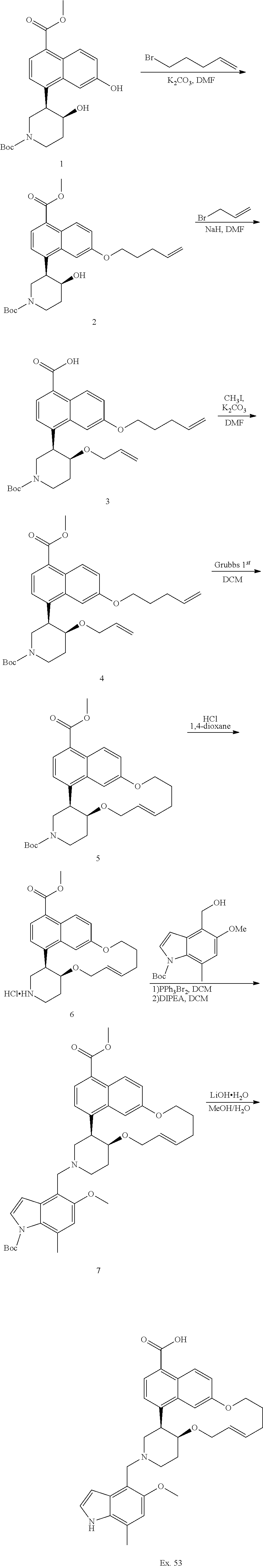

Step 1: tert-butyl (2S,4S)-4-hydroxy-2-(4-(methoxycarbonyl)-7-(pent-4-en-1-yloxy) naphthalen-1-yl)piperidine-1-carboxylate. To a mixture of tert-butyl (2S,4S)-4-hydroxy-2-(7-hydroxy-4-(methoxycarbonyl)naphthalen-1-yl)piperidine-1-carboxylate (200 mg, 0.5 mmol) in DMF (2 mL) was added K₂CO₃ (276 mg, 2.0 mmol) and 5-bromopent-1-ene (163 mg, 1.0 mmol) and the mixture was stirred at 25° C. overnight. The mixture was diluted with EtOAc, washed with water and brine, dried over anhydrous Na₂SO₄, filtered and concentrated under reduced pressure to dryness. The residue was purified by flash chromatography (PE:EtOAc=3:1) to give tert-butyl (2S,4S)-4-hydroxy-2-(4-(methoxycarbonyl)-7-(pent-4-en-1-yloxy)naphthalen-1-yl)piperidine-1-carboxylate (150 mg, yield 63.8%) as white solid. LC/MS (ESI) m/z: 470 (M+H)⁺.

Step 2: 4-((3S,4S)-4-(allyloxy)-1-(tert-butoxycarbonyl)piperidin-3-yl)-6-(pent-4-en-1-yloxy)-1-naphthoic acid. At 0° C., to a solution of tert-butyl (2S,4S)-4-hydroxy-2-(4-(methoxycarbonyl)-7-(pent-4-en-1-yloxy)naphthalen-1-yl)piperidine-1-carboxylate (150 mg, 0.32 mmol) in DMF (2 mL) was added NaH (76 mg, 1.92 mmol, 60% dispersion in mineral oil) in portions and the mixture was stirred under N₂ atmosphere at 0° C. for 0.5 hour. Then 3-bromoprop-1-ene (77 mg, 0.64 mmol) was added and the resulting mixture was stirred at 25° C. for 1 hour. The mixture was quenched with saturated NH₄Cl solution and extracted with EtOAc twice. The combined organic layers were washed with water and brine, dried over anhydrous Na₂SO₄, filtered and concentrated to dryness to give 4-((3S,4S)-4-(allyloxy)-1-(tert-butoxycarbonyl)piperidin-3-yl)-6-(pent-4-en-1-yloxy)-1-naphthoic acid (120 mg, crude) as yellow oil. LC/MS: m/z 496 (M+H)⁺.

Step 3: tert-butyl (3S,4S)-4-(allyloxy)-3-(4-(methoxycarbonyl)-7-(pent-4-en-1-yloxy)naphthalen-1-yl)piperidine-1-carboxylate. To a solution of 4-((3S,4S)-4-(allyloxy)-1-(tert-butoxycarbonyl)piperidin-3-yl)-6-(pent-4-en-1-yloxy)-1-naphthoic acid (120 mg, 0.24 mmol) in DMF (2 mL) was added K₂CO₃ (100 mg, 0.73 mmol) and iodomethane (204 mg, 1.44 mmol) and the mixture was stirred at room temperature for 1 hour. The mixture was extracted with EtOAc twice. The combined organic layers were washed with water and brine, dried over anhydrous Na₂SO₄, filtered and concentrated to dryness. The residue was purified by flash chromatography (PE:EtOAc=5:1) to give tert-butyl (3S,4S)-4-(allyloxy)-3-(4-(methoxycarbonyl)-7-(pent-4-en-1-yloxy)naphthalen-1-yl)piperidine-1-carboxylate (70 mg, yield 57.4%) as white solid. LC/MS: m/z 510 (M+H)⁺.

Step 4: 1¹-(tert-butyl) 2⁴-methyl (1²S,1⁴S,E)-3,10-dioxa-1(2,4)-piperidina-2(1,7)-naphthalenacyclodecaphan-7-ene-1¹,2⁴-dicarboxylate. To a solution of tert-butyl (2S,4S)-4-(allyloxy)-2-(4-(methoxycarbonyl)-7-(pent-4-en-1-yloxy)naphthalen-1-yl)piperidine-1-carboxylate (70 mg, 0.14 mmol) in dry DCM (70 mL) was added Grubbs 1^{st} catalyst (23 mg, 0.027 mmol) and the mixture was stirred under N₂ atmosphere at 25° C. for 18 hours. The mixture was concentrated under reduced pressure to dryness and the residue was purified by flash chromatography (PE:EtOAc=10:1) to give 1′-(tert-butyl) 2⁴-methyl (1²S,1⁴S,E)-3,10-dioxa-1(2,4)-piperidina-2(1,7)-naphthalenacyclodecaphan-7-ene-1¹,2⁴-dicarboxylate (15 mg, yield 22.7%) as brown solid. LC/MS (ESI) m/z: 482 (M+H)⁺.

Step 5: methyl (1²S,1⁴S,E)-3,10-dioxa-1(2,4)-piperidina-2(1,7)-naphthalenacyclodecaphan-7-ene-2⁴-carboxylate hydrochloride. A mixture of 1¹-(tert-butyl) 2⁴-methyl (1²,1⁴S,E)-3,10-dioxa-1(2,4)-piperidina-2(1,7)-naphthalenacyclodecaphan-7-ene-1¹,2⁴-dicarboxylate (15 mg, 0.031 mmol) in HCl/1,4-dioxane (1 mL, 4.0 mmol) was stirred at 25° C. for 1 hour. The mixture was concentrated to dryness to give methyl (1²S, 1⁴S,E)-3,10-dioxa-1 (2,4)-piperidina-2(1,7)-naphthalenacyclodecaphan-7-ene-2⁴-carboxylate hydrochloride (15 mg, crude) as white solid, which was used directly without purification. LC/MS (ESI) m/z: 382 (M+H)⁺.

Step 6: methyl (1²S,1⁴S,E)-1¹-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate. At 0° C., to a solution of tert-butyl 4-(hydroxymethyl)-5-methoxy-7-methyl-1H-indole-1-carboxylate (14 mg, 0.047 mmol) in dry DCM (1 mL) was added PPh₃Br₂ (22 mg, 0.051 mmol) in portions and the mixture stirred under N₂ atmosphere at 0° C. for 2 hours. Then methyl (1²S,1⁴S,Z)-10-oxa-1(2,4)-piperidina-2(1,2)-benzenacyclodecaphan-3-ene-2⁴-carboxylate hydrochloride (15 mg, 0.039 mmol) and DIPEA (15 mg, 0.117 mmol) in dry DCM (1 mL) was added and the mixture was stirred at 0° C. for 1.5 hours. The mixture was concentrated to dryness. The residue was purified by prep-HPLC to give to give methyl (1²S,1⁴S, E)-1¹-((1-(tert-butoxycarbonyl)-5-methoxy-7-methyl-1H-indol-4-yl)methyl)-3,10-dioxa-1 (2,4)-piperidina-2(1,7)-naphthalenacyclodecaphan-7-ene-2⁴-carboxylate (18 mg, yield 69.2%) as white solid. LC/MS (ESI) m/z: 655 (M+H)⁺.

Step 7: (1²S,1⁴S,E)-1¹-((5-methoxy-7-methyl-1H-indol-4-yl)methyl)-3,10-dioxa-1(2,4)-piperidina-2(1,7)-naphthalenacyclodecaphan-7-ene-2⁴-carboxylic acid (Example 53). To a mixture of methyl (1²S,1⁴S,E)-1¹-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate (18 mg, 0.027 mmol) and LiOH H₂O (4 mg, 0.082 mmol) in MeOH (0.75 mL)/water (0.25 mL) and the mixture was stirred at 50° C. overnight. The reaction mixture was acidified with 1 N aq.HCl solution to pH˜4 and extracted with EtOAc twice. The combined organic layers were washed with brine, dried over anhydrous Na₂SO₄, filtered and concentrated to dryness. The residue was purified by prep-HPLC to give (1²S,1⁴S,E)-1¹-((5-methoxy-7-methyl-1H-indol-4-yl)methyl)-3,10-dioxa-1 (2,4)-piperidina-2(1,7)-naphthalenacyclodecaphan-7-ene-2⁴-carboxylic acid (1 mg, yield 7.1%) as white solid. ¹HNMR (400 MHz, CD₃OD) δ 8.65 (d, J=9.3 Hz, 1H), 7.85-7.80 (m, 2H), 7.34 (d, J=2.1 Hz, 1H), 7.31 (d, J=3.1 Hz, 1H), 7.21 (dd, J=9.3, 2.3 Hz, 1H), 6.82 (s, 1H), 6.44 (d, J=3.1 Hz, 1H), 5.97 (dt, J=13.9, 6.8 Hz, 1H), 5.68-5.56 (m, 1H), 5.34 (d, J=10.9 Hz, 1H), 4.82 (d, J=12.8 Hz, 2H), 4.26-4.16 (m, 2H), 4.02-3.93 (m, 2H), 3.91 (d, J=5.1 Hz, 3H), 3.86 (dt, J=11.6, 3.9 Hz, 1H), 3.59 (dd, J=12.9, 1.4 Hz, 1H), 3.50-3.41 (m, 1H), 2.54 (d, J=13.6 Hz, 4H), 2.27-2.18 (m, 1H), 2.15-2.07 (m, 2H), 1.90-1.74 (m, 4H). LC/MS (ESI) m/z: 541 (M+H)⁺.

### Example 54: (1²S,1⁴S,E)-1¹-((5-methoxy-7-methyl-1H-indol-4-yl)methyl)-N-(methylsulfonyl)-3,10-dioxa-1(2,4)-piperidina-2(1,7)-naphthalenacyclodecaphan-7-ene-2⁴-carboxamide

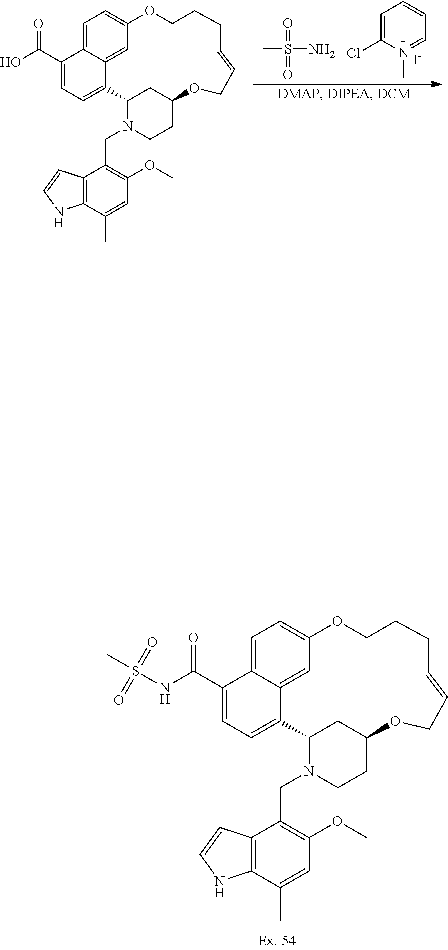

To a solution of (1²S,1⁴S,E)-1¹-((5-methoxy-7-methyl-1H-indol-4-yl)methyl)-3,10-dioxa-1(2,4)-piperidina-2(1,7)-naphthalenacyclodecaphan-7-ene-2⁴-carboxylic acid (18 mg, 33.3 umol) in anhydrous DCM (2 mL) was added methanesulfonamide (4 mg, 42.1 umol), 2-chloro-1-methylpyridin-1-ium iodide (6 mg, 46.6 umol), DIPEA (13 mg, 0.1 mmol) and DMAP (6 mg, 50 umol) at 0° C., and the mixture was stirred at room temperature overnight. The mixture was concentrated to dryness under reduced pressure. The residue was purified by prep-HPLC to give (1²S,1⁴S,E)-1¹-((5-methoxy-7-methyl-1H-indol-4-yl)methyl)-N-(methylsulfonyl)-3,10-dioxa-1(2,4)-piperidina-2(1,7)-naphthalenacyclodecaphan-7-ene-2⁴-carboxamide (11.0 mg, yield 53.5%) as white solid. ¹HNMR (400 MHz, CD₃OD) δ 8.39 (d, J=9.3 Hz, 1H), 7.87 (d, J=7.6 Hz, 1H), 7.78 (d, J=7.5 Hz, 1H), 7.46 (s, 1H), 7.27 (d, J=3.1 Hz, 1H), 7.23 (dd, J=9.3, 2.0 Hz, 1H), 6.74 (s, 1H), 6.40 (d, J=3.1 Hz, 1H), 6.10-6.00 (m, 1H), 5.78 (dd, J=12.3, 7.6 Hz, 1H), 5.60 (d, J=11.9 Hz, 1H), 4.68 (dd, J=19.8, 8.1 Hz, 1H), 4.38 (dd, J=14.1, 5.1 Hz, 1H), 4.28-4.18 (m, 3H), 3.93 (d, J=9.9 Hz, 1H), 3.88 (s, 1H), 3.81 (s, 3H), 3.76 (d, J=10.4 Hz, 1H), 3.46 (d, J=12.6 Hz, 1H), 3.34 (s, 3H), 2.49 (s, 3H), 2.32-2.09 (m, 5H), 2.08-2.00 (m, 2H), 1.96-1.89 (m, 1H). LC/MS (ESI) m/z: 618 (M+H)⁺.

### Example 55: (1²S,1⁴S)-1¹-((5-methoxy-7-methyl-1H-indol-4-yl)methyl)-2⁴-(1H-pyrazol-5-yl)-3,10-dioxa-1(2,4)-piperidina-2(1,2)-benzenacyclodecaphane

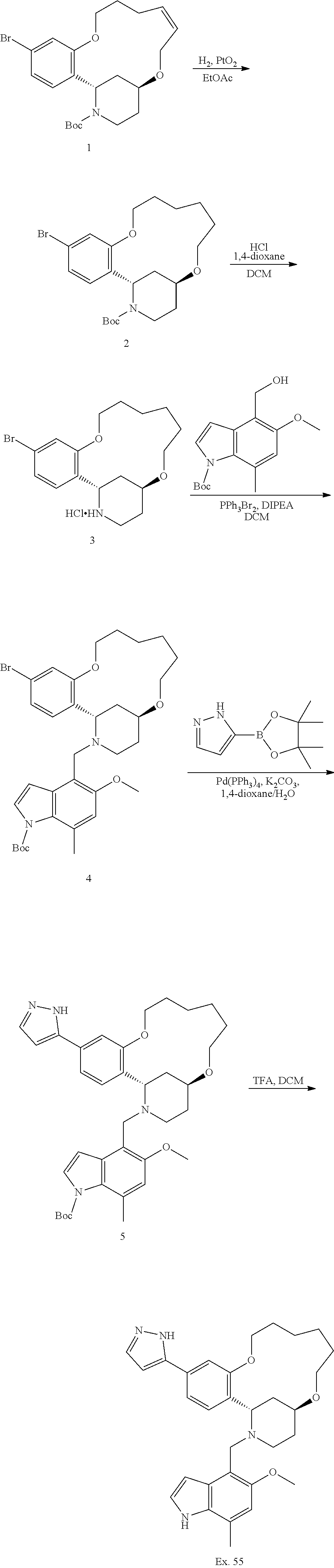

Step 1: tert-butyl (1²S,1⁴S)-2⁴-bromo-3,10-dioxa-1(2,4)-piperidina-2(1,2)-benzenacyclodecaphane-1¹-carboxylate: To a solution of tert-butyl (1²S,1⁴S,Z)-2⁴-bromo-3,10-dioxa-1(2,4)-piperidina-2(1,2)-benzenacyclodecaphan-7-ene-1′-carboxylate (250 mg, 0.55 mmol) in EtOAc (3 mL) was added PtO₂ (13 mg, 0.055 mmol), the mixture was degassed under N₂ atmosphere for three times and stirred under a H₂ balloon at room temperature for half an hour. The mixture was filtered and the filtrate was concentrated under reduced pressure to dryness. The residue was purified by flash column chromatography (PE:EtOAc=3:1) to give tert-butyl (1²S,1⁴S)-2⁴-bromo-3,10-dioxa-1(2,4)-piperidina-2(1,2)-benzenacyclodecaphane-11-carboxylate (184 mg, 73.9% yield) as colorless oil. LC/MS (ESI) m/z: 454 (M+H)⁺.

Step 2: (1²S,1⁴S)-2⁴-bromo-3,10-dioxa-1(2,4)-piperidina-2(1,2)-benzenacyclodecaphane hydrochloride. A solution of tert-butyl (1²S,1⁴S)-2⁴-bromo-3,10-dioxa-1(2,4)-piperidina-2(1,2)-benzenacyclodecaphane-1¹-carboxylate (184 mg, 0.41 mmol) in DCM (2 mL) was added HCl/1,4-dioxane (1 mL, 4M) and the mixture was stirred at 25° C. for 1 hour. The mixture was concentrated to dryness under reduced pressure to give (1²,1⁴S)-2⁴-bromo-3,10-dioxa-1(2,4)-piperidina-2(1,2)-benzenacyclodecaphane (150 mg, 100% yield) as white solid, which was used directly without purification. LC/MS (ESI) m/z: 354 (M+H)⁺.

Step 3: tert-butyl 4-(((1²S,1⁴S)-2⁴-bromo-3,10-dioxa-1(2,4)-piperidina-2(1,2)-benzenacyclodecaphane-1¹-yl)methyl)-5-methoxy-7-methyl-1H-indole-1-carboxylate. To a solution of 4-(hydroxymethyl)-5-methoxy-7-methyl-1H-indole-1-carboxylate (142 mg, 0.485 mmol) in anhydrous DCM (1.5 mL) was added PPh₃Br₂ (222 mg, 0.525 mmol) in portions at 0° C., and the mixture was stirred under N₂ atmosphere at 0° C. for 2 hours. Then a mixture of (1²S,1⁴S)-2⁴-bromo-3,10-dioxa-1(2,4)-piperidina-2(1,2)-benzenacyclodecaphane hydrochloride (150 mg, 0.41 mmol) and DIPEA (157 mg, 1.212 mmol) in anhydrous DCM (1.5 mL) was added and the resulting mixture was stirred at 0° C. for 1.5 hours. The mixture was concentrated to dryness under reduced pressure. The residue was purified by flash column chromatography on silica gel (DCM:MeOH=10:1) to give tert-butyl 4-(((1²S,1⁴S)-2⁴-bromo-3,10-dioxa-1(2,4)-piperidina-2(1,2)-benzenacyclodecaphane-1¹-yl)methyl)-5-methoxy-7-methyl-1H-indole-1-carboxylate (200 mg, 65.8% yield) as white solid. LC/MS (ESI) m/z: 627 (M+H)⁺.

Step 4: tert-butyl 4-(((1²S,14S)-2⁴-(1H-pyrazol-5-yl)-3,10-dioxa-1(2,4)-piperidina-2(1,2)-benzenacyclodecaphane-1¹-yl)methyl)-5-methoxy-7-methyl-1H-indole-1-carboxylate. To a solution of tert-butyl 4-(((1²,1⁴S)-2⁴-bromo-3,10-dioxa-1(2,4)-piperidina-2(1,2)-benzenacyclodecaphane-1¹-yl)methyl)-5-methoxy-7-methyl-1H-indole-1-carboxylate (60 mg, 0.096 mmol) in 1,4-dioxane (1.5 mL) and water (0.5 mL) was added 5-(4,4,5,5-tetramethyl-1,3,2-dioxaborolan-2-yl)-1H-pyrazole (28 mg, 0.144 mmol), Pd(PPh₃)₄(11 mg, 0.0096 mmol) and Na₂CO₃ (30 mg, 0.288 mmol) at room temperature, and the mixture was degassed under N₂ atmosphere for three times and stirred at 80° C. for 18 hours. The mixture was diluted with EtOAc, washed with water and brine, dried over Na₂SO₄, filtered and concentrated under reduced pressure to dryness. The residue was purified by column chromatography on silica gel (PE:EtOAc=1:1) to give tert-butyl 4-(((1²S,1⁴S)-2⁴-(1H-pyrazol-5-yl)-3,10-dioxa-1(2,4)-piperidina-2(1,2)-benzenacyclodecaphane-1¹-yl)methyl)-5-methoxy-7-methyl-1H-indole-1-carboxylate (50 mg, 87.7% yield) as light yellow solid. LC/MS (ESI) m/z: 615 (M+H)⁺.

Step 5: (1²S,14S)-1¹-((5-methoxy-7-methyl-1H-indol-4-yl)methyl)-2⁴-(1H-pyrazol-5-yl)-3,10-dioxa-1(2,4)-piperidina-2(1,2)-benzenacyclodecaphane. A solution of tert-butyl 4-(((1²S,1⁴S)-2⁴-(1H-pyrazol-5-yl)-3,10-dioxa-1(2,4)-piperidina-2(1,2)-benzenacyclodecaphane-1¹-yl)methyl)-5-methoxy-7-methyl-1H-indole-1-carboxylate (50 mg, 0.081 mmol) in DCM (1 mL) was added TFA (23 mg, 0.24 mmol) and the mixture was stirred at 25° C. for 1 hour. The mixture was concentrated to dryness under reduced pressure and the residue was purified by prep-HPLC to give (1²S,1⁴S)-1¹-((5-methoxy-7-methyl-1H-indol-4-yl)methyl)-2⁴-(1H-pyrazol-5-yl)-3,10-dioxa-1(2,4)-piperidina-2(1,2)-benzenacyclodecaphane (1.1 mg, 2.7% yield). ¹HNMR (400 MHz, CD₃OD) δ 8.53 (s, 1H), 7.74-7.67 (m, 2H), 7.64 (d, J=7.9 Hz, 1H), 7.56 (s, 1H), 7.33 (d, J=3.1 Hz, 1H), 6.82-6.78 (m, 2H), 6.46 (d, J=3.1 Hz, 1H), 5.39 (d, J=12.8 Hz, 1H), 4.55 (ddd, J=12.6, 10.4, 5.6 Hz, 3H), 4.13 (d, J=13.4 Hz, 1H), 4.01-3.94 (m, 1H), 3.88 (s, 3H), 3.75 (s, 1H), 3.65-3.59 (m, 1H), 3.56 (d, J=9.0 Hz, 1H), 3.42 (d, J=12.3 Hz, 1H), 2.52 (s, 3H), 2.35 (d, J=12.5 Hz, 1H), 2.00 (dt, J=25.7, 12.3 Hz, 6H), 1.83 (t, J=9.8 Hz, 1H), 1.63-1.45 (m, 4H). LC/MS (ESI) m/z: 515 (M+H)⁺.

### Example 56: (1²S,1⁴S)-1¹-((5-methoxy-7-methyl-1H-indol-4-yl)methyl)-N-(methylsulfonyl)-3,10-dioxa-1(2,4)-piperidina-2(1,7)-naphthalenacyclodecaphane-2⁴-carboxamide

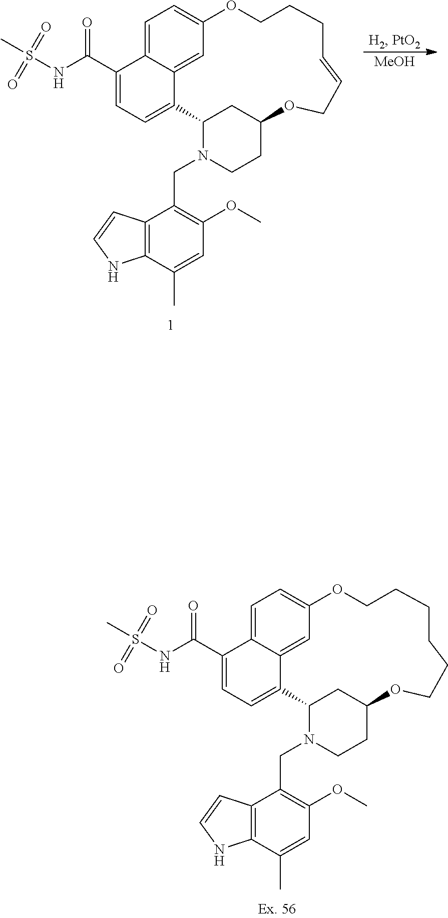

To a solution of (1²S,1⁴S,E)-1¹-((5-methoxy-7-methyl-1H-indol-4-yl)methyl)-N-(methylsulfonyl)-3,10-dioxa-1(2,4)-piperidina-2(1,7)-naphthalenacyclodecaphan-7-ene-2⁴-carboxamide (8 mg, 33.3 umol) in MeOH (4 mL) was added PtO₂ (4 mg, 42.1 umol) at 0° C., and the mixture was degassed under N₂ atmosphere for three times and stirred under a H₂ balloon at room temperature overnight. The mixture was filtered and the filtrated was concentrated to dryness. The residue was purified by prep-HPLC to give (1²S,1⁴S)_11-((5-methoxy-7-methyl-1H-indol-4-yl)methyl)-N-(methylsulfonyl)-3,10-dioxa-1(2,4)-piperidina-2(1,7)-naphthalenacyclodecaphane-2⁴-carboxamide (7.0 mg, yield 87.2%) as white solid. ¹HNMR (400 MHz, CD₃OD) δ 8.45 (d, J=9.3 Hz, 1H), 8.30 (s, 1H), 7.79 (dd, J=18.2, 7.6 Hz, 2H), 7.59 (s, 1H), 7.29 (d, J=3.1 Hz, 1H), 7.21 (dd, J=9.3, 2.1 Hz, 1H), 6.76 (s, 1H), 6.44 (d, J=3.1 Hz, 1H), 5.60 (d, J=9.6 Hz, 1H), 4.75-4.67 (m, 1H), 4.36-4.25 (m, 3H), 3.81 (d, J=10.7 Hz, 4H), 3.72 (s, 2H), 3.44 (t, J=9.0 Hz, 2H), 3.26 (s, 3H), 2.50 (s, 3H), 2.15-1.99 (m, 4H), 1.81 (d, J=7.3 Hz, 4H), 1.58 (d, J=4.0 Hz, 3H), 1.30 (s, 1H). LC/MS (ESI) m/z: 620 (M+H)⁺.

### Example 57: (1²S,1⁴S)-1¹-((5-methoxy-7-methyl-1H-indol-4-yl)methyl)-N-(phenylsulfonyl)-3,10-dioxa-1(2,4)-piperidina-2(1,2)-benzenacyclodecaphane-2⁴-carboxamide

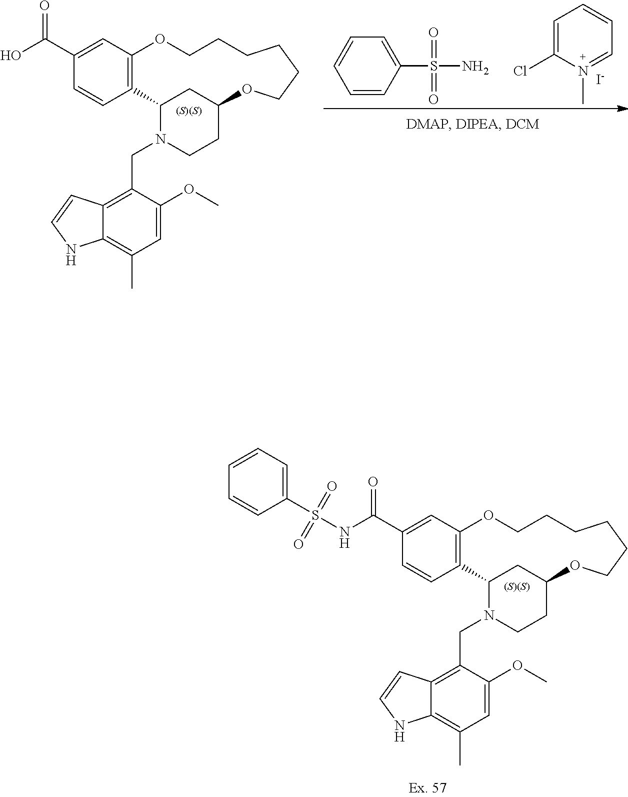

A mixture of (1²S,1⁴S)-1¹-((5-methoxy-7-methyl-1H-indol-4-yl)methyl)-3,10-dioxa-1(2,4)-piperidina-2(1,2)-benzenacyclodecaphane-2⁴-carboxylic acid (10 mg, 0.02 mmol), benzene sulfonamide (5 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)-11-((5-methoxy-7-methyl-1H-indol-4-yl)methyl)-N-(phenylsulfonyl)-3,10-dioxa-1(2,4)-piperidina-2(1,2)-benzenacyclodecaphane-2⁴-carboxamide (3.3 mg, yield 27.5%) as white solid. ¹HNMR (400 MHz, CD₃OD) δ 8.72 (s, 1H), 8.18 (s, 1H), 8.03 (dd, J=8.0, 1.4 Hz, 2H), 7.80 (dd, J=8.0, 1.1 Hz, 1H), 7.70 (s, 1H), 7.61 (d, J=8.0 Hz, 1H), 7.48 (td, J=9.1, 4.6 Hz, 3H), 7.31 (d, J=3.1 Hz, 1H), 6.77 (s, 1H), 6.40 (d, J=3.1 Hz, 1H), 5.35 (d, J=12.4 Hz, 1H), 4.44 (t, J=10.4 Hz, 2H), 4.06 (d, J=12.8 Hz, 1H), 3.89 (s, 1H), 3.83 (s, 3H), 3.69 (s, 1H), 3.52 (t, J=14.7 Hz, 3H), 3.39 (d, J=12.8 Hz, 1H), 2.50 (s, 3H), 2.25 (d, J=15.0 Hz, 1H), 2.04-1.86 (m, 6H), 1.78 (dd, J=14.9, 8.1 Hz, 1H), 1.61-1.40 (m, 4H). LC/MS (ESI) m/z: 632 (M+H)⁺.

### Example 58: (1²S,1⁴S, E)-1¹-((5-methoxy-7-methyl-1H-indol-4-yl)methyl)-N-(methylsulfonyl)-11-oxa-1(2,4)-piperidina-2(1,2)-benzenacycloundecaphan-3-ene-2⁴-carboxamide

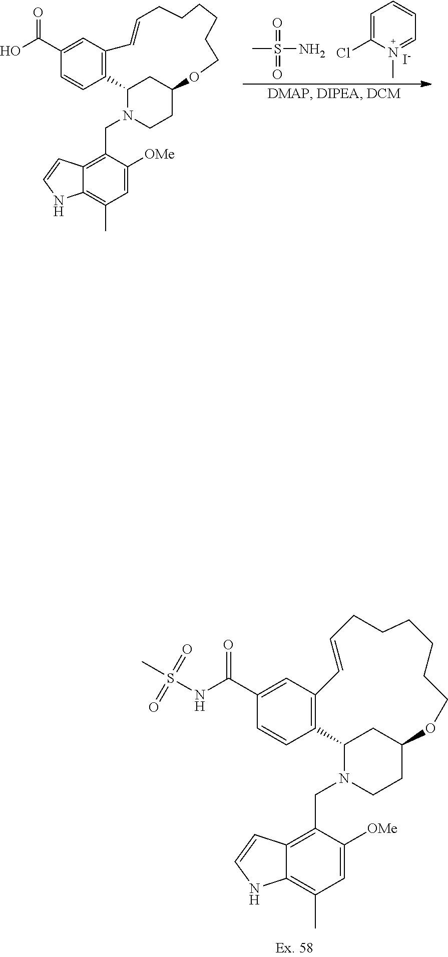

A mixture of (1²S,1⁴S,E)-1¹-((5-methoxy-7-methyl-1H-indol-4-yl)methyl)-11-oxa-1(2,4)-piperidina-2(1,2)-benzenacycloundecaphan-3-ene-2⁴-carboxylic acid (20 mg, 0.04 mmol), methane sulfonamide (5 mg, 0.05 mmol), 2-chloro-1-methylpyridin-1-ium iodide (13 mg, 0.05 mmol), DMAP (1 mg, 0.002 mmol) and DIPEA (16 mg, 0.12 mmol) in DCM (1 mL) was stirred at room temperature for 18 hours. The mixture was concentrated to dryness under reduced pressure and then the residue was purified by prep-HPLC to give (1²S,1⁴S,E)-1¹-((5-methoxy-7-methyl-1H-indol-4-yl)methyl)-N-(methylsulfonyl)-11-oxa-1(2,4)-piperidina-2(1,2)-benzenacycloundecaphan-3-ene-2⁴-carboxamide (11.9 mg, yield 51.7%) as white solid. ¹H NMR (400 MHz, CD₃OD) δ 8.14 (dd, J=8.2, 1.7 Hz, 1H), 8.09 (s, 1H), 7.67 (d, J=8.1 Hz, 1H), 7.34 (d, J=2.6 Hz, 1H), 7.02 (dd, J=15.2, 5.5 Hz, 1H), 6.80 (d, J=4.2 Hz, 1H), 6.50 (t, J=2.9 Hz, 1H), 5.95 (dd, J=15.3, 7.5 Hz, 1H), 5.25-5.13 (m, 1H), 4.46 (t, J=10.7 Hz, 1H), 4.34 (t, J=11.1 Hz, 1H), 3.85 (d, J=3.4 Hz, 3H), 3.76-3.39 (m, 6H), 3.17 (s, 3H), 2.52 (s, 3H), 2.42 (s, 2H), 2.16-1.83 (m, 7H), 1.68 (s, 2H), 1.52 (d, J=11.2 Hz, 2H). LC/MS (ESI) m/z: 580 (M+H)⁺.

### Example 59: (1²S,1⁴S)—N-(ethylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide

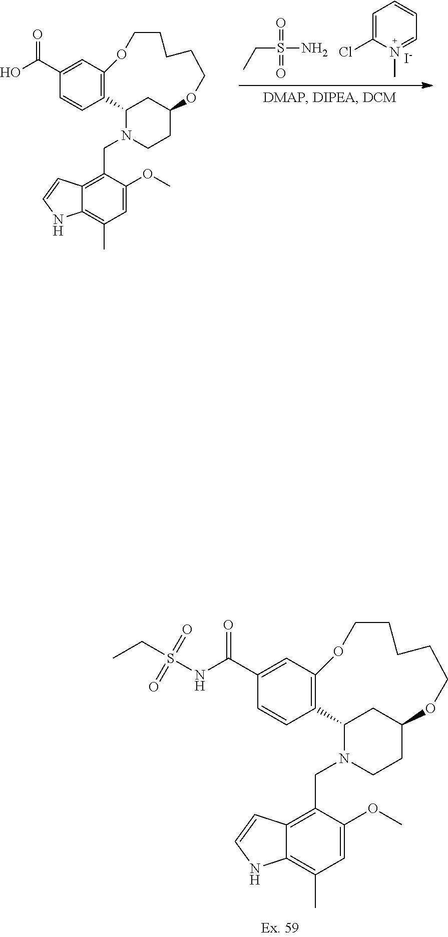

A mixture of (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol), ethane sulfonamide (3 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)—N-(ethylsulfonyl)-11-((5-methoxy-7-methyl-1H-indol-4-yl)methyl)-3,9-dioxa-1(2,4)-piperidina-2(1,2)-benzenacyclononaphane-2⁴-carboxamide (1.2 mg, yield 10.9%) as white solid. ¹HNMR (400 MHz, CD₃OD) δ 7.93 (d, J=8.1 Hz, 1H), 7.89 (s, 1H), 7.62 (d, J=8.1 Hz, 1H), 7.32 (d, J=3.1 Hz, 1H), 6.78 (s, 1H), 6.41 (d, J=3.1 Hz, 1H), 5.53 (d, J=11.1 Hz, 1H), 4.41 (d, J=12.6 Hz, 2H), 4.28-4.22 (m, 1H), 4.09 (d, J=12.8 Hz, 1H), 3.83 (s, 3H), 3.79 (s, 1H), 3.71-3.65 (m, 1H), 3.62-3.53 (m, 2H), 3.45-3.41 (m, 1H), 3.40-3.35 (m, 2H), 2.51 (s, 3H), 2.45 (d, J=5.7 Hz, 1H), 2.32 (d, J=15.9 Hz, 1H), 2.14-1.95 (m, 3H), 1.90 (d, J=15.5 Hz, 1H), 1.68-1.54 (m, 4H), 1.33 (t, J=7.4 Hz, 3H). LC/MS (ESI) m/z: 570 (M+H)⁺.

### Example 60: (1²S,1⁴S)—N-(cyclopropylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide

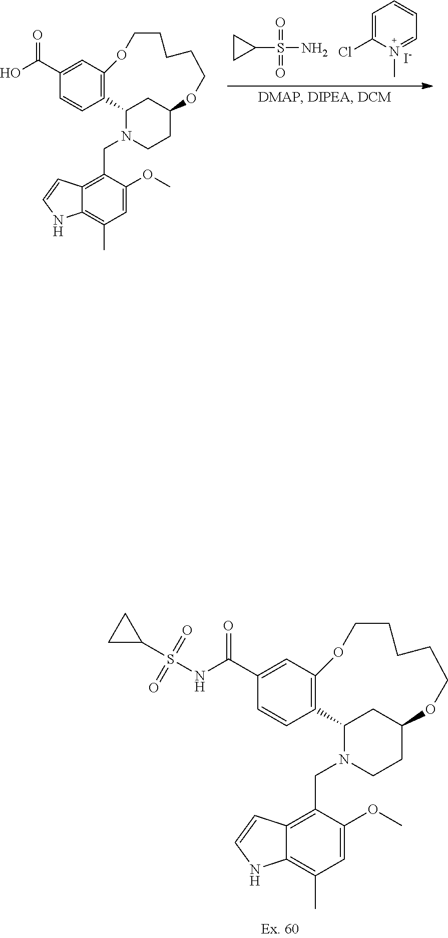

A mixture of (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol), cyclopropane sulfonamide (4 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)—N-(cyclopropylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide (1.8 mg, yield 15.5%) as white solid. ¹H NMR (400 MHz, CD₃OD) δ 8.29 (s, 1H), 7.92 (dd, J=8.1, 1.4 Hz, 1H), 7.88 (d, J=1.4 Hz, 1H), 7.63 (d, J=8.1 Hz, 1H), 7.32 (d, J=3.2 Hz, 1H), 6.78 (s, 1H), 6.41 (d, J=3.2 Hz, 1H), 5.53 (d, J=10.8 Hz, 1H), 4.44-4.37 (m, 2H), 4.25 (d, J=11.4 Hz, 1H), 4.09 (d, J=12.8 Hz, 1H), 3.84 (s, 3H), 3.79 (d, J=1.5 Hz, 1H), 3.68 (dt, J=8.3, 4.0 Hz, 1H), 3.58 (ddd, J=17.4, 10.4, 4.3 Hz, 2H), 3.46-3.39 (m, 1H), 3.12-3.06 (m, 1H), 2.51 (s, 3H), 2.45 (dd, J=8.4, 5.8 Hz, 1H), 2.33 (d, J=14.3 Hz, 1H), 2.12 (d, J=13.1 Hz, 1H), 2.03-1.85 (m, 3H), 1.66-1.53 (m, 4H), 1.20-1.15 (m, 2H), 0.95 (qd, J=5.1, 1.0 Hz, 2H). LC/MS (ESI) m/z: 582 (M+H)⁺.

### Example 61: (1²S,1⁴S)—N-(cyclopentylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide

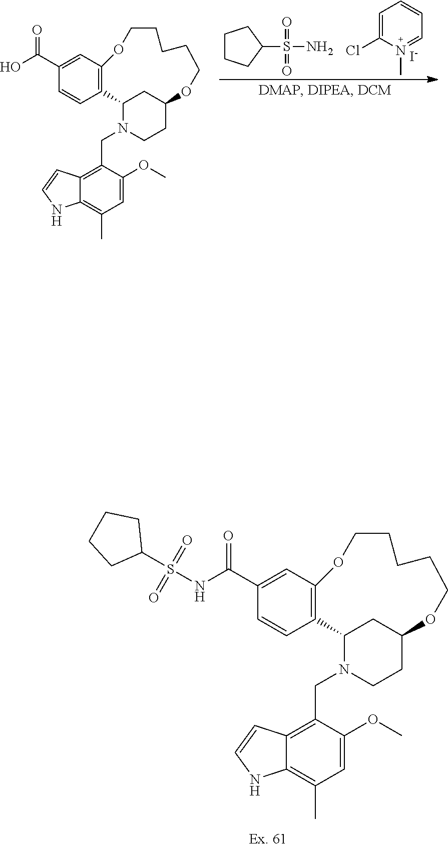

A mixture of (1²S,14S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol), cyclopentane sulfonamide (4 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)—N-(cyclopentylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide (3.2 mg, yield 26.2%) as white solid. ¹HNMR (400 MHz, CD₃OD) δ 8.31 (s, 1H), 7.91 (d, J=8.1 Hz, 1H), 7.88 (s, 1H), 7.62 (d, J=8.1 Hz, 1H), 7.32 (d, J=3.1 Hz, 1H), 6.78 (s, 1H), 6.41 (d, J=3.0 Hz, 1H), 5.53 (d, J=11.8 Hz, 1H), 4.40 (dd, J=13.9, 9.0 Hz, 2H), 4.28-4.16 (m, 2H), 4.09 (d, J=12.8 Hz, 1H), 3.83 (s, 3H), 3.79 (s, 1H), 3.71-3.66 (m, 1H), 3.62-3.54 (m, 2H), 3.42 (dd, J=12.5, 3.0 Hz, 1H), 2.51 (s, 3H), 2.44 (d, J=8.5 Hz, 1H), 2.33 (d, J=15.1 Hz, 1H), 2.11 (dd, J=12.9, 6.3 Hz, 3H), 2.05-1.73 (m, 8H), 1.66-1.61 (m, 4H), 1.55 (d, J=11.6 Hz, 1H). LC/MS (ESI) m/z: 610 (M+H)⁺.

### Example 62: (1²S,1⁴S)—N-(cyclopentylsulfonyl)-1¹-((5-methoxy-7-methyl-1H-indol-4-yl)methyl)-3,10-dioxa-1(2,4)-piperidina-2(1,2)-benzenacyclodecaphane-2⁴-carboxamide

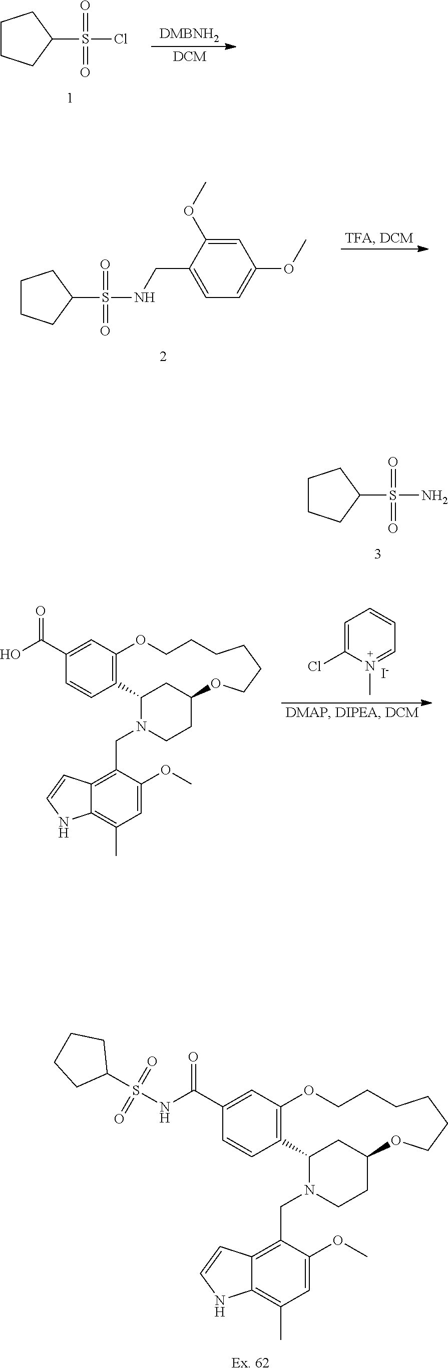

Step 1: N-(2,4-dimethoxybenzyl)cyclopentanesulfonamide. To a solution of cyclopentanesulfonyl chloride (170 mg, 1.01 mol) in DCM (2 mL) was added DMBNH₂ (339 mg, 2.02 mmol) and the mixture was stirred at room temperature for 6 hours. The mixture was concentrated to dryness under reduced pressure and the residue was purified by flash chromatography (PE:EtOAc=1:1) to give N-(2,4-dimethoxybenzyl)cyclopentane-sulfonamide (170 mg, yield 56.1%) as yellow oil. LC/MS (ESI) m/z: 300 (M+H)⁺.

Step 2: cyclopentanesulfonamide. To a solution of N-(2,4-dimethoxybenzyl)cyclopentanesulfonamide (120 mg, 0.40 mol) in DCM (2 mL) was added TFA (1 mL) and the mixture was stirred at room temperature for 2 hours. The mixture was concentrated to dryness under reduced pressure to give cyclopentanesulfonamide (130 mg, crude) as pink solid, which was used directly without purification. LC/MS (ESI) m/z: 150 (M+H)⁺.

Step 3: (1²S,1⁴S)—N-(cyclopentylsulfonyl)-1¹-((5-methoxy-7-methyl-1H-indol-4-yl)methyl)-3,10-dioxa-1(2,4)-piperidina-2(1,2)-benzenacyclodecaphane-2⁴-carboxamide. A mixture of (1²S,14S)-1¹-((5-methoxy-7-methyl-1H-indol-4-yl)methyl)-3,10-dioxa-1(2,4)-piperidina-2(1,2)-benzenacyclodecaphane-2⁴-carboxylic acid (10 mg, 0.02 mmol), cyclopentanesulfonamide (4 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)—N-(cyclopentylsulfonyl)-1¹-((5-methoxy-7-methyl-1H-indol-4-yl)methyl)-3,10-dioxa-1(2,4)-piperidina-2(1,2)-benzenacyclodecaphane-2⁴-carboxamide (4.6 mg, yield 36.9%) as white solid. ¹HNMR (400 MHz, CD₃OD) δ 8.29 (s, 1H), 7.84 (dd, J=8.0, 1.3 Hz, 1H), 7.74 (s, 1H), 7.66 (d, J=8.0 Hz, 1H), 7.32 (d, J=3.1 Hz, 1H), 6.79 (s, 1H), 6.43 (d, J=3.1 Hz, 1H), 5.42-5.35 (m, 1H), 4.49 (dd, J=16.0, 10.9 Hz, 2H), 4.20 (td, J=8.8, 4.4 Hz, 1H), 4.12 (d, J=12.8 Hz, 1H), 3.95-3.90 (m, 1H), 3.86 (s, 3H), 3.72 (s, 1H), 3.54 (dt, J=16.2, 6.7 Hz, 3H), 3.42 (dd, J=12.7, 2.4 Hz, 1H), 2.51 (s, 3H), 2.30 (d, J=15.2 Hz, 1H), 2.15-2.08 (m, 2H), 2.05-2.00 (m, 3H), 1.95 (dd, J=22.6, 8.6 Hz, 4H), 1.86-1.76 (m, 3H), 1.68-1.45 (m, 6H), 1.29 (dd, J=6.8, 2.7 Hz, 1H). LC/MS (ESI) m/z: 624 (M+H)⁺.

### Example 63: (1²S,1⁴S)—N-hydroxy-1¹-((5-methoxy-7-methyl-1H-indol-4-yl)methyl)-3,10-dioxa-1(2,4)-piperidina-2(1,2)-benzenacyclodecaphane-2⁴-carboxamide

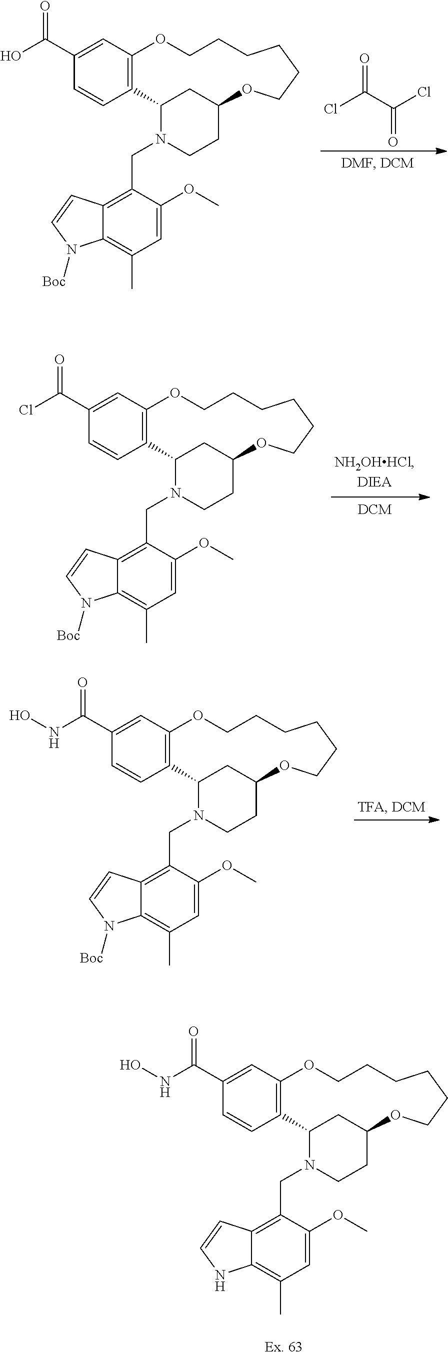 [SMILES: COc1cc(C)c2c(ccn2C(=O)OC(C)(C)C)c1CN1CC[C@H]2C[C@H]1c1ccc(C(=O)O)cc1OCCCCCCO2 ; O=C(Cl)C(=O)Cl ; COc1cc(C)c2c(ccn2C(=O)OC(C)(C)C)c1CN1CC[C@H]2C[C@H]1c1ccc(C(=O)Cl)cc1OCCCCCCO2 ; COc1cc(C)c2c(ccn2C(=O)OC(C)(C)C)c1CN1CC[C@H]2C[C@H]1c1ccc(C(=O)NO)cc1OCCCCCCO2 ; COc1cc(C)c2[nH]ccc2c1CN1CC[C@H]2C[C@H]1c1ccc(C(=O)NO)cc1OCCCCCCO2]

Step 1: tert-butyl 4-(((1²S,1⁴S)-2⁴-(chlorocarbonyl)-3,10-dioxa-1(2,4)-piperidina-2(1,2)-benzenacyclodecaphane-1¹-yl)methyl)-5-methoxy-7-methyl-1H-indole-1-carboxylate. To a solution of (1²S,1⁴S)-1¹-((1-(tert-butoxycarbonyl)-5-methoxy-7-methyl-1H-indol-4-yl)methyl)-3,10-dioxa-1(2,4)-piperidina-2(1,2)-benzenacyclodecaphane-2⁴-carboxylic acid (5 mg, 0.0084 mmol) in DCM (1 mL) was added one drop of DMF followed by drop-wise addition of oxalyl dichloride (0.1 mL) at 0° C. and the mixture was stirred at room temperature for 3 hours. The mixture was concentrated to dryness under reduced pressure to give tert-butyl 4-(((1²S,1⁴S)-2⁴-(chlorocarbonyl)-3,10-dioxa-1(2,4)-piperidina-2(1,2)-benzenacyclodecaphane-1¹-yl)methyl)-5-methoxy-7-methyl-1H-indole-1-carboxylate (10 mg, crude) as yellow oil, which was used directly without purification. LC/MS (ESI) m/z: 611 (M+H)⁺.

Step 2: tert-butyl 4-(((1²S,1⁴S)-2⁴-(hydroxycarbamoyl)-3,10-dioxa-1(2,4)-piperidina-2(1,2)-benzenacyclodecaphane-1¹-yl)methyl)-5-methoxy-7-methyl-1H-indole-1-carboxylate. To a solution of tert-butyl 4-(((1²S,1⁴S)-2⁴-(chlorocarbonyl)-3,10-dioxa-1(2,4)-piperidina-2(1,2)-benzenacyclodecaphane-1¹-yl)methyl)-5-methoxy-7-methyl-1H-indole-1-carboxylate (10 mg, 0.016 mmol) in DCM (1 mL) was added hydroxylamine hydrochloride (3 mg, 0.048 mmol) and DIEA (6 mg, 0.048 mmol) and the mixture was stirred at room temperature overnight. The mixture was concentrated under reduced pressure to dryness and the residue was purified by prep-HPLC to give tert-butyl 4-(((1²S,1⁴S)-2⁴-(hydroxycarbamoyl)-3,10-dioxa-1(2,4)-piperidina-2(1,2)-benzenacyclodecaphane-1′-yl)methyl)-5-methoxy-7-methyl-1H-indole-1-carboxylate (7 mg, yield 72.2%) as yellow oil. LC/MS (ESI) m/z: 608 (M+H)⁺.

Step 3: (1²S,1⁴S)—N-hydroxy-1¹-((5-methoxy-7-methyl-1H-indol-4-yl)methyl)-3,10-dioxa-1(2,4)-piperidina-2(1,2)-benzenacyclodecaphane-2⁴-carboxamide. To a solution of tert-butyl 4-(((1²S,1⁴S)-2⁴-(hydroxycarbamoyl)-3,10-dioxa-1(2,4)-piperidina-2(1,2)-benzenacyclodecaphane-1¹-yl)methyl)-5-methoxy-7-methyl-1H-indole-1-carboxylate (7 mg, 0.012 mol) in DCM (1 mL) was added TFA (1 mL) and the mixture was stirred at room temperature for 2 hours. The mixture was concentrated to dryness under reduced pressure and the residue was purified by prep-HPLC to give (1²S,1⁴S)—N-hydroxy-1′-((5-methoxy-7-methyl-1H-indol-4-yl)methyl)-3,10-dioxa-1(2,4)-piperidina-2(1,2)-benzenacyclodecaphane-2⁴-carboxamide (1.1 mg, yield 18.0%) as white solid. ¹H NMR (400 MHz, CD₃OD) δ 7.75 (d, J=8.1 Hz, 1H), 7.60 (d, J=8.0 Hz, 1H), 7.50 (s, 1H), 7.33 (d, J=3.1 Hz, 1H), 6.81 (s, 1H), 6.46 (d, J=3.2 Hz, 1H), 5.38 (d, J=10.6 Hz, 1H), 4.61-4.45 (m, 3H), 4.15 (d, J=12.8 Hz, 1H), 3.95 (t, J=9.1 Hz, 1H), 3.87 (s, 3H), 3.73 (s, 1H), 3.63-3.53 (m, 3H), 3.42 (d, J=11.1 Hz, 1H), 2.52 (s, 3H), 2.32 (d, J=13.9 Hz, 1H), 2.01 (d, J=13.4 Hz, 3H), 1.94 (d, J=13.2 Hz, 3H), 1.59 (d, J=10.5 Hz, 3H), 1.47 (t, J=12.4 Hz, 1H). LC/MS (ESI) m/z: 508 (M+H)⁺.

### Example 64: (1²S,1⁴S,E)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid

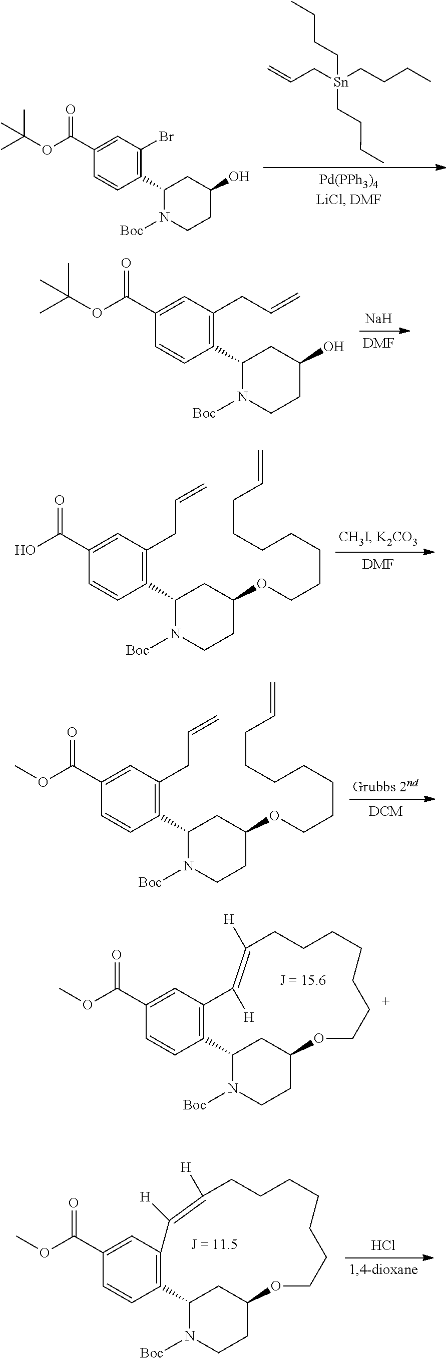

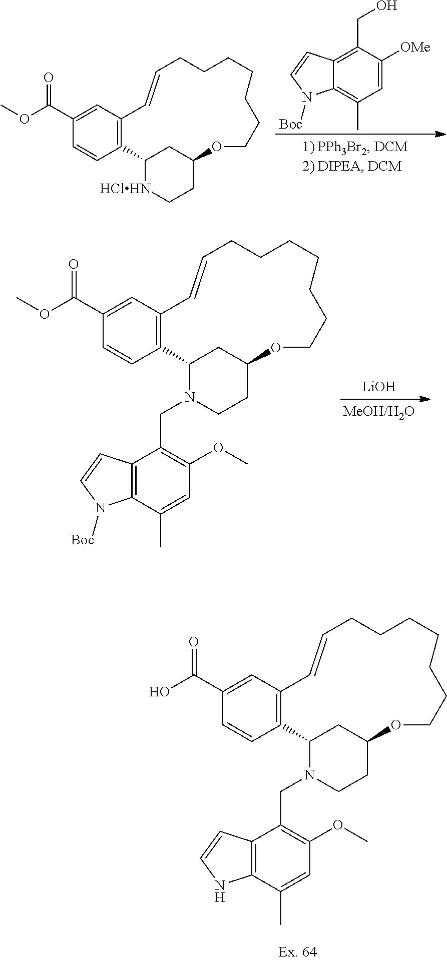

Step 1: tert-butyl (2S,4S)-2-(2-allyl-4-(tert-butoxycarbonyl)phenyl)-4-hydroxypiperidine-1-carboxylate. To a solution of tert-butyl (2S,4S)-2-(2-bromo-4-(tert-butoxycarbonyl)phenyl)-4-hydroxypiperidine-1-carboxylate (1.22 g, 2.86 mmol) in DMF (12 mL) was added allyltributylstannane (1.33 g, 4.02 mmol), Pd(PPh₃)₄(310 mg, 0.27 mmol) and LiCl (340 mg, 8.03 mmol) under N₂ atmosphere and the mixture was stirred under N₂ atmosphere at 80° C. for 3 hours. The mixture was quenched with saturated aq.KF solution and extracted with EtOAc twice. The combined organic layers was washed with brine, dried over anhydrous Na₂SO₄, filtered and concentrated to dryness under reduced pressure. The residue was purified by flash column chromatography on silica gel (PE:EtOAc=3:1) to give tert-butyl (2S,4S)-2-(2-allyl-4-(tert-butoxycarbonyl)phenyl)-4-hydroxypiperidine-1-carboxylate (880 mg, yield 59.4%). LC/MS (ESI) m/z: 518 (M+H)⁺.

Step 2: 3-allyl-4-((2S,4S)-1-(tert-butoxycarbonyl)-4-(non-8-en-1-yloxy)piperidin-2-yl)benzoic acid. To a solution of tert-butyl (2S,4S)-2-(2-allyl-4-(tert-butoxycarbonyl)phenyl)-4-hydroxypiperidine-1-carboxylate (230 mg, 0.55 mmol) in DMF (3 mL) was added NaH (132 mg, 3.3 mmol, 60% dispersion in mineral oil) in portions at 0° C. and the mixture was stirred under N₂ atmosphere for 0.5 hours. Then 9-bromonon-1-ene (226 mg, 1.1 mmol) was added and the mixture was stirred at 25° C. for 18 hours. The mixture was quenched with saturated aq.NH₄Cl solution and extracted with EtOAc twice.

The combined organic layers were washed with brine, dried over anhydrous Na₂SO₄, filtered and concentrated to dryness under reduced pressure. The residue was purified by flash column chromatography on silica gel (PE:EtOAc=1:1) to give 3-allyl-4-((2S,4S)-1-(tert-butoxycarbonyl)-4-(non-8-en-1-yloxy)piperidin-2-yl)benzoic acid (200 mg, yield 74.9%) as colorless oil. LC/MS: m/z 486 (M+H)⁺.

Step 3: tert-butyl (2S,4S)-2-(2-allyl-4-(methoxycarbonyl)phenyl)-4-(non-8-en-1-yloxy)piperidine-1-carboxylate. To a solution of 3-allyl-4-((2S,4S)-1-(tert-butoxycarbonyl)-4-(non-8-en-1-yloxy)piperidin-2-yl)benzoic acid (200 mg, 0.41 mmol) in DMF (2 mL) was added K₂CO₃ (170 mg, 1.23 mmol) and MeI (349 mg, 2.46 mmol) and the mixture was stirred at 25° C. for 1 hour. The mixture was diluted with EtOAc (10 mL), washed with water and brine, dried over anhydrous Na₂SO₄, filtered and concentrated under reduced pressure to dryness. The residue was purified by flash chromatography (PE:EtOAc=8:1) to give tert-butyl (2S,4S)-2-(2-allyl-4-(methoxycarbonyl)phenyl)-4-(non-8-en-1-yloxy)piperidine-1-carboxylate (135 mg, yield 65.9%) as colorless oil. LC/MS (ESI) m/z: 500 (M+H)⁺.

Step 4: 1¹-(tert-butyl) 2⁴-methyl (1²S,1⁴S,E)-12-oxa-1(2,4)-piperidina-2(1,2)-benzenacyclododecaphan-3-ene-1¹,2⁴-dicarboxylate and 11-(tert-butyl) 2⁴-methyl (1²S,1⁴S,Z)-12-oxa-1(2,4)-piperidina-2(1,2)-benzenacyclododecaphan-3-ene-1¹,2⁴-dicarboxylate. To a solution of tert-butyl (2S,4S)-2-(2-allyl-4-(methoxycarbonyl)phenyl)-4-(non-8-en-1-yloxy)piperidine-1-carboxylate (117 mg, 0.23 mmol) in dry DCM (117 mL) was added Grubbs 2^{nd} (40 mg, 0.047 mmol), the mixture was degassed under N₂ atmosphere for three times and stirred at 25° C. for 18 hours. The mixture was concentrated under reduced pressure to dryness and the residue was purified by flash chromatography (PE:EtOAc=10:1) to give 1¹-(tert-butyl) 2⁴-methyl (1²,1⁴S,E)-12-oxa-1(2,4)-piperidina-2(1,2)-benzenacyclododecaphan-3-ene-1¹,2⁴-dicarboxylate (45 mg, yield 42.9%) as brown oil and 1¹-(tert-butyl) 2⁴-methyl (1²,1⁴S,Z)-12-oxa-1(2,4)-piperidina-2(1,2)-benzenacyclododecaphan-3-ene-1¹,2⁴-dicarboxylate (10 mg, 9.5% yield) as brown oil. LC/MS (ESI) m/z: 458 (M+H)⁺.

Step 5: methyl (1²S,1⁴S,E)-12-oxa-1(2,4)-piperidina-2(1,2)-benzenacyclododecaphan-3-ene-2⁴-carboxylate hydrochloride. A solution of 1¹-(tert-butyl) 2⁴-methyl (1²S,1⁴S,E)-12-oxa-1(2,4)-piperidina-2(1,2)-benzenacyclododecaphan-3-ene-1¹,2⁴-dicarboxylate (45 mg, 0.098 mmol) in HCl/1,4-dioxane (1 mL) was stirred at 25° C. for 1 hour. The mixture was concentrated to dryness to give methyl (1²S,1⁴S,E)-12-oxa-1(2,4)-piperidina-2(1,2)-benzenacyclododecaphan-3-ene-2⁴-carboxylate hydrochloride (45 mg, crude) as white solid, which was used directly without purification. LC/MS (ESI) m/z: 358 (M+H)⁺.

Step 6: methyl (1²S,1⁴S,E)-1¹-((1-(tert-butoxycarbonyl)-5-methoxy-7-methyl-1H-indol-4-yl)methyl)-12-oxa-1(2,4)-piperidina-2(1,2)-benzenacyclododecaphan-3-ene-2⁴-carboxylate. To a solution of tert-butyl 4-(hydroxymethyl)-5-methoxy-7-methyl-1H-indole-1-carboxylate (44 mg, 0.15 mmol) in dry DCM (1 mL) was added PPh₃Br₂ (59 mg, 0.16 mmol) portion-wise and the mixture was stirred under N₂ atmosphere for 2 hours at 0° C. Then a mixture of methyl (1²S,1⁴S,E)-12-oxa-1(2,4)-piperidina-2(1,2)-benzenacyclododecaphan-3-ene-2⁴-carboxylate hydrochloride (45 mg, 0.12 mmol) and DIPEA (49 mg, 0.38 mmol) in dry DCM (1 mL) was added and the resulting mixture was stirred at 0° C. for 1.5 hours. The mixture was concentrated to dryness and the residue was purified by prep-HPLC to give methyl (1²S,1⁴S,E)-11-((1-(tert-butoxycarbonyl)-5-methoxy-7-methyl-1H-indol-4-yl)methyl)-12-oxa-1(2,4)-piperidina-2(1,2)-benzenacyclododecaphan-3-ene-2⁴-carboxylate (50 mg, 52.6% yield) as colorless oil. LC/MS (ESI) m/z: 631 (M+H)⁺.

Step 7: (1²S,1⁴S,E)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid. To a solution of methyl (1²S,1⁴S,E)-11-((1-(tert-butoxycarbonyl)-5-methoxy-7-methyl-1H-indol-4-yl)methyl)-12-oxa-1(2,4)-piperidina-2(1,2)-benzenacyclododecaphan-3-ene-2⁴-carboxylate (50 mg, 0.079 mmol) in MeOH (1 mL)/H₂O (0.3 mL) was added LiOH H₂O (7 mg, 0.16 mmol) and the mixture was stirred at 50° C. overnight. The mixture was acidified with 1.0 N aq.HCl to pH˜4 and extracted with EtOAc twice. The combined organic layers were washed with water and brine, dried over anhydrous Na₂SO₄, filtered and concentrated to dryness. The residue was purified by prep-HPLC to give (1²S,1⁴S,E)-1′-((5-methoxy-7-methyl-1H-indol-4-yl)methyl)-12-oxa-1(2,4)-piperidina-2(1,2)-benzenacyclododecaphan-3-ene-2⁴-carboxylic acid (15 mg, yield 37.5%) as white solid. ¹H NMR (400 MHz, CD₃OD) δ 8.36 (s, 1H), 8.10 (d, J=8.4 Hz, 2H), 7.71 (d, J=8.1 Hz, 1H), 7.34 (d, J=3.2 Hz, 1H), 6.96 (d, J=15.5 Hz, 1H), 6.80 (s, 1H), 6.44 (d, J=3.1 Hz, 1H), 6.17-6.07 (m, 1H), 5.14 (d, J=9.6 Hz, 1H), 4.44 (d, J=12.6 Hz, 1H), 4.30 (d, J=12.7 Hz, 1H), 3.83 (d, J=8.2 Hz, 3H), 3.76 (s, 1H), 3.66 (dd, J=9.4, 4.9 Hz, 1H), 3.59 (dd, J=12.9, 3.4 Hz, 1H), 3.43 (dt, J=8.8, 6.3 Hz, 2H), 2.52 (s, 3H), 2.39 (dd, J=13.1, 8.3 Hz, 2H), 2.19-2.14 (m, 1H), 2.11-2.04 (m, 2H), 1.99 (d, J=13.9 Hz, 1H), 1.75-1.67 (m, 3H), 1.65-1.53 (m, 3H), 1.51-1.43 (m, 4H). LC/MS (ESI) m/z: 517 (M+H)⁺.

### Example 65: (1²S,1⁴S)-1¹-((5-methoxy-7-methyl-1H-indol-4-yl)methyl)-N-(methylsulfonyl)-11-oxa-1(2,4)-piperidina-2(1,2)-benzenacycloundecaphane-2⁴-carboxamide

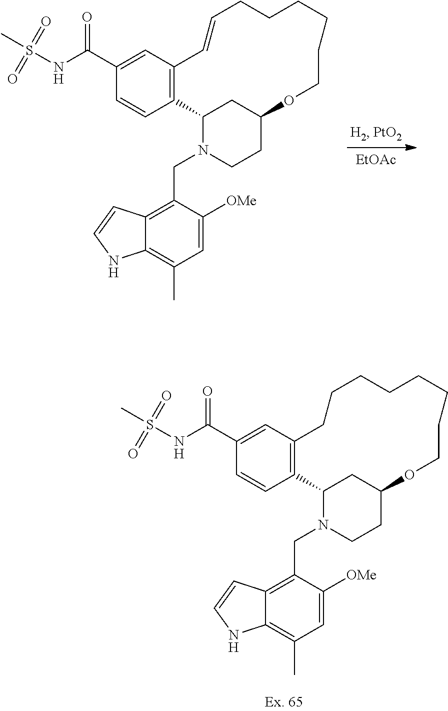 [SMILES: "EtOAc" ; COc1cc(C)c2[nH]ccc2c1CN1CC[C@H]2C[C@H]1c1ccc(C(=O)NS(C)(=O)=O)cc1/C=C/CCCCCCO2 ; COc1cc(C)c2[nH]ccc2c1CN1CC[C@H]2C[C@H]1c1ccc(C(=O)NS(C)(=O)=O)cc1CCCCCCCCO2]

To a solution of (1²S,1⁴S,E)-1¹-((5-methoxy-7-methyl-1H-indol-4-yl)methyl)-N-(methylsulfonyl)-11-oxa-1(2,4)-piperidina-2(1,2)-benzenacycloundecaphan-3-ene-2⁴-carboxamide (10 mg, 0.017 mmol) in EtOAc (1 mL) was added PtO₂ (1 mg, 0.0017 mmol) and the mixture was stirred under a H₂ balloon at room temperature for 0.5 hours. The mixture was filtrated and the filtrate was concentrated under reduced pressure to dryness. The residue was purified by prep-HPLC to give (1²S,1⁴S)—N-(cyclopropylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide (1.7 mg, yield 17.2%) as white solid. ¹H NMR (400 MHz, CD₃OD) δ 8.28 (s, 1H), 8.11 (dd, J=8.2, 1.5 Hz, 1H), 8.00 (d, J=1.3 Hz, 1H), 7.65 (d, J=8.2 Hz, 1H), 7.31 (d, J=3.1 Hz, 1H), 6.75 (s, 1H), 6.37 (d, J=3.1 Hz, 1H), 4.94 (dd, J=10.6, 4.2 Hz, 1H), 4.31-4.22 (m, 2H), 3.76 (s, 1H), 3.73 (s, 3H), 3.70-3.59 (m, 2H), 3.56-3.51 (m, 1H), 3.44 (d, J=12.5 Hz, 1H), 3.16 (s, 3H), 3.09-3.00 (m, 1H), 2.73-2.64 (m, 1H), 2.49 (s, 3H), 2.28-2.19 (m, 2H), 2.01 (td, J=13.5, 3.4 Hz, 3H), 1.89 (d, J=12.2 Hz, 1H), 1.74-1.59 (m, 4H), 1.57-1.40 (m, 4H), 1.35-1.30 (m, 2H). LC/MS (ESI) m/z: 582 (M+H)⁺.

### Example 66: (1²S,1⁴S)—N-(isopropylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide

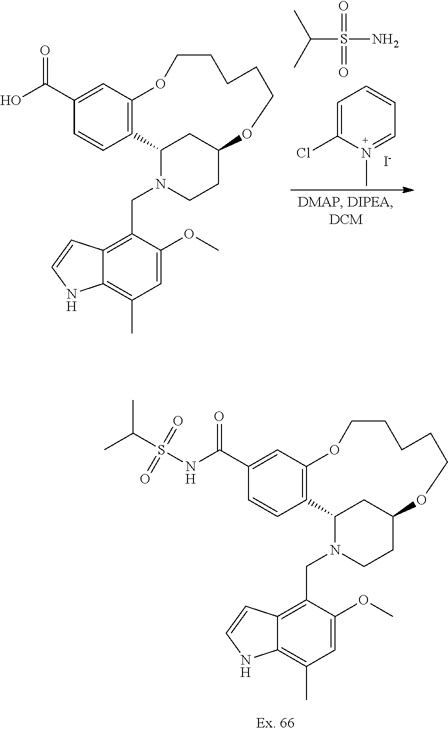

A mixture of (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol), propane-2-sulfonamide (4 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)—N-(isopropylsulfonyl)-1¹-((5-methoxy-7-methyl-1H-indol-4-yl)methyl)-3,9-dioxa-1(2,4)-piperidina-2(1,2)-benzenacyclononaphane-2⁴-carboxamide (1.8 mg, yield 15.4%) as white solid. ¹H NMR (400 MHz, CD₃OD) δ 7.96-7.88 (m, 2H), 7.61 (d, J=8.1 Hz, 1H), 7.32 (d, J=3.1 Hz, 1H), 6.78 (s, 1H), 6.41 (d, J=3.1 Hz, 1H), 5.53 (d, J=10.9 Hz, 1H), 4.39 (dd, J=17.7, 9.3 Hz, 2H), 4.24 (d, J=11.4 Hz, 1H), 4.09 (d, J=12.8 Hz, 1H), 3.87-3.81 (m, 4H), 3.78 (s, 1H), 3.70-3.64 (m, 1H), 3.61-3.54 (m, 2H), 3.42 (dd, J=12.9, 2.6 Hz, 1H), 2.51 (s, 3H), 2.43 (d, J=10.9 Hz, 1H), 2.32 (d, J=14.7 Hz, 1H), 2.14-2.06 (m, 1H), 1.94 (dd, J=37.4, 21.9 Hz, 3H), 1.60 (d, J=12.1 Hz, 3H), 1.37 (d, J=6.8 Hz, 6H), 1.29 (dd, J=9.4, 6.0 Hz, 1H). LC/MS (ESI) m/z: 584 (M+H)⁺.

### Example 67: (1²S,1⁴S)-1¹-((5-methoxy-7-methyl-1H-indol-4-yl)methyl)-N-((1-methylcyclopropyl)sulfonyl)-3,9-dioxa-1(2,4)-piperidina-2(1,2)-benzenacyclononaphane-2⁴-carboxamide

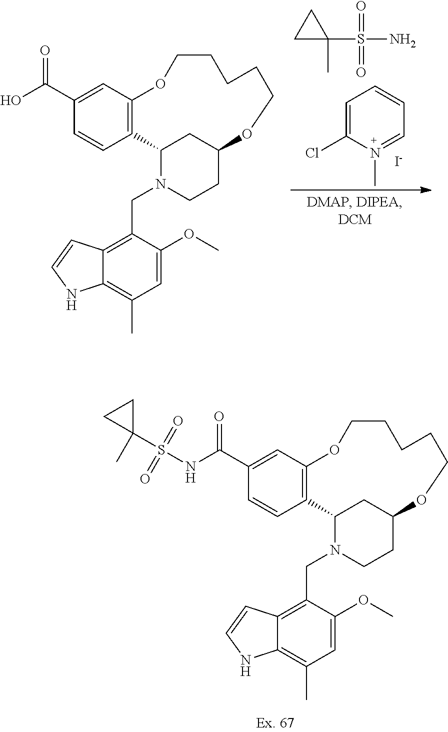

A mixture of (1²S,1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol), 1-methylcyclopropane-1-sulfonamide (4 mg, 0.026 mmol), 2-chloro-1-methylpyridin-1-ium iodide (7 mg, 0.026 mmol), DMAP (1 mg, 0.001 mmol) and DIPEA (8 mg, 0.06 mmol) in DCM (1 mL) was stirred at room temperature for 18 hours. The mixture was concentrated under reduced pressure to dryness and the residue was purified by prep-HPLC to give (1²S,1⁴S)_1¹-((5-methoxy-7-methyl-1H-indol-4-yl)methyl)-N-((1-methylcyclopropyl)sulfonyl)-3,9-dioxa-1(2,4)-piperidina-2(1,2)-benzenacyclononaphane-2⁴-carboxamide (2.6 mg, yield 21.8%) as white solid. ¹HNMR (400 MHz, CD₃OD) δ 7.93 (d, J=8.0 Hz, 1H), 7.88 (s, 1H), 7.63 (d, J=8.0 Hz, 1H), 7.32 (d, J=3.1 Hz, 1H), 6.78 (s, 1H), 6.41 (d, J=3.1 Hz, 1H), 5.53 (d, J=11.4 Hz, 1H), 4.44-4.36 (m, 2H), 4.25 (dd, J=7.3, 4.2 Hz, 1H), 4.10 (d, J=12.8 Hz, 1H), 3.83 (s, 3H), 3.78 (s, 1H), 3.70-3.64 (m, 1H), 3.57 (dt, J=13.5, 6.6 Hz, 2H), 3.42 (dd, J=12.8, 2.7 Hz, 1H), 2.51 (s, 3H), 2.43 (d, J=5.3 Hz, 1H), 2.31 (d, J=15.3 Hz, 1H), 2.14-2.06 (m, 1H), 2.04-1.86 (m, 3H), 1.62 (s, 3H), 1.55 (s, 6H), 0.80 (d, J=1.5 Hz, 2H). LC/MS (ESI) m/z: 596 (M+H)⁺.

### Example 68: (1²S,1⁴S,Z)-1¹-((5-methoxy-7-methyl-1H-indol-4-yl)methyl) —N-(methylsulfonyl)-10-oxa-1(2,4)-piperidina-2(1,2)-benzenacyclodecaphan-3-ene-24-carboxamide

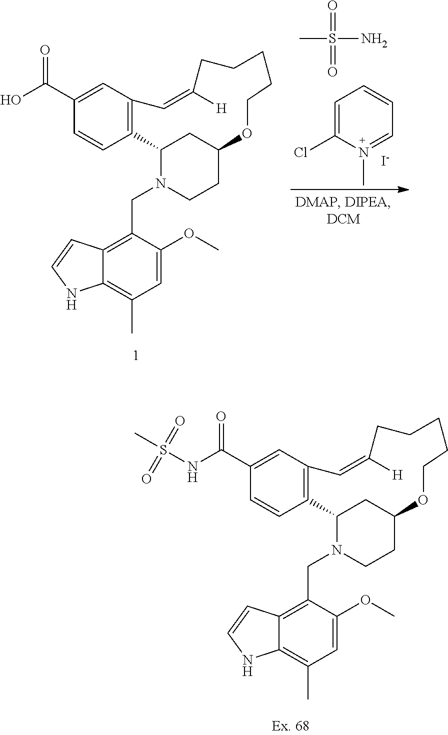

To a mixture of (1²S,1⁴S,Z)-1¹-((5-methoxy-7-methyl-1H-indol-4-yl)methyl)-10-oxa-1(2,4)-piperidina-2(1,2)-benzenacyclodecaphan-3-ene-2⁴-carboxylic acid (25 mg, 0.051 mmol) and methanesulfonamide (7 mg, 0.077 mmol) in DCM (3 mL) was added 2-chloro-1-methylpyridin-1-ium iodide (20 mg, 0.077 mmol) followed by DMAP (2 mg, 0.01 mmol) and DIPEA (20 mg, 0.15 mmol) and the mixture was stirred at room temperature for 18 hours.

The mixture was concentrated to dryness and the residue was purified by prep-HPLC to give (12S,14S,Z)-11-((5-methoxy-7-methyl-1H-indol-4-yl)methyl)-N-(methylsulfonyl)-10-oxa-1(2,4)-piperidina-2(1,2)-benzenacyclodecaphan-3-ene-24-carboxamide (6.0 mg, yield 20.8%) as light yellow solid. ¹H NMR (400 MHz, CD₃OD) δ 8.25 (s, 1H), 8.18 (d, J=8.4 Hz, 1H), 7.90 (s, 1H), 7.68 (d, J=8.4 Hz, 1H), 7.30 (d, J=3.2 Hz, 1H), 6.75 (s, 1H), 6.68 (d, J=11.6 Hz, 1H), 6.29 (d, J=2.8 Hz, 1H), 6.08-6.01 (m, 1H), 5.04 (d, J=10.8 Hz, 1H), 4.35 (d, J=12.4 Hz, 1H), 4.20 (d, J=12.4 Hz, 1H), 3.82-3.76 (m, 2H), 3.70 (s, 3H), 3.59-3.44 (m, 4H), 3.15 (s, 3H), 2.49 (s, 3H), 2.42-2.28 (m, 2H), 2.09-1.95 (m, 4H), 1.82-1.74 (m, 1H), 1.66-1.55 (m, 2H), 1.48-1.43 (m, 2H).LC/MS (ESI) m/z: 566 (M+H)⁺.

### Example 69: (1²S,1⁴S,E)-1¹-((5-methoxy-7-methyl-1H-indol-4-yl)methyl)-N-(methylsulfonyl)-10-oxa-1(2,4)-piperidina-2(1,2)-benzenacyclodecaphan-3-ene-2⁴-carboxamide

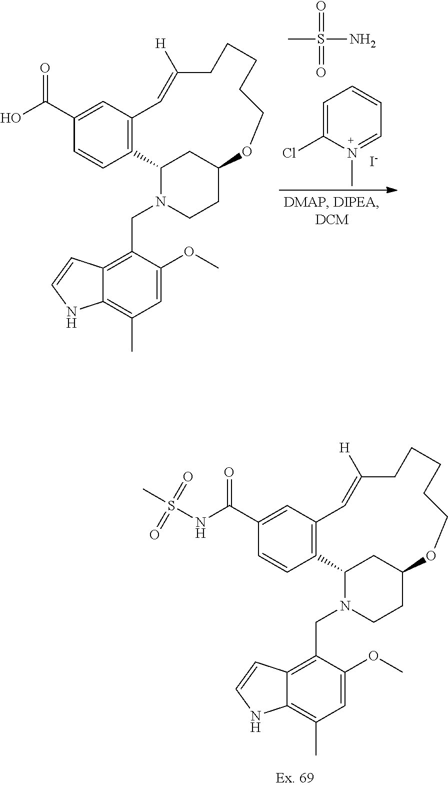

To a mixture of (1²S,1⁴S,E)-1¹-((5-methoxy-7-methyl-1H-indol-4-yl)methyl)-10-oxa-1(2,4)-piperidina-2(1,2)-benzenacyclodecaphan-3-ene-2⁴-carboxylic acid (25 mg, 0.051 mmol) and methanesulfonamide (7 mg, 0.077 mmol) in DCM (3 mL) was added 2-chloro-1-methylpyridin-1-ium iodide (20 mg, 0.077 mmol) followed by DMAP (2 mg, 0.01 mmol) and DIPEA (20 mg, 0.15 mmol) and the mixture was stirred at room temperature for 18 hours. The mixture was concentrated to dryness and the residue was purified by prep-HPLC to give (1²S,1⁴S,E)-1¹-((5-methoxy-7-methyl-1H-indol-4-yl)methyl)-N-(methylsulfonyl)-10-oxa-1(2,4)-piperidina-2(1,2)-benzenacyclodecaphan-3-ene-2⁴-carboxamide (6.0 mg, yield 20.8%) as light yellow solid. ¹H NMR (400 MHz, CD₃OD) δ 8.25 (s, 1H), 8.15 (dd, J=8.0, 8.0 Hz, 1H), 7.95 (d, J=1.2 Hz, 1H), 7.71 (d, J=8.0 Hz, 1H), 7.34 (d, J=3.2 Hz, 1H), 6.81 (t, J=8.0 Hz, 2H), 6.49 (d, J=3.2 Hz, 1H), 5.85-5.74 (m, 1H), 5.18-5.10 (m, 1H), 4.51 (d, J=12.8 Hz, 1H), 4.24 (d, J=12.8 Hz, 1H), 3.88 (s, 3H), 3.73-3.67 (m, 1H), 3.62-3.54 (m, 2H), 3.52-3.46 (m, 1H), 3.43-3.37 (m, 1H), 3.16 (s, 3H), 2.52 (s, 3H), 2.47-2.39 (m, 1H), 2.30-2.22 (m, 1H), 2.12-1.99 (m, 2H), 1.94-1.87 (m, 3H), 1.81-1.71 (m, 1H), 1.62-1.51 (m, 1H), 1.47-1.37 (m, 1H), 1.33-1.26 (m, 1H).LC/MS (ESI) m/z: 566 (M+H)⁺.

### Example 70: (1²S,1⁴S)-1¹-((5-methoxy-7-methyl-1H-indol-4-yl)methyl)-N-(phenylsulfonyl)-3,9-dioxa-1(2,4)-piperidina-2(1,2)-benzenacyclononaphane-2⁴-carboxamide

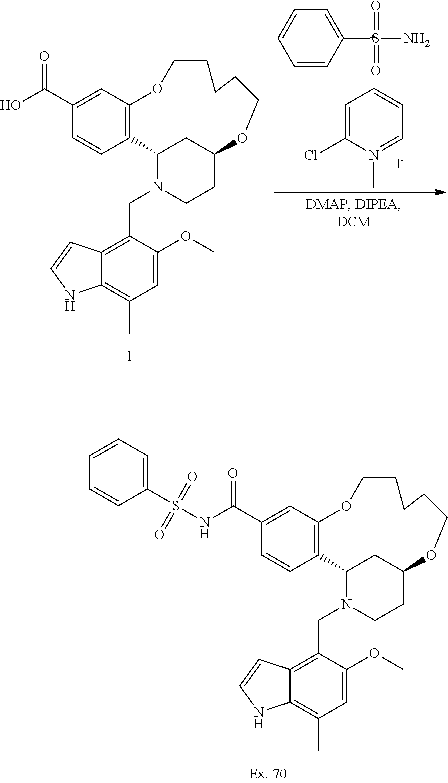

To a mixture of (1²S, 1⁴S)-1¹-((5-methoxy-7-methyl-1H-indol-4-yl)methyl)-3,9-dioxa-1(2,4)-piperidina-2(1,2)-benzenacyclononaphane-2⁴-carboxylic acid (10 mg, 0.02 mmol) and benzene-sulfonamide (5 mg, 0.03 mmol) in DCM (2 mL) was added 2-chloro-1-methylpyridin-1-ium (8 mg, 0.03 mmol) followed by DMAP (0.6 mg, 0.005 mmol) and DIPEA (8 mg, 0.06 mmol) and the mixture was stirred at room temperature for 18 hours. The mixture was concentrated to dryness and the residue was purified by prep-HPLC to give (1²S,1⁴S)-1¹-((5-methoxy-7-methyl-1H-indol-4-yl)methyl)-N-(phenylsulfonyl)-3,9-dioxa-1(2,4)-piperidina-2(1,2)-benzenacyclononaphane-2⁴-carboxamide (1.2 mg, yield 9.3%) as white solid. ¹H NMR (400 MHz, CD₃OD) δ 8.00 (dd, J=7.9, 1.7 Hz, 2H), 7.89-7.84 (m, 2H), 7.57 (d, J=8.1 Hz, 1H), 7.47 (t, J=6.9 Hz, 3H), 7.31 (d, J=3.1 Hz, 1H), 6.78 (s, 1H), 6.40 (d, J=3.2 Hz, 1H), 4.44-4.34 (m, 3H), 4.23 (d, J=11.8 Hz, 1H), 4.08 (d, J=12.6 Hz, 1H), 3.81 (s, 3H), 3.78 (s, 1H), 3.69-3.65 (m, 1H), 3.60 (d, J=8.3 Hz, 2H), 3.47 (s, 1H), 3.41 (d, J=4.6 Hz, 2H), 2.50 (s, 3H), 2.31 (d, J=14.7 Hz, 1H), 2.01 (dd, J=31.3, 19.5 Hz, 4H), 1.61 (s, 4H). LC/MS (ESI) m/z: 618 (M+H)⁺.

### Example 71: (1²S,1⁴S)-1¹-((5-methoxy-7-methyl-1H-indol-4-yl)methyl)-N-(methylsulfonyl)-10-oxa-1(2,4)-piperidina-2(1,2)-benzenacyclodecaphane-2⁴-carboxamide

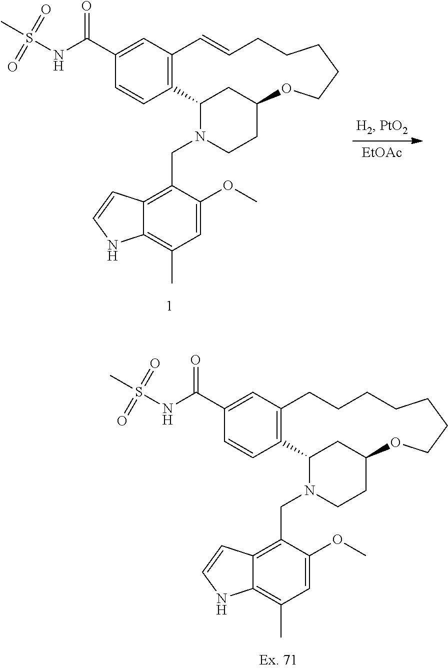 [SMILES: "EtOAc" ; COc1cc(C)c2[nH]ccc2c1CN1CC[C@H]2C[C@H]1c1ccc(C(=O)NS(C)(=O)=O)cc1/C=C/CCCCCO2 ; COc1cc(C)c2[nH]ccc2c1CN1CC[C@H]2C[C@H]1c1ccc(C(=O)NS(C)(=O)=O)cc1CCCCCCCO2]

To a solution of (1²S,1⁴S,E)-1¹-((5-methoxy-7-methyl-1H-indol-4-yl)methyl)-N-(methylsulfonyl)-10-oxa-1 (2,4)-piperidina-2(1,2)-benzenacyclodecaphan-3-ene-2⁴-carboxamide (10 mg, 0.018 mmol) in MeOH (2 mL) was added PtO₂ (2 mg, 0.0088 mmol) and the mixture was stirred under a H₂ balloon at room temperature for 1 hour. The mixture was filtered and the filtrate was concentrated under reduced pressure to dryness. The residue was purified by prep-HPLC to give (1²S,1⁴S)-1′-((5-methoxy-7-methyl-1H-indol-4-yl)methyl)-N-(methylsulfonyl)-10-oxa-1(2,4)-piperidina-2(1,2)-benzenacyclodecaphane-2⁴-carboxamide (2.1 mg, yield 21.0%) as white solid. ¹H NMR (400 MHz, CD₃OD) δ 8.11 (dd, J=8.4, 8.4 Hz, 1H), 8.01 (d, J=1.6 Hz, 1H), 7.65 (d, J=8.0 Hz, 1H), 7.33 (d, J=3.2 Hz, 1H), 6.78 (s, 1H), 6.44 (d, J=3.2 Hz, 1H), 5.12 (d, J=10.0 Hz, 1H), 4.33 (d, J=12.8 Hz, 1H), 4.20 (d, J=12.4 Hz, 1H), 3.83-3.77 (m, 5H), 3.68-3.60 (m, 1H), 3.52-3.43 (m, 2H), 3.15 (s, 3H), 3.04-2.94 (m, 1H), 2.81-2.72 (m, 1H), 2.51 (s, 3H), 2.33-2.14 (m, 2H), 2.06-1.96 (m, 2H), 1.86-1.71 (m, 4H), 1.63-1.45 (m, 6H). LC/MS (ESI) m/z: 568 (M+H)⁺.

### Example 72: (1²S,1⁴S)-1¹-((5-methoxy-7-methyl-1H-indol-4-yl)methyl)-12-oxa-1(2,4)-piperidina-2(1,2)-benzenacyclododecaphane-2⁴-carboxylic acid

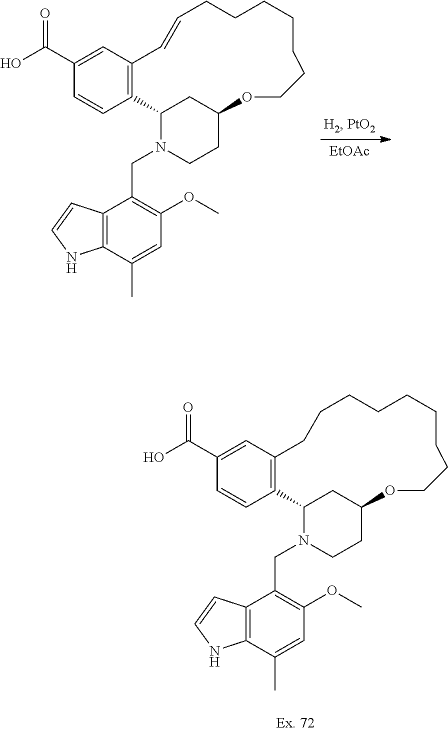 [SMILES: "EtOAc" ; COc1cc(C)c2[nH]ccc2c1CN1CC[C@H]2C[C@H]1c1ccc(C(=O)O)cc1/C=C/CCCCCCCO2 ; COc1cc(C)c2[nH]ccc2c1CN1CC[C@H]2C[C@H]1c1ccc(C(=O)O)cc1CCCCCCCCCO2]

To a solution of (1²S,1⁴S,E)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid (10 mg, 0.019 mmol) in EtOAc (1 mL) was added PtO₂ (1 mg, 0.0019 mmol) and the mixture was stirred under a H₂ balloon at room temperature for 0.5 hours. The mixture was filtrated and the filtrate was concentrated under reduced pressure to dryness. The residue was purified by prep-HPLC to give (1²S,1⁴S)-1¹-((5-methoxy-7-methyl-1H-indol-4-yl)methyl)-12-oxa-1(2,4)-piperidina-2(1,2)-benzenacyclododecaphane-2⁴-carboxylic acid (2.0 mg, yield 17.2%) as white solid. ¹H NMR (400 MHz, CD₃OD) δ 8.43 (s, 1H), 8.06 (d, J=8.1 Hz, 1H), 7.96 (s, 1H), 7.66 (d, J=8.1 Hz, 1H), 7.32 (d, J=3.1 Hz, 1H), 6.76 (s, 1H), 6.37 (d, J=2.6 Hz, 1H), 4.97 (d, J=10.7 Hz, 1H), 4.31-4.23 (m, 2H), 3.78 (d, J=9.4 Hz, 2H), 3.75 (s, 3H), 3.56 (dd, J=18.4, 7.8 Hz, 1H), 3.43 (dd, J=11.9, 3.8 Hz, 2H), 3.11-3.03 (m, 1H), 2.72-2.65 (m, 1H), 2.50 (s, 3H), 2.31 (t, J=13.7 Hz, 1H), 2.19-2.12 (m, 2H), 1.94 (t, J=14.2 Hz, 1H), 1.73-1.59 (m, 8H), 1.57-1.45 (m, 6H). LC/MS (ESI) m/z: 519 (M+H)⁺.

### Example 73: (1²S,1⁴S,Z)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid

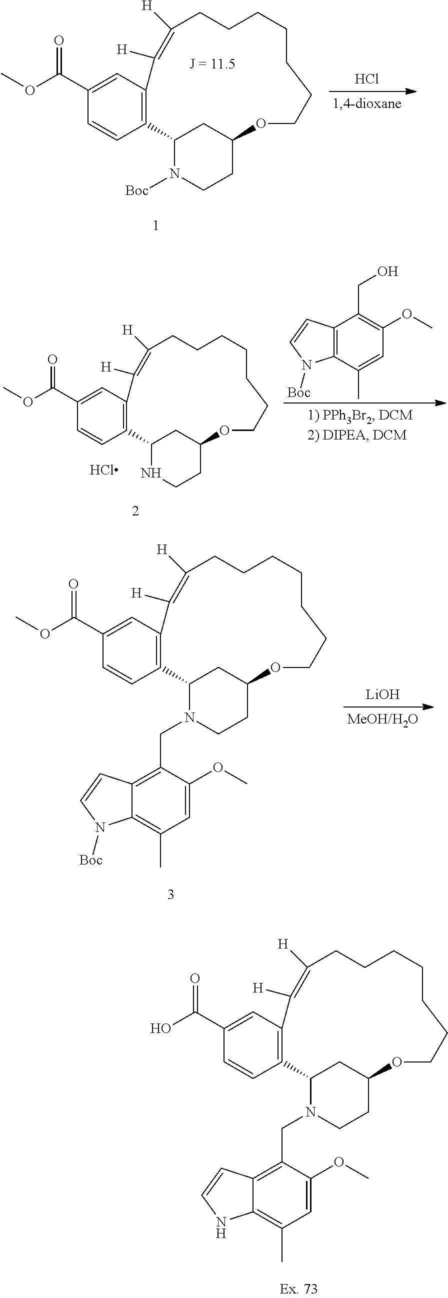

Step 1: methyl (1²S,1⁴S,Z)-12-oxa-1(2,4)-piperidina-2(1,2)-benzenacyclododecaphan-3-ene-2⁴-carboxylate hydrochloride. A solution of 1¹-(tert-butyl) 2⁴-methyl (1²S,1⁴S,Z)-12-oxa-1(2,4)-piperidina-2(1,2)-benzenacyclododecaphan-3-ene-1¹,2⁴-dicarboxylate (10 mg, 0.022 mmol) in HCl/1,4-dioxane (1 mL) was stirred at 25° C. for 1 hour. The mixture was concentrated to dryness to give methyl(1²S,1⁴S,Z)-12-oxa-1(2,4)-piperidina-2(1,2)-benzenacyclododecaphan-3-ene-2⁴-carboxylate hydrochloride (10 mg, crude) as white solid, which was used directly without purification. LC/MS (ESI) m/z: 358 (M+H)⁺.

Step 2: methyl (1²S,1⁴S,Z)-1¹-((1-(tert-butoxycarbonyl)-5-methoxy-7-methyl-1H-indol-4-yl)methyl)-12-oxa-1(2,4)-piperidina-2(1,2)-benzenacyclododecaphan-3-ene-2⁴-carboxylate. At 0° C., a solution of tert-butyl 4-(hydroxymethyl)-5-methoxy-7-methyl-1H-indole-1-carboxylate (10 mg, 0.034 mmol) in dry DCM (1 mL) was added PPh₃Br₂ (15 mg, 0.036 mmol) portion-wise and the mixture was stirred under N₂ atmosphere for 2 hours. Then a mixture of methyl (1²,1⁴S,Z)-12-oxa-1(2,4)-piperidina-2(1,2)-benzenacyclododecaphan-3-ene-2⁴-carboxylate hydrochloride (10 mg, 0.028 mmol) and DIPEA (11 mg, 0.084 mmol) in dry DCM (1 mL) was added and the resulting mixture was stirred at 0° C. for 1.5 hours. The mixture was concentrated to dryness and the residue was purified by prep-HPLC to give methyl (1²S,1⁴S,Z)-1¹-((1-(tert-butoxycarbonyl)-5-methoxy-7-methyl-1H-indol-4-yl)methyl)-12-oxa-1(2,4)-piperidina-2(1,2)-benzenacyclododecaphan-3-ene-2⁴-carboxylate (10 mg, yield 46.5%) as colorless oil. LC/MS (ESI) m/z: 631 (M+H)⁺.

Step 3: (1²S,1⁴S,Z)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid. To a solution of methyl (1²S,1⁴S,Z)-1¹-((1-(tert-butoxycarbonyl)-5-methoxy-7-methyl-1H-indol-4-yl)methyl)-12-oxa-1 (2,4)-piperidina-2(1,2)-benzenacyclododecaphan-3-ene-2⁴-carboxylate (10 mg, 0.016 mmol) in MeOH (1 mL)/water (0.3 mL) was added LiOH·H₂O (2 mg, 0.032 mmol) and the mixture was stirred at 50° C. overnight. The mixture was acidified with 1.0 N aq.HCl to pH˜4 and extracted with EtOAc twice. The combined organic layers were washed with water and brine, dried over anhydrous Na₂SO₄, filtered and concentrated to dryness.

The residue was purified by prep-HPLC to give (1²S,1⁴S,Z)-1¹-((5-methoxy-7-methyl-1H-indol-4-yl)methyl)-12-oxa-1(2,4)-piperidina-2(1,2)-benzenacyclododecaphan-3-ene-2⁴-carboxylic acid (1.5 mg, yield 18.1%) as white solid. ¹H NMR (400 MHz, CD₃OD) δ 8.42 (s, 1H), 8.13 (d, J=8.1 Hz, 1H), 7.88 (s, 1H), 7.71 (d, J=8.2 Hz, 1H), 7.31 (d, J=3.1 Hz, 1H), 6.75 (d, J=8.2 Hz, 2H), 6.39 (s, 1H), 6.09-6.02 (m, 1H), 5.02 (d, J=12.2 Hz, 1H), 4.33 (dd, J=40.4, 12.4 Hz, 2H), 3.75 (d, J=15.1 Hz, 5H), 3.58 (d, J=11.4 Hz, 1H), 3.44 (d, J=10.6 Hz, 1H), 2.50 (s, 3H), 2.32 (d, J=14.1 Hz, 1H), 2.17 (dt, J=16.1, 11.5 Hz, 3H), 2.10-1.90 (m, 3H), 1.65 (s, 3H), 1.46 (d, J=5.0 Hz, 2H), 1.41-1.31 (m, 6H). LC/MS (ESI) m/z: 517 (M+H)⁺.

### Example 74: (1²S,1⁴S)-1¹-((5-methoxy-7-methyl-1H-indol-4-yl)methyl)-2⁴-(1H-tetrazol-5-yl)-3,10-dioxa-1(2,4)-piperidina-2(1,2)-benzenacyclodecaphane

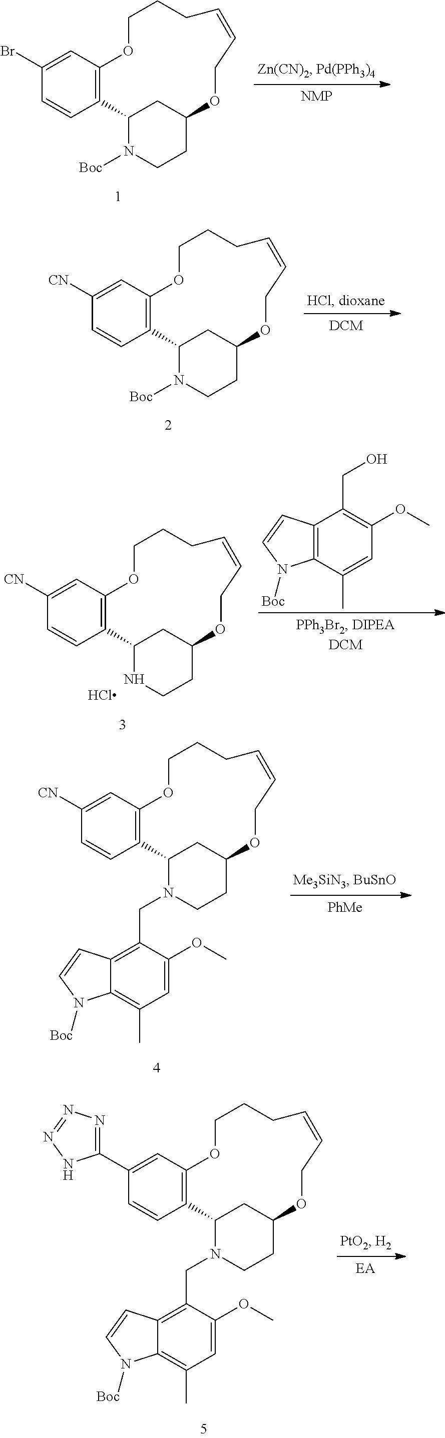

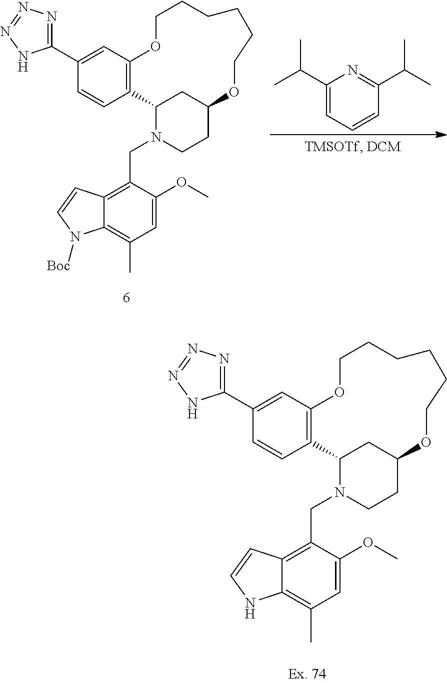

Step 1: tert-butyl (1²S,1⁴S,Z)-2⁴-cyano-3,10-dioxa-1(2,4)-piperidina-2(1,2)-benzenacyclodecaphan-7-ene-1′-carboxylate. To a solution of tert-butyl (1²S,1⁴S,Z)-2⁴-bromo-3,10-dioxa-1(2,4)-piperidina-2(1,2)-benzenacyclodecaphan-7-ene-1′-carboxylate (230 mg, 0.51 mmol) in NMP (3 mL) was added Zn(CN)₂ (120 mg, 1.02 mmol) and Pd(PPh₃)₄(118 mg, 0.102 mmol) under N₂ atmosphere and the mixture was stirred under N₂ atmosphere at 130° C. for 5 hours. The mixture was washed with water and extracted with EtOAc twice. The combined organic layers were washed with brine, dried over anhydrous Na₂SO₄, filtered and concentrated to dryness under reduced pressure. The residue was purified by column chromatography on silica gel (PE:EtOAc=3:1) to give tert-butyl (1²S,1⁴S,Z)-2⁴-cyano-3,10-dioxa-1(2,4)-piperidina-2(1,2)-benzenacyclodecaphan-7-ene-1′-carboxylate (100 mg, yield 49.3%). LC/MS (ESI) m/z: 399 (M+H)⁺.

Step 2: (1²S,1⁴S,Z)-3,10-dioxa-1(2,4)-piperidina-2(1,2)-benzenacyclodecaphan-7-ene-2⁴-carbonitrile hydrochloride. A solution of tert-butyl (1²S,1⁴S,Z)-2⁴-cyano-3,10-dioxa-1(2,4)-piperidina-2(1,2)-benzenacyclodecaphan-7-ene-1¹-carboxylate (50 mg, 0.13 mmol) in HCl/1,4-dioxane (1 mL) was stirred at 25° C. for 1 hour. The mixture was concentrated to dryness to give (1²S,1⁴S,Z)-3,10-dioxa-1(2,4)-piperidina-2(1,2)-benzenacyclodecaphan-7-ene-2⁴-carbonitrile hydrochloride (50 mg, crude) as white solid, which was used directly without purification. LC/MS (ESI) m/z: 299 (M+H)⁺.

Step 3: tert-butyl 4-(((1²S,1⁴S,Z)-2⁴-cyano-3,10-dioxa-1(2,4)-piperidina-2(1,2)-benzenacyclodecaphan-7-en-1′-yl)methyl)-5-methoxy-7-methyl-1H-indole-1-carboxylate. At 0° C., a solution of tert-butyl 4-(hydroxymethyl)-5-methoxy-7-methyl-1H-indole-1-carboxylate (59 mg, 0.201 mmol) in dry DCM (1 mL) was added PPh₃Br₂ (92 mg, 0.217 mmol) portionwise and the mixture was stirred under N₂ atmosphere for 2 hours. Then a mixture of (1²,1⁴S,Z)-3,10-dioxa-1(2,4)-piperidina-2(1,2)-benzenacyclodecaphan-7-ene-2⁴-carbonitrile hydrochloride (50 mg, 0.167 mmol) and DIPEA (65 mg, 0.501 mmol) in dry DCM (1 mL) was added and the resulting mixture was stirred at 0° C. for 1.5 hours. The mixture was concentrated to dryness and the residue was purified by prep-HPLC to give tert-butyl 4-(((1²,1⁴S,Z)-2⁴-cyano-3,10-dioxa-1(2,4)-piperidina-2(1,2)-benzenacyclodecaphan-7-en-1′-yl)methyl)-5-methoxy-7-methyl-1H-indole-1-carboxylate (60 mg, 62.5% yield) as colorless oil. LC/MS (ESI) m/z: 572 (M+H)⁺.

Step 4: tert-butyl 4-(((1²S,1⁴S,Z)-2⁴-(1H-tetrazol-5-yl)-3,10-dioxa-1(2,4)-piperidina-2(1,2)-benzenacyclodecaphan-7-en-1′-yl)methyl)-5-methoxy-7-methyl-1H-indole-1-carboxylate. To a solution of 4-(((1²S,1⁴S,Z)-2⁴-cyano-3,10-dioxa-1(2,4)-piperidina-2(1,2)-benzenacyclodecaphan-7-en-1′-yl)methyl)-5-methoxy-7-methyl-1H-indole-1-carboxylate (60 mg, 0.105 mmol) in toluene (2 mL) was added Me₃SiN₃ (36 mg, 0.315 mmol) and Bu₂SnO (78 mg, 0.315 mmol) under N₂ atmosphere and the mixture was stirred under N₂ atmosphere at 130° C. for 4 hours. The mixture was concentrated to dryness and the residue was purified by prep-HPLC to give tert-butyl 4-(((1²,1⁴S,Z)-2⁴-(1H-tetrazol-5-yl)-3,10-dioxa-1(2,4)-piperidina-2(1,2)-benzenacyclodecaphan-7-en-1′-yl)methyl)-5-methoxy-7-methyl-1H-indole-1-carboxylate (30 mg, 46.2% yield) as brown oil. LC/MS (ESI) m/z: 615 (M+H)⁺.

Step 5: tert-butyl 4-(((1²S,1⁴S)-2⁴-(1H-tetrazol-5-yl)-3,10-dioxa-1(2,4)-piperidina-2(1,2)-benzenacyclodecaphane-1¹-yl)methyl)-5-methoxy-7-methyl-1H-indole-1-carboxylate. To a solution of tert-butyl 4-(((1²S,1⁴S,Z)-2⁴-(1H-tetrazol-5-yl)-3,10-dioxa-1(2,4)-piperidina-2(1,2)-benzenacyclodecaphan-7-en-1′-yl)methyl)-5-methoxy-7-methyl-1H-indole-1-carboxylate (30 mg, 0.05 mmol) in EtOAc (1 mL) was added PtO₂ (4 mg, 0.01 mmol) and the mixture was stirred under a H₂ balloon at room temperature for 0.5 hours. The mixture was filtrated and the filtrate was concentrated under reduced pressure to dryness. The residue was purified by prep-HPLC to give tert-butyl 4-(((1²S,1⁴S)-2⁴-(1H-tetrazol-5-yl)-3,10-dioxa-1(2,4)-piperidina-2(1,2)-benzenacyclodecaphane-1¹-yl)methyl)-5-methoxy-7-methyl-1H-indole-1-carboxylate (25 mg, yield 83.3%) as white solid. LC/MS (ESI) m/z: 617 (M+H)⁺.

Step 6: (1²S,1⁴S)-1¹-((5-methoxy-7-methyl-1H-indol-4-yl)methyl)-2⁴-(1H-tetrazol-5-yl)-3,10-dioxa-1(2,4)-piperidina-2(1,2)-benzenacyclodecaphane. A mixture of tert-butyl 4-(((1²S,1⁴S)-2⁴-(1H-tetrazol-5-yl)-3,10-dioxa-1(2,4)-piperidina-2(1,2)-benzenacyclodecaphane-1¹-yl)methyl)-5-methoxy-7-methyl-1H-indole-1-carboxylate (25 mg, 0.041 mmol), TMSOTf (18 mg, 0.082 mmol) and 2,6-diisopropylpyridine (25 mg, 0.041 mmol) was stirred at room temperature for 18 hours. The mixture was concentrated to dryness and the residue was purified by prep-HPLC to give (1²S,1⁴S)-1′-((5-methoxy-7-methyl-1H-indol-4-yl)methyl)-2⁴-(1H-tetrazol-5-yl)-3,10-dioxa-1(2,4)-piperidina-2(1,2)-benzenacyclodecaphane (1.0 mg, 4.8% yield) as white solid. ¹HNMR (400 MHz, CD₃OD) δ 8.31 (s, 1H), 7.90 (d, J=8.0 Hz, 1H), 7.83 (s, 1H), 7.72 (d, J=8.0 Hz, 1H), 7.33 (d, J=3.1 Hz, 1H), 6.82 (s, 1H), 6.46 (d, J=3.1 Hz, 1H), 5.44 (d, J=10.1 Hz, 1H), 4.55 (s, 3H), 4.18 (d, J=12.8 Hz, 1H), 4.00 (t, J=8.9 Hz, 1H), 3.89 (s, 3H), 3.76 (s, 1H), 3.60 (d, J=20.7 Hz, 3H), 3.48 (s, 1H), 2.52 (s, 3H), 2.37 (d, J=12.5 Hz, 1H), 2.04 (dd, J=9.9, 5.6 Hz, 3H), 2.00-1.91 (m, 3H), 1.64-1.58 (m, 3H), 1.48 (dd, J=12.7, 4.3 Hz, 1H). LC/MS (ESI) m/z: 517 (M+H)⁺.

### Example 75: (1²S,1⁴S,Z)-1′-((5-methoxy-7-methyl-1H-indol-4-yl)methyl)-3,8-dioxa-1(2,4)-piperidina-2(1,7)-naphthalenacyclooctaphan-5-ene-2⁴-carboxylic acid

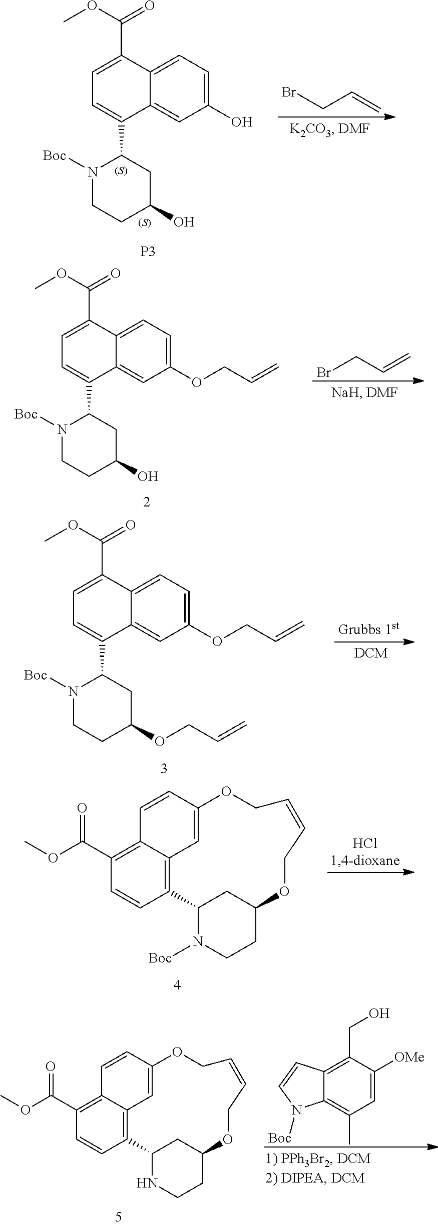

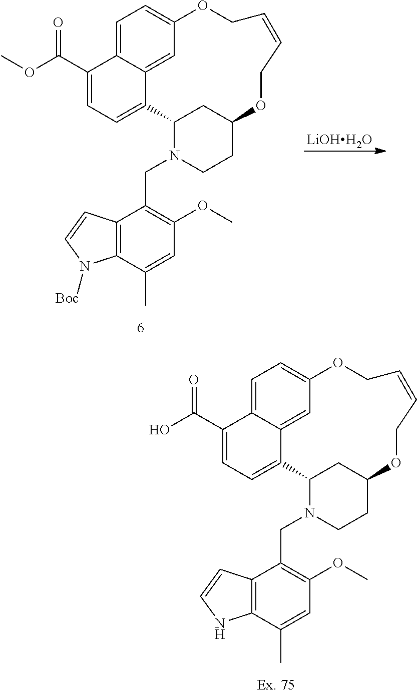 [SMILES: COC(=O)c1ccc2c3cc(ccc13)OC/C=C\CO[C@H]1CCN(Cc3c(OC)cc(C)c4c3ccn4C(=O)OC(C)(C)C)[C@H]2C1 ; "LiOH·H2O" ; COc1cc(C)c2[nH]ccc2c1CN1CC[C@H]2C[C@H]1c1ccc(C(=O)O)c3ccc(cc13)OC/C=C\CO2]

Step 1: tert-butyl (2S,4S)-2-(7-(allyloxy)-4-(methoxycarbonyl)naphthalen-1-yl)-4-hydroxypiperidine-1-carboxylate (2). To a mixture of tert-butyl (2S,4S)-4-hydroxy-2-(7-hydroxy-4-(methoxycarbonyl)naphthalen-1-yl)piperidine-1-carboxylate (201 mg, 0.5 mmol) in DMF (2 mL) was added K₂CO₃ (207 mg, 1.5 mmol) and 3-bromoprop-1-ene (302 mg, 2.5 mmol) and the mixture was stirred at 25° C. overnight. The mixture was diluted with EtOAc, washed with water and brine, dried over anhydrous Na₂SO₄, filtered and concentrated under reduced pressure to dryness. The residue was purified by flash chromatography (PE:EtOAc=3:1) to give tert-butyl (2S,4S)-2-(7-(allyloxy)-4-(methoxycarbonyl)naphthalen-1-yl)-4-hydroxypiperidine-1-carboxylate (198 mg, yield 89.7%) as white solid. LC/MS (ESI) m/z: 442 (M+H)⁺.

Step 2: tert-butyl (2S,4S)-4-(allyloxy)-2-(7-(allyloxy)-4-(methoxycarbonyl) naphthalen-1-yl)piperidine-1-carboxylate (3): At 0° C., to a solution of tert-butyl (2S,4S)-2-(7-(allyloxy)-4-(methoxycarbonyl)naphthalen-1-yl)-4-hydroxypiperidine-1-carboxylate (178 mg, 0.40 mmol) in DMF (2 mL) was added NaH (32 mg, 0.80 mmol, 60% dispersion in mineral oil) in portions and the mixture was stirred under N₂ atmosphere at 0° C. for 0.5 hours. Then 3-bromoprop-1-ene (97 mg, 0.8 mmol) was added and the resulting mixture was stirred at 25° C. for 1 hour. The mixture was quenched with saturated aq.NH₄Cl solution and extracted with EtOAc twice. The combined organic layers were washed with water and brine, dried over anhydrous Na₂SO₄, filtered and concentrated to dryness. The residue was purified by column chromatography on silica gel (PE:EtOAc=3:1) to give tert-butyl (2S,4S)-4-(allyloxy)-2-(7-(allyloxy)-4-(methoxycarbonyl)naphthalen-1-yl)piperidine-1-carboxylate (110 mg, yield 57.1%) as white solid. LC/MS (ESI) m/z: 482 (M+H)⁺.

Step 3: 1¹-(tert-butyl) 2⁴-methyl (1²S,1⁴S,Z)-3,8-dioxa-1(2,4)-piperidina-2(1,7)-naphthalenacyclooctaphan-5-ene-1¹,2⁴-dicarboxylate (4): To a solution of tert-butyl (2S,4S)-4-(allyloxy)-2-(7-(allyloxy)-4-(methoxycarbonyl)naphthalen-1-yl)piperidine-1-carboxylate (110 mg, 0.23 mmol) in dry DCM (110 mL) was added Grubbs 1^{st} catalyst (22 mg) and the mixture was stirred under N₂ atmosphere at 25° C. for 18 hours. The mixture was concentrated under reduced pressure to dryness and the residue was purified by flash chromatography (PE:EtOAc=10:1) to give 1¹-(tert-butyl) 2⁴-methyl (1²S,1⁴S,Z)-3,8-dioxa-1(2,4)-piperidina-2(1,7)-naphthalenacyclooctaphan-5-ene-1¹,2⁴-dicarboxylate (70 mg, yield 67.1%) as brown solid. LC/MS (ESI) m/z: 454 (M+H)⁺.

Step 4: methyl (1²S,1⁴S,Z)-3,8-dioxa-1(2,4)-piperidina-2(1,7)-naphthalenacyclooctaphan-5-ene-2⁴-carboxylate hydrochloride (5). A mixture of 1¹-(tert-butyl) 2⁴-methyl (1²,1⁴S,Z)-3,8-dioxa-1(2,4)-piperidina-2(1,7)-naphthalenacyclooctaphan-5-ene-1¹,2⁴-dicarboxylate (70 mg, 0.15 mmol) in HCl/1,4-dioxane (1 mL, 4.0 mmol) was stirred at 25° C. for 1 hour. The mixture was concentrated to dryness to give methyl (1²S,1⁴S,Z)-3,8-dioxa-1(2,4)-piperidina-2(1,7)-naphthalenacyclooctaphan-5-ene-2⁴-carboxylate hydrochloride (58 mg, yield 100%) as white solid, which was used directly without purification. LC/MS (ESI) m/z: 354 (M+H)⁺.

Step 5: Methyl (1²S,1⁴S,E)-1¹-((1-(tert-butoxycarbonyl)-5-methoxy-7-methyl-1H-indol-4-yl)methyl)-3,10-dioxa-1(2,4)-piperidina-2(1,7)-naphthalenacyclodecaphan-7-ene-2⁴-carboxylate (6). At 0° C., to a solution of tert-butyl 4-(hydroxymethyl)-5-methoxy-7-methyl-1H-indole-1-carboxylate (53 mg, 0.18 mmol) in dry DCM (1 mL) was added PPh₃Br₂ (84 mg, 0.2 mmol) in portions and the mixture stirred under N₂ atmosphere at 0° C. for 2 hours. Then a mixture of methyl (1², 1⁴S,Z)-3,8-dioxa-1(2,4)-piperidina-2(1,7)-naphthalenacyclooctaphan-5-ene-2⁴-carboxylate hydrochloride (60 mg, 0.15 mmol) and DIPEA (78 mg, 0.6 mmol) in dry DCM (1 mL) was added and the mixture was stirred at 0° C. for 1.5 hours. The mixture was concentrated to dryness and was purified by flash chromatography on silica gel (DCM:MeOH=97:3) to give methyl (1²S,1⁴S,Z)-1′-((1-(tert-butoxycarbonyl)-5-methoxy-7-methyl-1H-indol-4-yl)methyl)-3,8-dioxa-1(2,4)-piperidina-2(1,7)-naphthalenacyclooctaphan-5-ene-2⁴-carboxylate (42 mg, yield 44.7%) as white solid. LC/MS (ESI) m/z: 627 (M+H)⁺.

Step 6: (1²S,1⁴S,Z)-1¹-((5-methoxy-7-methyl-1H-indol-4-yl)methyl)-3,8-dioxa-1(2,4)-piperidina-2(1,7)-naphthalenacyclooctaphan-5-ene-2⁴-carboxylic acid (Example 75). To a mixture of methyl (1²S,1⁴S,Z)-1¹-((1-(tert-butoxycarbonyl)-5-methoxy-7-methyl-1H-indol-4-yl)methyl)-3,8-dioxa-1(2,4)-piperidina-2(1,7)-naphthalenacyclooctaphan-5-ene-2⁴-carboxylate (42 mg, 0.07 mmol) and LiOH·H₂O (9 mg, 0.21 mmol) in MeOH (2 mL)/water (0.5 mL) and the mixture was stirred at 60° C. overnight. The reaction mixture was acidified with 1N aq.HCl solution to pH˜4 and extracted with EtOAc twice. The combined organic layers were washed with brine, dried over anhydrous Na₂SO₄, filtered and concentrated to dryness. The residue was purified by prep-HPLC to give (1²S,1⁴S,Z)-1¹-((5-methoxy-7-methyl-1H-indol-4-yl)methyl)-3,8-dioxa-1(2,4)-piperidina-2(1,7)-naphthalenacyclooctaphan-5-ene-2⁴-carboxylic acid (23 mg, yield 67.0%) as white solid. ¹HNMR (400 MHz, CD₃OD) b 8.63 (d, J=9.3 Hz, 1H), 8.32 (s, 1H), 7.95 (d, J=7.6 Hz, 1H), 7.88 (d, J=7.6 Hz, 1H), 7.79 (d, J=2.2 Hz, 1H), 7.39 (d, J=3.1 Hz, 1H), 7.22 (dd, J=9.3, 2.3 Hz, 1H), 6.89 (s, 1H), 6.61 (d, J=3.1 Hz, 1H), 6.54-6.40 (m, 1H), 5.91-5.79 (m, 1H), 5.47 (d, J=10.2 Hz, 1H), 5.09 (d, J=13.0 Hz, 2H), 4.97 (dd, J=13.8, 9.6 Hz, 1H), 4.53 (dd, J=31.7, 12.6 Hz, 2H), 4.00 (s, 3H), 3.98-3.91 (m, 1H), 3.84 (s, 1H), 3.70 (t, J=11.7 Hz, 1H), 3.48 (d, J=11.7 Hz, 1H), 2.57 (s, 3H), 2.50 (d, J=13.3 Hz, 1H), 2.12-1.94 (m, 2H), 1.66 (t, J=14.0 Hz, 1H). LC/MS (ESI) m/z: 513 (M+H)⁺.

### Example 76: (1²S,1⁴S)-1¹-((5-methoxy-7-methyl-1H-indol-4-yl)methyl)-3,9-dioxa-1(2,4)-piperidina-2(1,7)-naphthalenacyclononaphane-2⁴-carboxylic acid

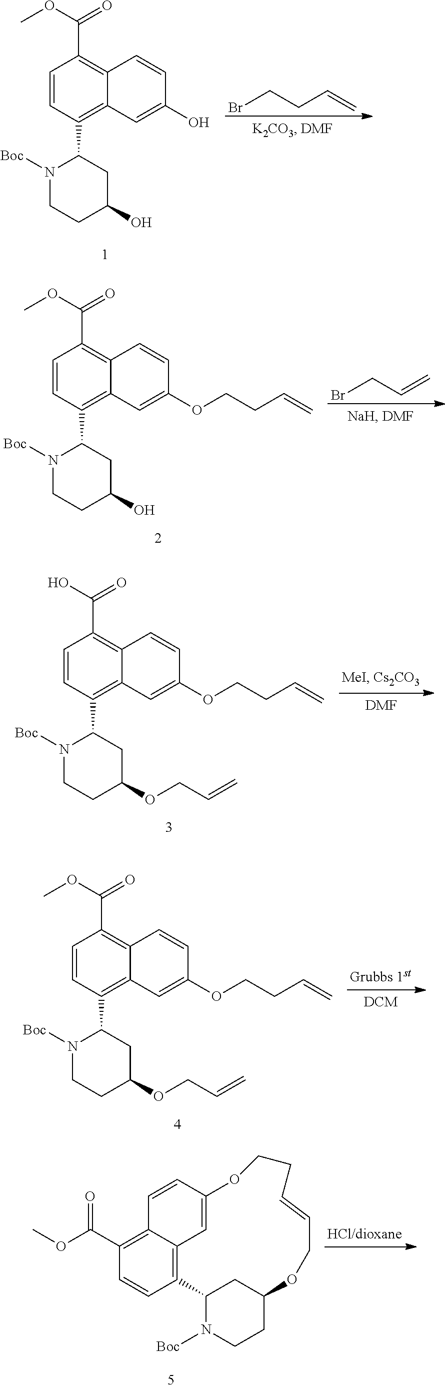

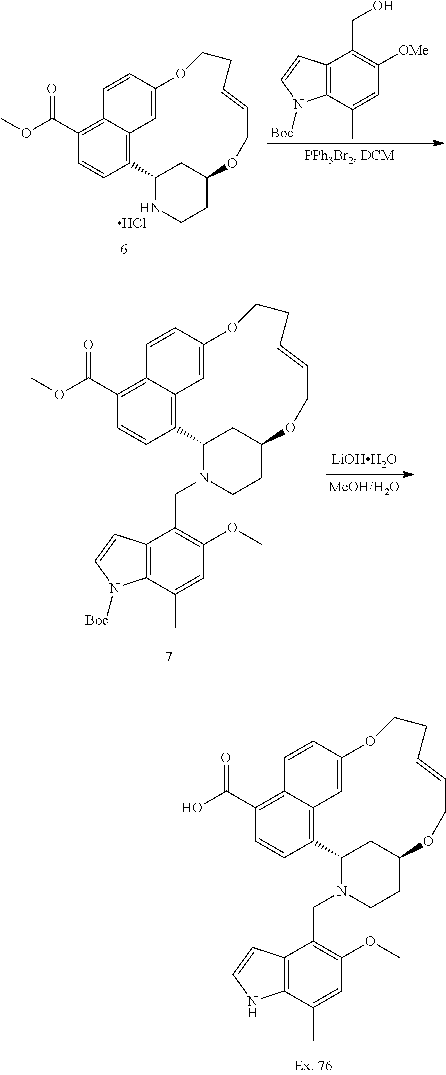

Step 1: tert-butyl (2S,4S)-2-(7-(but-3-en-1-yloxy)-4-(methoxycarbonyl)naphthalen-1-yl)-4-hydroxypiperidine-1-carboxylate. To a mixture of tert-butyl (2S,4S)-4-hydroxy-2-(7-hydroxy-4-(methoxycarbonyl)naphthalen-1-yl)piperidine-1-carboxylate (200 mg, 0.50 mmol) and 4-bromobut-1-ene (2.5 g, 16 mmol) in DMF (4 mL) was added K₂CO₃ (276 mg, 2.0 mmol) and the mixture was stirred at room temperature for 16 hours. The mixture was diluted with water and extracted with EtOAc twice. The combined organic layers were washed with brine, dried over anhydrous Na₂SO₄, filtered and concentrated under reduced pressure to dryness. The residue was purified by column chromatography on silica gel (PE:EtOAc=2:1) to give tert-butyl (2S,4S)-2-(7-(but-3-en-1-yloxy)-4-(methoxycarbonyl)naphthalen-1-yl)-4-hydroxypiperidine-1-carboxylate (140 mg, yield 61.5%) as yellow oil. LCMS (ESI) m/z: 456 (M+H)⁺.

Step 2: 4-((2S,4S)-4-(allyloxy)-1-(tert-butoxycarbonyl)piperidin-2-yl)-6-(but-3-en-1-yloxy)-1-naphthoic acid. To a mixture of tert-butyl (2S,4S)-2-(7-(but-3-en-1-yloxy)-4-(methoxycarbonyl)naphthalen-1-yl)-4-hydroxypiperidine-1-carboxylate (140 mg, 0.31 mmol) and 3-bromoprop-1-ene (208 mg, 1.9 mmol) in DMF (2 mL) was added NaH (46 mg, 1.9 mmol, 60% dispersion in mineral oil) at 0° C. and the mixture was stirred at room temperature for 16 hours. After quenched with ice-water, the mixture was acidified with 1N aq.HCl to pH˜4 and extracted with EtOAc twice. The combined organic layers were washed with brine, dried over anhydrous Na₂SO₄, filtered and concentrated under reduced pressure to dryness to give 4-((2S,4S)-4-(allyloxy)-1-(tert-butoxycarbonyl)piperidin-2-yl)-6-(but-3-en-1-yloxy)-1-naphthoic acid (120 mg, yield 80.6%) as yellow oil, which was directly used in the next reaction without purification. LCMS (ESI) m/z: 482 (M+H)⁺.

Step 3: tert-butyl (2S,4S)-4-(allyloxy)-2-(7-(but-3-en-1-yloxy)-4-(methoxycarbonyl)naphthalen-1-yl)piperidine-1-carboxylate. To a mixture of 4-((2S,4S)-4-(allyloxy)-1-(tert-butoxycarbonyl)piperidin-2-yl)-6-(but-3-en-1-yloxy)-1-naphthoic acid (120 mg, 0.25 mmol) and MeI (212 mg, 1.5 mmol) in DMF (2 mL) was added Cs₂CO₃ (326 mg, 1.0 mmol) and the mixture was stirred at room temperature for 16 hours. The mixture was diluted with water and extracted with EtOAc twice. The combined organic layers were washed with brine, dried over anhydrous Na₂SO₄, filtered and concentrated under reduced pressure to dryness. The residue was purified by column chromatography on silica gel (PE:EtOAc=7:1) to give tert-butyl (2S,4S)-2-(7-(but-3-en-1-yloxy)-4-(methoxycarbonyl)naphthalen-1-yl)-4-hydroxypiperidine-1-carboxylate (80 mg, yield 64.6%) as yellow solid. LCMS (ESI) m/z: 496 (M+H)⁺.

Step 4: 1¹-(tert-butyl) 2⁴-methyl (1²S,1⁴S,E)-3,9-dioxa-1(2,4)-piperidina-2(1,7)-naphthalenacyclononaphan-6-ene-1¹,2⁴-dicarboxylate. To a solution of tert-butyl (2S,4S)-4-(allyloxy)-2-(7-(but-3-en-1-yloxy)-4-(methoxycarbonyl)naphthalen-1-yl)piperidine-1-carboxylate (80 mg, 0.16 mmol) in anhydrous DCM (50 mL) was added Grubbs 1^{st} catalyst (130 mg, 0.16 mmol). The mixture was degassed under N₂ atmosphere for three times and stirred at room temperature for 16 hours. The mixture was concentrated under reduced pressure to dryness and the residue was purified by column chromatography on silica gel (PE:EtOAc=2:1) to give 1′-(tert-butyl) 2⁴-methyl (1²S,1⁴S,E)-3,9-dioxa-1(2,4)-piperidina-2(1,7)-naphthalenacyclononaphan-6-ene-1¹,2⁴-dicarboxylate (25 mg, yield 33.4%) as yellow oil. LCMS (ESI) m/z: 468 (M+H)⁺.

Step 5: methyl (1²S,1⁴S,E)-3,9-dioxa-1(2,4)-piperidina-2(1,7)-naphthalenacyclononaphan-6-ene-2⁴-carboxylate hydrochloride. A solution of 1¹-(tert-butyl) 24-methyl (1²S,1⁴S,E)-3,9-dioxa-1(2,4)-piperidina-2(1,7)-naphthalenacyclononaphan-6-ene-1¹,2⁴-dicarboxylate (25 mg, 54 umol) in HCl/1,4-dioxane (1 mL) was stirred at 25° C. for 16 hours. The mixture was concentrated to dryness to give methyl (1²S,1⁴S,E)-3,9-dioxa-1(2,4)-piperidina-2(1,7)-naphthalenacyclononaphan-6-ene-2⁴-carboxylate hydrochloride (17 mg, yield 86.5%) as white solid, which was used directly without purification. LC/MS (ESI) m/z: 368 (M+H)⁺.

Step 6: methyl (1²S,1⁴S,E)-1¹-((1-(tert-butoxycarbonyl)-5-methoxy-7-methyl-1H-indol-4-yl)methyl)-3,9-dioxa-1(2,4)-piperidina-2(1,7)-naphthalenacyclononaphan-6-ene-2⁴-carboxylate. At 0° C., to a solution of methyl (1²S,1⁴S,E)-3,9-dioxa-1(2,4)-piperidina-2(1,7)-naphthalenacyclononaphan-6-ene-2⁴-carboxylate (17 mg, 46 umol) in anhydrous DCM (1 mL) was added PPh₃Br₂ (30 mg, 71 umol) in portions and the mixture was stirred under N₂ atmosphere for 2 hours. Then a mixture of tert-butyl 4-(hydroxymethyl)-5-methoxy-7-methyl-1H-indole-1-carboxylate (17 mg, 56 umol) and DIPEA (21 mg, 163 umol) in anhydrous DCM (1 mL) was added and the resulting mixture was stirred at 0° C. for 1.5 hours. The mixture was concentrated to dryness and the residue was purified by prep-HPLC to give methyl (12S,14S,E)-11-((1-(tert-butoxycarbonyl)-5-methoxy-7-methyl-1H-indol-4-yl)methyl)-3,9-dioxa-1(2,4)-piperidina-2(1,7)-naphthalenacyclononaphan-6-ene-24-carboxylate (10 mg, yield 34.8%) as white solid. LC/MS (ESI) m/z: 641 (M+H)⁺.

Step 7: (1²S,1⁴S,E)-1¹-((5-methoxy-7-methyl-1H-indol-4-yl)methyl)-3,9-dioxa-1(2,4)-piperidina-2(1,7)-naphthalenacyclononaphan-6-ene-2⁴-carboxylic acid. To a solution of methyl (1²,1⁴S,E)-11-((1-(tert-butoxycarbonyl)-5-methoxy-7-methyl-1H-indol-4-yl)methyl)-3,9-dioxa-1(2,4)-piperidina-2(1,7)-naphthalenacyclononaphan-6-ene-2⁴-carboxylate (10 mg, 16 umol) in MeOH (2 mL) and water (1 mL) was added LiOH·H₂O (10 mg, 250 umol) at 0° C. and the mixture was stirred at 60° C. overnight. The mixture was concentrated to dryness and the residue was purified by prep-HPLC to give (1²S,1⁴S,E)-11-((5-methoxy-7-methyl-1H-indol-4-yl)methyl)-3,9-dioxa-1 (2,4)-piperidina-2(1,7)-naphthalenacyclononaphan-6-ene-2⁴-carboxylic acid (2.8 mg, yield 33.2%) as white solid. ¹H NMR (400 MHz, CD₃OD) δ 8.54 (dd, J=21.2, 9.3 Hz, 1H), 8.45 (s, 1H), 7.85 (t, J=7.5 Hz, 1H), 7.80-7.76 (m, 1H), 7.39-7.26 (m, 2H), 7.22 (d, J=9.3 Hz, 1H), 6.82 (d, J=8.1 Hz, 1H), 6.51 (t, J=3.1 Hz, 1H), 6.04-5.63 (m, 3H), 4.74 (d, J=12.7 Hz, 1H), 4.61-4.53 (m, 2H), 4.30 (dd, J=27.3, 11.6 Hz, 2H), 3.92 (d, J=16.7 Hz, 3H), 3.84 (d, J'2 3.5 Hz, 1H), 3.77-3.68 (m, 2H), 3.46 (d, J=13.4 Hz, 1H), 3.23-3.13 (m, 1H), 2.54 (d, J=11.5 Hz, 4H), 2.34-2.12 (m, 2H), 2.04-1.96 (m, 2H). LC/MS (ESI) m/z: 527 (M+H)⁺.

### Example 77: (1²S,1⁴S)-1¹-((5-methoxy-7-methyl-1H-indol-4-yl)methyl)-3-oxa-1(2,4)-piperidina-2(1,2)-benzenacyclodecaphane-2⁴-carboxylic acid

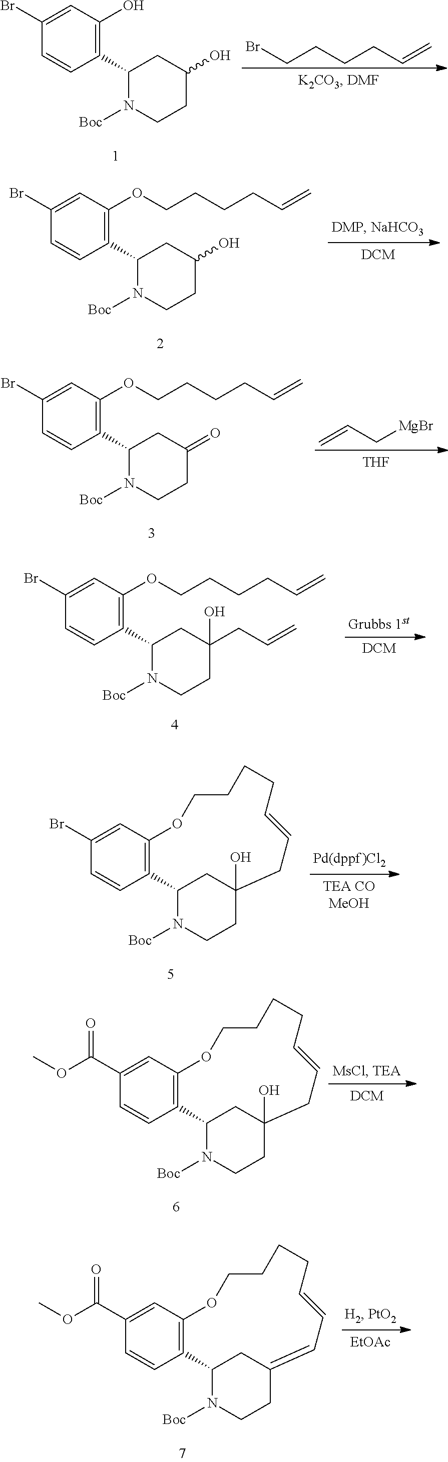

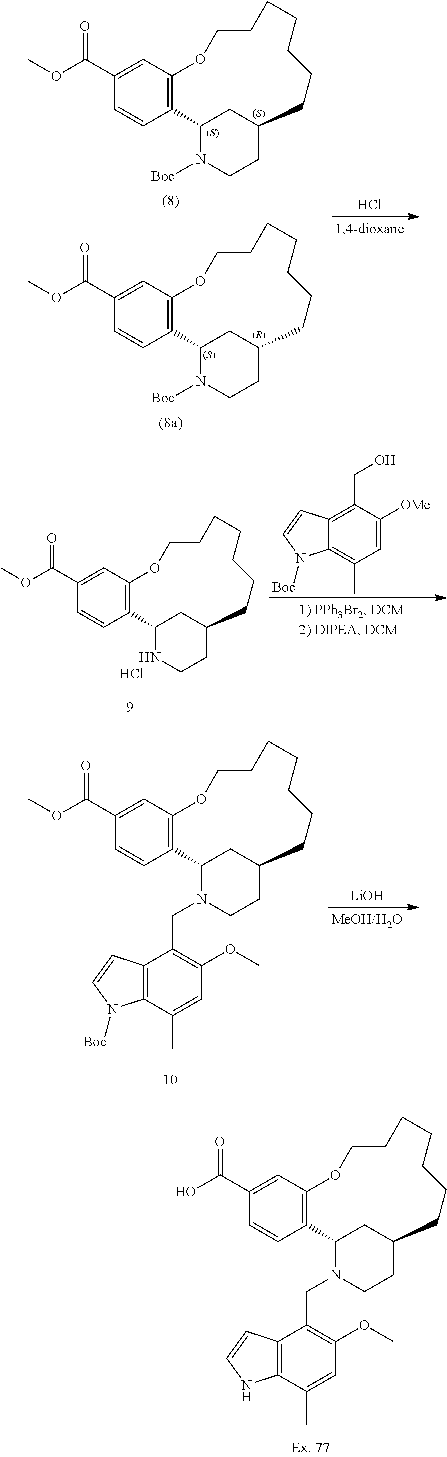

Step 1: tert-butyl (2S)-2-(4-bromo-2-(hex-5-en-1-yloxy)phenyl)-4-hydroxypiperidine-1-carboxylate(1). To a solution of tert-butyl (2S)-2-(4-bromo-2-hydroxyphenyl)-4-hydroxypiperidine-1-carboxylate (2.0 g, 5.39 mmol) in DMF (20 mL) was added K₂CO₃ (2.23 g, 16.17 mmol) and 6-bromohex-1-ene (1.44 mL, 10.78 mmol) and the mixture was stirred at 35° C. for 16 hours. The mixture was diluted with EtOAc, washed with water and brine, dried over anhydrous Na₂SO₄, filtered and concentrated under reduced pressure to dryness. The residue was purified by flash chromatography (PE:EtOAc=10:1) to give tert-butyl (2S)-2-(4-bromo-2-(hex-5-en-1-yloxy)phenyl)-4-hydroxypiperidine-1-carboxylate (1.2 g, yield 49.2%) as yellow oil. LC/MS (ESI) m/z: 454/456 (M+H)⁺.

Step 2: tert-butyl (S)-2-(4-bromo-2-(hex-5-en-1-yloxy)phenyl)-4-oxopiperidine-1-carboxylate (3). To a solution of tert-butyl (2S)-2-(4-bromo-2-(hex-5-en-1-yloxy)phenyl)-4-hydroxypiperidine-1-carboxylate (1.2 g, 2.65 mmol) in DCM (12 mL) was added NaHCO₃ (445 mg, 5.3 mmol) and DMP (2.25 g, 5.3 mmol) at 0° C., and the mixture was stirred at room temperature for 10 hours. The mixture was quenched with ice-water and extracted with DCM twice. The combined organic layer were washed with brine, dried over anhydrous Na₂SO₄, filtered and concentrated to dryness under reduced pressure. The residue was purified by flash column chromatography on silica gel (PE:EtOAc=15:1) to give tert-butyl (S)-2-(4-bromo-2-(hex-5-en-1-yloxy)phenyl)-4-oxopiperidine-1-carboxylate (1.02 g, yield 85.4%) as light oil. LC/MS: m/z 452 (M+H)⁺.

Step 3: tert-butyl (2S)-4-allyl-2-(4-bromo-2-(hex-5-en-1-yloxy)phenyl)-4-hydroxypiperidine-1-carboxylate(4). To a solution of tert-butyl (S)-2-(4-bromo-2-(hex-5-en-1-yloxy)phenyl)-4-oxopiperidine-1-carboxylate (1.0 g, 2.22 mmol) in DMF (10 mL) was added allyl magnesium bromide (4.43 mL, 4.43 mmol) and the mixture was stirred at room temperature for 16 hours. The mixture was diluted with DCM, washed with water and brine, dried over anhydrous Na₂SO₄, filtered and concentrated under reduced pressure to dryness. The residue was purified by flash chromatography (PE:EtOAc=60:1) to give tert-butyl (2S)-4-allyl-2-(4-bromo-2-(hex-5-en-1-yloxy)phenyl)-4-hydroxypiperidine-1-carboxylate (370 mg, yield 26.4%) as light oil. LC/MS: m/z 494 (M+H)⁺.

Step 4: tert-butyl (1²S,E)-2⁴-bromo-1⁴-hydroxy-3-oxa-1(2,4)-piperidina-2(1,2)-benzenacyclodecaphan-8-ene-1′-carboxylate(5). To a solution of tert-butyl (2S)-4-allyl-2-(4-bromo-2-(hex-5-en-1-yloxy)phenyl)-4-hydroxypiperidine-1-carboxylate (370 mg, 0.75 mmol) in dry DCM (300 mL) was added Grubbs 1^{st} (185 mg, 0.22 mmol), the mixture was degassed under N₂ atmosphere for three times and stirred at 25° C. for 2 days. The mixture was concentrated under reduced pressure to dryness and the residue was purified by flash chromatography (PE:EtOAc=50:1) to give tert-butyl (1²S,E)-2⁴-bromo-1⁴-hydroxy-3-oxa-1(2,4)-piperidina-2(1,2)-benzenacyclodecaphan-8-ene-1′-carboxylate (230 mg, yield 65.9%) as brown oil. LC/MS (ESI) m/z: 466 (M+H)⁺.

Step 5: 1¹-(tert-butyl) 2⁴-methyl (1²S,E)-14-hydroxy-3-oxa-1(2,4)-piperidina-2(1,2)-benzenacyclodecaphan-8-ene-1¹,2⁴-dicarboxylate(6). To a solution of tert-butyl (1²S,E)-2⁴-bromo-1⁴-hydroxy-3-oxa-1(2,4)-piperidina-2(1,2)-benzenacyclodecaphan-8-ene-1′-carboxylate (190 mg, 0.41 mmol) in MeOH (20 mL) was added triethylamine (0.17 mL, 1.22 mmol) and Pd(dppf)Cl₂ (60 mg, 0.082 mmol) at 0° C., and the mixture was stirred at 75° C. for 16 hours under CO atmosphere. The mixture was diluted with water and extracted with EtOAc twice. The combined organic layers were washed with brine, dried over anhydrous Na₂SO₄, filtered and concentrated to dryness under reduced pressure. The residue was purified by prep-TLC (PE:EtOAc=5:1) to give 1¹-(tert-butyl) 2⁴-methyl (1²S,E)-1⁴-hydroxy-3-oxa-1(2,4)-piperidina-2(1,2)-benzenacyclodecaphan-8-ene-1¹,2⁴-dicarboxylate (98 mg, yield 53.9%) as yellow oil. LC/MS: m/z 446(M+H)⁺.

Step 6: 1¹-(tert-butyl) 2⁴-methyl (S,1⁴Z,8E)-3-oxa-1(2,4)-piperidina-2(1,2)-benzenacyclodecaphan-8-ene-1¹,2⁴-dicarboxylate(7). To a solution of 1¹-(tert-butyl) 2⁴-methyl (1²S,E)-1⁴-hydroxy-3-oxa-1(2,4)-piperidina-2(1,2)-benzenacyclodecaphan-8-ene-1¹,2⁴-dicarboxylate (98 mg, 0.22 mmol) in DCM (4 mL) was added triethylamine (0.22 mL, 1.54 mmol) at 0° C. followed by methanesulfonyl chloride (76 mg, 0.66 mmol). The reaction mixture was stirred at 0° C. for 2 hours under N₂ atmosphere. The mixture was quenched with ice-water and extracted with EtOAc twice. The combined organic layers were washed with brine, dried over anhydrous Na₂SO₄, filtered and concentrated to dryness under reduced pressure. The residue was purified by chromatography on silica gel (PE:EtOAc=20:1) to give 1¹-(tert-butyl) 2⁴-methyl (S,1⁴Z,8E)-3-oxa-1(2,4)-piperidina-2(1,2)-benzenacyclodecaphan-8-ene-1¹,2⁴-dicarboxylate (84 mg, yield 89.3%) as light oil. LC/MS (ESI) m/z: 428 (M+H)⁺.

Step 7: 1¹-(tert-butyl) 2⁴-methyl (1²S,1⁴S*)-3-oxa-1(2,4)-piperidina-2(1,2)-benzenacyclodecaphane-1¹,2⁴-dicarboxylate (8) and 1¹-(tert-butyl) 2⁴-methyl (1²S,1⁴R*)-3-oxa-1(2,4)-piperidina-2(1,2)-benzenacyclodecaphane-1¹,2⁴-dicarboxylate (8a). To a solution of 1¹-(tert-butyl) 2⁴-methyl (S,1⁴Z,8E)-3-oxa-1(2,4)-piperidina-2(1,2)-benzenacyclodecaphan-8-ene-1¹,2⁴-dicarboxylate (84 mg, 0.20 mmol) in EtOAc (3 mL) was added PtO₂ (22 mg, 0.098 mmol) and AcOH (one drop) at 0° C., and the mixture was degassed under N₂ atmosphere for three times and stirred under a H₂ balloon at 35° C. for 1.5 hours. The mixture was filtered and the filtrate was concentrated under reduced pressure to dryness. The residue was purified by chromatography silica gel (PE:EtOAc=10:1) and SFC to give 1¹-(tert-butyl) 2⁴-methyl (1²S,1⁴S*)-3-oxa-1(2,4)-piperidina-2(1,2)-benzenacyclodecaphane-1¹,2⁴-dicarboxylate (13 mg, yield 15.3%) and 1′-(tert-butyl) 2⁴-methyl (1²S,1⁴R*)-3-oxa-1(2,4)-piperidina-2(1,2)-benzenacyclodecaphane-1¹,2⁴-dicarboxylate (50 mg, yield 59.0%) as light oil. LC/MS (ESI) m/z: 432 (M+H)⁺.

Step 8: methyl (1²S,1⁴S)-3-oxa-1(2,4)-piperidina-2(1,2)-benzenacyclodecaphane-2⁴-carboxylate hydrochloride (9). A solution of 1¹-(tert-butyl) 2⁴-methyl (1²,1⁴S)-3-oxa-1(2,4)-piperidina-2(1,2)-benzenacyclodecaphane-1¹,2⁴-dicarboxylate (50 mg, 0.12 mmol) in HCl/1,4-dioxane (2 mL) was stirred at room temperature for 1.5 hours. The mixture was concentrated to dryness to give methyl (1²S,1⁴S)-3-oxa-1(2,4)-piperidina-2(1,2)-benzenacyclodecaphane-24-carboxylate hydrochloride (38 mg, yield 99.0%) as white solid, which was used directly without further purification. LC/MS (ESI) m/z: 332 (M+H)⁺.

Step 9: methyl (1²S,1⁴S)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate. To a solution of methyl (1²S,1⁴S)-3-oxa-1(2,4)-piperidina-2(1,2)-benzenacyclodecaphane-24-carboxylate hydrochloride (0.043 g, 0.149 mmol) in dry DCM (3 mL) was added PPh₃Br₂ (68 mg, 0.16 mmol) in portions at 0° C., and the mixture stirred at 0° C. for 1.5 hours. Then DIPEA (0.06 mL, 0.34 mmol) and a solution of methyl (1²S,1⁴S)-3-oxa-1(2,4)-piperidina-2(1,2)-benzenacyclodecaphane-2⁴-carboxylate (38 mg, 0.12 mmol) in DCM (2 mL) was added and the mixture was stirred at 0° C. for 16 hours. The reaction mixture was quenched with ice water and extracted with DCM twice, and the combined organic layers were washed with brine, dried over anhydrous Na₂SO₄ and concentrated to dryness under reduced pressure. The residue was purified by chromatography on silica gel (PE:EtOAc=40:1) to give methyl (1²S,1⁴S)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate (59 mg, yield 85.1%) as white solid. LC/MS (ESI) m/z: 605 (M+H)⁺.

Step 10: (1²S,1⁴S)-1¹-((5-methoxy-7-methyl-1H-indol-4-yl)methyl)-3-oxa-1(2,4)-piperidina-2(1,2)-benzenacyclodecaphane-2⁴-carboxylic acid (Example 77). To a solution of methyl (1²S,1⁴S)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate (58 mg, 0.096 mmol) in MeOH (3 mL)/H₂O (1 mL) was added LiOH H₂O (12 mg, 0.29 mmol) and the mixture was stirred at 55° C. for 2 days. The mixture was acidified with 1 N aq. HCl to pH˜4 and extracted with EtOAc twice. The combined organic layers were washed with water and brine, dried over anhydrous Na₂SO₄, filtered and concentrated to dryness under reduced pressure. The residue was purified by prep-HPLC to give (1²S,1⁴S)-1′-((5-methoxy-7-methyl-1H-indol-4-yl)methyl)-3-oxa-1(2,4)-piperidina-2(1,2)-benzenacyclodecaphane-2⁴-carboxylic acid (14 mg, yield 29.8%) as white solid. ¹HNMR (400 MHz, CD₃OD) δ 8.38 (d, J=6.8 Hz, 1H), 7.80 (d, J=8.0 Hz, 1H), 7.72-7.65 (m, 2H), 7.33 (d, J=2.8 Hz, 1H), 6.80 (s, 1H), 6.53 (d, J=2.0 Hz, 1H), 5.24 (d, J=11.2 Hz, 1H), 4.58-4.48 (m, 1H), 4.43-4.36 (m, 1H), 4.26-4.17 (m, 1H), 3.98-3.89 (m, 1H), 3.87 (s, 3H), 3.59-3.47 (m, 1H), 3.45-3.36 (m, 1H), 2.52 (s, 3H), 2.20-1.95 (m, 5H), 1.90-1.44 (m, 11H), 1.35-1.27 (m, 1H). LC/MS (ESI) m/z: 491 (M+H)⁺.

### Example 78: (1²S,1⁴R)-1¹-((5-methoxy-7-methyl-1H-indol-4-yl)methyl)-3-oxa-1(2,4)-piperidina-2(1,2)-benzenacyclodecaphane-2⁴-carboxylic acid

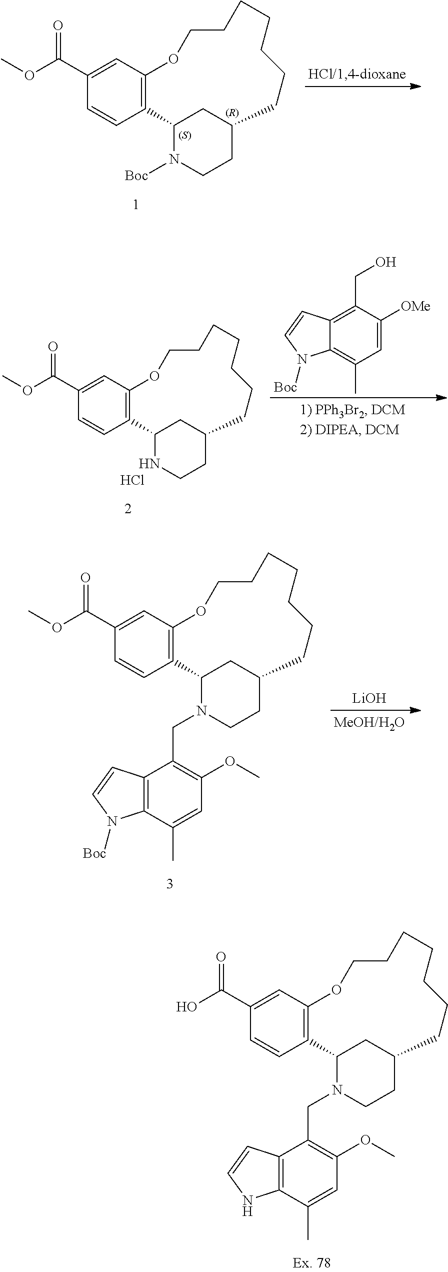

Step 1: methyl (1²S,1⁴R)-3-oxa-1(2,4)-piperidina-2(1,2)-benzenacyclodecaphane-2⁴-carboxylate hydrochloride (2). A solution of 1¹-(tert-butyl) 2⁴-methyl (1²S,1⁴R)-3-oxa-1(2,4)-piperidina-2(1,2)-benzenacyclodecaphane-1¹,2⁴-dicarboxylate (13 mg, 0.03 mmol) in HCl/1,4-dioxane (1 mL) was stirred at room temperature for 1.5 hours. The mixture was concentrated to dryness to give methyl (1²S,1⁴R)-3-oxa-1(2,4)-piperidina-2(1,2)-benzenacyclodecaphane-2⁴-carboxylate hydrochloride (10 mg, yield 100.0%) as white solid, which was used directly without purification. LC/MS (ESI) m/z: 332 (M+H)⁺.

Step 2: methyl (1²S,1⁴R)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate. To a solution of tert-butyl 4-(hydroxymethyl)-5-methoxy-7-methylindole-1-carboxylate (11 mg, 0.039 mmol) in dry DCM (2 mL) was added PPh₃Br₂ (18 mg, 0.042 mmol) in portions at 0° C., to and the mixture stirred at 0° C. for 1.5 hours. Then DIPEA (0.02 mL, 0.091 mmol) and a solution of methyl (1²S,1⁴R)-3-oxa-1(2,4)-piperidina-2(1,2)-benzenacyclodecaphane-2⁴-carboxylate (10 mg, 0.03 mmol) in DCM (1 mL) was added and the mixture was stirred at 0° C. for 16 hours. The reaction mixture was quenched with ice water and extracted with DCM twice, and the combined organic layers were washed with brine, dried over anhydrous Na₂SO₄ and concentrated to dryness under reduced pressure.

The residue was purified by chromatography on silica gel (PE:EtOAc=40:1) to give methyl (1²S,1⁴R)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate (14 mg, yield 76.7%) as white solid. LC/MS (ESI) m/z: 605 (M+H)⁺.

Step 3: (1²S,1⁴R)-1¹-((5-methoxy-7-methyl-1H-indol-4-yl)methyl)-3-oxa-1(2,4)-piperidina-2(1,2)-benzenacyclodecaphane-2⁴-carboxylic acid (Example 78). To a solution of methyl (1²S,1⁴R)-1¹-((1-(tert-butoxycarbonyl)-5-methoxy-7-methyl-1H-indol-4-yl)methyl)-3-oxa-1(2,4)-piperidina-2(1,2)-benzenacyclodecaphane-2⁴-carboxylate (14 mg, 0.023 mmol) in MeOH (3 mL)/H₂O (1 mL) was added LiOH H₂O (3 mg, 0.069 mmol) and the mixture was stirred at 55° C. for 24 hours. The mixture was acidified with 1 N aq. HCl to pH˜4 and extracted with EtOAc twice. The combined organic layers were washed with water and brine, dried over anhydrous Na₂SO₄, filtered and concentrated to dryness under reduced pressure. The residue was purified by prep-HPLC to give (1²S,1⁴R)-1¹-((5-methoxy-7-methyl-1H-indol-4-yl)methyl)-3-oxa-1(2,4)-piperidina-2(1,2)-benzenacyclodecaphane-2⁴-carboxylic acid (2.3 mg, yield 20.3%) as white solid. ¹HNMR (400 MHz, CD₃OD) δ 8.41 (s, 1H), 7.79 (s, 2H), 7.66 (s, 1H), 7.35 (d, J=2.0 Hz, 1H), 6.87 (d, J=1.2 Hz, 1H), 6.55 (d, J=1.2 Hz, 1H), 5.01-4.91 (m, 1H), 4.38-4.34 (m, 1H), 4.22-4.16 (m, 1H), 4.04-3.95 (m, 4H), 3.55-3.41 (m, 2H), 2.60-2.53 (m, 4H), 2.14-2.07 (m, 1H), 2.02-1.93 (m, 1H), 1.84-1.74 (m, 2H), 1.67-1.38 (m, 11H), 1.25-1.10 (m, 2H).LC/MS (ESI) m/z: 491 (M+H)⁺.

### Example 79: (1²S,1⁴S)-1¹-((5-methoxy-7-methyl-1H-indol-4-yl)methyl)-N-methyl-3,10-dioxa-1(2,4)-piperidina-2(1,2)-benzenacyclodecaphane-2⁴-sulfonamide

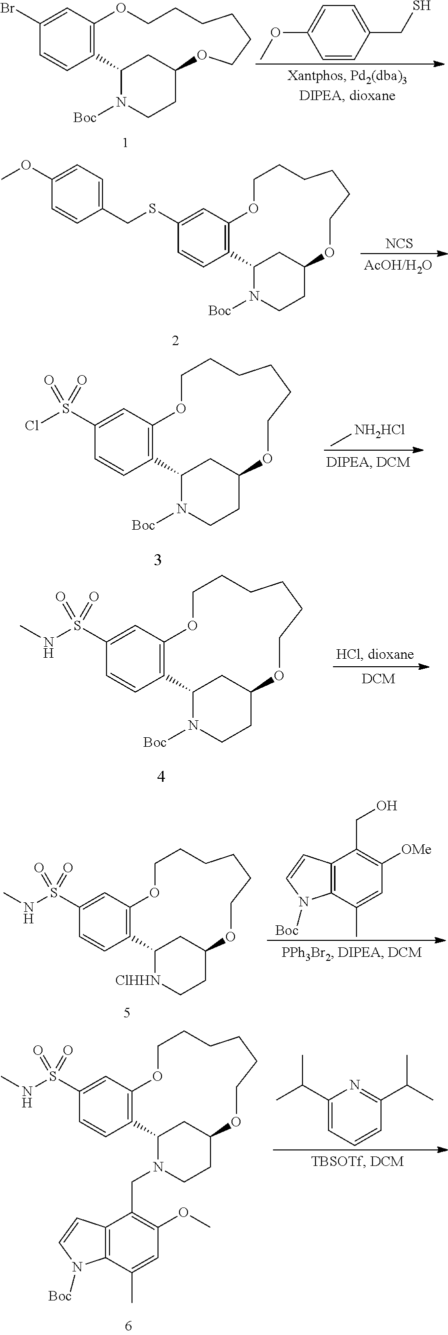

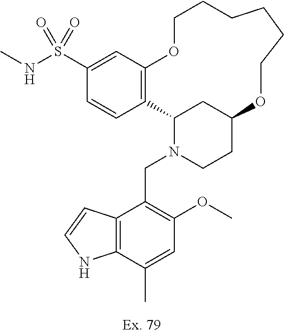 [SMILES: CNS(=O)(=O)c1ccc2c(c1)OCCCCCCO[C@H]1CCN(Cc3c(OC)cc(C)c4[nH]ccc34)[C@H]2C1]

Step 1: tert-butyl (1²S,1⁴S)-2⁴-((4-methoxybenzyl)thio)-3,10-dioxa-1(2,4)-piperidina-2(1,2)-benzenacyclodecaphane-1¹-carboxylate. To a solution of tert-butyl (1²S,1⁴S)-2⁴-bromo-3,10-dioxa-1(2,4)-piperidina-2(1,2)-benzenacyclodecaphane-1¹-carboxylate (110 mg, 0.24 mmol) in 1,4-dioxane (2 mL) was added (4-methoxyphenyl)methanethiol (75 mg, 0.48 mmol), DIPEA (156 mg, 1.21 mmol), Xantphos (28 mg, 0.048 mmol) and Pd₂(dba)₃ (44 mg, 0.048 mmol) under N₂ atmosphere and the mixture was stirred under N₂ atmosphere at 115° C. for 18 hours. The mixture was extracted with EtOAc twice, and the combined organic layers were washed with water and brine, dried over anhydrous Na₂SO₄, filtered and concentrated under reduced pressure to dryness. The residue was purified by prep-TLC (PE:EtOAc=1:1) to give tert-butyl (1²S,1⁴S)-2⁴-((4-methoxybenzyl)thio)-3,10-dioxa-1(2,4)-piperidina-2(1,2)-benzenacyclodecaphane-1¹-carboxylate (100 mg, yield 78.7%) as yellow oil. LC/MS (ESI) m/z: 528 (M+H)⁺.

Step 2: tert-butyl (1²S,1⁴S)-2⁴-(chlorosulfonyl)-3,10-dioxa-1(2,4)-piperidina-2(1,2)-benzenacyclodecaphane-1¹-carboxylate. To a solution of tert-butyl (1²S,1⁴S)-2⁴-((4-methoxybenzyl)thio)-3,10-dioxa-1(2,4)-piperidina-2 (1,2)-benzenacyclodecaphane-1¹-carboxylate (95 mg, 0.18 mmol) in AcOH (1 mL) and H₂O (1 mL) was added NCS (36 mg, 0.27 mmol) and the mixture was stirred at room temperature for 18 hours. The mixture was concentrated under reduced pressure to dryness. The residue was purified by prep-TLC (PE:EtOAc=5:1) to give tert-butyl (1²S,1⁴S)-2⁴-(chlorosulfonyl)-3,10-dioxa-1(2,4)-piperidina-2(1,2)-benzenacyclodecaphane-1¹-carboxylate (50 mg, yield 58.8%) as yellow oil. LC/MS (ESI) m/z: 474 (M+H)⁺.

Step 3: tert-butyl (1²S,1⁴S)-2⁴-(N-methylsulfamoyl)-3,10-dioxa-1(2,4)-piperidina-2(1,2)-benzenacyclodecaphane-1¹-carboxylate. To a solution of tert-butyl (1²S,1⁴S)-2⁴-(chlorosulfonyl)-3,10-dioxa-1(2,4)-piperidina-2(1,2)-benzenacyclodecaphane-1¹-carboxylate (50 mg, 0.11 mmol) in DCM (2 mL) was added methanamine hydrochloride (15 mg, 0.22 mmol) and DIPEA (43 mg, 0.33 mmol), and the mixture was stirred at room temperature for 3 hours. The mixture was concentrated under reduced pressure to dryness. The residue was purified by prep-TLC (DCM:MeOH=20:1) to give tert-butyl (1²S, 1⁴S)-2⁴-(N-methylsulfamoyl)-3,10-dioxa-1(2,4)-piperidina-2(1,2)-benzenacyclodecaphane-1¹-carboxylate (33 mg, yield 63.5%) as yellow oil. LC/MS (ESI) m/z: 469 (M+H)⁺.

Step 4: (1²S,1⁴S)—N-methyl-3,10-dioxa-1(2,4)-piperidina-2(1,2)-benzenacyclodecaphane-2⁴-sulfonamide hydrochloride. To a solution of tert-butyl (1²S,1⁴S)-2⁴-(N-methylsulfamoyl)-3,10-dioxa-1(2,4)-piperidina-2 (1,2)-benzenacyclodecaphane-1¹-carboxylate (33 mg, 0.07 mmol) in DCM (1 mL) was added HCl/1,4-dioxane (1 mL) at 0° C., and the reaction mixture was stirred at 25° C. for 1 hour. The mixture was concentrated to dryness under reduced pressure to give (1²S,1⁴S)—N-methyl-3,10-dioxa-1(2,4)-piperidina-2(1,2)-benzenacyclodecaphane-2⁴-sulfonamide hydrochloride (30 mg, crude) as white solid. LC/MS (ESI) m/z: 369 (M+H)⁺.

Step 5: tert-butyl 5-methoxy-7-methyl-4-(((1²S,1⁴S)-2⁴-(N-methylsulfamoyl)-3,10-dioxa-1 (2,4)-piperidina-2(1,2)-benzenacyclodecaphane-1¹-yl)methyl)-1H-indole-1-carboxylate. To a solution of tert-butyl 4-(hydroxymethyl)-5-methoxy-7-methyl-1H-indole-1-carboxylate (30 mg, 0.10 mmol) in dry DCM (1 mL) was added PPh₃Br₂ (45 mg, 0.11 mmol) portion-wise at 0° C., and the mixture was stirred under N₂ atmosphere for 2 hours. Then a mixture of (1², 1⁴S)—N-methyl-3,10-dioxa-1 (2,4)-piperidina-2(1,2)-benzenacyclodecaphane-2⁴-sulfonamide hydrochloride (30 mg, 0.074 mmol) and DIPEA (30 mg, 0.23 mmol) in dry DCM (1 mL) was added and the resulting mixture was stirred at 0° C. for 1.5 hours. The mixture was concentrated to dryness under reduced pressure and the residue was purified by prep-TLC (DCM:MeOH=20:1) to give tert-butyl 5-methoxy-7-methyl-4-(((1²S,1⁴S)-2⁴-(N-methylsulfamoyl)-3,10-dioxa-1(2,4)-piperidina-2(1,2)-benzenacyclodecaphane-1¹-yl)methyl)-1H-indole-1-carboxylate (23 mg, yield 47.9%) as colorless oil. LC/MS (ESI) m/z: 642 (M+H)⁺.

Step 6: (1²S,1⁴S)-1¹-((5-methoxy-7-methyl-1H-indol-4-yl)methyl)-N-methyl-3,10-dioxa-1 (2,4)-piperidina-2(1,2)-benzenacyclodecaphane-2⁴-sulfonamide. A mixture of tert-butyl 5-methoxy-7-methyl-4-(((1², 1⁴S)-2⁴-(N-methylsulfamoyl)-3,10-dioxa-1(2,4)-piperidina-2(1,2)-benzenacyclodecaphane-1¹-yl)methyl)-1H-indole-1-carboxylate (18 mg, 0.028 mmol), TMSOTf (18 mg, 0.082 mmol) and 2,6-diisopropylpyridine (50 mg, 0.042 mmol) was stirred at room temperature for 18 hours. The mixture was concentrated to dryness and the residue was purified by prep-HPLC to give ((1²S,1⁴S)-1′-((5-methoxy-7-methyl-1H-indol-4-yl)methyl)-N-methyl-3,10-dioxa-1(2,4)-piperidina-2(1,2)-benzenacyclodecaphane-2⁴-sulfonamide (1.1 mg, yield 7.3%) as white solid. ¹H NMR (400 MHz, CD₃OD) δ 8.51 (s, 1H), 7.88 (d, J=8.1 Hz, 1H), 7.63 (d, J=8.1 Hz, 1H), 7.49 (s, 1H), 7.31 (d, J=3.0 Hz, 1H), 6.79 (s, 1H), 6.47 (d, J=3.0 Hz, 1H), 5.20 (dd, J=11.0, 5.9 Hz, 1H), 4.48-4.40 (m, 2H), 3.94 (t, J=9.0 Hz, 1H), 3.86 (s, 3H), 3.70 (s, 1H), 3.61-3.53 (m, 2H), 3.40-3.35 (m, 1H), 2.58 (s, 3H), 2.52 (s, 3H), 2.29 (d, J=14.2 Hz, 1H), 2.12-1.97 (m, 4H), 1.88 (dd, J=23.4, 13.7 Hz, 4H), 1.60 (dd, J=15.6, 8.5 Hz, 3H), 1.46 (t, J=13.3 Hz, 2H). LC/MS (ESI) m/z: 542 (M+H)⁺.

### Biological Example 1: Human Factor B Assay

CVF-Bb complex, prepared from purified cobra venom factor, (3 nM) is incubated with a compound of the disclosure at various concentrations for 10 minutes at room temperature in PBS (pH 7.4) containing 10 mM MgCl₂ and 0.05% (w/v) CHAPS. Human Complement C₃ substrate is added to a final concentration of 1 μM. After 1 hour incubation at room temperature, the enzyme reaction is stopped by addition of a cocktail of concentrated pan-protease inhibitors. The product of the reaction, C₃a, is quantified by means of an enzyme-linked-immunosorbent assay and/or denaturing gel electrophoresis (SDS-PAGE). IC₅₀ values are calculated from percentage of inhibition of CVF-Bb activity as a function of test compound concentration. IC₅₀ values for several examples of the disclosure are provided in Table 1.

TABLE 1

| Compound | IC₅₀ (μM) | RT min (Method A) | MS (M + 1) |
|---|---|---|---|
| Compar. Ex. 1 | 0.135 | 0.73 | 480 |
| Compar. Ex. 2 | 0.395 | 1.15 | 571 |
| Ex. 3 | 0.263 | 1.37 | 453 |
| Ex. 4 | 3.55 | 1.23 | 453 |
| Ex. 5 | 0.262 | 1.64 | 493 |
| Ex. 6 | 0.313 | 1.24 | 524 |
| Ex. 7 | 0.384 | 1.86 | 507 |
| Ex. 8 | 0.397 | 1.44 | 479 |
| Ex. 9 | 0.342 | 1.41 | 477 |
| Ex. 10 | 0.384 | 1.72 | 505 |
| Ex. 11 | 1.2 | 1.58 | 491 |
| Ex. 12 | 0.28 | 0.97 | 518 |
| Ex. 13 | 1.11 | 1.00 | 532 |
| Ex. 30 | 4.91 | 2.20 | 535 |
| Ex. 31 | 2.87 | 2.34 | 549 |
| Ex. 32 | >10 | 2.45 | 563 |
| Ex. 33 | 0.096 | 1.75 | 570 |
| Ex. 34 | >10 | 2.16 | 505 |
| Ex. 25 | >10 | 1.83 | 477 |
| Ex. 35 | >10 | 1.96 | 491 |
| Ex. 36 | 0.241 | 1.69 | 489 |
| Ex. 37 | 0.263 | 1.65 | 489 |
| Ex. 38 | 0.16 | 1.86 | 503 |
| Ex. 39 | 0.251 | 1.82 | 491 |
| Ex. 40 | 0.309 | 1.46 | 556 |
| Ex. 41 | 0.129 | 1.94 | 505 |
| Ex. 42 | 1.06 | 1.42 | 477 |
| Ex. 43 | 0.122 | 1.26 | 500 |
| Ex. 44 | 0.138 | 1.34 | 514 |
| Ex. 45 | 0.396 | 1.74 | 587 |
| Ex. 46 | 0.311 | 1.86 | 610 |
| Ex. 47 | 0.649 | 1.84 | 598 |
| Ex. 48 | 0.0837 | 1.72 | 541 |
| Ex. 49 | 0.208 | 1.83 | 543 |
| Ex. 50 | >10 | 1.42 | 513 |
| Ex. 51 | >10 | 1.74 | 541 |
| Ex. 52 | >10 | 1.84 | 543 |
| Ex. 53 | >10 | 1.65 | 541 |
| Ex. 54 | 0.303 | 1.83 | 618 |
| Ex. 55 | 2.89 | 1.88 | 515 |
| Ex. 56 | 0.375 | 1.88 | 620 |
| Ex. 57 | 0.153 | 2.05 | 632 |
| Ex. 58 | 1.06 | 2.02 | 580 |
| Ex. 59 | 0.244 | 1.63 | 570 |
| Ex. 60 | 0.231 | 1.68 | 582 |
| Ex. 61 | 0.331 | 1.89 | 610 |
| Ex. 62 | 0.293 | 2.02 | 624 |
| Ex. 63 | 2.45 | 1.48 | 508 |
| Ex. 64 | 0.127 | 2.12 | 517 |
| Ex. 65 | 0.139 | 2.02 | 582 |
| Ex. 66 | 0.411 | 1.72 | 584 |
| Ex. 67 | 0.33 | 1.75 | 596 |
| Ex. 68 | 0.674 | 1.75 | 566 |
| Ex. 69 | 0.581 | 1.81 | 566 |
| Ex. 70 | 0.543 | 1.94 | 618 |
| Ex. 71 | 0.258 | 1.89 | 568 |
| Ex. 72 | 0.129 | 2.20 | 519 |
| Ex. 73 | 0.271 | 2.09 | 517 |
| Ex. 74 | 1.25 | 1.77 | 517 |
| Ex. 75 | 0.352 | 1.55 | 513 |
| Ex. 76 | 0.251 | 1.65 | 527 |
| Ex. 77 | 0.0856 | 1.90 | 491 |
| Ex. 78 | 0.731 | 1.90 | 491 |
| Ex. 79 | >10 | 1.60 | 542 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1-144. (canceled)

145. A compound of the formula (I), (IV), (V), or (VI): 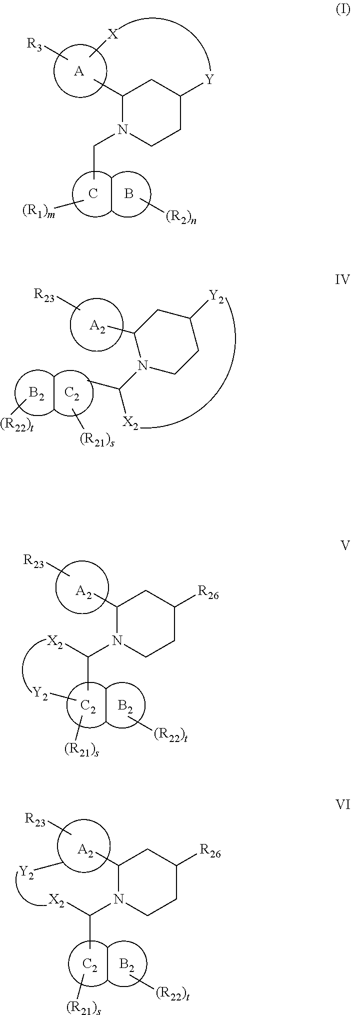
or a pharmaceutically acceptable salt thereof, wherein
ring A represents a phenyl or a naphthyl ring;
ring B and ring C form a bicyclic heteroaryl, bicyclic heterocyclyl, or bicyclic cycloalkyl moiety, where ring B is a monocyclic heteroaryl, monocyclic heterocyclyl, or monocyclic cycloalkyl ring, and ring C is a phenyl or monocyclic 6-membered heteroaryl ring;
m is an integer 0, 1, 2, or 3;
n is an integer 0, 1, or 2;
R₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₂ is independently selected from halogen, —CN, C₁-C₆ alkyl, C₁-C₆ haloalkyl, C₁-C₆ alkoxy, and C₁-C₆ haloalkoxy;
R₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₅, —SO₂OH, —SO₂(C₁-C₆ alkyl), —SO₂N(R₅)₂, —SO₂NH—COCH₃, —S(O)(NR₅)R₅, —NH—SO₂R₅, —NHCO—NHSO₂R₅, —PO(OH)₂, —PO(OH)R₅, pyrazolyl, and tetrazolyl, wherein
each R₅ is independently selected from the group consisting of hydrogen, C₁-C₆ alkyl, C₁-C₆ haloalkyl, cycloalkyl optionally substituted with C₁-C₄ alkyl, and phenyl, and monocyclic heteroaryl; and
X is CH₂, CH, O, S, or NH, and Y is O, S, or NH, where X and Y, together with the atoms to which they are attached, form a 10- to 16-member macrocycle, the macrocycle optionally substituted with one or more R₄, wherein
each R₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
or two R₄ groups, together with the carbon to which they are attached, form a ═O;
ring A₂ represents a phenyl or a naphthyl ring;
ring B₂ and ring C₂ form a bicyclic heteroaryl, bicyclic heterocyclyl, or bicyclic cycloalkyl moiety, where ring B₂ is a monocyclic heteroaryl, monocyclic heterocyclyl, or monocyclic cycloalkyl ring, and ring C₂ is a phenyl or monocyclic 6-membered heteroaryl ring;
s is an integer 0, 1, or 2;
t is an integer 0, 1, or 2;
R₂₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₂₂ is independently selected from halogen, —CN, C₁-C₆ alkyl, C₁-C₆ haloalkyl, C₁-C₆ alkoxy, and C₁-C₆ haloalkoxy;
R₂₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₂₅, —SO₂OH, —SO₂(C₁-C₆ alkyl), —SO₂N(R₂₅)₂, —SO₂NH—COCH₃, —S(O)(NR₂₅)R₂₅, —NH—SO₂R₂₅, —NHCO—NHSO₂R₂₅, —PO(OH)₂, —PO(OH)R₂₅, pyrazolyl, and tetrazolyl, wherein
each R₂₅ is independently selected from the group consisting of hydrogen, C₁-C₆ alkyl, C₁-C₆ haloalkyl;
R₂₆ is —OH, C₁-C₆ alkoxy, —NH₂, —NH(C₁-C₆ alkyl), or —N(C₁-C₆ alkyl)₂; and
X is CH₂, CH, O, S, or NH, and Y is O, S, or NH, where X and Y, together with the atoms to which they are attached, form a 10- to 16-member macrocycle, the macrocycle optionally substituted with one or more R₂₄, wherein
each R₂₄ is independently selected from the group consisting of halogen, —NO₂, —CN, C₁-C₆alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
or two R₂₄ groups, together with the carbon to which they are attached, form a ═O.

146. A compound of the formula (I): 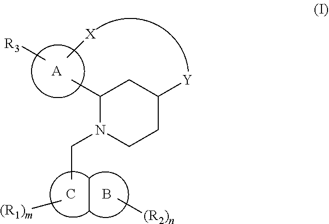
or a pharmaceutically acceptable salt thereof, wherein
ring A represents a phenyl or a naphthyl ring;
ring B and ring C form a bicyclic heteroaryl, bicyclic heterocyclyl, or bicyclic cycloalkyl moiety, where ring B is a monocyclic heteroaryl, monocyclic heterocyclyl, or monocyclic cycloalkyl ring, and ring C is a phenyl or monocyclic 6-membered heteroaryl ring;
m is an integer 0, 1, 2, or 3;
n is an integer 0, 1, or 2;
R₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₂ is independently selected from halogen, —CN, C₁-C₆ alkyl, C₁-C₆ haloalkyl, C₁-C₆ alkoxy, and C₁-C₆ haloalkoxy;
R₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₅, —SO₂OH, —SO₂(C₁-C₆ alkyl), —SO₂N(R₅)₂, —SO₂NH—COCH₃, —S(O)(NR₅)R₅, —NH—SO₂R₅, —NHCO—NHSO₂R₅, —PO(OH)₂, —PO(OH)R₅, pyrazolyl, and tetrazolyl, wherein
each R₅ is independently selected from the group consisting of hydrogen, C₁-C₆ alkyl, C₁-C₆ haloalkyl, cycloalkyl optionally substituted with C₁-C₄ alkyl, and phenyl, and monocyclic heteroaryl; and
X is CH₂, CH, O, S, or NH, and Y is O, S, or NH, where X and Y, together with the atoms to which they are attached, form a 10- to 16-member macrocycle, the macrocycle optionally substituted with one or more R₄, wherein
each R₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
or two R₄ groups, together with the carbon to which they are attached, form a ═O;

147. The compound of claim 146, wherein ring A represents a phenyl ring.

148. The compound of claim 146, wherein ring A represents a naphthyl ring.

149. The compound of claim 146, wherein ring C is phenyl.

150. The compound of claim 146, wherein ring B is a monocyclic heteroaryl selected from pyrrole, pyrazole, imidazole, isoxazole, or oxazole.

151. The compound of claim 146, wherein m is 1 or 2 and n is 0 or 1.

152. The compound of claim 146, wherein R₁ is independently selected from halogen, C₁-C₆ alkyl, C₁-C₆ haloalkyl, —NH₂, —NH(C₁-C₆ alkyl), —N(C₁-C₆ alkyl)₂, —OH, and C₁-C₆ alkoxy.

153. The compound of claim 146, wherein R₂ is independently selected from halogen, C₁-C₆ alkyl, C₁-C₆ haloalkyl, and C₁-C₆ alkoxy.

154. The compound of claim 146, which is of formula:

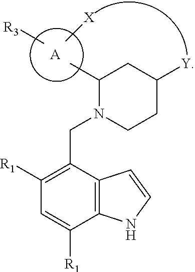

155. The compound of claim 146, wherein R₃ is selected from —CO₂H, —CO₂(C₁-C₆ alkyl), —CONH₂, —CONH(C₁-C₆ alkyl), —CON(C₁-C₆ alkyl)₂, —CONH—S(O)₂R₅, —SO₂OH, —SO₂(C₁-C₆alkyl), —SO₂N(R₅)₂, —S(O)(NR₅)R₅, —NH—SO₂R₅, —NHCO—NHSO₂R₅, —PO(OH)₂, and —PO(OH)R₅.

156. The compound of claim 146, wherein each R₄ is independently selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₄ groups, together with the carbon to which they are attached, form ═O.

157. The compound of claim 146, wherein X and Y, together with the atoms to which they are attached, form a 12- to 14-member macrocycle optionally fused with a triazole, the macrocycle optionally substituted with one or more R₄.

158. The compound of claim 146, wherein the [symbol] portion of the macrocycle has the following structure, 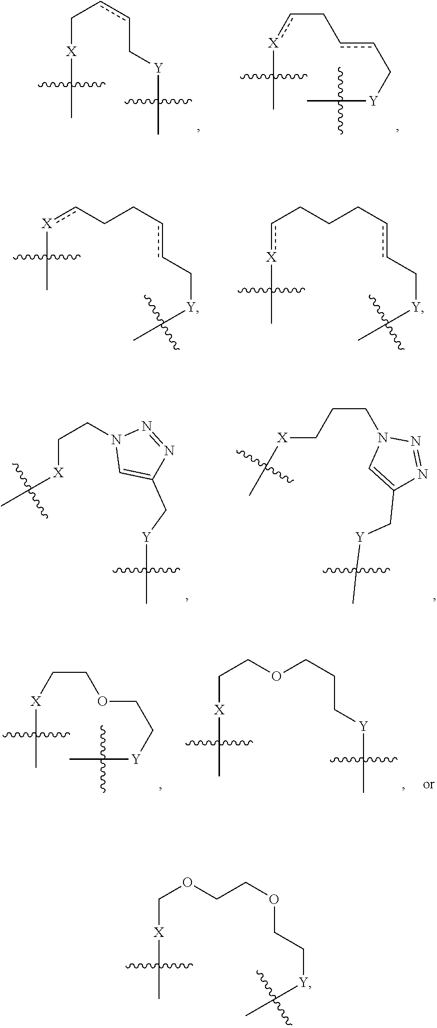
each optionally substituted with one or more R₄.

159. The compound of claim 146, wherein X is O and Y is O.

160. The compound of claim 146, which is

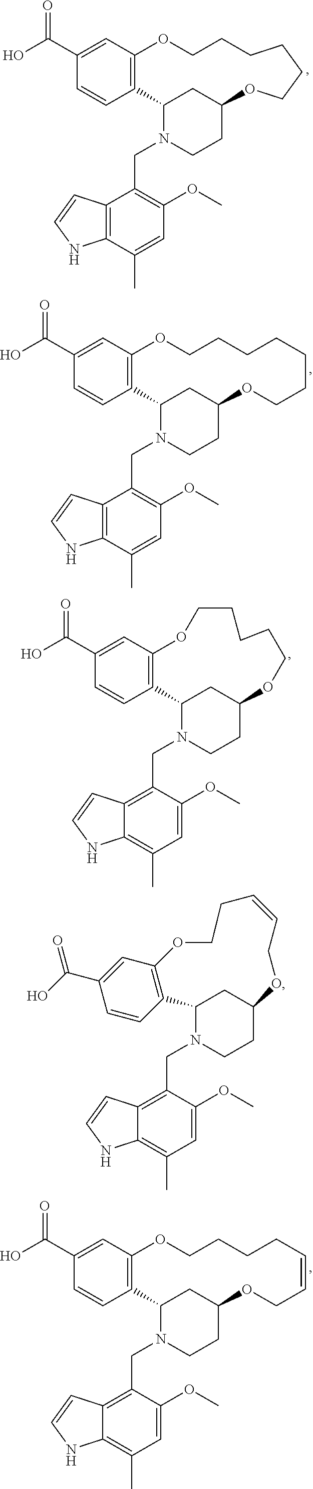 [SMILES: COc1cc(C)c2[nH]ccc2c1CN1CC[C@H]2C[C@H]1c1ccc(C(=O)O)cc1OCCCCCCO2 ; COc1cc(C)c2[nH]ccc2c1CN1CC[C@H]2C[C@H]1c1ccc(C(=O)O)cc1OCCCCCCCO2 ; COc1cc(C)c2[nH]ccc2c1CN1CC[C@H]2C[C@H]1c1ccc(C(=O)O)cc1OCCCCCO2 ; COc1cc(C)c2[nH]ccc2c1CN1CC[C@H]2C[C@H]1c1ccc(C(=O)O)cc1OCC/C=C\CO2 ; COc1cc(C)c2[nH]ccc2c1CN1CC[C@H]2C[C@H]1c1ccc(C(=O)O)cc1OCCCC/C=C\CO2]

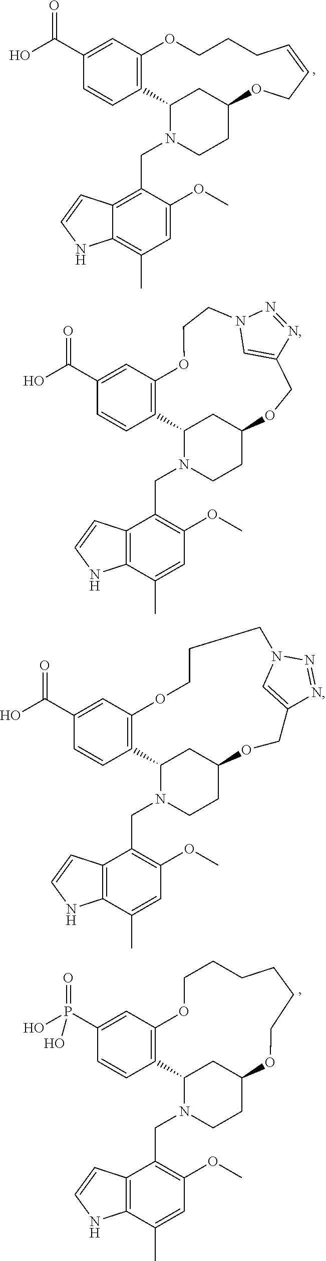 [SMILES: COc1cc(C)c2[nH]ccc2c1CN1CC[C@H]2C[C@H]1c1ccc(C(=O)O)cc1OCCC/C=C\CO2 ; COc1cc(C)c2[nH]ccc2c1CN1CC[C@H]2C[C@H]1c1ccc(C(=O)O)cc1OCCn1cc(nn1)CO2 ; COc1cc(C)c2[nH]ccc2c1CN1CC[C@H]2C[C@H]1c1ccc(C(=O)O)cc1OCCCn1cc(nn1)CO2 ; COc1cc(C)c2[nH]ccc2c1CN1CC[C@H]2C[C@H]1c1ccc(P(=O)(O)O)cc1OCCCCCCO2]

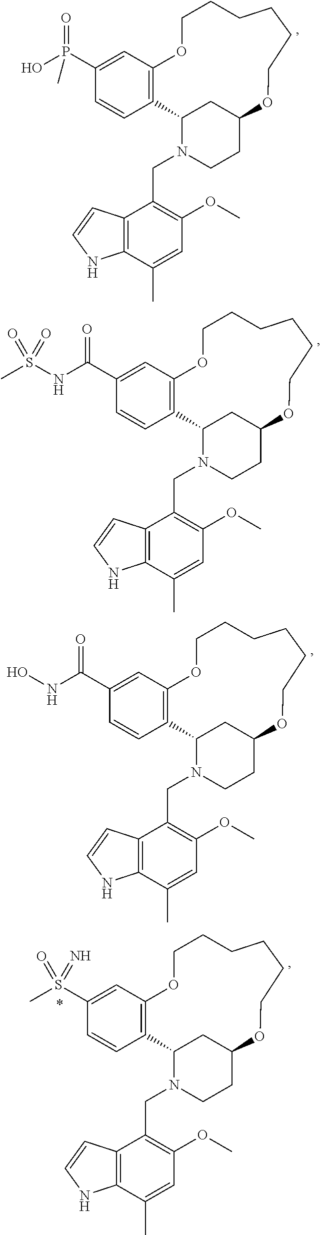

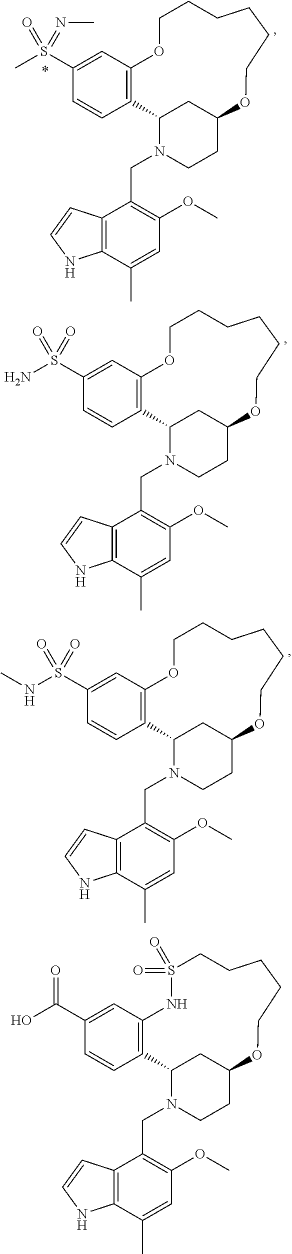

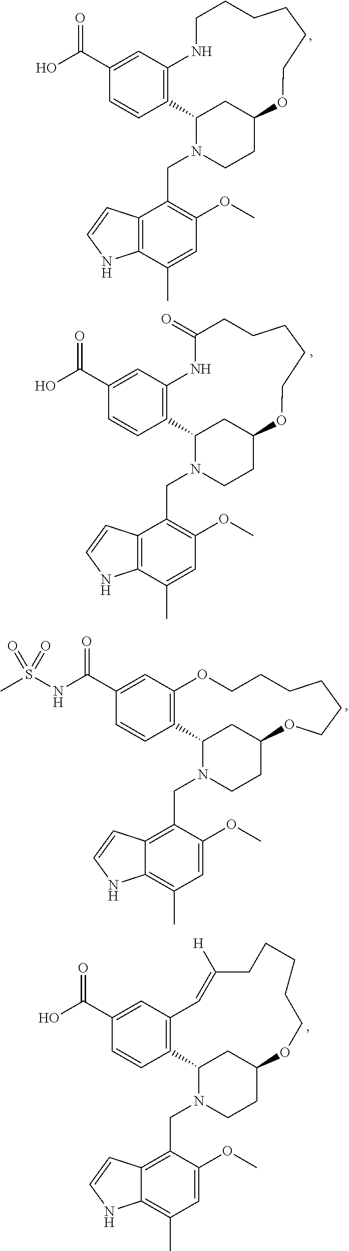

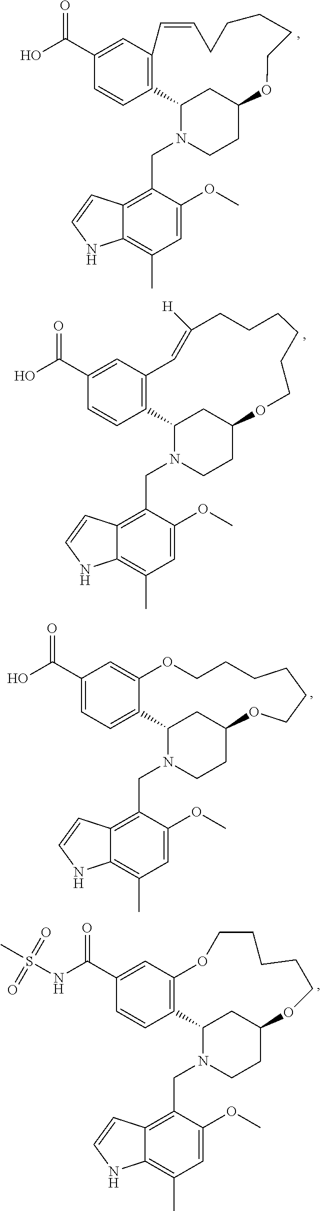

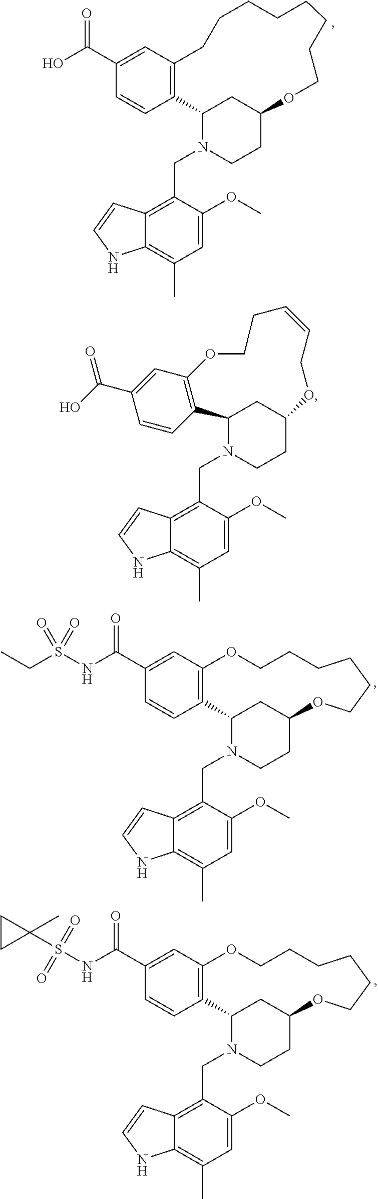 [SMILES: COc1cc(C)c2[nH]ccc2c1CN1CC[C@H]2C[C@H]1c1ccc(C(=O)O)cc1CCCCCCCCO2 ; COc1cc(C)c2[nH]ccc2c1CN1CC[C@@H]2C[C@@H]1c1ccc(C(=O)O)cc1OCC/C=C\CO2 ; CCS(=O)(=O)NC(=O)c1ccc2c(c1)OCCCCCCO[C@H]1CCN(Cc3c(OC)cc(C)c4[nH]ccc34)[C@H]2C1 ; COc1cc(C)c2[nH]ccc2c1CN1CC[C@H]2C[C@H]1c1ccc(C(=O)NS(=O)(=O)C3(C)CC3)cc1OCCCCCCO2]

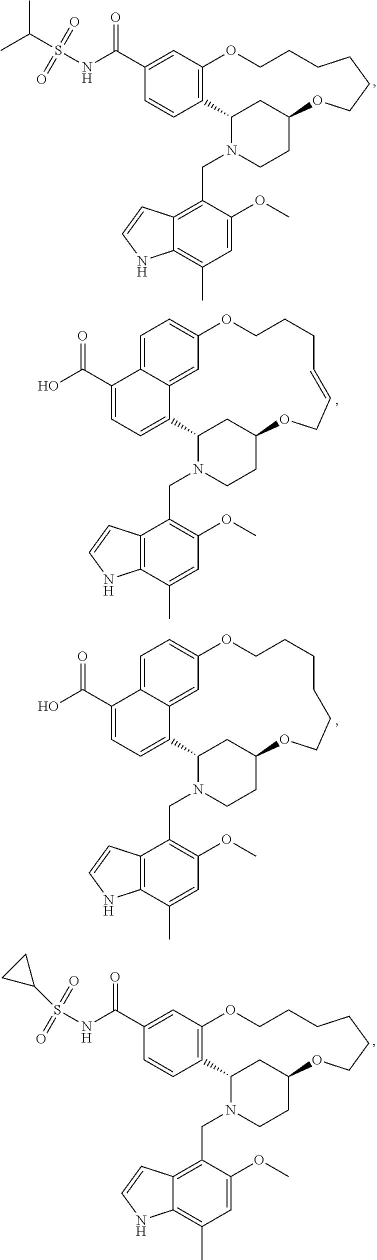

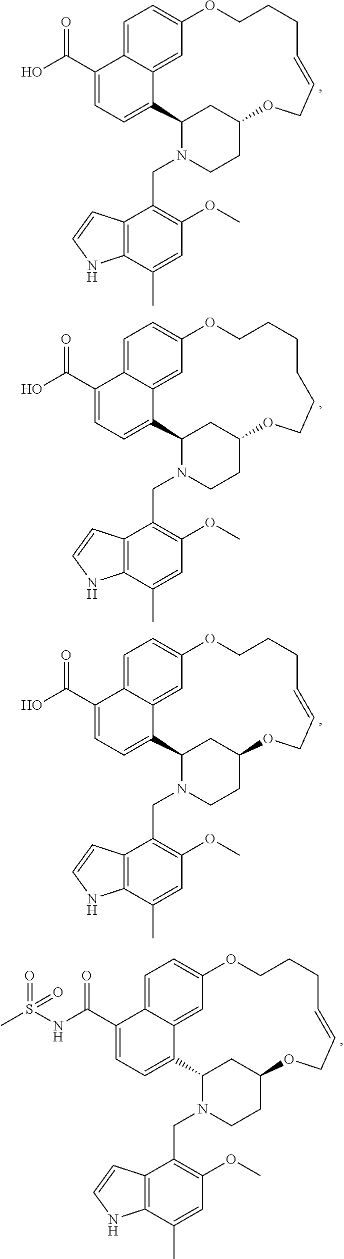

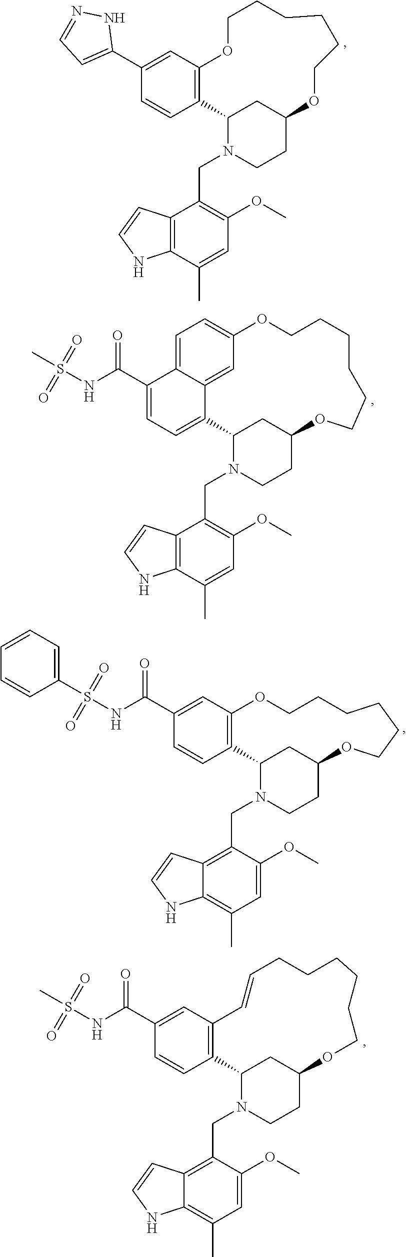

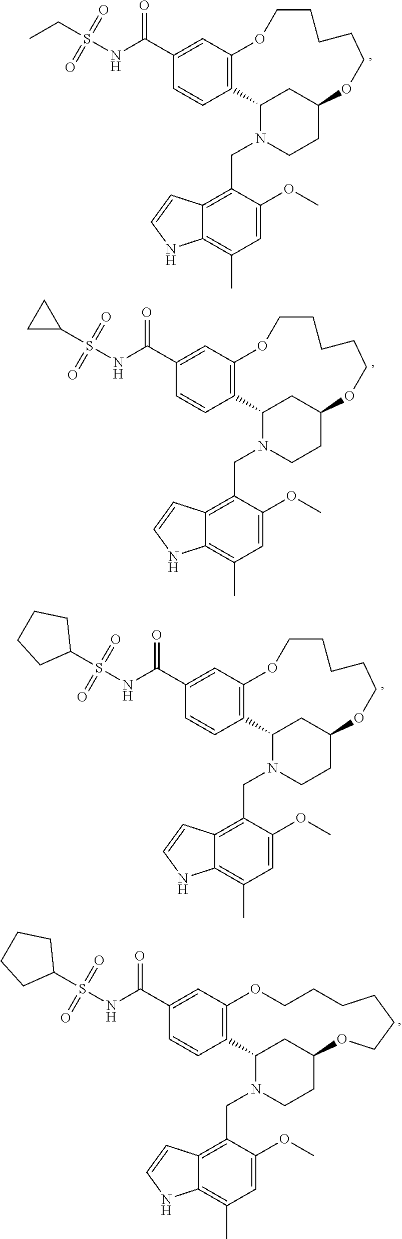 [SMILES: CCS(=O)(=O)NC(=O)c1ccc2c(c1)OCCCCCO[C@H]1CCN(Cc3c(OC)cc(C)c4[nH]ccc34)[C@H]2C1 ; COc1cc(C)c2[nH]ccc2c1CN1CC[C@H]2C[C@H]1c1ccc(C(=O)NS(=O)(=O)C3CC3)cc1OCCCCCO2 ; COc1cc(C)c2[nH]ccc2c1CN1CC[C@H]2C[C@H]1c1ccc(C(=O)NS(=O)(=O)C3CCCC3)cc1OCCCCCO2 ; COc1cc(C)c2[nH]ccc2c1CN1CC[C@H]2C[C@H]1c1ccc(C(=O)NS(=O)(=O)C3CCCC3)cc1OCCCCCCO2]

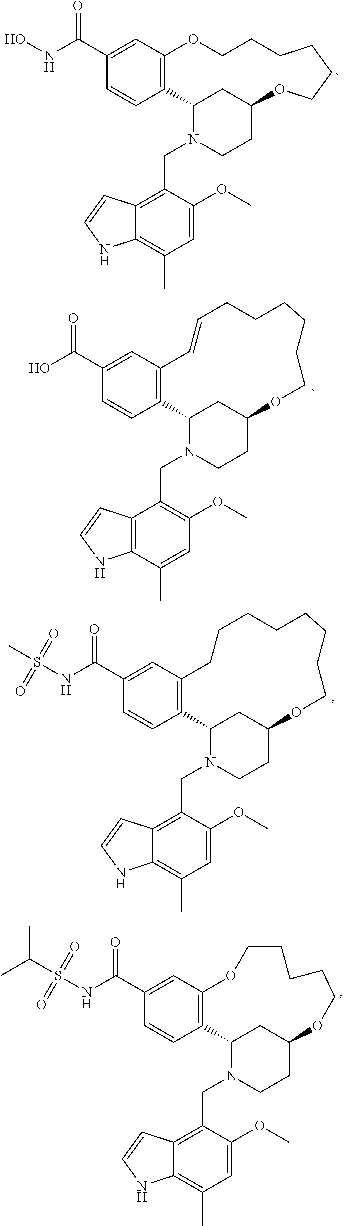

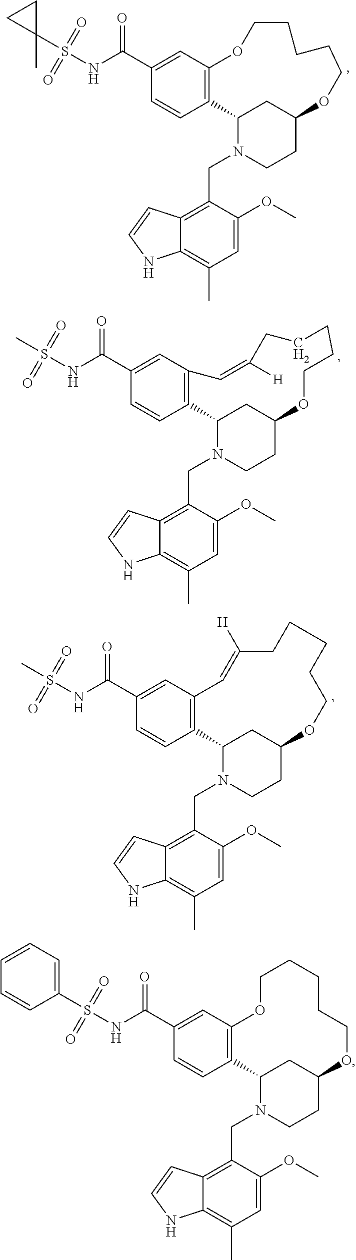

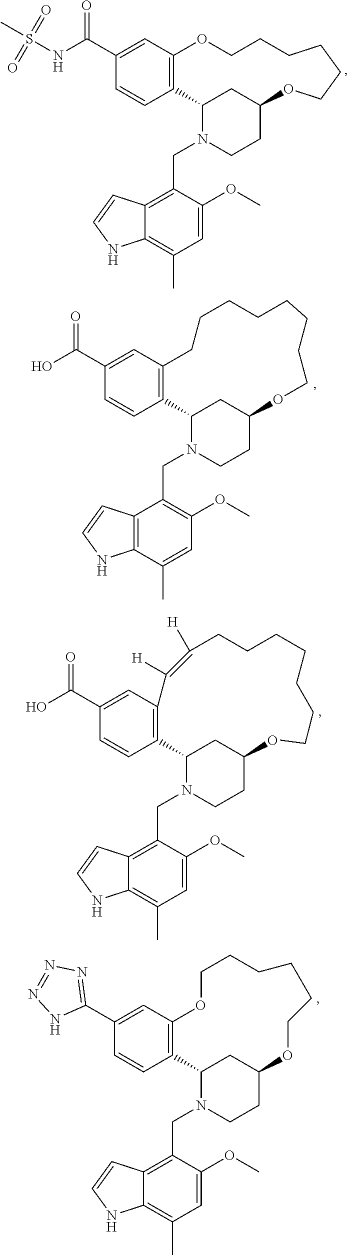

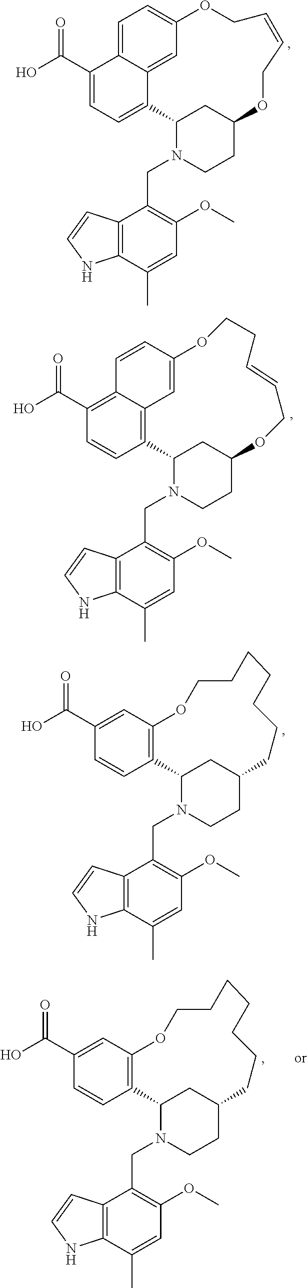

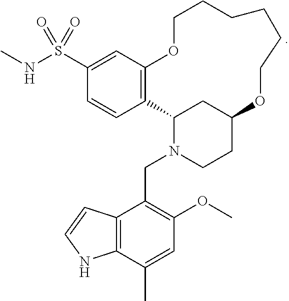 [SMILES: CNS(=O)(=O)c1ccc2c(c1)OCCCCCCO[C@H]1CCN(Cc3c(OC)cc(C)c4[nH]ccc34)[C@H]2C1]

161. A compound of the formula (II) or (Ill): 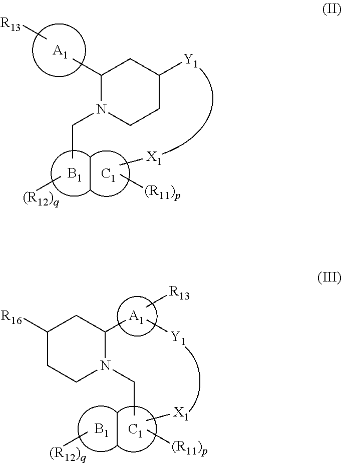
or a pharmaceutically acceptable salt thereof, wherein
ring A₁ represents a phenyl or a naphthyl ring;
ring B₁ and ring C₁ form a bicyclic heteroaryl, bicyclic heterocyclyl, or bicyclic cycloalkyl moiety, where ring B₁ is a monocyclic heteroaryl, monocyclic heterocyclyl, or monocyclic cycloalkyl ring, and ring C₁ is a phenyl or monocyclic 6-membered heteroaryl ring;
p is an integer 0, 1, or 2;
q is an integer 0, 1, or 2;
R₁₁ is independently selected from halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl); or
R₁₂ is independently selected from halogen, —CN, C₁-C₆ alkyl, C₁-C₆ haloalkyl, C₁-C₆ alkoxy, and C₁-C₆ haloalkoxy;
R₁₃ is selected from halogen, —CO₂H, —CO₂(C₁-C₆ alkyl), —CO(C₁-C₆ alkyl), —CONH₂, —CONH(C₁-C₆ alkyl), —CON(C₁-C₆ alkyl)₂, —CONH—OH, —CONH—OCO(C₁-C₆ alkyl), —CONH—NH₂, —CONH—S(O)₂R₁₅, —SO₂OH, —SO₂(C₁-C₆ alkyl), —SO₂N(R₁₅)₂, —SO₂NH—COCH₃, —S(O)(NR₁₅)R₁₅, —NH—SO₂R₁₅, —NHCO—NHSO₂R₁₅, —PO(OH)₂, —PO(OH)R₁₅, pyrazolyl, and tetrazolyl, wherein each R₁₅ is independently selected from the group consisting of hydrogen, C₁-C₆ alkyl, C₁-C₆ haloalkyl, cycloalkyl optionally substituted with C₁-C₄ alkyl, phenyl, and monocyclic heteroaryl;
R₁₆ is —OH, C₁-C₆ alkoxy, —NH₂, —NH(C₁-C₆ alkyl), or —N(C₁-C₆ alkyl)₂; and
X is CH₂, CH, O, S, or NH, and Y is O, S, or NH, where X and Y, together with the atoms to which they are attached, form a 10- to 16-member macrocycle, the macrocycle optionally substituted with one or more R₁₄, wherein
each R₁₄ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), and amino(C₁-C₆ alkyl),
or two R₁₄ groups, together with the carbon to which they are attached, form a═O.

162. A pharmaceutical composition comprising a compound according to claim 145 and a pharmaceutically acceptable carrier, solvent, adjuvant or diluent.

163. A method of treating a disease or disorder mediated by Complement Factor B, the method comprising administering to a subject in need of such treatment one or more compounds according to claim 145.

164. The method of claim 163, wherein the disease or disorder is age-related macular degeneration (AMD), geographic atrophy (GA), retinal degeneration, ophthalmic disease, multiple sclerosis, arthritis, chronic obstructive pulmonary disease (COPD), an ophthalmic disease, rheumatoid arthritis, paroxysmal nocturnal hemoglobinuria (PNH), a respiratory disease, a cardiovascular disease, atypical or typical hemolytic uremic syndrome (HUS), C₃ glomerulopathy (C3G), IgA nephropathy (IgAN), or a nephropathy with evidence of glomerular C₃ deposition such as membranous nephropathy (MN) and E. coli induced hemolytic uremic syndrome (HUS).
